(19) United States
(12) Patent Application Publication
(10) Pub. No.: US 2017/0121728 A1
(43) Pub. Date: May 4, 2017

(54) PLANT GLUTAMINE PHENYLPYRUVATE TRANSAMINASE GENE AND TRANSGENIC PLANTS CARRYING SAME

(71) Applicants: Los Alamos National Security, LLC, Los Alamos, NM (US); University of Maine System Board of Trustees, Bangor, ME (US)

(72) Inventors: Pat J. Unkefer, Los Alamos, NM (US); Penelope S. Anderson, Los Alamos, NM (US); Thomas J. Knight, Raymond, ME (US)

(21) Appl. No.: 15/233,879

(22) Filed: Aug. 10, 2016

Related U.S. Application Data
(63) Continuation-in-part of application No. 13/718,214, filed on Dec. 18, 2012, now abandoned.
Application No. 13/718,214 is a continuation of application No. 12/660,508, filed on Feb. 26, 2010, now abandoned.
Application No. 12/660,508 is a continuation-in-part of application No. 12/551,320, filed on Aug. 31, 2009, now abandoned.
Application No. 12/551,320 is a continuation-in-part of application No. 13/714,948, filed on Dec. 14, 2012, now Pat. No. 9,434,956.
Application No. 13/714,948 is a continuation of application No. 12/660,501, filed on Feb. 26, 2010, now abandoned.
Application No. 12/660,501 is a continuation-in-part of application No. 12/551,271, filed on Aug. 31, 2009, now abandoned.
Application No. 12/551,271 is a continuation-in-part of application No. 15/064,329, filed on Mar. 8, 2016.
Application No. 15/064,329 is a continuation of application No. 13/734,688, filed on Jan. 4, 2013, now Pat. No. 9,296,998.
Application No. 13/734,688 is a continuation of application No. 12/660,506, filed on Feb. 26, 2010, now abandoned.
Application No. 12/660,506 is a continuation-in-part of application No. 12/551,193, filed on Aug. 31, 2009, now abandoned.
(60) Provisional application No. 61/190,581, filed on Aug. 29, 2008.
(60) Provisional application No. 61/190,520, filed on Aug. 29, 2008.
(60) Provisional application No. 61/190,520, filed on Aug. 29, 2008.
(60) Provisional application No. 61/190,581, filed on Aug. 29, 2008.

(51) Int. Cl. C12N 15/82; A01H 4/00

(52) U.S. Cl. CPC C12N 15/8261; C12N 15/827; C12Y 206/01064; A01H 4/008

(57) ABSTRACT

The invention relates to transgenic plants exhibiting enhanced growth rates, seed and fruit yields, and overall biomass yields, as well as methods for generating growth-enhanced transgenic plants. In one embodiment, transgenic plants engineered to over-express glutamine phenylpyruvate transaminase (GPT) are provided.

### STATEMENT AS TO RIGHTS TO INVENTIONS MADE UNDER FEDERALLY SPONSORED RESEARCH OR DEVELOPMENT

This invention was made with government support under Contract No. W-7405-ENG-36 awarded by the United States Department of Energy to The Regents of The University of California, and Contract No. DE-AC52-06NA25396, awarded by the United States Department of Energy to Los Alamos National Security, LLC. The government has certain rights in this invention.

### BACKGROUND OF THE INVENTION

As the human population increases worldwide, and available farmland continues to be destroyed or otherwise compromised, the need for more effective and sustainable agriculture systems is of paramount interest to the human race. Improving crop yields, protein content, and plant growth rates represent major objectives in the development of agriculture systems that can more effectively respond to the challenges presented.

In recent years, the importance of improved crop production technologies has only increased as yields for many well-developed crops have tended to plateau. Many agricultural activities are time sensitive, with costs and returns being dependent upon rapid turnover of crops or upon time to market. Therefore, rapid plant growth is an economically important goal for many agricultural businesses that involve high-value crops such as grains, vegetables, berries and other fruits.

Genetic engineering has and continues to play an increasingly important yet controversial role in the development of sustainable agriculture technologies. A large number of genetically modified plants and related technologies have been developed in recent years, many of which are in widespread use today (Factsheet: Genetically Modified Crops in the United States, Pew Initiative on Food and Biotechnology, August 2004, http://pewagbiotech.org/resources/factsheets). The adoption of transgenic plant varieties is now very substantial and is on the rise, with approximately 250 million acres planted with transgenic plants in 2006.

While acceptance of transgenic plant technologies may be gradually increasing, particularly in the United States, Canada and Australia, many regions of the World remain slow to adopt genetically modified plants in agriculture, notably Europe. Therefore, consonant with pursuing the objectives of responsible and sustainable agriculture, there is a strong interest in the development of genetically engineered plants that do not introduce toxins or other potentially problematic substances into plants and/or the environment. There is also a strong interest in minimizing the cost of achieving objectives such as improving herbicide tolerance, pest and disease resistance, and overall crop yields. Accordingly, there remains a need for transgenic plants that can meet these objectives.

The goal of rapid plant growth has been pursued through numerous studies of various plant regulatory systems, many of which remain incompletely understood. In particular, the plant regulatory mechanisms that coordinate carbon and nitrogen metabolism are not fully elucidated. These regulatory mechanisms are presumed to have a fundamental impact on plant growth and development.

The metabolism of carbon and nitrogen in photosynthetic organisms must be regulated in a coordinated manner to assure efficient use of plant resources and energy. Current understanding of carbon and nitrogen metabolism includes details of certain steps and metabolic pathways which are subsystems of larger systems. In photosynthetic organisms, carbon metabolism begins with CO₂ fixation, which proceeds via two major processes, termed C-3 and C-4 metabolism. In plants with C-3 metabolism, the enzyme ribulose bisphosphate carboxylase (RuBisCo) catalyzes the combination of CO₂ with ribulose bisphosphate to produce 3-phosphoglycerate, a three carbon compound (C-3) that the plant uses to synthesize carbon-containing compounds. In plants with C-4 metabolism, CO₂ is combined with phosphoenol pyruvate to form acids containing four carbons (C-4), in a reaction catalyzed by the enzyme phosphoenol pyruvate carboxylase. The acids are transferred to bundle sheath cells, where they are decarboxylated to release CO₂, which is then combined with ribulose bisphosphate in the same reaction employed by C-3 plants.

Numerous studies have found that various metabolites are important in plant regulation of nitrogen metabolism. These compounds include the organic acid malate and the amino acids glutamate and glutamine. Nitrogen is assimilated by photosynthetic organisms via the action of the enzyme glutamine synthetase (GS) which catalyzes the combination of ammonia with glutamate to form glutamine. GS plays a key role in the assimilation of nitrogen in plants by catalyzing the addition of ammonium to glutamate to form glutamine in an ATP-dependent reaction (Miflin and Habash, 2002, Journal of Experimental Botany, Vol. 53, No. 370, pp. 979-987). GS also reassimilates ammonia released as a result of photorespiration and the breakdown of proteins and nitrogen transport compounds. GS enzymes may be divided into two general classes, one representing the cytoplasmic form (GS1) and the other representing the plastidic (i.e., chloroplastic) form (GS2).

Previous work has demonstrated that increased expression levels of GS1 result in increased levels of GS activity and plant growth, although reports are inconsistent. For example, Fuentes et al. reported that CaMV S35 promoter-driven overexpression of Alfalfa GS1 (cytoplasmic form) in tobacco resulted in increased levels of GS expression and GS activity in leaf tissue, increased growth under nitrogen starvation, but no effect on growth under optimal nitrogen fertilization conditions (Fuentes et al., 2001, J. Exp. Botany 52: 1071-81). Temple et al. reported that transgenic tobacco plants overexpressing the full length Alfalfa GS1 coding sequence contained greatly elevated levels of GS transcript, and GS polypeptide which assembled into active enzyme, but did not report phenotypic effects on growth (Temple et al., 1993, Molecular and General Genetics 236: 315-325). Corruzi et al. have reported that transgenic tobacco overexpressing a pea cytosolic GS1 transgene under the control of the CaMV S35 promoter show increased GS activity, increased cytosolic GS protein, and improved growth characteristics (U.S. Pat. No. 6,107,547). Unkefer et al. have more recently reported that transgenic tobacco plants overexpressing the Alfalfa GS1 in foliar tissues, which had been screened for increased leaf-to-root GS activity following genetic segregation by selfing to achieve increased GS1 transgene copy number, were found to produce increased 2-hydroxy-5-oxoproline levels in their foliar portions, which was found to lead to markedly increased growth rates over wildtype tobacco plants (see, U.S. Pat. Nos. 6,555,500; 6,593,275; and 6,831,040).

Unkefer et al. have further described the use of 2-hydroxy-5-oxoproline (also known as 2-oxoglutaramate) to improve plant growth (U.S. Pat. Nos. 6,555,500; 6,593,275; 6,831,040). In particular, Unkefer et al. disclose that increased concentrations of 2-hydroxy-5-oxoproline in foliar tissues (relative to root tissues) triggers a cascade of events that result in increased plant growth characteristics. Unkefer et al. describe methods by which the foliar concentration of 2-hydroxy-5-oxoproline may be increased in order to trigger increased plant growth characteristics, specifically, by applying a solution of 2-hydroxy-5-oxoproline directly to the foliar portions of the plant and over-expressing glutamine synthetase preferentially in leaf tissues.

A number of transaminase and hydrolyase enzymes known to be involved in the synthesis of 2-hydroxy-5-oxoproline in animals have been identified in animal liver and kidney tissues (Cooper and Meister, 1977, CRC Critical Reviews in Biochemistry, pages 281-303; Meister, 1952, J. Biochem. 197: 304). In plants, the biochemical synthesis of 2-hydroxy-5-oxoproline has been known but has been poorly characterized. Moreover, the function of 2-hydroxy-5-oxoproline in plants and the significance of its pool size (tissue concentration) are unknown. Finally, the art provides no specific guidance as to precisely what transaminase(s) or hydrolase(s) may exist and/or be active in catalyzing the synthesis of 2-hydroxy-5-oxoproline in plants, and no such plant transaminases have been reported, isolated or characterized.

### SUMMARY OF THE INVENTION

The present invention discloses for the first time that plants contain a glutamine phenylpyruvate transaminase enzyme which is directly functional in the synthesis of the signal metabolite 2-hydroxy-5-oxoproline, and provides the protein and gene coding sequences for a number of plant GPTs as well as a highly structurally-related non-plant GPT. The invention further provides strong evidence that plant GPTs are highly conserved and are involved in directly catalyzing 2-oxoglutaramate synthesis. Until now, no plant glutamine phenylpyruvate transaminase with a defined function has been described.

The invention relates to plant glutamine phenylpyruvate transaminase (GPT) proteins, nucleic acid molecules encoding GPT proteins, and uses thereof. Defined herein are various GPT proteins and GPT gene coding sequences isolated from a number of plant species. As disclosed herein, GPT proteins share remarkable structural similarity within plant species, and are active in catalyzing the synthesis of 2-hydroxy-5-oxoproline (2-oxoglutaramate), a powerful signal metabolite which regulates the function of a large number of genes involved in the photosynthesis apparatus, carbon fixation and nitrogen metabolism.

The invention also relates to transgenic plants exhibiting enhanced growth rates, seed and fruit yields, and overall biomass yields, as well as methods for generating growth-enhanced transgenic plants. In one embodiment, transgenic plants engineered to over-express glutamine phenylpyruvate transaminase (GPT) are provided. In general, these plants out-grow their wild-type counterparts by about 50%.

Applicants have identified the enzyme glutamine phenylpyruvate transaminase (GPT) as a catalyst of 2-hydroxy-5-oxoproline (2-oxoglutaramate) synthesis in plants. 2-oxoglutaramate is a powerful signal metabolite which regulates the function of a large number of genes involved in the photosynthesis apparatus, carbon fixation and nitrogen metabolism.

By preferentially increasing the concentration of the signal metabolite 2-oxoglutaramate (i.e., in foliar tissues), the transgenic plants of the invention are capable of producing higher overall yields over shorter periods of time, and therefore may provide agricultural industries with enhanced productivity across a wide range of crops. Importantly, unlike many transgenic plants described to date, the invention utilizes natural plant genes encoding a natural plant enzyme. The enhanced growth characteristics of the transgenic plants of the invention are achieved essentially by introducing additional GPT capacity into the plant. Thus, the transgenic plants of the invention do not express any toxic substances, growth hormones, viral or bacterial gene products, and are therefore free of many of the concerns that have heretofore impeded the adoption of transgenic plants in certain parts of the World.

In one embodiment, the invention provides a transgenic plant comprising a GPT transgene, wherein the GPT transgene is operably linked to a plant promoter. In a specific embodiment, the GPT transgene encodes a polypeptide having an amino acid sequence selected from the group consisting of (a) SEQ ID NO: 2; SEQ ID NO: 4, SEQ ID NO: 10, SEQ ID NO: 14, SEQ ID NO: 16, SEQ ID NO: 19, SEQ ID NO: 25, SEQ ID NO: 26, SEQ ID NO: 27, SEQ ID NO: 28, SEQ ID NO: 29, SEQ ID NO: 30, SEQ ID NO: 41, or SEQ ID NO: 44, and (b) an amino acid sequence that is at least 75% identical to any one of SEQ ID NO: 2; SEQ ID NO: 4, SEQ ID NO: 10, SEQ ID NO: 14, SEQ ID NO: 16, SEQ ID NO: 19, SEQ ID NO: 25, SEQ ID NO: 26, SEQ ID NO: 27, SEQ ID NO: 28, SEQ ID NO: 29, SEQ ID NO: 30, SEQ ID NO: 41, or SEQ ID NO: 44 and has GPT activity.

In a particular aspect of the invention, the GPT transgene is incorporated into the genome of a plant selected from the group consisting of: maize, rice, sugar cane, wheat, oats, Sorghum, switch grass, soya bean, tubers (such as potatoes), canola, lupins or cotton.

The invention also provides progeny of any generation of the transgenic plants of the invention, wherein the progeny comprises a GPT transgene, as well as a seed of any generation of the transgenic plants of the invention, wherein the seed comprises the GPT transgene. The transgenic plants of the invention may display one or more enhanced growth characteristics when compared to an analogous wild-type or untransformed plant, including without limitation increased growth rate, increased biomass yield, increased seed yield, increased flower or flower bud yield, increased fruit or pod yield, larger leaves, and increased levels of GPT activity and/or increased levels of 2-oxoglutaramate. In some embodiments, the transgenic plants of the invention display increased nitrogen use efficiency.

In a further aspect of the invention there is provided a transplastomic plant or cell line carrying a GPT transgene expression cassette, the expression cassette being flanked by sequences from the plant or plant cell's plastome.

Further still, the invention provides a method for preparing a transplastomic plant or cell line carrying a GPT transgene construct, the method comprising the steps of: (a) inserting into at least one expression cassette at least a GPT transgene wherein the expression cassette is flanked by sequences from the plant or plant cell's plastome.

Methods for producing the transgenic plants of the invention and seeds thereof are also provided, including methods for producing a plant having enhanced growth characteristics, increased nitrogen use efficiency and increased tolerance to germination or growth in salt or saline conditions, relative to an analogous wild type or untransformed plant.

The invention also provides isolated nucleic acid molecules encoding GPT. Exemplary GPT polynucleotides and GPT polypeptides are provided herein. In one embodiment, the invention provides an isolated GPT polynucleotide having a sequence selected from the group consisting of (a) the nucleotide sequence of SEQ ID NO: 1; (b) a nucleotide sequence having at least 75% identity to SEQ ID NO: 1, and encoding a polypeptide having GPT activity; (c) a nucleotide sequence encoding the polypeptide of SEQ ID NO: 2, or a polypeptide having at least 75% sequence identity thereto which has GPT activity; and, (d) a nucleotide sequence encoding the polypeptide of SEQ ID NO: 2 truncated at its amino terminus by between 30 to 56 amino acid residues, or a polypeptide having at least 75% sequence identity thereto which has GPT activity. In specific embodiments, the isolated GPT polynucleotide comprises the nucleotide sequence of SEQ ID NO: 18, SEQ ID NO: 29, SEQ ID NO: 45 or SEQ ID NO: 48, or a nucleotide sequence having at least 75% identity to SEQ ID NO: 18, SEQ ID NO: 29, SEQ ID NO: 45 or SEQ ID NO: 48.

In another aspect, the invention provides an isolated GPT polynucleotide encoding a polypeptide having an amino acid sequence selected from the group consisting of (a) SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 46 and SEQ ID NO: 49, and (b) an amino acid sequence that is at least 75% identical to any one of SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO:31, SEQ ID NO: 32, SEQ ID NO: 33, SEQ ID NO: 34, SEQ ID NO: 35, SEQ ID NO: 36, SEQ ID NO: 46 and SEQ ID NO: 49 and has GPT activity.

In another aspect, the invention provides a nucleic acid construct comprising a plant promoter operably linked to a GPT polynucleotide. In one embodiment, the plant promoter is a heterologous promoter. In another embodiment, the plant promoter is a heterologous tissue-specific promoter. Related aspects include a vector comprising such a nucleic acid construct, and a host cell comprising such a vector or nucleic acid construct. In one embodiment, the host cell is a plant cell. In another embodiment, the host cell is a plant cell which expresses the GPT polynucleotide. In yet another embodiment, the host cell is a plant cell which expresses the GPT polynucleotide, wherein polynucleotide so expressed has GPT activity. The invention further provides a plant organ, embryo or seed comprising such a nucleic acid construct or vector, wherein the plant organ, embryo or seed expresses the GPT polynucleotide. In one embodiment, the GPT polynucleotide expressed has GPT activity. In another aspect, the invention provides a transgenic plant comprising such a nucleic acid construct or vector, wherein the transgenic plant expresses the polynucleotide, which in one embodiment has GPT activity. Progeny and seed of such a transgenic plant, wherein the progeny or seed comprises the GPT polynucleotide, are also provided.

### BRIEF DESCRIPTION OF THE DRAWINGS

FIG. 1. Nitrogen assimilation and 2-oxoglutaramate biosynthesis: schematic of metabolic pathway.

FIG. 2A. Multiple sequence alignment of the amino acid sequences of several putative plant, algal and animal GPT proteins, showing a high degree of structural identity and conservation (green shading indicates amino acid residues which are identical in all sequences aligned, and yellow shading indicates amino acids that are identical in all but one or two sequences aligned). This alignment compares (in order from top to bottom in each block) the plant GPTs from barley (Hordeum vulgare), rice (Orzya sativa), corn (Zea mays), cotton (Gossypium hirsutum), grape (Vitis vinifera), castor oil plant (Ricinus communis), California poplar (Populus trichocarpa), soybean (Glycine max), Zebra fish (Danio rerio), arabidopsis (Arabidopsis thaliana), a Bryophyte moss (Physcomitrella patens), and a green algae (Chlamydomonas sp.). The alignment includes the presumed amino-terminal targeting sequence, if known.

FIG. 2B. A continuation of the multiple sequence alignment of FIG. 2A.

FIG. 3A. Subset of the multiple sequence alignment of the of FIG. 2, showing a very high degree of structural identity and conservation (green shading indicates amino acid residues which are identical in all sequences aligned, and yellow shading indicates amino acids that are identical in all but one or two sequences aligned). This alignment includes all sequences aligned and displayed in FIG. 2, except the Physcomitrella and Chlamydomonas sequences. As will be appreciated, relative to the alignment of FIG. 2, a substantial increase in amino acid sequence identity was achieved by eliminating those two sequences, as can be seen by the increase in the number of identical residues among the ten GPT sequences aligned in this figure, nine of which are plant GPTs, and interestingly, the remaining sequence being from Zebra fish.

FIG. 3B. A continuation sheet of the multiple sequence alignment of FIG. 3A.

FIG. 4. Photograph showing comparison of transgenic tobacco plants over-expressing GPT, compared to wild type tobacco plant. From left to right: wild type plant, Alfalfa GS1 transgene, Arabidopsis GPT transgene. See Example 3, infra.

FIG. 5A. Photograph showing wild type tomato plant in comparison to transgenic Micro-Tom tomato plants over-expressing GPT shown in FIG. 5B. See Example 4, infra.

FIG. 5B Photograph showing transgenic Micro-Tom tomato plant (Arabidopsis GPT transgene) in comparison to wild type tomato plant in FIG. 5A. See Example 4, infra.

FIG. 6. Photograph showing comparisons of leaf sizes between wild type (top leaf) and GPT transgenic (bottom leaf) tobacco plants.

FIG. 7A. Photograph showing comparison of transgenic tobacco plants generated from cross 2 between GS1 and GPT transgenic tobacco lines with wild type and single transgene plants. See Example 7, infra.

FIG. 7B. Photograph showing comparison of transgenic tobacco plants generated from cross 3 between GS1 and GPT transgenic tobacco lines with wild type and single transgene plants. See Example 7, infra.

FIG. 7C. Photograph showing comparison of transgenic tobacco plants generated from cross 7 between GS1 and GPT transgenic tobacco lines with wild type and single transgene plants. See Example 7, infra.

FIG. 8A. Photograph showing comparison between leaves from GSXGPT Cross 3 (bottom leaf) and wild type (top leaf). See Example 7, infra.

FIG. 8B: Photograph showing comparison between leaves from GSXGPT Cross 7 (bottom leaf) and wild type (top leaf). See Example 7, infra.

FIG. 9. Photograph of transgenic pepper plant (right) and wild type control pepper plant (left), showing larger pepper fruit yield in the transgenic plant relative to the wild type control plant. See Example 8, infra.

FIG. 10. Transgenic bean plants compared to wild type control bean plants (several transgenic lines expressing Arabidopsis GPT and GS transgenes). Upper Left: plant heights on various days; Upper right: flower bud numbers; Lower left: flower numbers; Lower right: bean pod numbers. Wildtype is the control, and lines 2A, 4A and 5B are all transgenic plant lines. See Example 9, infra.

FIG. 11. Photograph of transgenic bean plant (right) and wild type control bean plant (left), showing increased growth in the transgenic plant relative to the wild type control plant. Transgenic line expressing Arabidopsis GPT and GS transgenes. See Example 9, infra.

FIG. 12. Transgenic bean plants pods, flowers and flower buds compared to wild type control bean plants (transgenic line expressing grape GPT and Arabidopsis GS transgenes). See Example 10, infra.

FIG. 13. Photograph of transgenic bean plant (right) and wild type control bean plant (left), showing increased growth in the transgenic plant relative to the wild type control plant. Transgenic line expressing Grape GPT and Arabidopsis GS transgenes. See Example 10, infra.

FIG. 14A. Transgenic Cowpea Line A plants compared to wild type control Cowpea plants (transgenic line expressing Arabidopsis GPT and GS transgenes), showing relative height and longest leaf measurements. Transgenic plants grow faster and flower and set pods sooner than wild type control plants—see also FIGS. 14B and 14C. See Example 11, infra.

FIG. 14B. Transgenic Cowpea Line A plants compared to wild type control Cowpea plants (transgenic line expressing Arabidopsis GPT and GS transgenes), showing relative trifolate leafs and flower buds. See Example 11, infra.

FIG. 14C. Transgenic Cowpea Line A plants compared to wild type control Cowpea plants (transgenic line expressing Arabidopsis GPT and GS transgenes), showing relative numbers of flowers, flower buds and pea pods. See Example 11, infra.

FIG. 15. Photograph of transgenic Cowpea Line A plant (right) and wild type control Cowpea plant (left), showing increased growth in the transgenic plant relative to the wild type control plant. Transgenic line expressing Arabidopsis GPT and GS transgenes. See Example 11, infra.

FIG. 16A. Transgenic Cowpea Line G plants compared to wild type control Cowpea plants (transgenic line expressing Grape GPT and Arabidopsis GS transgenes), showing relative plant heights. The transgenic plants grow faster and flower and set pods sooner than wild type control plants—see also FIGS. 16B and 16C. See Example 12, infra.

FIG. 16B. Transgenic Cowpea Line G plants compared to wild type control Cowpea plants (transgenic line expressing Grape GPT and Arabidopsis GS transgenes), showing relative flowers and pea pod numbers. See Example 12, infra.

FIG. 16C. Transgenic Cowpea Line G plants compared to wild type control Cowpea plants (transgenic line expressing Grape GPT and Arabidopsis GS transgenes), showing relative leaf bud and trifolate numbers. See Example 12, infra.

FIG. 17. Photograph of transgenic Cowpea Line G plant (right) and wild type control Cowpea plant (left), showing increased growth in the transgenic plant relative to the wild type control plant. Transgenic line expressing Grape GPT and Arabidopsis GS transgenes. See Example 12, infra.

FIG. 18. Photograph of transgenic Cantaloupe plant (right) and wild type control Cantaloupe plant (left), showing increased growth in the transgenic plant relative to the wild type control plant. Transgenic line expressing Arabidopsis GPT and GS transgenes. See Example 14, infra.

FIG. 19. Photograph of transgenic Pumpkin plants (right) and wild type control Pumpkin plants (left), showing increased growth in the transgenic plants relative to the wild type control plants. Transgenic lines expressing Arabidopsis GPT and GS transgenes. See Example 15, infra.

FIG. 20. Photograph of transgenic Arabidopsis plants (right) and wild type control Arabidopsis plants (left), showing increased growth in the transgenic plants relative to the wild type control plants. Transgenic lines expressing Arabidopsis GPT and GS transgenes. See Example 16, infra.

FIG. 21A. Transgenic tomato plants expressing Arabidopsis GPT and GS transgenes compared to control tomato plants. Photograph of transgenic tomato plant leaves (right) vs. wild type control leaves (left) showing larger leaves in the transgenic plant. See Example 17, infra.

FIG. 21B Transgenic tomato plants expressing Arabidopsis GPT and GS transgenes compared to control tomato plants. Photograph of transgenic tomato plants (right) and wild type control plants (left), showing increased growth in the transgenic plants relative to the wild type control plants. See Example 17, infra.

FIG. 22. Photograph of transgenic Camelina plant (right) and wild type control Camelina plant (left), showing increased growth in the transgenic plant relative to the wild type control plant. Transgenic line expressing Arabidopsis GPT and GS transgenes. See Example 18, infra.

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition, 2006). As appropriate, procedures involving the use of commercially available kits and reagents are generally carried out in accordance with manufacturer defined protocols and/or parameters unless otherwise noted.

Each document, reference, patent application or patent cited in this text is expressly incorporated herein in its entirety by reference, and each should be read and considered as part of this specification. That the document, reference, patent application or patent cited in this specification is not repeated herein is merely for conciseness.

The term “nucleic acid” refers to deoxyribonucleotides or ribonucleotides and polymers thereof (“polynucleotides”) in either single- or double-stranded form. Unless specifically limited, the term “polynucleotide” encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

The term “promoter” refers to a nucleic acid control sequence or sequences that direct transcription of an operably linked nucleic acid. As used herein, a “plant promoter” is a promoter that functions in plants. Promoters include necessary nucleic acid sequences near the start site of transcription, such as, in the case of a polymerase II type promoter, a TATA element. A promoter also optionally includes distal enhancer or, repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “plant” includes whole plants, plant organs (e.g., leaves, stems, flowers, roots, reproductive organs, embryos and parts thereof, etc.), seedlings, seeds and plant cells and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous.

The terms “GPT polynucleotide” and “GPT nucleic acid” are used interchangeably herein, and refer to a full length or partial length polynucleotide sequence of a gene which encodes a polypeptide involved in catalyzing the synthesis of 2-oxoglutaramate, and includes polynucleotides containing both translated (coding) and un-translated sequences, as well as the complements thereof. The term “GPT coding sequence” refers to the part of the gene which is transcribed and encodes a GPT protein. The term “targeting sequence” refers to the amino terminal part of a protein which directs the protein into a subcellular compartment of a cell, such as a chloroplast in a plant cell. GPT polynucleotides are further defined by their ability to hybridize under defined conditions to the GPT polynucleotides specifically disclosed herein, or to PCR products derived therefrom.

A “GPT transgene” is a nucleic acid molecule comprising a GPT polynucleotide which is exogenous to transgenic plant, or plant embryo, organ or seed, harboring the nucleic acid molecule, or which is exogenous to an ancestor plant, or plant embryo, organ or seed thereof, of a transgenic plant harboring the GPT polynucleotide. More particularly, the exogenous GPT transgene will be heterogeneous with any GPT polynucleotide sequence present in wild-type plant, or plant embryo, organ or seed into which the GPT transgene is inserted. To this extent the scope of the heterogeneity required need only be a single nucleotide difference. However, preferably the heterogeneity will be in the order of an identity between sequences selected from the following identities: 0.01%, 0.05%, 0.1%, 0.5%, 1%, 5%, 10%, 15%, and 20%.

Exemplary GPT polynucleotides of the invention are presented herein, and include GPT coding sequences for Arabidopsis, Rice, Barley, Bamboo, Soybean, Grape, Clementine orange and Zebra Fish GPTs.

Partial length GPT polynucleotides include polynucleotide sequences encoding N- or C-terminal truncations of GPT, mature GPT (without targeting sequence) as well as sequences encoding domains of GPT. Exemplary GPT polynucleotides encoding N-terminal truncations of GPT include Arabidopsis-30, -45 and -56 constructs, in which coding sequences for the first 30, 45, and 56, respectively, amino acids of the full length GPT structure of SEQ ID NO: 2 are eliminated.

In employing the GPT polynucleotides of the invention in the generation of transformed cells and transgenic plants, one of skill will recognize that the inserted polynucleotide sequence need not be identical, but may be only “substantially identical” to a sequence of the gene from which it was derived, as further defined below. The term “GPT polynucleotide” specifically encompasses such substantially identical variants. Similarly, one of skill will recognize that because of codon degeneracy, a number of polynucleotide sequences will encode the same polypeptide, and all such polynucleotide sequences are meant to be included in the term GPT polynucleotide. In addition, the term specifically includes those sequences substantially identical (determined as described below) with an GPT polynucleotide sequence disclosed herein and that encode polypeptides that are either mutants of wild type GPT polypeptides or retain the function of the GPT polypeptide (e.g., resulting from conservative substitutions of amino acids in a GPT polypeptide). The term “GPT polynucleotide” therefore also includes such substantially identical variants.

The term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine.

Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “isolated” refers to material which is substantially or essentially free from components which normally accompany the material as it is found in its native or natural state. However, the term “isolated” is not intended refer to the components present in an electrophoretic gel or other separation medium. An isolated component is free from such separation media and in a form ready for use in another application or already in use in the new application/milieu. An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “heterologous” when used with reference to portions of a nucleic acid indicates that the nucleic acid comprises two or more subsequences that are not found in the same relationship to each other in nature. For instance, a nucleic acid is typically recombinantly produced, having two or more sequences from unrelated genes arranged to make a new functional nucleic acid, e.g., a nucleic acid encoding a protein from one source and a nucleic acid encoding a peptide sequence from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection. This definition also refers to the complement of a test sequence, which has substantial sequence or subsequence complementarity when the test sequence has substantial identity to a reference sequence. This definition also refers to the complement of a test sequence, which has substantial sequence or subsequence complementarity when the test sequence has substantial identity to a reference sequence.

When percentage of sequence identity is used in reference to polypeptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the polypeptide. Where sequences differ in conservative substitutions, the percent sequence identity may be adjusted upwards to correct for the conservative nature of the substitution.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. BLAST and BLAST 2.0 are used, typically with the default parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tm) for the specific sequence at a defined ionic strength pH. Low stringency conditions are generally selected to be about 15-30° C. below the Tm. Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

Genomic DNA or cDNA comprising GPT polynucleotides may be identified in standard Southern blots under stringent conditions using the GPT polynucleotide sequences disclosed here. For this purpose, suitable stringent conditions for such hybridizations are those which include a hybridization in a buffer of 40% formamide, 1M NaCl, 1% SDS at 37° C., and at least one wash in 0.2×SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions may be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

Transgenic Plants:

The invention provides novel transgenic plants exhibiting substantially enhanced growth and other agronomic characteristics, including without limitation faster growth, greater mature plant fresh weight and total biomass, earlier and more abundant flowering, and greater fruit and seed yields. The transgenic plants of the invention are generated by introducing into a plant one or more expressible genetic constructs capable of driving the expression of one or more polynucleotides encoding glutamine phenylpyruvate transaminase (GPT). The invention is exemplified, for example, by the generation of transgenic tobacco plants carrying and expressing the heterologous Arabidopsis GPT gene (Example 2, infra). It is expected that all plant species also contain a GPT homolog which functions in the same metabolic pathway, namely the biosynthesis of the signal metabolite 2-hydroxy-5-oxoproline. Thus, in the practice of the invention, any plant gene encoding a GPT homolog or functional variants thereof may be useful in the generation of transgenic plants of this invention.

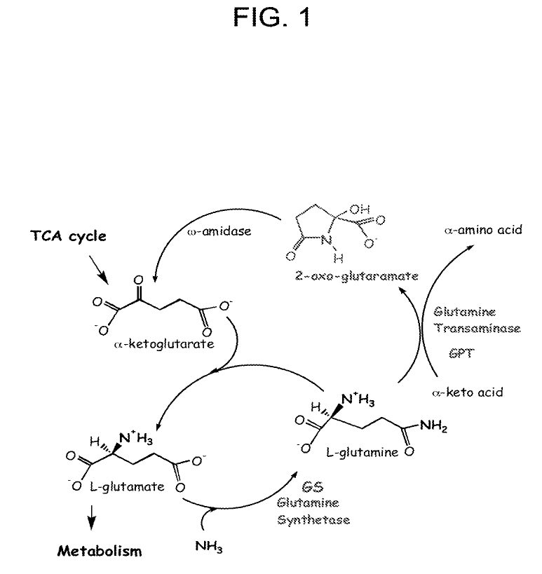

In stable transformation embodiments of the invention, one or more copies of the expressible genetic construct become integrated into the host plant genome, thereby providing increased GPT enzyme capacity into the plant, which serves to mediate increased synthesis of 2-oxoglutaramate, which in turn signals metabolic gene expression, resulting in increased plant growth and the enhancement of plant growth and other agronomic characteristics. 2-oxoglutaramate is a metabolite which is an extremely potent effector of gene expression, metabolism and plant growth (U.S. Pat. No. 6,555,500), and which may play a pivotal role in the coordination of the carbon and nitrogen metabolism systems (Lancien et al., 2000, Enzyme Redundancy and the Importance of 2-Oxoglutarate in Higher Plants Ammonium Assimilation, Plant Physiol. 123: 817-824). See, also, the schematic of the 2-oxoglutaramate pathway shown in FIG. 1.

In one aspect of the invention, applicants have isolated a nucleic acid molecule encoding the Arabidopsis glutamine phenylpyruvate transaminase (GPT) enzyme (see Example 1, infra), and have demonstrated for the first time that the expressed recombinant enzyme is active and capable of catalyzing the synthesis of the signal metabolite, 2-oxoglutaramate (Example 2, infra). Further, applicants have demonstrated for the first time that over-expression of the Arabidopsis glutamine transaminase gene in a transformed heterologous plant results in enhanced CO₂ fixation rates and increased growth characteristics (Example 3, infra).

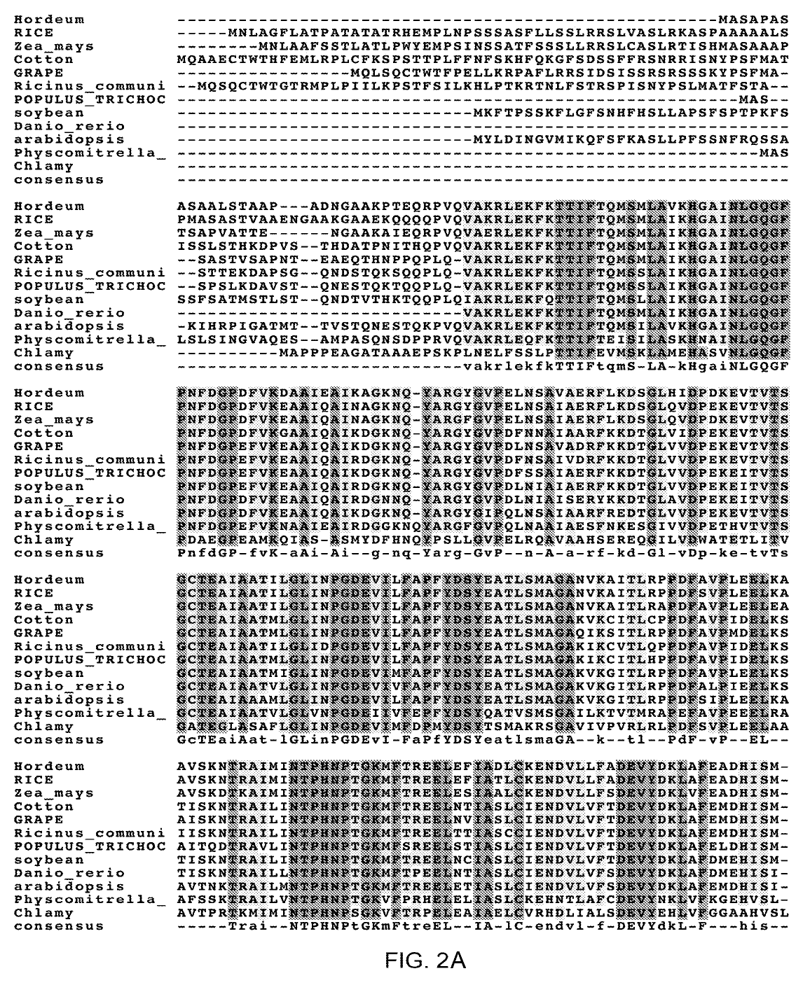

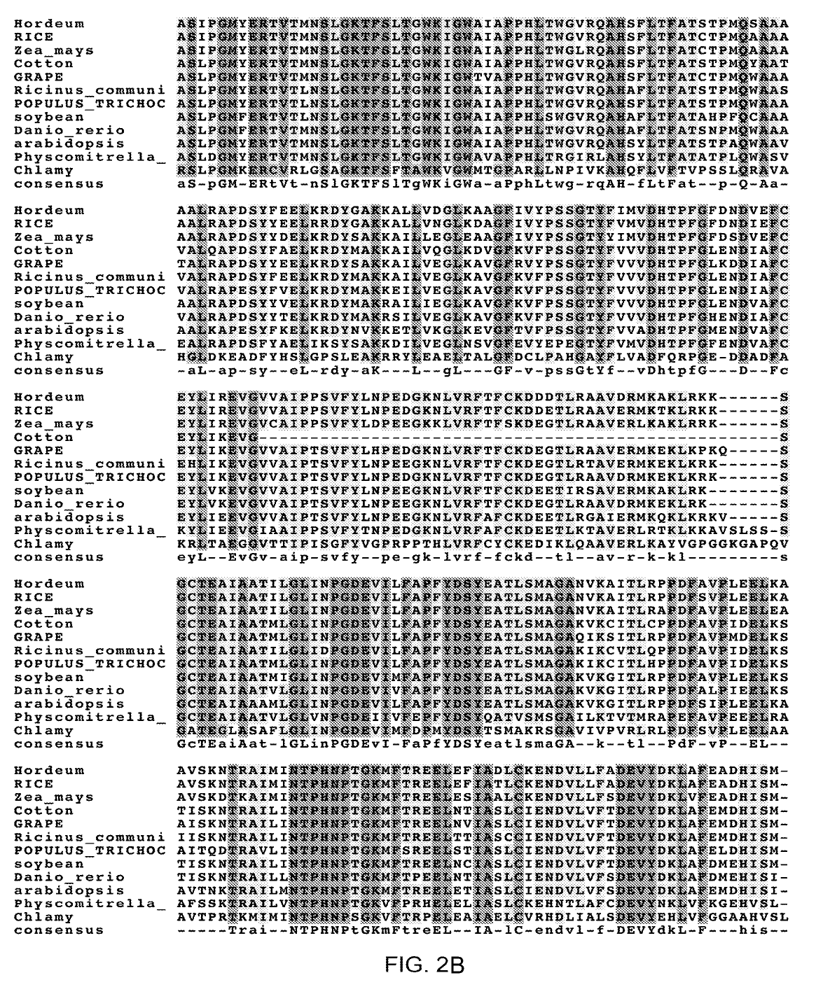

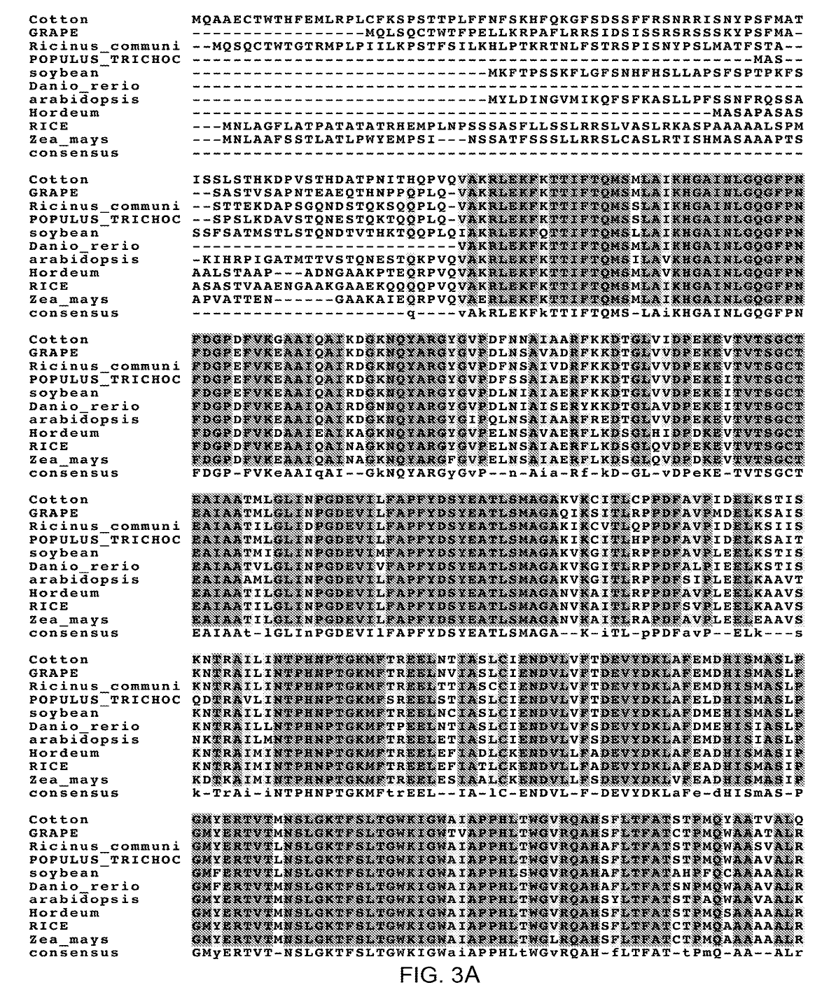

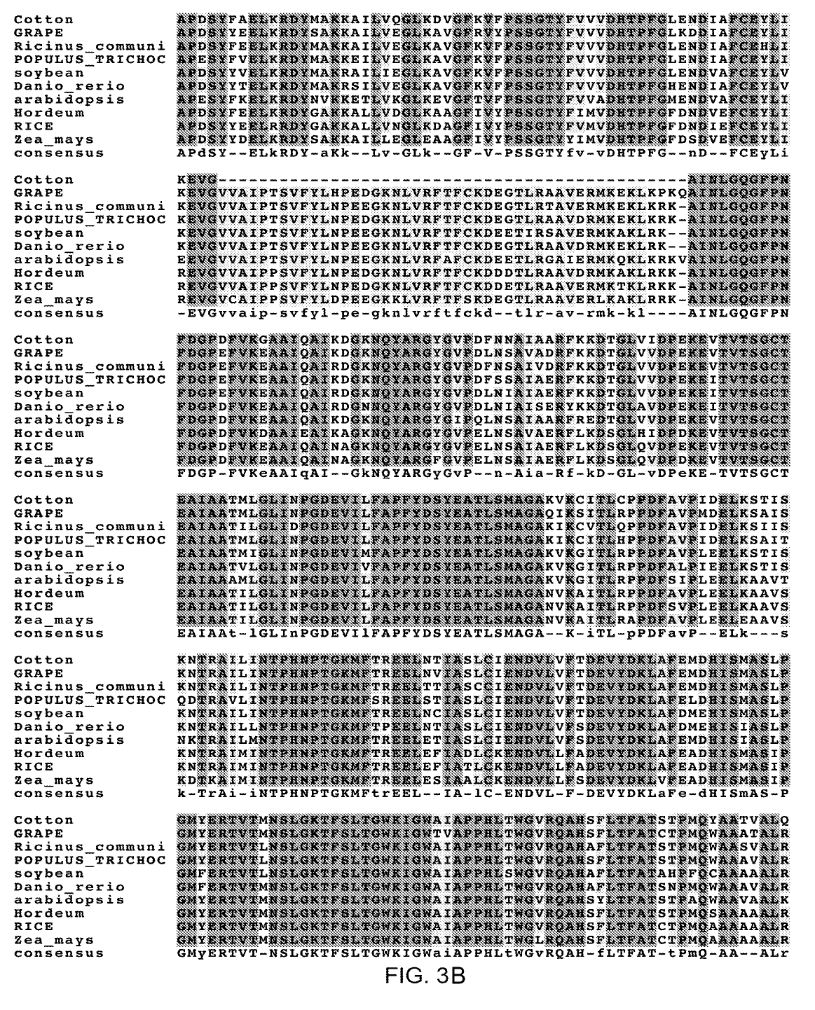

As disclosed herein (see Example 3, infra), over-expression of a transgene comprising the full-length Arabidopsis GPT coding sequence in transgenic tobacco plants also results in faster CO₂ fixation, and increased levels of total protein, glutamine and 2-oxoglutaramate. These transgenic plants also grow faster than wild-type plants (FIGS. 2A-2B). Similarly, in studies conducted with tomato plants (see Example 4, infra), tomato plants transformed with the Arabidopsis GPT transgene showed significant enhancement of growth rate, flowering, and seed yield in relation to wild type control plants (FIGS. 3A-3B and Example 4, infra).

In addition to the transgenic tobacco plants referenced above, various other species of transgenic plants comprising GPT and showing enhanced growth characteristics have been generated in two species of Tomato, Pepper, Beans, Cowpea, Alfalfa, Cantaloupe, Pumpkin, Arabidopsis and Camilena (see U.S. application Ser. No. 12/551,271, filed Aug. 31, 2009, the contents of which are incorporated herein by reference in its entirety). The foregoing transgenic plants were generated using a variety of transformation methodologies, including Agrobacterium-mediated callus, floral dip, seed inoculation, pod inoculation, and direct flower inoculation, as well as combinations thereof, and via sexual crosses of single transgene plants, using various GPT transgenes.

The transgenic plants of the invention may be any vascular plant of the phylum Tracheophyta, including angiosperms and gymnosperms. Angiosperms may be a monocotyledonous (monocot) or a dicotyledonous (dicot) plant. Important monocots include those of the grass families, such as the family Poaceae and Gramineae, including plants of the genus Avena (Avena sativa, oats), genus Hordeum (i.e., Hordeum vulgare, Barley), genus Oryza (i.e., Oryza sativa, rice, cultivated rice varieties), genus Panicum (Panicum spp., Panicum virgatum, Switchgrass), genus Phleum (Phleum pratense, Timothy-grass), genus Saccharum (i.e., Saccharum officinarum, Saccharum spontaneum, hybrids thereof, Sugarcane), genus Secale (i.e., Secale cereale, Rye), genus Sorghum (Sorghum vulgare, Sorghum), genus Triticum (wheat, various classes, including T. aestivum and T. durum), genus Fagopyrum (buckwheat, including F. esculentum), genus Triticosecale (Triticale, various hybrids of wheat and rye), genus Chenopodium (quinoa, including C. quinoa), genus Zea (i.e., Zea mays, numerous varieties) as well as millets (i.e., Pennisetum glaucum) including the genus Digitaria (D. exilis).

Important dicots include those of the family Solanaceae, such as plants of the genus Lycopersicon (Lycopersicon esculentum, tomato), genus Capiscum (Capsicum annuum, peppers), genus Solanum (Solanum tuberosum, potato, S. lycopersicum, tomato); genus Manihot (cassava, M. esculenta), genus Ipomoea (sweet potato, I. batatas), genus Olea (olives, including O. europaea); plants of the Gossypium family (i.e., Gossypium spp., G. hirsutum, G. herbaceum, cotton); the Legumes (family Fabaceae), such as peas (Pisum spp, P. sativum), beans (Glycine spp., Glycine max (soybean); Phaseolus vulgaris, common beans, Vigna radiata, mung bean), chickpeas (Cicer arietinum)), lentils (Lens culinaris), peanuts (Arachis hypogaea); coconuts (Cocos nucifera) as well as various other important crops such as camelina (Camelina sativa, family Brassicaceae), citrus (Citrus spp, family Rutaceae), coffee (Coffea spp, family Rubiaceae), melon (Cucumis spp, family Cucurbitaceae), squash (Cucurbita spp, family Cucurbitaceae), roses (Rosa spp, family Rosaceae), sunflower (Helianthus annuus, family Asteraceae), sugar beets (Beta spp, family Amaranthaceae), including sugarbeet, B. vulgaris), genus Daucus (carrots, including D. carota), genus Pastinaca (parsnip, including P. sativa), genus Raphanus (radish, including R. sativus), genus Dioscorea (yams, including D. rotundata and D. cayenensis), genus Armoracia (horseradish, including A. rusticana), genus Elaeis (Oil palm, including E. guineensis), genus Linum (flax, including L. usitatissimum), genus Carthamus (safflower, including C. tinctorius L.), genus Sesamum (sesame, including S. indicum), genus Vitis (grape, including Vitis vinifera), and plants of the genus Brassica (family Brassicaceae, i.e., broccoli, brussel sprouts, cabbage, swede, turnip, rapeseed B. napus, and cauliflower).

Other specific plants which may be transformed to generate the transgenic plants of the invention include various other fruits and vegetables, such as apples, asparagus, avocado, banana, blackberry, blueberry, brussel sprout, cabbage, cotton, canola, carrots, radish, cucumbers, cherries, cranberries, cantaloupes, eggplant, grapefruit, lemons, limes, nectarines, oranges, peaches, pineapples, pears, plums, tangelos, tangerines, papaya, mango, strawberry, raspberry, lettuce, onion, grape, kiwi fruit, okra, parsnips, pumpkins, and spinach. In addition various flowering plants, trees and ornamental plants may be used to generate transgenic varietals, including without limitation lily, carnation, chrysanthemum, petunia, geranium, violet, gladioli, lupine, orchid and lilac.

The invention also provides methods of generating a transgenic plant having enhanced growth and other agronomic characteristics. In one embodiment, a method of generating a transgenic plant having enhanced growth and other agronomic characteristics comprises introducing into a plant cell an expression cassette comprising a nucleic acid molecule encoding a GPT transgene, under the control of a suitable promoter capable of driving the expression of the transgene, so as to yield a transformed plant cell, and obtaining a transgenic plant which expresses the encoded GPT. In another embodiment, a method of generating a transgenic plant having enhanced growth and other agronomic characteristics comprises introducing into a plant cell one or more nucleic acid constructs or expression cassettes comprising nucleic acid molecules encoding a GPT transgene, under the control of one or more suitable promoters (and, optionally, other regulatory elements) capable of driving the expression of the transgenes, so as to yield a plant cell transformed thereby, and obtaining a transgenic plant which expresses the GPT transgene to produce a biologically active GPT protein.

Any number of GPT polynucleotides may be used to generate the transgenic plants of the invention. GPT proteins are highly conserved among various plant species, and it is evident from the experimental data disclosed herein that closely-related non-plant GPTs may be used as well (e.g., Danio rerio GPT). With respect to GPT, numerous GPT polynucleotides derived from different species have been shown to be active and useful as GPT transgenes.

In one embodiment, the present invention relates to a method for producing a plant having enhanced growth properties relative to an analogous wild type or untransformed plant, including (a) introducing into the plant a glutamine phenylpyruvate transaminase (GPT) transgene that encodes a GPT polypeptide having GPT catalytic activity; and (b) expressing the GPT transgene in the transgenic plant or progeny thereof, wherein GPT polypeptides are overexpressed in the transgenic plant or the progeny thereof relative to an analogous wild type or untransformed plant of the same species, where the transgenic plant has an increased biomass yield relative to an analogous wild type or untransformed plant of the same species.

In another embodiment, the present invention relates to a method for producing a plant having enhanced growth properties relative to an analogous wild type or untransformed plant, including (a) introducing into the plant a glutamine phenylpyruvate transaminase (GPT) transgene that encodes a GPT polypeptide having an amino acid sequence that has at least 80% sequence identity to SEQ ID NO: 2, SEQ ID NO: 11, SEQ ID NO: 13, or SEQ ID NO: 15 and GPT catalytic activity; and (b) expressing the GPT transgene in the transgenic plant or progeny thereof, wherein GPT polypeptides are overexpressed in the transgenic plant or the progeny thereof relative to an analogous wild type or untransformed plant of the same species, where the transgenic plant has an increased biomass yield relative to an analogous wild type or untransformed plant of the same species. The resulting plant may have at least one additional enhanced growth property including one or more of increased nitrogen use efficiency, earlier flowering, earlier budding, increased plant height, increased flowering, increased budding, larger leaves, increased fruit or pod yield and increased seed yield. The method may further include propagating the plant and harvesting a seed therefrom. The transgenic plant produced by the method may produce more 2-oxo-glutaramate relative to an analogous wild type or untransformed plant of the same species. The transgenic plant produced by the method may have an increased leaf-to-root ratio of GS activity in comparison to an analogous wild type or untransformed plant of the same species. The transgenic plant produced by the method may have an increased leaf-to-root ratio of 2-oxo-glutaramate in comparison to an analogous wild type or untransformed plant of the same species.

In some implementations, the glutamine phenylpyruvate transaminase (GPT) transgene may encode a GPT polypeptide having an amino acid sequence that has at least 85% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a GPT polypeptide having an amino acid sequence that has at least 90% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a GPT polypeptide having an amino acid sequence that has at least 95% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a polypeptide having an amino acid sequence selected from the group consisting of (a) SEQ ID NO: 2, SEQ ID NO: 11, SEQ ID NO: 13, and SEQ ID NO: 15. The GPT transgene may be incorporated into the genome of the plant.

In one embodiment, the present invention relates to a method for generating and selecting transgenic plants having increased production of 2-oxo-glutaramate, including (a) introducing into a plurality of plant cells a glutamine phenylpyruvate transaminase (GPT) transgene that encodes a GPT polypeptide having an amino acid sequence that has at least 80% sequence identity to SEQ ID NO: 2, SEQ ID NO: 11, SEQ ID NO: 13, or SEQ ID NO: 15 and has GPT catalytic activity, (b) generating a plurality of transgenic plants from the plurality of plant cells; and (c) selecting a transgenic plant from the plurality of transgenic plants that produces more 2-oxo-glutaramate relative to an analogous wild type or untransformed plant. The resulting plant may have at least one additional enhanced growth property including one or more of increased nitrogen use efficiency, earlier flowering, earlier budding, increased plant height, increased flowering, increased budding, larger leaves, increased fruit or pod yield and increased seed yield. The method may further include propagating the plant and harvesting a seed therefrom. The transgenic plant produced by the method may produce more 2-oxo-glutaramate relative to an analogous wild type or untransformed plant of the same species. The transgenic plant produced by the method may have an increased leaf-to-root ratio of GS activity in comparison to an analogous wild type or untransformed plant of the same species. The transgenic plant produced by the method may have an increased leaf-to-root ratio of 2-oxo-glutaramate in comparison to an analogous wild type or untransformed plant of the same species.

In some implementations, the glutamine phenylpyruvate transaminase (GPT) transgene may encode a GPT polypeptide having an amino acid sequence that has at least 85% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a GPT polypeptide having an amino acid sequence that has at least 90% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a GPT polypeptide having an amino acid sequence that has at least 95% sequence identity to SEQ ID NO: 2, SEQ ID NO: 11, SEQ ID NO: 13, or SEQ ID NO: 15 and GPT catalytic activity. In some implementations, the glutamine phenylpyruvate transaminase (GPT) transgene may encode a polypeptide having an amino acid sequence selected from the group consisting of (a) SEQ ID NO: 2, SEQ ID NO: 11, SEQ ID NO: 13, and SEQ ID NO: 15. The GPT transgene may be incorporated into the genome of the plant.

In another embodiment, the GPT transgene is a GPT polynucleotide encoding an Arabidopsis derived GPT, such as the GPT of SEQ ID NO: 2, SEQ ID NO: 21 and SEQ ID NO: 30. The GPT transgene may be encoded by the nucleotide sequence of SEQ ID NO: 1; a nucleotide sequence having at least 75% and more preferably at least 80% identity to SEQ ID NO: 1, and encoding a polypeptide having GPT activity; a nucleotide sequence encoding the polypeptide of SEQ ID NO: 2, or a polypeptide having at least 75% and more preferably at least 80% sequence identity thereto which has GPT activity; or a nucleotide sequence encoding the polypeptide of SEQ ID NO: 2 truncated at its amino terminus by between 30 to 56 amino acid residues, or a polypeptide having at least 75% and more preferably at least 80% sequence identity thereto which has GPT activity.

In another specific embodiment, the GPT transgene is a GPT polynucleotide encoding a Grape derived GPT, such as the Grape GPTs of SEQ ID NO: 4 and SEQ ID NO: 26. The GPT transgene may be encoded by the nucleotide sequence of SEQ ID NO: 3; a nucleotide sequence having at least 75% and more preferably at least 80% identity to SEQ ID NO: 3, and encoding a polypeptide having GPT activity; or a nucleotide sequence encoding the polypeptide of SEQ ID NO: 4 or SEQ ID NO: 26, or a polypeptide having at least 75% and more preferably at least 80% sequence identity thereto which has GPT activity.

In yet another specific embodiment, the GPT transgene is a GPT polynucleotide encoding a Rice derived GPT, such as the Rice GPTs of SEQ ID NO: 6 and SEQ ID NO: 27. The GPT transgene may be encoded by the nucleotide sequence of SEQ ID NO: 5; a nucleotide sequence having at least 75% and more preferably at least 80% identity to SEQ ID NO: 5, and encoding a polypeptide having GPT activity; or a nucleotide sequence encoding the polypeptide of SEQ ID NO: 6 or SEQ ID NO: 27, or a polypeptide having at least 75% and more preferably at least 80% sequence identity thereto which has GPT activity.

In yet another specific embodiment, the GPT transgene is a GPT polynucleotide encoding a Soybean derived GPT, such as the Soybean GPTs of SEQ ID NO: 8 or SEQ ID NO: 28 with a further Isoleucine at the N-terminus of the sequence. The GPT transgene may be encoded by the nucleotide sequence of SEQ ID NO: 7; a nucleotide sequence having at least 75% and more preferably at least 80% identity to SEQ ID NO: 7, and encoding a polypeptide having GPT activity; or a nucleotide sequence encoding the polypeptide of SEQ ID NO: 8 or SEQ ID NO: 28 with a further Isoleucine at the N-terminus of the sequence, or a polypeptide having at least 75% and more preferably at least 80% sequence identity thereto which has GPT activity.

In yet another specific embodiment, the GPT transgene is a GPT polynucleotide encoding a Barley derived GPT, such as the Barley GPTs of SEQ ID NO: 15 and SEQ ID NO: 34. The GPT transgene may be encoded by the nucleotide sequence of SEQ ID NO: 9; a nucleotide sequence having at least 75% and more preferably at least 80% identity to SEQ ID NO: 9, and encoding a polypeptide having GPT activity; or a nucleotide sequence encoding the polypeptide of SEQ ID NO: 10, SEQ ID NO: 29 or SEQ ID NO: 40, or a polypeptide having at least 75% and more preferably at least 80% sequence identity thereto which has GPT activity.

In yet another specific embodiment, the GPT transgene is a GPT polynucleotide encoding a Zebra fish derived GPT, such as the Zebra fish GPTs of SEQ ID NO: 12 and SEQ ID NO: 30. The GPT transgene may be encoded by the nucleotide sequence of SEQ ID NO: 11; a nucleotide sequence having at least 75% and more preferably at least 80% identity to SEQ ID NO: 11, and encoding a polypeptide having GPT activity; or a nucleotide sequence encoding the polypeptide of SEQ ID NO: 12 or SEQ ID NO: 30, or a polypeptide having at least 75% and more preferably at least 80% sequence identity thereto which has GPT activity.

In yet another specific embodiment, the GPT transgene is a GPT polynucleotide encoding a Bamboo derived GPT, such as the Bamboo GPT of SEQ ID NO: 19 or SEQ ID NO: 31. The GPT transgene may be encoded by the nucleotide sequence of SEQ ID NO: 18; a nucleotide sequence having at least 75% and more preferably at least 80% identity to SEQ ID NO: 18; or a nucleotide sequence encoding a polypeptide having GPT activity encoded by a nucleotide sequence encoding the polypeptide of SEQ ID NO: 36, or a polypeptide having at least 75% and more preferably at least 80% sequence identity thereto which has GPT activity.

As will be appreciated by one skilled in the art, other GPT polynucleotides suitable for use as GPT transgenes in the practice of the invention may be obtained by various means, and tested for the ability to direct the expression of a GPT with GPT activity in a recombinant expression system (i.e., E. coli (see Examples 20-23), in a transient in planta expression system (see Example 19), or in a transgenic plant (see Examples 1-18).

Transgene Constructs/Expression Vectors

In order to generate the transgenic plants of the invention, the gene coding sequence for the desired transgene(s) must be incorporated into a nucleic acid construct (also interchangeably referred to herein as a/an (transgene) expression vector, expression cassette, expression construct or expressible genetic construct), which can direct the expression of the transgene sequence in transformed plant cells. Such nucleic acid constructs carrying the transgene(s) of interest may be introduced into a plant cell or cells using a number of methods known in the art, including but not limited to electroporation, DNA bombardment or biolistic approaches, microinjection, and via the use of various DNA-based vectors such as Agrobacterium tumefaciens and Agrobacterium rhizogenes vectors. Once introduced into the transformed plant cell, the nucleic acid construct may direct the expression of the incorporated transgene(s) (i.e., GPT), either in a transient or stable fashion. Stable expression is preferred, and is achieved by utilizing plant transformation vectors which are able to direct the chromosomal integration of the transgene construct. Once a plant cell has been successfully transformed, it may be cultivated to regenerate a transgenic plant.

A large number of expression vectors suitable for driving the constitutive or induced expression of inserted genes in transformed plants are known. In addition, various transient expression vectors and systems are known. To a large extent, appropriate expression vectors are selected for use in a particular method of gene transformation (see, infra). Broadly speaking, a typical plant expression vector for generating transgenic plants will comprise the transgene of interest under the expression regulatory control of a promoter, a selectable marker for assisting in the selection of transformants, and a transcriptional terminator sequence.

More specifically, the basic elements of a nucleic acid construct for use in generating the transgenic plants of the invention are: a suitable promoter capable of directing the functional expression of the transgene(s) in a transformed plant cell, the transgene(s) (i.e., GPT coding sequence) operably linked to the promoter, preferably a suitable transcription termination sequence (i.e., nopaline synthetic enzyme gene terminator) operably linked to the transgene, and sometimes other elements useful for controlling the expression of the transgene, as well as one or more selectable marker genes suitable for selecting the desired transgenic product (i.e., antibiotic resistance genes).

As Agrobacterium tumefaciens is the primary transformation system used to generate transgenic plants, there are numerous vectors designed for Agrobacterium transformation. For stable transformation, Agrobacterium systems utilize “binary” vectors that permit plasmid manipulation in both E. coli and Agrobacterium, and typically contain one or more selectable markers to recover transformed plants (Hellens et al., 2000, Technical focus: A guide to Agrobacterium binary Ti vectors. Trends Plant Sci 5:446-451). Binary vectors for use in Agrobacterium transformation systems typically comprise the borders of T-DNA, multiple cloning sites, replication functions for Escherichia coli and A. tumefaciens, and selectable marker and reporter genes.

So-called “super-binary” vectors provide higher transformation efficiencies, and generally comprise additional virulence genes from a Ti (Komari et al., 2006, Methods Mol. Biol. 343: 15-41). Super binary vectors are typically used in plants which exhibit lower transformation efficiencies, such as cereals. Such additional virulence genes include without limitation virB, virE, and virG (Vain et al., 2004, The effect of additional virulence genes on transformation efficiency, transgene integration and expression in rice plants using the pGreen/pSoup dual binary vector system. Transgenic Res. 13: 593-603; Srivatanakul et al., 2000, Additional virulence genes influence transgene expression: transgene copy number, integration pattern and expression. J. Plant Physiol. 157, 685-690; Park et al., 2000, Shorter T-DNA or additional virulence genes improve Agrobacterium-mediated transformation. Theor. Appl. Genet. 101, 1015-1020; Jin et al., 1987, Genes responsible for the supervirulence phenotype of Agrobacterium tumefaciens A281. J. Bacteriol. 169: 4417-4425).

In the embodiments exemplified herein (see Examples, infra), expression vectors which place the inserted transgene(s) under the control of the constitutive CaMV 35S promoter are employed. A number of expression vectors which utilize the CaMV 35S promoter are known and/or commercially available. However, numerous promoters suitable for directing the expression of the transgene are known and may be used in the practice of the invention, as further described, infra.

Plant Promoters

A large number of promoters which are functional in plants are known in the art. In constructing GPT transgene constructs, the selected promoter(s) may be constitutive, non-specific promoters such as the Cauliflower Mosaic Virus 35S ribosomal promoter (CaMV 35S promoter), which is widely employed for the expression of transgenes in plants. Examples of other strong constitutive promoters include without limitation the rice actin 1 promoter, the CaMV 19S promoter, the Ti plasmid nopaline synthase promoter, the alcohol dehydrogenase promoter and the sucrose synthase promoter.

Alternatively, in some embodiments, it may be desirable to select a promoter based upon the desired plant cells to be transformed by the transgene construct, the desired expression level of the transgene, the desired tissue or subcellular compartment for transgene expression, the developmental stage targeted, and the like.

For example, when expression in photosynthetic tissues and compartments is desired, a promoter of the ribulose bisphosphate carboxylase (RuBisCo) gene may be employed. When the expression in seeds is desired, promoters of various seed storage protein genes may be employed. For expression in fruits, a fruit-specific promoter such as tomato 2A11 may be used. Examples of other tissue specific promoters include the promoters encoding lectin (Vodkin et al., 1983, Cell 34:1023-31; Lindstrom et al., 1990, Developmental Genetics 11:160-167), corn alcohol dehydrogenase 1 (Vogel et al, 1989, J. Cell. Biochem. (Suppl. 0) 13:Part D; Dennis et al., 1984, Nucl. Acids Res., 12(9): 3983-4000), corn light harvesting complex (Simpson, 1986, Science, 233: 34-38; Bansal et al., 1992, Proc. Natl. Acad. Sci. USA, 89: 3654-3658), corn heat shock protein (Odell et al., 1985, Nature, 313: 810-812; Rochester et al., 1986, EMBO J., 5: 451-458), pea small subunit RuBP carboxylase (Poulsen et al., 1986, Mot. Gen. Genet., 205(2): 193-200; Cashmore et al., 1983, Gen. Eng. Plants, Plenum Press, New York, pp 29-38), Ti plasmid mannopine synthase and Ti plasmid nopaline synthase (Langridge et al., 1989, Proc, Natl. Acad. Sci. USA, 86: 3219-3223), petunia chalcone isomerase (Van Tunen et al., 1988, EMBO J. 7(5): 1257-1263), bean glycine rich protein 1 (Keller et al., 1989, EMBO J. 8(5): 1309-1314), truncated CaMV 35s (Odell et al., 1985, supra), potato patatin (Wenzler et al., 1989, Plant Mol. Biol. 12: 41-50), root cell (Conkling et al., 1990, Plant Physiol. 93: 1203-1211), maize zein (Reina et al., 1990, Nucl. Acids Res. 18(21): 6426; Kriz et al., 1987, Mol. Gen. Genet. 207(1): 90-98; Wandelt and Feix, 1989, Nuc. Acids Res. 17(6): 2354; Langridge and Feix, 1983, Cell 34: 1015-1022; Reina et al., 1990, Nucl. Acids Res. 18(21): 6426), globulin-1 (Belanger and Kriz, 1991, Genetics 129: 863-872), α-tubulin (Carpenter et al., 1992, Plant Cell 4(5): 557-571; Uribe et al., 1998, Plant Mol. Biol. 37(6): 1069-1078), cab (Sullivan, et al., 1989, Mol. Gen. Genet. 215(3): 431-440), PEPCase (Hudspeth and Grula, 1989, Plant Mol. Biol. 12: 579-589), R gene complex (Chandler et al., 1989, The Plant Cell 1: 1175-1183), chalcone synthase (Franken et al., 1991, EMBO J. 10(9): 2605-2612) and glutamine synthetase promoters (U.S. Pat. No. 5,391,725; Edwards et al., 1990, Proc. Natl. Acad. Sci. USA 87: 3459-3463; Brears et al., 1991, Plant J. 1(2): 235-244).

In addition to constitutive promoters, various inducible promoter sequences may be employed in cases where it is desirable to regulate transgene expression as the transgenic plant regenerates, matures, flowers, etc. Examples of such inducible promoters include promoters of heat shock genes, protection responding genes (i.e., phenylalanine ammonia lyase; see, for example Bevan et al., 1989, EMBO J. 8(7): 899-906), wound responding genes (i.e., cell wall protein genes), chemically inducible genes (i.e., nitrate reductase, chitinase) and dark inducible genes (i.e., asparagine synthetase; see, for example U.S. Pat. No. 5,256,558). Also, a number of plant nuclear genes are activated by light, including gene families encoding the major chlorophyll a/b binding proteins (cab) as well as the small subunit of ribulose-1,5-bisphosphate carboxylase (rbcS) (see, for example, Tobin and Silverthorne, 1985, Annu. Rev. Plant Physiol. 36: 569-593; Dean et al., 1989, Annu. Rev. Plant Physiol. 40: 415-439).

Other inducible promoters include ABA- and turgor-inducible promoters, the auxin-binding protein gene promoter (Schwob et al., 1993, Plant J. 4(3): 423-432), the UDP glucose flavonoid glycosyl-transferase gene promoter (Ralston et al., 1988, Genetics 119(1): 185-197); the MPI proteinase inhibitor promoter (Cordero et al., 1994, Plant J. 6(2): 141-150), the glyceraldehyde-3-phosphate dehydrogenase gene promoter (Kohler et al., 1995, Plant Mol. Biol. 29(6): 1293-1298; Quigley et al., 1989, J. Mol. Evol. 29(5): 412-421; Martinez et al., 1989, J. Mol. Biol. 208(4): 551-565) and light inducible plastid glutamine synthetase gene from pea (U.S. Pat. No. 5,391,725; Edwards et al., 1990, supra).

For a review of plant promoters used in plant transgenic plant technology, see Potenza et al., 2004, In Vitro Cell. Devel. Biol—Plant, 40(1): 1-22. For a review of synthetic plant promoter engineering, see, for example, Venter, M., 2007, Trends Plant Sci., 12(3): 118-124.

Glutamine Phenylpyruvate Transaminase (GPT) Transgene

The present invention discloses for the first time that plants contain a glutamine phenylpyruvate transaminase (GPT) enzyme which is directly functional in the synthesis of the signal metabolite 2-hydroxy-5-oxoproline. Until now, no plant transaminase with a defined function has been described. Applicants have isolated and tested GPT polynucleotide coding sequences derived from several plant and animal species, and have successfully incorporated the gene into heterologous transgenic host plants which exhibit markedly improved growth characteristics, including faster growth, higher foliar protein content, and faster CO₂ fixation rates.

It is expected that all plant species contain a GPT which functions in the same metabolic pathway, involving the biosynthesis of the signal metabolite 2-hydroxy-5-oxoproline, a powerful signal metabolite which regulates the function of a large number of genes involved in the photosynthesis apparatus, carbon fixation and nitrogen metabolism. Thus, in the practice of the invention, any plant gene encoding a GPT homolog or functional variants thereof may be useful in the generation of transgenic plants of this invention. Moreover, given the structural similarity between various plant GPT protein structures and the putative (and biologically active) GPT homolog from Danio rerio (Zebra fish) (see Example 22), other non-plant GPT homologs may be used in preparing GPT transgenes for use in generating the transgenic plants of the invention.

Defined herein are various GPT proteins and GPT gene coding sequences isolated from a number of plant species. As disclosed herein, GPT proteins share remarkable structural similarity within plant species, and are active in catalyzing the synthesis of 2-hydroxy-5-oxoproline (2-oxoglutaramate). The invention provides the sequences of various GPT polynucleotides encoding GPT proteins, as well as the sequences of various GPT polypeptides which may be encoded by GPT polynucleotides, including GPTs derived from Arabidopsis, Grape, Rice, Soybean, Barley, Bamboo and a non-plant homolog from Zebra fish, all but one of which (Bamboo) have been expressed as recombinant GPTs and confirmed as having GPT activity. In addition, the beginning of the mature plant GPT structure, absent the targeting sequence, has been determined, and GPT polynucleotide constructs in which all or part of the coding sequence of the GPT targeting sequence have been deleted have been expressed in transgenic plants and/or in E. coli to establish that the encoded GPT protein is expressed as an active GPT (see Examples herein).

In addition, using the GPT polynucleotide and protein sequences disclosed herein, several additional putative GPTs have been identified, including without limitation those derived from cotton, castor, poplar, moss and algae, all of which show significant to high structural identity and homology to the aforementioned GPT protein sequences.

Presented in FIG. 2 is a multiple sequence alignment of the amino acid sequences of several putative plant, algal and animal GPT proteins, showing a high degree of structural identity and conservation. Interestingly, whereas a high degree of structural conservation is seen beginning at alignment residue 90, likely at or near the amino-terminus of a mature GPT protein following proteolytic cleavage of the target sequence (sequence beginning with VAKR in all but two sequences), little structural homology is seen in the presumed targeting sequences. With respect to the plant sequences, this may be a consequence of the natural variability in chloroplast targeting sequences among different plants. The first ten of these aligned sequences terminate (C-terminus) at alignment residue position 473-475. When individually compared (by BLAST alignment) to the Arabidopsis mature protein sequence provided in SEQ ID NO: 30, the following sequence identities and homologies (BLAST “positives”, including similar amino acids) were obtained for the following mature GPT protein sequences:

| [SEQ ID] | ORIGIN | % IDENTITY | % POSITIVE |
|---|---|---|---|
| [31] | Grape | 84 | 93 |
| [32] | Rice | 83 | 91 |
| [33] | Soybean | 83 | 93 |
| [34] | Barley | 82 | 91 |
| [35] | Zebra fish | 83 | 92 |
| [36] | Bamboo | 81 | 90 |
| | Corn | 79 | 90 |
| | Castor | 84 | 93 |
| | Poplar | 85 | 93 |

Underscoring the conserved nature of the structure of the GPT protein across most plant species, the conservation seen within the above plant species extends to the non-human putative GPTs from Zebra fish and Chlamydomonas. In the case of Zebra fish, the extent of identity is very high (83% amino acid sequence identity with the mature Arabidopsis GPT of SEQ ID NO: 30, and 92% homologous taking similar amino acid residues into account). The Zebra fish mature GPT was confirmed by expressing it in E. coli and demonstrating biological activity (synthesis of 2-oxoglutaramate).

In order to determine whether putative GPT homologs would be suitable for generating the growth-enhanced transgenic plants of the invention, one may express the coding sequence thereof in E. coli or another suitable host and determine whether the 2-oxoglutaramate signal metabolite is synthesized at increased levels (see Examples 19-23). Where such an increase is demonstrated, the coding sequence may then be introduced into both homologous plant hosts and heterologous plant hosts, and growth characteristics evaluated. Any assay that is capable of detecting 2-oxoglutaramate with specificity may be used for this purpose, including without limitation the NMR and HPLC assays described in Example 2, infra. In addition, assays which measure GPT activity directly may be employed.

Any plant GPT with 2-oxoglutaramate synthesis activity may be used to transform plant cells in order to generate transgenic plants of the invention. There appears to be a high level of structural homology among plant species, which appears to extend beyond plants, as evidenced by the close homology between various plant GPT proteins and the putative Zebra fish GPT homolog. Therefore, various plant GPT genes may be used to generate growth-enhanced transgenic plants in a variety of heterologous plant species. In addition, GPT transgenes expressed in a homologous plant would be expected to result in the desired enhanced-growth characteristics as well (i.e., rice glutamine transaminase over-expressed in transgenic rice plants), although it is possible that regulation within a homologous cell may attenuate the expression of the transgene in some fashion that may not be operable in a heterologous cell.

With the benefit of the various GPT polynucleotides exemplified herein, one of ordinary skill in the art may obtain additional GPT polynucleotides from other plant and non-plant sources using standard molecular cloning and recombinant DNA methodologies. In one approach, oligonucleotide probes based on the sequences of the GPT polynucleotides disclosed herein can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as ovules, and a cDNA library which contains the GPT gene transcript is prepared from the mRNA. Alternatively, cDNA may be prepared from mRNA extracted from other tissues in which GPT genes or homologs are expressed.

cDNA or genomic libraries may be screened using a probe based upon the sequence of a GPT polynucleotide disclosed herein. Probes may be used to hybridize with genomic DNA or cDNA sequences to isolate homologous genes in the same or different plant species. Alternatively, antibodies raised against a GPT polypeptide can be used to screen an mRNA expression library.

GPT polynucleotides may also be amplified from nucleic acid samples using nucleic acid amplification techniques, such as polymerase chain reaction (PCR), which may be used to amplify the sequences of GPT genes directly from genomic DNA, from cDNA, from genomic libraries or cDNA libraries. PCR and other amplification methods may also be useful, for example, to clone GPT polynucleotide encoding GPT proteins for expression, prepare transgene constructs and expression vectors, generate transgenic plants, make oligonucleotide probes for detecting the presence of GPT mRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990).

Appropriate primers and probes for identifying GPT polynucleotides from plant tissues may be generated from the GPT polynucleotide sequences provided herein. Alignments of one or more of the GPT polynucleotides (genes) disclosed herein, and/or alignments of one or more of the GPT protein amino acid sequences disclosed herein, may be used to identify conserved regions in the GPT structure suitable for preparing the appropriate primer and probe sequences. Primers that specifically hybridize to conserved regions in one of the plant GPT polynucleotides disclosed herein may be used to amplify sequences from widely divergent plant species. Indeed, the sequence similarity seen among the several here exemplified GPT genes is very high, and many regions of perfect identity within the GPT protein primary structure are seen (see, for example, the sequence alignments shown in FIGS. 2A-B and 3A-B).

GPT polynucleotides may be tested for their ability to direct the expression of a functional, biologically active GPT protein by expressing the GPT polynucleotide in a cell and assaying for GPT activity or the presence of increased levels of 2-oxoglutaramate. Assays for GPT activity and 2-oxoglutaramate are disclosed herein (see Examples). In addition, GPT polypeptides may be tested in transgenic plants, following protocols in the Examples which follow. Plants expressing a GPT transgene will show increased levels of GPT activity, higher levels of 2-oxoglutaramate, and/or enhanced growth characteristics, relative to wild type plants (see Examples following).

The GPT polynucleotides are useful in directing the expression of recombinant GPT polypeptides in recombinant expression systems, as is generally known.

The GPT polynucleotides are useful in generating transgenic plants with increased levels of GPT activity, upregulated 2-oxoglutaramate levels, and enhanced growth characteristics. As consistently shown in the examples which follow, numerous species of transgenic plants containing a GPT transgene showed enhanced growth characteristics, including increased biomass, earlier and more productive flowering, increased fruit or pod yields, larger leaf sizes, taller heights, tolerance to high salt germination and faster growth.

In order to generate the transgenic plants of the invention, the gene coding sequence for the desired transgene(s) must be incorporated into a nucleic acid construct (also interchangeably referred to herein as a/an (transgene) expression vector, expression cassette, expression construct or expressible genetic construct), which can direct the expression of the transgene sequence in transformed plant cells. Such nucleic acid constructs carrying the transgene(s) of interest may be introduced into a plant cell or cells using a number of methods known in the art, including but not limited to electroporation, DNA bombardment or biolistic approaches, microinjection, and via the use of various DNA-based vectors such as Agrobacterium tumefaciens and Agrobacterium rhizogenes vectors. Once introduced into the transformed plant cell, the nucleic acid construct may direct the expression of the incorporated transgene(s) (i.e., GPT), either in a transient or stable fashion. Stable expression is preferred, and is achieved by utilizing plant transformation vectors which are able to direct the chromosomal integration of the transgene construct. Once a plant cell has been successfully transformed, it may be cultivated to regenerate a transgenic plant.

A large number of expression vectors suitable for driving the constitutive or induced expression of inserted genes in transformed plants are known. In addition, various transient expression vectors and systems are known. To a large extent, appropriate expression vectors are selected for use in a particular method of gene transformation (see, infra). Broadly speaking, a typical plant expression vector for generating transgenic plants will comprise the transgene of interest under the expression regulatory control of a promoter, a selectable marker for assisting in the selection of transformants, and a transcriptional terminator sequence.

More specifically, the basic elements of a nucleic acid construct for use in generating the transgenic plants of the invention are: a suitable promoter capable of directing the functional expression of the transgene(s) in a transformed plant cell, the transgene (s) (i.e., GPT coding sequence) operably linked to the promoter, preferably a suitable transcription termination sequence (i.e., nopaline synthetic enzyme gene terminator) operably linked to the transgene, and sometimes other elements useful for controlling the expression of the transgene, as well as one or more selectable marker genes suitable for selecting the desired transgenic product (i.e., antibiotic resistance genes).

Based on the results disclosed herein, it is clear that any number of GPT polynucleotides may be used to generate the transgenic plants of the invention. GPT proteins are highly conserved among various plant species, and it is evident from the experimental data disclosed herein that closely-related non-plant GPTs may be used as well (e.g., Danio rerio GPT).

GPT polynucleotides suitable for use as GPT transgenes in the practice of the invention may be obtained by various means, as will be appreciated by one skilled in the art, tested for the ability to direct the expression of a GPT with GPT activity in a recombinant expression system, i.e., E. coli (see Examples 20-23), in a transient in planta expression system (see Example 19), or in a transgenic plant (see Examples 1-18).

The invention also provides methods of generating a transgenic plant having enhanced growth and other agronomic characteristics. In one embodiment, a method of generating a transgenic plant having enhanced growth and other agronomic characteristics comprises introducing into a plant cell an expression cassette comprising a nucleic acid molecule encoding a GPT transgene, under the control of a suitable promoter capable of driving the expression of the transgene, so as to yield a transformed plant cell, and obtaining a transgenic plant which expresses the encoded GPT.

As exemplified herein, transgenic plants showing enhanced growth characteristics have been generated in two species of Tomato (see Examples 4 and 17), Pepper (Example 8), Beans (Examples 9 and 10), Cowpea (Examples 11 and 12), Alfalfa (Example 13), Cantaloupe (Example 14), Pumpkin (Example 15), Arabidopsis (Example 16) and Camilena (Example 18). These transgenic plants of the invention were generated using a variety of transformation methodologies, including Agrobacterium-mediated callus, floral dip, seed inoculation, pod inoculation, and direct flower inoculation, as well as combinations thereof, and via sexual crosses of, single transgene plants, as exemplified herein. Different GPT transgenes were successfully employed in generating the transgenic plants of the invention, as exemplified herein.

As Agrobacterium tumefaciens is the primary transformation system used to generate transgenic plants, there are numerous vectors designed for Agrobacterium transformation. For stable transformation, Agrobacterium systems utilize “binary” vectors that permit plasmid manipulation in both E. coli and Agrobacterium, and typically contain one or more selectable markers to recover transformed plants (Hellens et al., 2000, Technical focus: A guide to Agrobacterium binary Ti vectors. Trends Plant Sci 5:446-451). Binary vectors for use in Agrobacterium transformation systems typically comprise the borders of T-DNA, multiple cloning sites, replication functions for Escherichia coli and A. tumefaciens, and selectable marker and reporter genes.

So-called “super-binary” vectors provide higher transformation efficiencies, and generally comprise additional virulence genes from a Ti (Komari et al., 2006, Methods Mol. Biol. 343: 15-41). Super binary vectors are typically used in plants which exhibit lower transformation efficiencies, such as cereals. Such additional virulence genes include without limitation virB, virE, and virG (Vain et al., 2004, The effect of additional virulence genes on transformation efficiency, transgene integration and expression in rice plants using the pGreen/pSoup dual binary vector system. Transgenic Res. 13: 593-603; Srivatanakul et al., 2000, Additional virulence genes influence transgene expression: transgene copy number, integration pattern and expression. J. Plant Physiol. 157, 685-690; Park et al., 2000, Shorter T-DNA or additional virulence genes improve Agrobacterium-mediated transformation. Theor. Appl. Genet. 101, 1015-1020; Jin et al., 1987, Genes responsible for the supervirulence phenotype of Agrobacterium tumefaciens A281. J. Bacteriol. 169: 4417-4425).

In the embodiments exemplified herein (see Examples, infra), expression vectors which place the inserted transgene(s) under the control of the constitutive CaMV 35S promoter are employed. A number of expression vectors which utilize the CaMV 35S promoter are known and/or commercially available. However, numerous promoters suitable for directing the expression of the transgene are known and may be used in the practice of the invention, as further described, supra.

Transcription Terminators:

In preferred embodiments, a 3′ transcription termination sequence is incorporated downstream of the transgene in order to direct the termination of transcription and permit correct polyadenylation of the mRNA transcript. Suitable transcription terminators are those which are known to function in plants, including without limitation, the nopaline synthase (NOS) and octopine synthase (OCS) genes of Agrobacterium tumefaciens, the T7 transcript from the octopine synthase gene, the 3′ end of the protease inhibitor I or II genes from potato or tomato, the CaMV 35S terminator, the tmI terminator and the pea rbcS E9 terminator. In addition, a gene's native transcription terminator may be used. In specific embodiments, described by way of the Examples, infra, the nopaline synthase transcription terminator is employed.

Selectable Markers:

Selectable markers are typically included in transgene expression vectors in order to provide a means for selecting transformants. While various types of markers are available, various negative selection markers are typically utilized, including those which confer resistance to a selection agent that inhibits or kills untransformed cells, such as genes which impart resistance to an antibiotic (such as kanamycin, gentamycin, anamycin, hygromycin and hygromycinB) or resistance to a herbicide (such as sulfonylurea, gulfosinate, phosphinothricin and glyphosate). Screenable markers include, for example, genes encoding β-glucuronidase (Jefferson, 1987, Plant Mol. Biol. Rep 5: 387-405), genes encoding luciferase (Ow et al., 1986, Science 234: 856-859) and various genes encoding proteins involved in the production or control of anthocyanin pigments (See, for example, U.S. Pat. No. 6,573,432). The E. coli glucuronidase gene (gus, gusA or uidA) has become a widely used selection marker in plant transgenics, largely because of the glucuronidase enzyme's stability, high sensitivity and ease of detection (e.g., fluorometric, spectrophotometric, various histochemical methods). Moreover, there is essentially no detectable glucuronidase in most higher plant species.

Transformation Methodologies and Systems:

Various methods for introducing the transgene expression vector constructs of the invention into a plant or plant cell are well known to those skilled in the art, and any capable of transforming the target plant or plant cell may be utilized.

Agrobacterium-mediated transformation is perhaps the most common method utilized in plant transgenics, and protocols for Agrobacterium-mediated transformation of a large number of plants are extensively described in the literature (see, for example, Agrobacterium Protocols, Wan, ed., Humana Press, 2nd edition, 2006). Agrobacterium tumefaciens is a Gram negative soil bacteria that causes tumors (Crown Gall disease) in a great many dicot species, via the insertion of a small segment of tumor-inducing DNA (“T-DNA”, ‘transfer DNA’) into the plant cell, which is incorporated at a semi-random location into the plant genome, and which eventually may become stably incorporated there. Directly repeated DNA sequences, called T-DNA borders, define the left and the right ends of the T-DNA. The T-DNA can be physically separated from the remainder of the Ti-plasmid, creating a ‘binary vector’ system.

Agrobacterium transformation may be used for stably transforming dicots, monocots, and cells thereof (Rogers et al., 1986, Methods Enzymol., 118: 627-641; Hernalsteen et al., 1984, EMBO J., 3: 3039-3041; Hoykass-Van Slogteren et al., 1984, Nature, 311: 763-764; Grimsley et al., 1987, Nature 325: 167-1679; Boulton et al., 1989, Plant Mol. Biol. 12: 31-40; Gould et al., 1991, Plant Physiol. 95: 426-434). Various methods for introducing DNA into Agrobacteria are known, including electroporation, freeze/thaw methods, and triparental mating. The most efficient method of placing foreign DNA into Agrobacterium is via electroporation (Wise et al., 2006, Three Methods for the Introduction of Foreign DNA into Agrobacterium, Methods in Molecular Biology, vol. 343: Agrobacterium Protocols, 2/e, volume 1; Ed., Wang, Humana Press Inc., Totowa, N.J., pp. 43-53). In addition, given that a large percentage of T-DNAs do not integrate, Agrobacterium-mediated transformation may be used to obtain transient expression of a transgene via the transcriptional competency of unincorporated transgene construct molecules (Helens et al., 2005, Plant Methods 1:13).

A large number of Agrobacterium transformation vectors and methods have been described (Karimi et al., 2002, Trends Plant Sci. 7(5): 193-5), and many such vectors may be obtained commercially (for example, Invitrogen, Carlsbad, Calif.). In addition, a growing number of “open-source” Agrobacterium transformation vectors are available (for example, pCambia vectors; Cambia, Canberra, Australia). See, also, subsection herein on TRANSGENE CONSTRUCTS, supra. In a specific embodiment described further in the Examples, a pMON316-based vector was used in the leaf disc transformation system of Horsch et. al. (Horsch et al., 1995, Science 227:1229-1231) to generate growth enhanced transgenic tobacco and tomato plants.

Other commonly used transformation methods that may be employed in generating the transgenic plants of the invention include, without limitation, microprojectile bombardment, or biolistic transformation methods, protoplast transformation of naked DNA by calcium, polyethylene glycol (PEG) or electroporation (Paszkowski et al., 1984, EMBO J. 3: 2727-2722; Potrykus et al., 1985, Mol. Gen. Genet. 199: 169-177; Fromm et al., 1985, Proc. Nat. Acad. Sci. USA 82: 5824-5828; Shimamoto et al., 1989, Nature, 338: 274-276.

Biolistic transformation involves injecting millions of DNA-coated metal particles into target cells or tissues using a biolistic device (or “gene gun”), several kinds of which are available commercially. Once inside the cell, the DNA elutes off the particles and a portion may be stably incorporated into one or more of the cell's chromosomes (for review, see Kikkert et al., 2005, Stable Transformation of Plant Cells by Particle Bombardment/Biolistics, in: Methods in Molecular Biology, vol. 286: Transgenic Plants: Methods and Protocols, Ed. L. Pena, Humana Press Inc., Totowa, N.J.).

Electroporation is a technique that utilizes short, high-intensity electric fields to permeabilize reversibly the lipid bilayers of cell membranes (see, for example, Fisk and Dandekar, 2005, Introduction and Expression of Transgenes in Plant Protoplasts, in: Methods in Molecular Biology, vol. 286: Transgenic Plants: Methods and Protocols, Ed. L. Pena, Humana Press Inc., Totowa, N.J., pp. 79-90; Fromm et al., 1987, Electroporation of DNA and RNA into plant protoplasts, in Methods in Enzymology, Vol. 153, Wu and Grossman, eds., Academic Press, London, UK, pp. 351-366; Joersbo and Brunstedt, 1991, Electroporation: mechanism and transient expression, stable transformation and biological effects in plant protoplasts. Physiol. Plant. 81, 256-264; Bates, 1994, Genetic transformation of plants by protoplast electroporation. Mol. Biotech. 2: 135-145; Dillen et al., 1998, Electroporation-mediated DNA transfer to plant protoplasts and intact plant tissues for transient gene expression assays, in Cell Biology, Vol. 4, ed., Cells, Academic Press, London, UK, pp. 92-99). The technique operates by creating aqueous pores in the cell membrane, which are of sufficiently large size to allow DNA molecules (and other macromolecules) to enter the cell, where the transgene expression construct (as T-DNA) may be stably incorporated into plant genomic DNA, leading to the generation of transformed cells that can subsequently be regenerated into transgenic plants.

Newer transformation methods include so-called “floral dip” methods, which offer the promise of simplicity, without requiring plant tissue culture, as is the case with all other commonly used transformation methodologies (Bent et al., 2006, Arabidopsis thaliana Floral Dip Transformation Method, Methods Mol Biol, vol. 343: Agrobacterium Protocols, 2/e, volume 1; Ed., Wang, Humana Press Inc., Totowa, N.J., pp. 87-103; Clough and Bent, 1998, Floral dip: a simplified method for Agrobacterium-mediated transformation of Arabidopsis thaliana, Plant J. 16: 735-743). However, with the exception of Arabidopsis, these methods have not been widely used across a broad spectrum of different plant species. Briefly, floral dip transformation is accomplished by dipping or spraying flowering plants in with an appropriate strain of Agrobacterium tumefaciens. Seeds collected from these To plants are then germinated under selection to identify transgenic Ti individuals. Example 16 demonstrated floral dip inoculation of Arabidopsis to generate transgenic Arabidopsis plants.

Other transformation methods include those in which the developing seeds or seedlings of plants are transformed using vectors such as Agrobacterium vectors. For example, such vectors may be used to transform developing seeds by injecting a suspension or mixture of the vector (i.e., Agrobacteria) directly into the seed cavity of developing pods (Wang and Waterhouse, 1997, Plant Mol. Biol. Reporter 15: 209-215). Seedlings may be transformed as described in Yasseem, 2009, Plant Mol. Biol. Reporter 27: 20-28. Germinating seeds may be transformed as described in Chee et al., 1989, Plant Pysiol. 91: 1212-1218. Intra-fruit methods, in which the vector is injected into fruit or developing fruit, may be also be used. Still other transformation methods include those in which the flower structure is targeted for vector inoculation, such as the flower inoculation methods.

In addition, although transgenes are most commonly inserted into the nuclear DNA of plant cells, trangenes may also be inserted into plastidic DNA (i.e., into the plastome of the chloroplast). In most flowering plants, plastids do not occur in the pollen cells, and therefore transgenic DNA incorporated within a plastome will not be passed on through propagation, thereby restricting the trait from migrating to wild type plants. Plastid transformation, however, is more complex than cell nucleus transformation, due to the presence of many thousands of plastomes per cell (as high as 10,000). Transplastomic lines are genetically stable only if all plastid copies are modified in the same way, i.e. uniformly. This is typically achieved through repeated regeneration under certain selection conditions to eliminate untransformed plastids, by segregating transplastomic and untransformed plastids, resulting in the selection of homoplasmic cells carrying the transgene construct and the selectable marker stably integrated therein. Plastid transformation has been successfully performed in various plant species, including tobacco, potatoes, oilseed rape, rice and Arabidopsis.

To generate transplastomic lines carrying GPT transgenes, the transgene expression cassette is inserted into flanking sequences from the plastome. Using homologous recombination, the transgene expression cassette becomes integrated into the plastome via a natural recombination process. The basic DNA delivery techniques for plastid transformation include particle bombardment of leaves or polyethylene glycol-mediated DNA transformation of protoplasts. Transplastomic plants carrying transgenes in the plastome may be expressed at very high levels, due to the fact that many plastids (i.e., chloroplasts) per cell, each carrying many copies of the plastome. This is particularly the case in foliar tissue, where a single mature leaf cell may contain over 10,000 copies of the plastome. Following a successful transformation of the plastome, the transplastomic events carry the transgene on every copy of the plastid genetic material. This can result in the transgene expression levels representing as much as half of the total protein produced in the cell.

Plastid transformation methods and vector systems are described, for example, in recent U.S. Pat. Nos. 7,528,292; 7,371,923; 7,235,711; and, 7,193,131. See also U.S. Pat. Nos. 6,680,426 and 6,642,053.

The foregoing plant transformation methodologies may be used to introduce transgenes into a number of different plant cells and tissues, including without limitation, whole plants, tissue and organ explants including chloroplasts, flowering tissues and cells, protoplasts, meristem cells, callus, immature embryos and gametic cells such as microspores, pollen, sperm and egg cells, tissue cultured cells of any of the foregoing, any other cells from which a fertile regenerated transgenic plant may be generated. Callus is initiated from tissue sources including, but not limited to, immature embryos, seedling apical meristems, microspores and the like. Cells capable of proliferating as callus are also recipient cells for genetic transformation.

Methods of regenerating individual plants from transformed plant cells, tissues or organs are known and are described for numerous plant species.

As an illustration, transformed plantlets (derived from transformed cells or tissues) are cultured in a root-permissive growth medium supplemented with the selective agent used in the transformation strategy (i.e., an antibiotic such as kanamycin). Once rooted, transformed plantlets are then transferred to soil and allowed to grow to maturity. Upon flowering, the mature plants are preferably selfed (self-fertilized), and the resultant seeds harvested and used to grow subsequent generations. Examples 3-6 describe the regeneration of transgenic tobacco and tomato plants.

To transgenic plants may be used to generate subsequent generations (e.g., T₁, T₂, etc.) by selfing of primary or secondary transformants, or by sexual crossing of primary or secondary transformants with other plants (transformed or untransformed). For example, as described in Example 7, infra, individual plants over expressing the Alfalfa GS1 gene and outperforming wildtype plants were crossed with individual plants over-expressing the Arabidopsis GPT gene and outperforming wildtype plants, by simple sexual crossing using manual pollen transfer. Reciprocal crosses were made such that each plant served as the male in a set of crosses and each plant served as the female in a second set of crosses. During the mature plant growth stage, the plants are typically examined for growth phenotype, CO₂ fixation rate, etc. (see following subsection).

Selection of Growth-Enhanced Transgenic Plants:

Transgenic plants may be selected, screened and characterized using standard methodologies. The preferred transgenic plants of the invention will exhibit one or more phenotypic characteristics indicative of enhanced growth and/or other desirable agronomic properties. Transgenic plants are typically regenerated under selective pressure in order to select transformants prior to creating subsequent transgenic plant generations. In addition, the selective pressure used may be employed beyond To generations in order to ensure the presence of the desired transgene expression construct or cassette.

T₀ transformed plant cells, calli, tissues or plants may be identified and isolated by selecting or screening for the genetic composition of and/or the phenotypic characteristics encoded by marker genes contained in the transgene expression construct used for the transformation. For example, selection may be conducted by growing potentially-transformed plants, tissues or cells in a growth medium containing a growth-repressive amount of antibiotic or herbicide to which the transforming genetic construct can impart resistance. Further, the transformed plant cells, tissues and plants can be identified by screening for the activity of marker genes (i.e., β-glucuronidase) which may be present in the transgene expression construct.

Various physical and biochemical methods may be employed for identifying plants containing the desired transgene expression construct, as is well known. Examples of such methods include Southern blot analysis or various nucleic acid amplification methods (i.e., PCR) for identifying the transgene, transgene expression construct or elements thereof, Northern blotting, Si RNase protection, reverse transcriptase PCR (RT-PCR) amplification for detecting and determining the RNA transcription products, and protein gel electrophoresis, Western blotting, immunoprecipitation, enzyme immunoassay, and the like may be used for identifying the protein encoded and expressed by the transgene.

In another approach, expression levels of genes, proteins and/or metabolic compounds that are know to be modulated by transgene expression in the target plant may be used to identify transformants. In one embodiment of the present invention, increased levels of the signal metabolite 2-oxoglutaramate may be used to screen for desirable transformants, as exemplified in the Examples. Similarly, increased levels of GPT and/or GS activity may be assayed, as exemplified in the Examples.

Ultimately, the transformed plants of the invention may be screened for enhanced growth and/or other desirable agronomic characteristics. Indeed, some degree of phenotypic screening is generally desirable in order to identify transformed lines with the fastest growth rates, the highest seed yields, etc., particularly when identifying plants for subsequent selfing, cross-breeding and back-crossing. Various parameters may be used for this purpose, including without limitation, growth rates, total fresh weights, dry weights, seed and fruit yields (number, weight), seed and/or seed pod sizes, seed pod yields (e.g., number, weight), leaf sizes, plant sizes, increased flowering, time to flowering, overall protein content (in seeds, fruits, plant tissues), specific protein content (i.e., GS), nitrogen content, free amino acid, and specific metabolic compound levels (i.e., 2-oxoglutaramate). Generally, these phenotypic measurements are compared with those obtained from a parental identical or analogous plant line, an untransformed identical or analogous plant, or an identical or analogous wild-type plant (i.e., a normal or parental plant). Preferably, and at least initially, the measurement of the chosen phenotypic characteristic(s) in the target transgenic plant is done in parallel with measurement of the same characteristic(s) in a normal or parental plant. Typically, multiple plants are used to establish the phenotypic desirability and/or superiority of the transgenic plant in respect of any particular phenotypic characteristic.

Preferably, initial transformants are selected and then used to generate Ti and subsequent generations by selfing (self-fertilization), until the transgene genotype breeds true (i.e., the plant is homozygous for the transgene). In practice, this is accomplished by screening at each generation for the desired traits and selfing those individuals, often repeatedly (i.e., 3 or 4 generations). As exemplified herein, transgenic plant lines propagated through at least one sexual generation (Tobacco, Arabidopsis, Tomato) demonstrated higher transgene product activities compared to lines that did not have the benefit of sexual reproduction and the concomitant increase in transgene copy number.

Stable transgenic lines may be crossed and back-crossed to create varieties with any number of desired traits, including those with stacked transgenes, multiple copies of a transgene, etc. Various common breeding methods are well known to those skilled in the art (see, e.g., Breeding Methods for Cultivar Development, Wilcox J. ed., American Society of Agronomy, Madison Wis. (1987)). Additionally, stable transgenic plants may be further modified genetically, by transforming such plants with further transgenes or additional copies of the parental transgene. Also contemplated are transgenic plants created by single transformation events which introduce multiple copies of a given transgene or multiple transgenes.

### EXAMPLES

Various aspects of the invention are further described and illustrated by way of the several examples which follow, none of which are intended to limit the scope of the invention.

### Example 1: Isolation of Arabidopsis Glutamine Phenylpyruvate Transaminase (GPT) Gene

In an attempt to locate a plant enzyme that is directly involved in the synthesis of the signal metabolite 2-oxoglutaramate, applicants hypothesized that the putative plant enzyme might bear some degree of structural relationship to a human protein that had been characterized as being involved in the synthesis of 2-oxoglutaramate. The human protein, glutamine transaminase K (E.C. 2.6.1.64) (also referred in the literature as cysteine conjugate β-lyase, kyneurenine aminotransferase, glutamine phenylpyruvate transaminase, and other names), had been shown to be involved in processing of cysteine conjugates of halogenated xenobiotics (Perry et al., 1995, FEBS Letters 360:277-280). Rather than having an activity involved in nitrogen assimilation, however, human cysteine conjugate β-lyase has a detoxifying activity in humans, and in animals (Cooper and Meister, 1977, supra). Nevertheless, the potential involvement of this protein in the synthesis of 2-oxoglutaramate was of interest.

Using the protein sequence of human cysteine conjugate β-lyase, a search against the TIGR Arabidopsis plant database of protein sequences identified one potentially related sequence, a polypeptide encoded by a partial sequence at the Arabidopsis gene locus at At1q77670, sharing approximately 36% sequence homology/identity across aligned regions.

The full coding region of the gene was then amplified from an Arabidopsis cDNA library (Stratagene) with the following primer pair:

[SEQ ID NO: 32]
5′-CCCATCGATGTACCTGGACATAAATGGTGTGATG-3′
[SEQ ID NO: 33]
5′-GATGGTACCTCAGACTTTTCTCTTAAGCTTCTGCTTC-3′

These primers were designed to incorporate Cla I (ATCGAT) and Kpn I (GGTACC) restriction sites to facilitate subsequent subcloning into expression vectors for generating transgenic plants. Takara ExTaq DNA polymerase enzyme was used for high fidelity PCR using the following conditions: initial denaturing 94° C. for 4 minutes, 30 cycles of 94° C. for 30 second, annealing at 55° C. for 30 seconds, extension at 72° C. for 90 seconds, with a final extension of 72° C. for 7 minutes. The amplification product was digested with Cla I and Kpn 1 restriction enzymes, isolated from an agarose gel electrophoresis and ligated into vector pMon316 (Rogers, et. al. 1987 Methods in Enzymology 153:253-277) which contains the cauliflower mosaic virus (CaMV) 35S constitutive promoter and the nopaline synthase (NOS) 3′ terminator. The ligation product was transformed into DH5α cells and transformants sequenced to verify the insert.

A 1.3 kb cDNA was isolated and sequenced, and found to encode a full length protein of 440 amino acids in length, including a putative chloroplast signal sequence.

### Example 2: Production of Biologically Active Arabidopsis Glutamine Phenylpyruvate Transaminase

To test whether the protein encoded by the cDNA isolated as described in Example 1, supra, is capable of catalyzing the synthesis of 2-oxoglutaramate, the cDNA was expressed in E. coli, purified, and assayed for its ability to synthesize 2-oxoglutaramate using a standard method.

NMR Assay for 2-Oxoglutaramate

Briefly, the resulting purified protein was added to a reaction mixture containing 150 mM Tris-HCl, pH 8.5, 1 mM beta mercaptoethanol, 200 mM glutamine, 100 mM glyoxylate and 200 μM pyridoxal 5′-phosphate. The reaction mixture without added test protein was used as a control. Test and control reaction mixtures were incubated at 37° C. for 20 hours, and then clarified by centrifugation to remove precipitated material. Supernatants were tested for the presence and amount of 2-oxoglutaramate using ¹³C NMR with authentic chemically synthesized 2-oxoglutaramate as a reference. The products of the reaction are 2-oxoglutaramate and glycine, while the substrates (glutamine and glyoxylate) diminish in abundance. The cyclic 2-oxoglutaramate gives rise to a distinctive signal allowing it to be readily distinguished from the open chain glutamine precursor.

HPLC Assay for 2-Oxoglutaramate

An alternative assay for GPT activity uses HPLC to determine 2-oxoglutaramate production, following a modification of Calderon et al., 1985, J Bacteriol 161(2): 807-809. Briefly, a modified extraction buffer consisting of 25 mM Tris-HCl pH 8.5, 1 mM EDTA, 20 μM FAD, 10 mM Cysteine, and -1.5% (v/v) Mercaptoethanol. Tissue samples from the test material (i.e., plant tissue) are added to the extraction buffer at approximately a 1/3 ratio (w/v), incubated for 30 minutes at 37° C., and stopped with 200 μl of 20% TCA. After about 5 minutes, the assay mixture is centrifuged and the supernatant used to quantify 2-oxoglutaramate by HPLC, using an ION-300 7.8 mm ID×30 cm L column, with a mobile phase in 0.01 N H₂SO₄, a flow rate of approximately 0.2 ml/min, at 40° C. Injection volume is approximately 20 μl, and retention time between about 38 and 39 minutes. Detection is achieved with 210 nm UV light.

Results Using NMR Assay:

This experiment revealed that the test protein was able to catalyze the synthesis of 2-oxoglutaramate. Therefore, these data indicate that the isolated cDNA encodes a glutamine phenylpyruvate transaminase that is directly involved in the synthesis of 2-oxoglutaramate in plants. Accordingly, the test protein was designated Arabidopsis glutamine phenylpyruvate transaminase, or “GPT”.

The nucleotide sequence of the Arabidopsis GPT coding sequence is shown in the Table of Sequences, SEQ ID NO. 1. The translated amino acid sequence of the GPT protein is shown in SEQ ID NO. 2.

### Example 3: Creation of Transgenic Tobacco Plants Over-Expressing Arabidopsis GPT

Generation of Plant Expression Vector pMON-PJU:

Briefly, the plant expression vector pMon316-PJU was constructed as follows. The isolated cDNA encoding Arabidopsis GPT (Example 1) was cloned into the ClaI-KpnI polylinker site of the pMON316 vector, which places the GPT gene under the control of the constitutive cauliflower mosaic virus (CaMV) 35S promoter and the nopaline synthase (NOS) transcriptional terminator. A kanamycin resistance gene was included to provide a selectable marker.

Agrobacterium-Mediated Plant Transformations:

pMON-PJU and a control vector pMon316 (without inserted DNA) were transferred to Agrobacterium tumefaciens strain pTiTT37ASE using a standard electroporation method (McCormac et al., 1998, Molecular Biotechnology 9:155-159), followed by plating on LB plates containing the antibiotics spectinomycin (100 micro gm/ml) and kanamycin (50 micro gm/ml). Antibiotic resistant colonies of Agrobacterium were examined by PCR to assure that they contained plasmid.

Nicotiana tabacum cv. Xanthi plants were transformed with pMON-PJU transformed Agrobacteria using the leaf disc transformation system of Horsch et. al. (Horsch et al., 1995, Science 227:1229-1231). Briefly, sterile leaf disks were inoculated and cultured for 2 days, then transferred to selective MS media containing 100 μg/ml kanamycin and 500 μg/ml clafaran. Transformants were confirmed by their ability to form roots in the selective media.

Generation of GPT Transgenic Tobacco Plants:

Sterile leaf segments were allowed to develop callus on Murashige & Skoog (M&S) media from which the transformant plantlets emerged. These plantlets were then transferred to the rooting-permissive selection medium (M&S medium with kanamycin as the selection agent). The healthy, and now rooted, transformed tobacco plantlets were then transferred to soil and allowed to grow to maturity and upon flowering the plants were selfed and the resultant seeds were harvested. During the growth stage the plants had been examined for growth phenotype and the CO₂ fixation rate was measured for many of the young transgenic plants.

Production of T1 and T2 Generation GPT Transgenic Plants:

Seeds harvested form the T₀ generation of the transgenic tobacco plants were germinated on M&S media containing kanamycin (100 mg/L) to enrich for the transgene. At least one fourth of the seeds did not germinate on this media (kanamycin is expected to inhibit germination of the seeds without resistance that would have been produced as a result of normal genetic segregation of the gene) and more than half of the remaining seeds were removed because of demonstrated sensitivity (even mild) to the kanamycin.

The surviving plants (T₁ generation) were thriving and these plants were then selfed to produce seeds for the T₂ generation. Seeds from the T₁ generation were germinated on MS media supplemented for the transformant lines with kanamycin (10 mg/liter). After 14 days they were transferred to sand and provided quarter strength Hoagland's nutrient solution supplemented with 25 mM potassium nitrate. They were allowed to grow at 24° C. with a photoperiod of 16 h light and 8 hr dark with a light intensity of 900 micromoles per meter squared per second. They were harvested 14 days after being transferred to the sand culture.

Characterization of GPT Transgenic Plants:

Harvested transgenic plants (both GPT transgenes and vector control transgenes) were analyzed for glutamine sythetase activity in root and leaf, whole plant fresh weight, total protein in root and leaf, and CO₂ fixation rate (Knight et al., 1988, Plant Physiol. 88: 333). Non-transformed, wild-type A. tumefaciens plants were also analyzed across the same parameters in order to establish a baseline control.

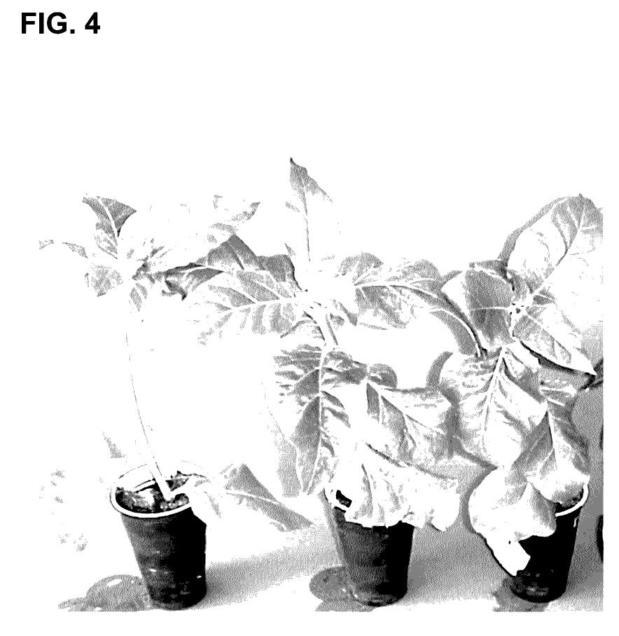

Growth characteristic results are tabulated below in Table I. Additionally, a photograph of the GPT transgenic plant compared to a wild type control plant is shown in FIG. 4 (together with GS1 transgenic tobacco plant, see Example 5). Across all parameters evaluated, the GPT transgenic tobacco plants showed enhanced growth characteristics. In particular, the GPT transgenic plants exhibited a greater than 50% increase in the rate of CO₂ fixation, and a greater than two-fold increase in glutamine synthetase activity in leaf tissue, relative to wild type control plants. In addition, the leaf-to-root GS ratio increased by almost three-fold in the transaminase transgenic plants relative to wild type control. Fresh weight and total protein quantity also increased in the transgenic plants, by about 50% and 80% (leaf), respectively, relative to the wild type control. These data demonstrate that tobacco plants overexpressing the Arabidopsis GPT transgene achieve significantly enhanced growth and CO₂ fixation rates.

TABLE I

| | Leaf | Root |
|---|---|---|
| Protein mg/gram fresh weight | | |
| Wild type - control | 8.3 | 2.3 |
| Line PN1-8 a second control | 8.9 | 2.98 |
| Line PN9-9 | 13.7 | 3.2 |
| Glutamine Synthetase activity, micromoles/min/mg protein | | |
| Wild type (Ratio of leaf:root = 4.1:1) | 4.3 | 1.1 |
| PN1-8 (Ratio of leaf:root = 4.2:1) | 5.2 | 1.3 |
| PN9-9 (Ratio of leaf:root = 10.9:1) | 10.5 | 0.97 |
| Whole Plant Fresh Weight, g | | |

| | |
|---|---|
| Wild type | 21.7 |
| PN1-8 | 26.1 |
| PN9-9 | 33.1 |
| CO₂ Fixation Rate, μmole/m²/sec | |
| Wild type | 8.4 |
| PN1-8 | 8.9 |
| PN9-9 | 12.9 |

Data = average of three plants
Wild type - Control plants; not regenerated or transformed.
PN1 lines were produced by regeneration after transformation using a construct without inserted gene. A control against the processes of regeneration and transformation.
PN 9 lines were produced by regeneration after transformation using a construct with the Arabidopsis GPT gene.

### Example 4: Generation of Transgenic Tomato Plants Carrying Arabidopsis GPT Transgene

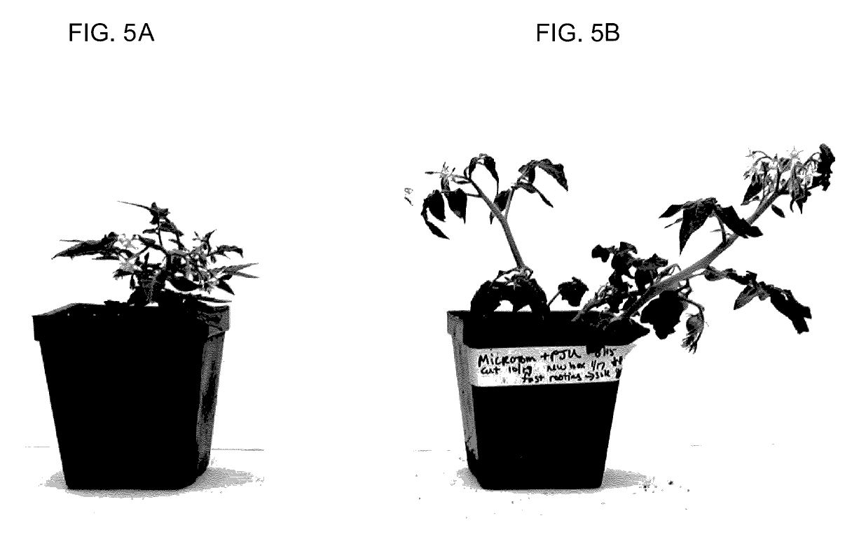

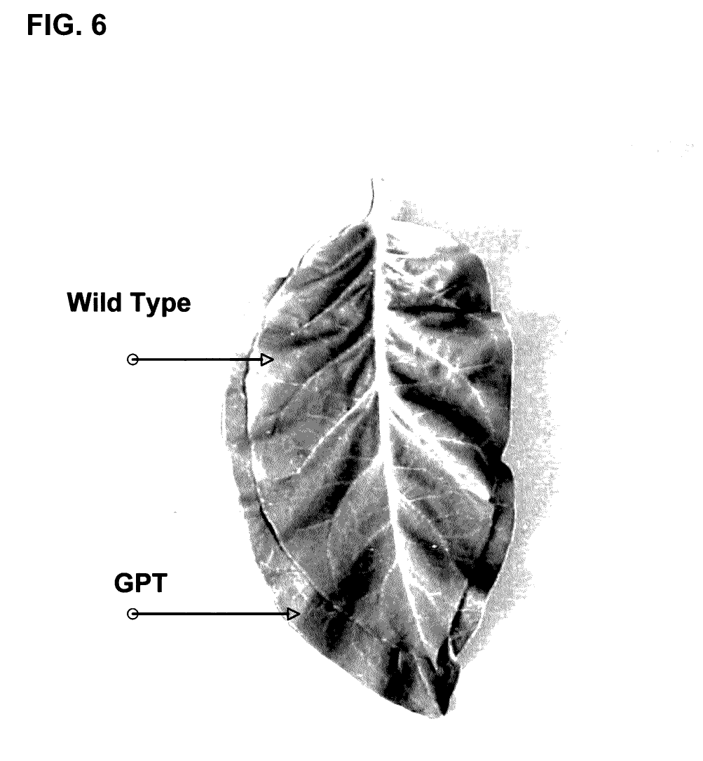

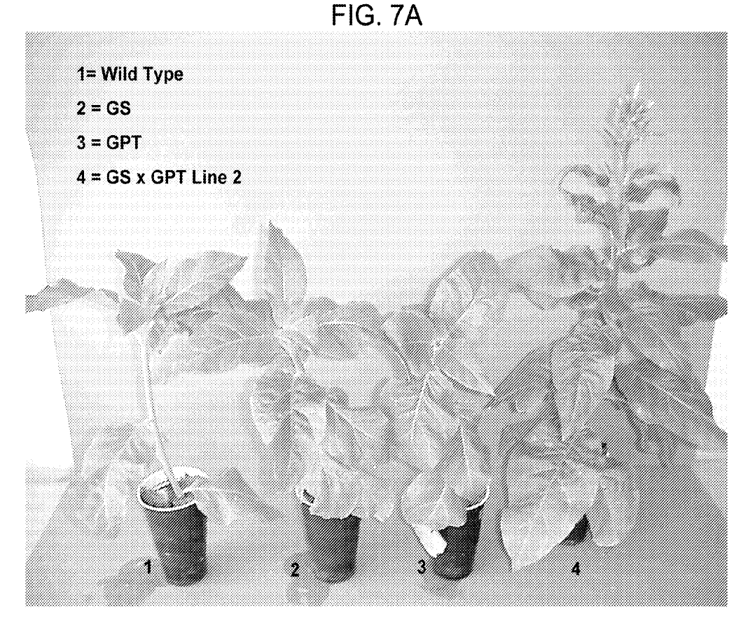

Transgenic Lycopersicon esculentum (Micro-Tom Tomato) plants carrying the Arabidopsis GPT transgene were generated using the vectors and methods described in Example 3. To transgenic tomato plants were generated and grown to maturity. Initial growth characteristic data of the GPT transgenic tomato plants is presented in Table II. The transgenic plants showed significant enhancement of growth rate, flowering, and seed yield in relation to wild type control plants. In addition, the transgenic plants developed multiple main stems, whereas wild type plants developed with a single main stem. A photograph of a GPT transgenic tomato plant compared to a wild type plant is presented in FIGS. 5A-5B (together with GS1 transgenic tomato plants, see Example 6).

TABLE II

| Growth | Wildtype | GPT Transgenic |
|---|---|---|
| Characteristics | Tomato | Tomato |
| Stem height, cm | 6.5 | 18, 12, 11 major stems |
| Stems | 1 | 3 major, 0 other |
| Buds | 2 | 16 |
| Flowers | 8 | 12 |
| Fruit | 0 | 3 |

### Example 5: Generation of Transgenic Tobacco Plants Overexpressing Alfalfa GS1

Generation of Plant Expression Vector pGS111:

Transgenic tobacco plants overexpressing the Alfalfa GS1 gene were generated as previously described (Temple et al., 1993, Mol. Gen. Genetics 236: 315-325). Briefly, the plant expression vector pGS111 was constructed by inserting the entire coding sequence together with extensive regions of both the 5′ and 3′ untranslated regions of the Alfalfa GS1 gene [SEQ ID NO: 3] (DasSarma at al., 1986, Science, Vol 232, Issue 4755, 1242-1244) into pMON316 (Rogers et al., 1987, supra), placing the transgene under the control of the constitutive cauliflower mosaic virus (CaMV) 35S promoter and the nopaline synthase (NOS) transcriptional terminator. A kanamycin resistance gene was included to provide a selectable marker.

Generation of GS1 Transformants:

pGS111 was transferred to Agrobacterium tumefaciens strain pTiTT37ASE using triparental mating as described (Rogers et al., 1987, supra; Unkefer et al., U.S. Pat. No. 6,555,500). Nicotiana tabacum cv. Xanthi plants were transformed with pGS111 transformed Agrobacteria using the leaf disc transformation system of Horsch et. al. (Horsch et al., 1995, Science 227:1229-1231). Transformants were selected and regenerated on MS medium containing 100 μg/ml kanamycin. Shoots were rooted on the same medium (with kanamycin, absent hormones) and transferred to potting soil:perlite:vermiculite (3:1:1), grown to maturity, and allowed to self. Seeds were harvested from this T₀ generation, and subsequent generations produced by selfing and continuing selection with kanamycin. The best growth performers were used to yield a T₃ progeny for crossing with the best performing GPT over-expressing lines identified as described in Example 3. A photograph of the GS1 transgenic plant compared to a wild type control plant is shown in FIG. 4 (together with GPT transgenic tobacco plant, see Example 3)

### Example 6: Generation of Transgenic Tomato Plants Carrying Alfalfa GS1 Transgene

Transgenic Lycopersicon esculentum (Micro-Tom Tomato) plants carrying the Alfalfa GS1 transgene were generated using the vector described in Example 5 and a transformation protocol essentially as described (Sun et al., 2006. Plant Cell Physiol. 46(3) 426-31). To transgenic tomato plants were generated and grown to maturity. Initial growth characteristic data of the GPT transgenic tomato plants is presented in Table III. The transgenic plants showed significant enhancement of growth rate, flowering, and seed yield in relation to wild type control plants. In addition, the transgenic plants developed multiple main stems, whereas wild type plants developed with a single main stem. A photograph of a GS1 transgenic tomato plant compared to a wild type plant is presented in FIGS. 5A-5B (together with GPT transgenic tomato plant, see Example 4).

TABLE III

| Growth | Wildtype | GS1 Transgenic |
|---|---|---|
| Characteristics | Tomato | Tomato |
| Stem height, cm | 6.5 | 16, 7, 5 major stems |
| Stems | 1 | 3 major, 3 med, 1 sm |
| Buds | 2 | 2 |
| Flowers | 8 | 13 |
| Fruit | 0 | 4 |

### Example 7: Generation of Double Transgenic Tobacco Plants Carrying GS1 and GPT Transgenes

In an effort to determine whether the combination of GS1 and GPT transgenes in a single transgenic plant might improve the extent to which growth and other agronomic characteristics may be enhanced, a number of sexual crosses between high producing lines of the single transgene (GS1 or GPT) transgenic plants were carried out. The results obtained are dramatic, as these crosses repeatedly generated progeny plants having surprising and heretofore unknown increases in growth rates, biomass yield, and seed production.

Materials and Methods:

Single-transgene, transgenic tobacco plants overexpressing GPT or GS1 were generated as described in Examples 3 and 4, respectively. Several of fastest growing T₂ generation GPT transgenic plant lines were crossed with the fastest growing T₃ generation GS1 transgenic plant lines using reciprocal crosses. The progeny were then selected on kanamycin containing M&S media as described in Example 3, and their growth, flowering and seed yields examined.

Tissue extractions for GPT and GS activities: GPT activity was extracted from fresh plant tissue after grinding in cold 100 mM Tris-HCl, pH 7.6, containing 1 mm ethylenediaminetetraacetic, 200 mM pyridoxal phosphate and 6 mM mercaptoethanol in a ratio of 3 ml per gram of tissue. The extract was clarified by centrifugation and used in the assay. GS activity was extracted from fresh plant tissue after grinding in cold 50 mM Imidazole, pH 7.5 containing 10 mM MgCl₂, and 12.5 mM mercaptoethanol in a ratio of 3 ml per gram of tissue. The extract was clarified by centrifugation and used in the assay. GPT activity was assayed as described in Calderon and Mora, 1985, Journal Bacteriology 161:807-809. GS activity was measured as described in Shapiro and Stadtmann, 1970, Methods in Enzymology 17A: 910-922. Both assays involve an incubation with substrates and cofactor at the proper pH. Detection was by HPLC.

Results:

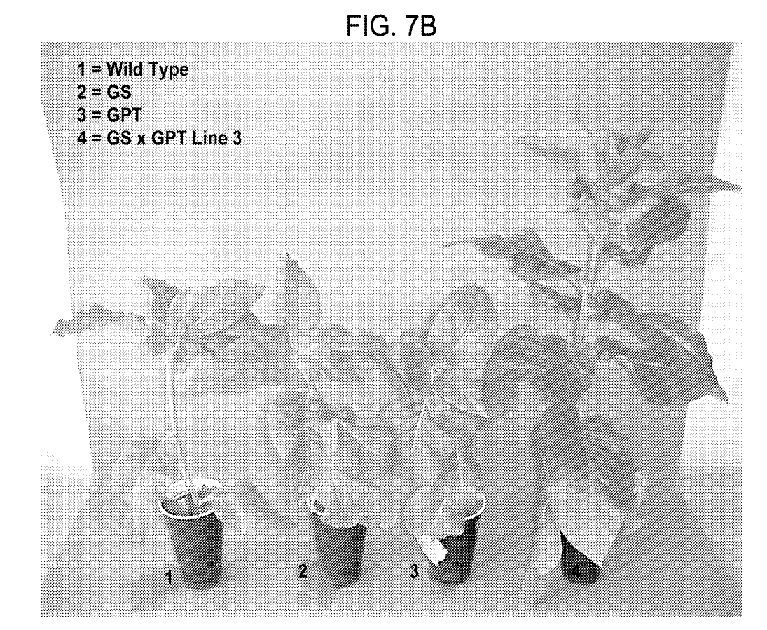

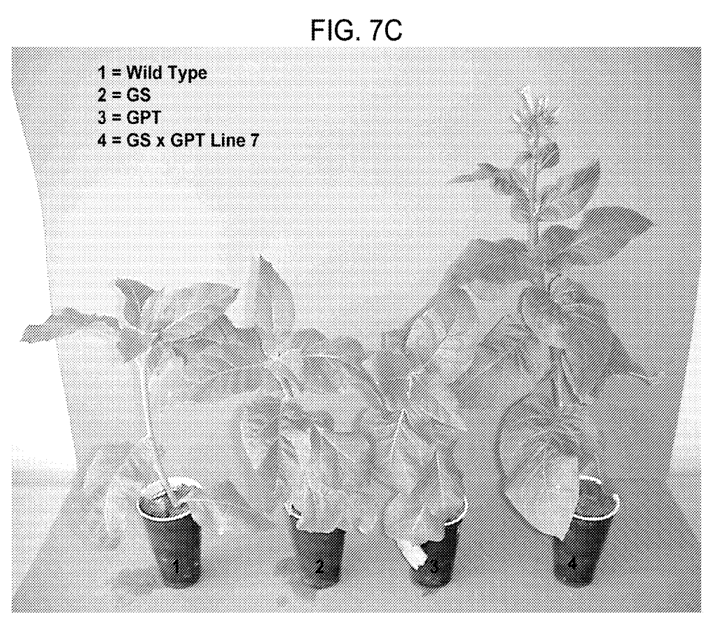

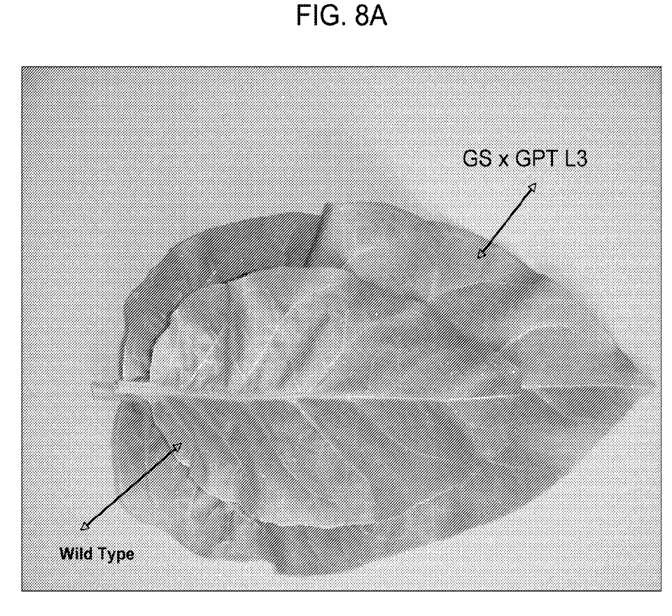

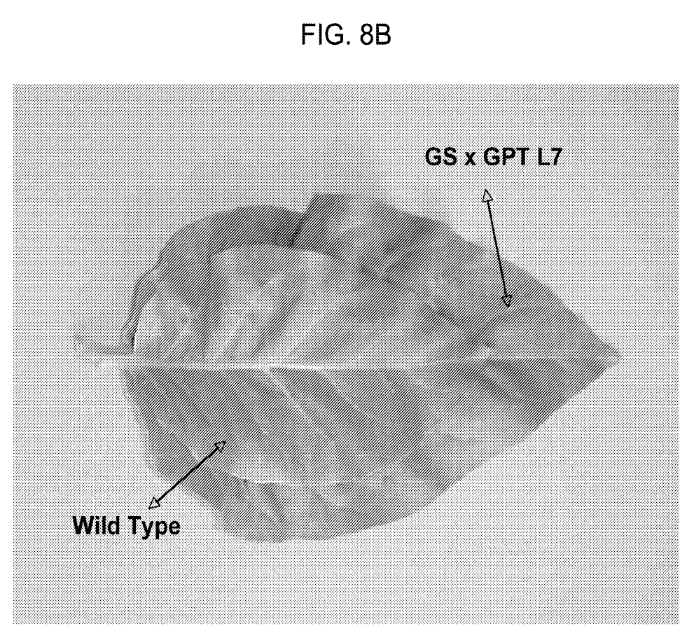

The results are presented in two ways. First, specific growth characteristics are tabulated in Tables IV.A and IV.B (biomass, seed yields, growth rate, GS activity, GPT activity, 2-oxoglutaramate activity, etc). Second, photographs of progeny plants and their leaves are shown in comparison to single-transgene and wild type plants and leaves are presented in FIGS. 7A-7C and FIGS. 8A-8B, which show much larger whole plants, larger leaves, and earlier and/or more abundant flowering in comparison to the parental single-transgene plants and wild type control plants.

Referring to Table IV.A, double-transgene progeny plants form these crosses showed tremendous increases total biomass (fresh weight), with fresh weights ranging from 45-89 grams per individual progeny plant, compared to a range of only 19-24 grams per individual wild type plant, representing on average, about a two- to three-fold increase over wild type plants, and representing at the high end, an astounding four-fold increase in, biomass over wild type plants. Taking the 24 individual double-transgene progeny plants evaluated, the average individual plant biomass was about 2.75 times that of the average wild type control plant. Four of the progeny lines showed approximately 2.5 fold greater average per plant fresh weights, while two lines showed over three-fold greater fresh weights in comparison to wild type plants.

In comparison to the single-transgene parental lines, the double-transgene progeny plants also showed far more than an additive growth enhancement. Whereas GPT single-transgene lines show as much as about a 50% increase over wild type biomass, and GS1 single-transgene lines as much as a 66% increase, progeny plants averaged almost a 200% increase over wild type plants.

Similarly, the double transgene progeny plants flowered earlier and more prolifically than either the wild type or single transgene parental lines, and produced a far greater number of seed pods as well as total number of seeds per plant. Referring again to Table IV.A, on average, the double-transgene progeny produced over twice the number of seed pods produced by wild type plants, with two of the high producer plants generating over three times the number of seed pods compared to wild type. Total seed yield in progeny plants, measured on a per plant weight basis, ranged from about double to nearly quadruple the number produced in wild type plants.

TABLE IV.A

| | FRESH WEIGHT | SEED PODS | SEED YIELD | | GS ACTIVITY | L/R |
|---|---|---|---|---|---|---|
| PLANT LINE | g/whole plant | #pods/plant | g/plant | LEAF | ROOT | RATIO |
| Wild Type Tobacco | | | | | | |
| Wild type 1 | 18.73 | 26 | 0.967 | | | |
| Wild type 2 | 24.33 | 24 | 1.07 | | | |
| Wild type 3 | 23.6 | 32 | 0.9 | | | |
| Wild type 4 | 18.95 | 32 | 1.125 | | | |
| WT Average | 21.4025 | 28.5 | 1.0155 | 7.75 | 1.45 | 5.34 |
| Cross 1 | | | | | | |
| X1L1a × PA9-9ff | | | | | | |
| 1 | 59.21 | 62 | 2.7811 | | | |
| 2 | 65.71 | 56 | | | | |
| 3 | 55.36 | 72 | | | | |
| 4 | 46.8 | 56 | | | | |
| Cross 1 Average | 56.77 | 61.5 | | 14.98 | 1.05 | 14.27 |
| Compared to WT | +265% | +216% | +274% | +193% | −28% | +267% |
| Cross 2 | | | | | | |
| PA9-2 × L9 | | | | | | |
| 1 | 70.83 | 61 | 1.76 | | | |
| 2 | 49.17 | 58 | 3.12 | | | |
| 3 | 50.23 | 90 NA | | | | |
| 4 | 45.77 | | | | | |
| Cross 2 Average | 54 | 58.3 | 2.44 | 16.32 | 1.81 | 9.02 |
| Compared to WT | +252% | +205% | +240% | +211% | +125% | +169% |
| Cross 3 | | | | | | |
| PA9-9ff × L1a | | | | | | |
| 1 | 89.1 | 77 | 3.687 | | | |
| 2 | 78.18 | | | | | |
| 3 | 58.34 | | | | | |
| 4 | 61.79 | | | | | |
| Cross 3 Average | 71.85 | 77 | 3.678 | 15.92 | 1.38 | 11.54 |
| Compared to WT | +336% | (one plant) | (one plant) | +205% | −5% | +216% |
| | | +270% | +362% | | | |
| Cross 5 | | | | | | |
| PA9-10aa × L1a | | | | | | |
| 1 | 65.34 | 45 | 2.947 | | | |
| 2 | 53.28 | 64 | 3.3314 | | | |
| 3 | 49.85 | 42 | 1.5667 | | | |
| 4 | 44.63 | 42 | 2.5013 | | | |
| Cross 5 Average | 53.275 | 48.25 | 2.86928 | 13.03 | 1.8 | 7.24 |
| Compared to WT | +244% | +169% | +283% | +168% | | |
| Cross 6 | | | | | | |
| PA9-17b × L1a | | | | | | |
| 1 | 56.7 | 64 | 2.492 | | | |
| 2 | 55.05 | 66 | 2.162 | | | |
| 3 | 51.51 | 59 | 1.8572 | | | |
| 4 | 45.38 | 72 | 4.742 | | | |
| Cross 6 Average | 52.16 | 65.25 | 2.8133 | 14.114.752 | 1.1.1124 | 13.29 |
| Compared to WT | +244% | +229% | +277% | | | |
| Cross 7 | | | | | | |
| PA9-20aa × L1b | | | | | | |
| 1 | 76.26 | 67 | 2.0535 | | | |
| 2 | 66.27 | 42 | 1.505 | | | |
| 3 | 72.26 | 72 | 2.3914 | | | |
| 4 | 63.91 | 91 | 2.87 | | | |
| Cross 7 Average | 69.675 | 68 | 2.204975 | 14.12 | 1.24 | 11.39 |
| Compared to WT | +326% | +239% | +217% | | | |
| Control PA9-9ff | | | | | | |
| 1 | 32.18 | N/A | | | | |
| 2 | 32.64 | N/A | | | | |
| 3 | 34.67 | N/A | | | | |
| 4 | 25.18 | N/A | | | | |
| Average | 31.17 | N/A | | 11.57 | 1.14 | 10.15 |
| Compared to WT | +148% | | | | | |
| Control GS L1a | | | | | | |
| 1 | 41.74 | N/A | | | | |
| 2 | 36.24 | N/A | | | | |
| 3 | 33.8 | N/A | | | | |
| 4 | 30.48 | N/A | | | | |
| Average | 35.57 | N/A | | 13.15 | 1.23 | 10.69 |
| Compared to WT | +166% | | | | | |

Table IV.B shows growth rate, biomass and yield, and biochemical characteristics of Line XX (Line 3 further selfed) compared to the single transgene line expressing GS1 and wild type control tobacco. All parameters are greatly increased in the double transgenic plant (Line XX). Notably, 2-oxoglutaramate activity was almost 17-fold higher, and seed yield and foliar biomass was three-fold higher, in Line XX plants versus control plants

TABLE IV.B

| | Specific | | | | GS | GPT | | |
|---|---|---|---|---|---|---|---|---|
| | Growth | Foliar | Fruit/ | | Activity | Activity | | Trans |
| Plant | Rate | Biomass | Flowers/ | Seed | umol/ | nmol/h/ | 2-oxoglutaramate | Gene |
| Type | mg/g/d | FWt, g | Buds | Yield g | min/gFWt | gFWt | nmol/gFWt | Assay |
| Wildtype, | 228 | 21.40 | 28.5 | 1.02 | 7.75 | 16.9 | 68.9 | No |
| avg | | | | | | | | |
| Line 1 GS | 269 | 35.57 | NM | NM | 11.6 | NM | 414 | Yes |
| Line XX | 339 | 59.71 | 62.9 | 2.94 | 16.3 | 243.9 | 1,153.6 | Yes |

NM Not Measured

### Example 8: Generation of Double Transgenic Pepper Plants Carrying GS1 and GPT Transgenes

In this example, Big Jim chili pepper plants (New Mexico varietal) were transformed with the Arabidopsis GPT full length coding sequence of SEQ ID NO: 1 under the control of the CMV 35S promoter, and the Arabidopsis GS1 coding sequence included in SEQ ID NO: 6 under the control of the RuBisCo promoter, using Agrobacterium-mediated transfer to seed pods. After 3 days, seeds were harvested and used to generate T0 plants and screened for transformants. The resulting double-transgenic plants showed higher pod yields, faster growth rates, and greater biomass yields in comparison to the control plants.

Materials and Methods:

Solanaceae Capisicum Pepper plants (“Big Jim” varietal) were transformed with the Arabidopsis GPT full length coding sequence of SEQ ID NO: 1 under the control of the CMV 35S promoter within the expression vector pMON (see Example 3), and the Arabidopsis GS1 coding sequence included in SEQ ID NO: 6 under the control of the RuBisCo promoter within the expression vector pCambia 1201 (Tomato rubisco rbcS3C promoter: Kyozulka et al., 1993, Plant Physiol. 103: 991-1000; SEQ ID NO: 22; vector construct of SEQ ID NO: 6), using Agrobacterium-mediated transfer to seed pods.

For this and all subsequent examples, the Cambia 1201 or 1305.1 vectors were constructed according to standard cloning methods (Sambrook et al., 1989, supra, Saiki et al., 1988, Science 239: 487-491). The vector is supplied with a 35S CaMV promoter; that promoter was replaced with RcbS-3C promoter from tomato to control the expression of the target gene. The Cambia 1201 vectors contain bacterial chlorophenicol and plant hygromycin resistance selectable marker genes. The Cambia 1305.1 vectors contain bacterial chlorophenicol and hygromycin resistance selectable marker genes.

The transgene expression vectors pMON (GPT transgene) and pCambia 1201 (GS transgene) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of either streptamycin for pMON constructs or chloroamphenicol for the Cambia constructs. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Pepper plants were then transformed with a mixture of the resulting Agrobacterium cell suspensions using a transformation protocol in which the Agrobacterium is injected directly into the seed cavity of developing pods. Briefly, developing pods were injected with the 200 ml mixture in order to inoculate immature seeds with the Agrobacteria essentially as described (Wang and Waterhouse, 1997, Plant Mol. Biol. Reporter 15: 209-215). In order to induce Agrobacteria virulence and improve transformation efficiencies, 10 μg/ml acetosyringonone was added to the Agrobacteria cultures prior to pod inoculations (see, Sheikholeslam and Weeks, 1986, Plant Mol. Biol. 8: 291-298).

Using a syringe, pods were injected with a liberal quantity of the Agrobacterium vector mixture, and left to incubate for about 3 days. Seeds were then harvested and germinated, and developing plants observed for phenotypic characteristics including growth and antibiotic resistance. Plants carrying the transgenes were green, whereas untransformed plants showed signs of chlorosis in leaf tips. Vigorously growing transformants were further cultivated and compared to wild type pepper plants grown under identical conditions.

Results:

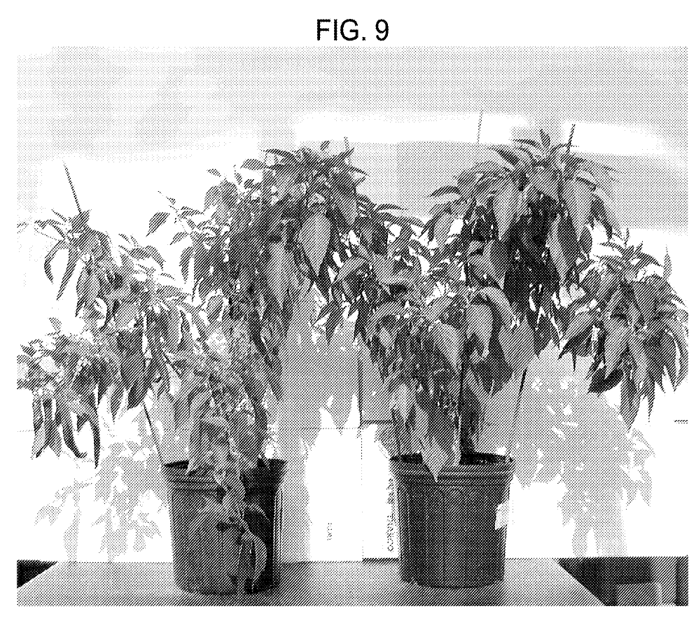

The results are presented in FIG. 9 and Table V. FIG. 9 shows a photograph of a GPT+GS double transgenic pepper plant compared to a control plant grown for the same time under identical conditions. This photograph shows tremendous pepper yield in the transgenic line compared to the control plant.

Table V presents biomass yield and GS activity, as well as transgene genotyping, in the transgenic lines compared to the wild type control. Referring to Table V, double-transgene progeny plants showed tremendous increases total biomass (fresh weight), with fresh weights, ranging from 393-662 grams per individual transgenic plant, compared to an average of 328 grams per wild type plant. Transgenic line A5 produced more than twice the total biomass of the controls. Moreover, pepper yields in the transgenic lines were greatly improved over wild type plants, and were 50% greater than control plants (on average). Notably, one of the transgene lines produced twice as many peppers as the control plant average.

TABLE V
TRANSGENIC PEPPER GROWTH/BIOMASS
AND REPRODUCTION

| | | | GS | |
|---|---|---|---|---|
| | Biomass, | Yield | activity | Transgene |
| | Foliar Fresh | Peppers, | Umoles/ | Presence |
| Plant type | Wt, g | g DWt | min/gFWt | Assay |
| Wildtype, avg | 328.2 | 83.7 | 1.09 | Negative |
| Line A2 | 457.3 | 184.2 | 1.57 | GPT - Yes |
| Line A5 | 661.7 | 148.1 | 1.8 | GPT - Yes |
| Line B1 | 493.4 | 141.0 | 1.3 | GPT - Yes |
| Line B4 | 393.1 | 136.0 | 1.6 | GPT - Yes |
| Line C1 | 509.4 | 152.9 | 1.55 | GPT - Yes |

FWt Fresh Weight;
DWt Dry Weight

### Example 9: Generation of Double Transgenic Bean Plants Carrying Arabidopsis GS1 and GPT Transgenes

In this example, yellow wax bean plants (Phaseolus vulgaris) were transformed with the Arabidopsis GPT full length coding sequence of SEQ ID NO: 1 under the control of the CMV 35S promoter within the expression vector pCambia 1201, and the Arabidopsis GS1 coding sequence included in SEQ ID NO: 6 under the control of the RuBisCo promoter within the expression vector pCambia 1201, using Agrobacterium-mediated transfer into flowers.

Materials and Methods:

The transgene expression vectors pCambia 1201-GPT (including construct of SEQ ID NO: 27) and pCambia 1201-GS (including construct of SEQ ID NO: 6) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of chloroamphenicol. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Bean plants were then transformed with a mixture of the resulting Agrobacterium cell suspensions using a transformation protocol in which the Agrobacteria is injected directly into the flower structure (Yasseem, 2009, Plant Mol. Biol. Reporter 27: 20-28). In order to induce Agrobacteria virulence and improve transformation efficiencies, 10 μg/ml acetosyringonone was added to the Agrobacteria cultures prior to flower inoculation. Briefly, once flowers bloomed, the outer structure encapsulating the reproductive organs was gently opened with forceps in order to permit the introduction of the Agrobacteria mixture, which was added to the flower structure sufficient to flood the anthers.

Plants were grown until bean pods developed, and seeds were harvested and used to generate transgenic plants. Transgenic plants were then grown together with control bean plants under identical conditions, photographed and phenotypically characterized. Growth rates were measured for both transgenic and control plants. In this and all examples, Glutamine synthetase (GS) activity was assayed according to the methods in Shapiro and Stadtmann, 1970, Methods in Enzymology 17A: 910-922; and, Glutamine phenylpyruvate transaminase (GPT) activity was assayed according to the methods in Calderon et al., 1985, J. Bacteriol. 161: 807-809. See details in Example 7, Methods, supra.

Results:

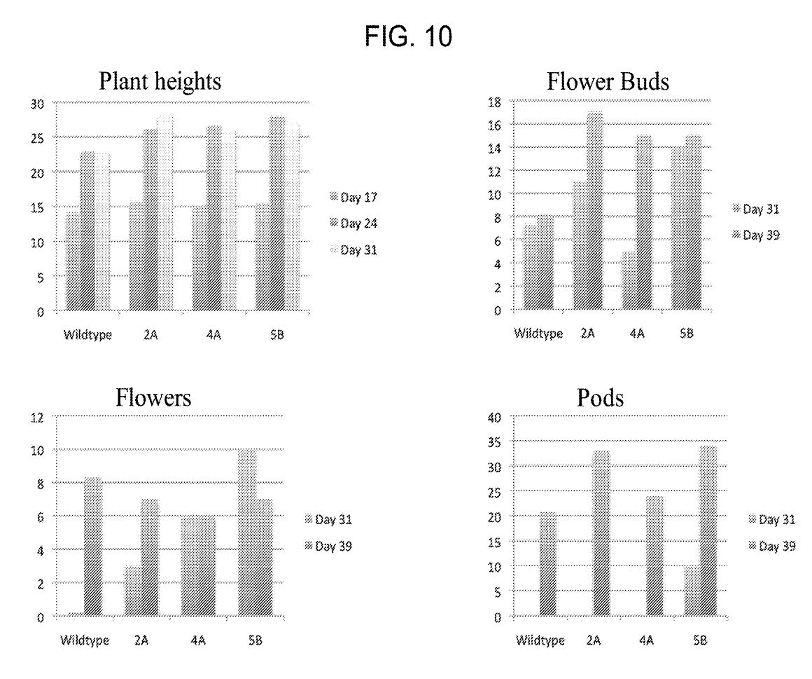

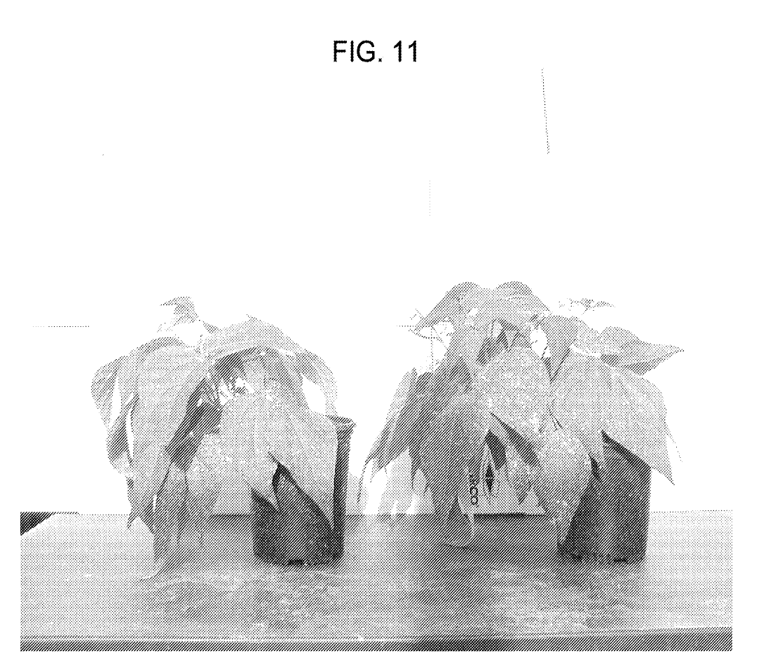

The results are presented in FIG. 10, FIG. 11 and Table VI.

FIG. 10 shows GPT+GS transgenic bean line A growth rate data relative to control plants, including plant heights on various days into cultivation, as well as numbers of flower buds, flowers, and bean pods. These data show that the GPT+GS double transgenic bean plants outgrew their counterpart control plants. The transgenic plants grew taller, flowered earlier and produced more flower buds and flowers, and developed bean pods and produced more bean pods that the wild type control plants.

TABLE VI
TRANSGENIC BEANS LINE A

| | | GPT | GS | |
|---|---|---|---|---|
| | Bean Pod | Activity | Activity | |
| | Yield | nmoles/ | umoles/ | Antibiotic |
| Plant Type | FWt, g | h/gFWt | min/gFWt | Resistance |
| Wildtype, avg | 126.6 | 101.9 | 25.2 | Negative |
| 2A | 211.5 | NM | NM | + |
| 4A | 207.7 | NM | NM | + |
| 5B | 205.7 | 984.7 | 101.3 | + |

WT Wildtype;
FWt Fresh Weight;
NM Not Measured

Table VI presents bean pod yield, GPT and GS activity, as well as antibiotic resistance status, in the transgenic lines compared to the wild type control (average of several robust control plants; control plants that did not grow well were excluded from the analyses). Referring to Table VI, double-transgene progeny plants showed substantial bean pod biomass increases (fresh pod weight) in comparison to the control plants, with bean pod biomass yields consistently above 200 grams per individual transgenic plant, compared to an average of 127 grams per wild type plant, representing an over 60% increase in pod yield in the double transgene lines relative to control plant(s).

Lastly, FIG. 11 shows a photograph of a GPT+GS double transgenic bean plant compared to a control plant grown for the same time under identical conditions, showing increased growth in the transgenic plant.

### Example 10: Generation of Double Transgenic Bean Plants Carrying Arabidopsis GS1 and Grape GPT Transgenes

In this example, yellow wax bean plants (Phaseolus vulgaris) were transformed with the Grape GPT full length coding sequence included in SEQ ID NO: 8 under the control of the RuBisCo promoter within the expression vector pCambia 1305.1, and the Arabidopsis GS1 coding sequence included in SEQ ID NO: 6 under the control of the RuBisCo promoter within the expression vector pCambia 1201, using Agrobacterium-mediated transfer into developing pods.

Materials and Methods:

The transgene expression vectors pCambia 1201-GPT(grape) (including construct of SEQ ID NO: 8) and pCambia 1201-GS (including construct of SEQ ID NO: 6) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of chloroamphenicol. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Bean plants were then transformed with a mixture of the resulting Agrobacterium cell suspensions using a transformation protocol in which the Agrobacteria is injected directly into the flower structure. In order to induce Agrobacteria virulence and improve transformation efficiencies, 10 μg/ml acetosyringonone was added to the Agrobacteria cultures prior to flower inoculation. Briefly, once flowers bloomed, the outer structure encapsulating the reproductive organs was gently opened with forceps in order to permit the introduction of the Agrobacteria mixture, which was added to the flower structure sufficient to flood the anthers.

Plants were grown until bean pods developed, and seeds were harvested and used to generate transgenic plants. Transgenic plants were then grown together with control bean plants under identical conditions, photographed and phenotypically characterized. Growth rates were measured for both transgenic and control plants.

Results:

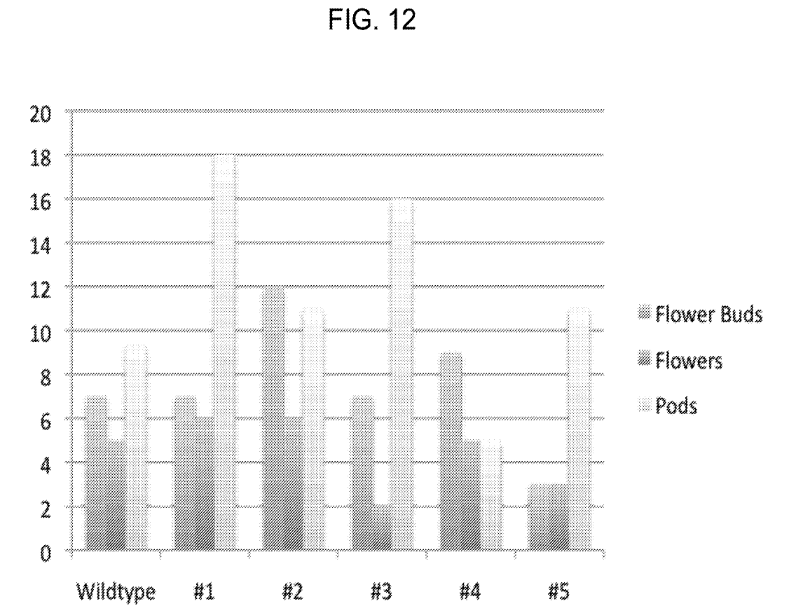

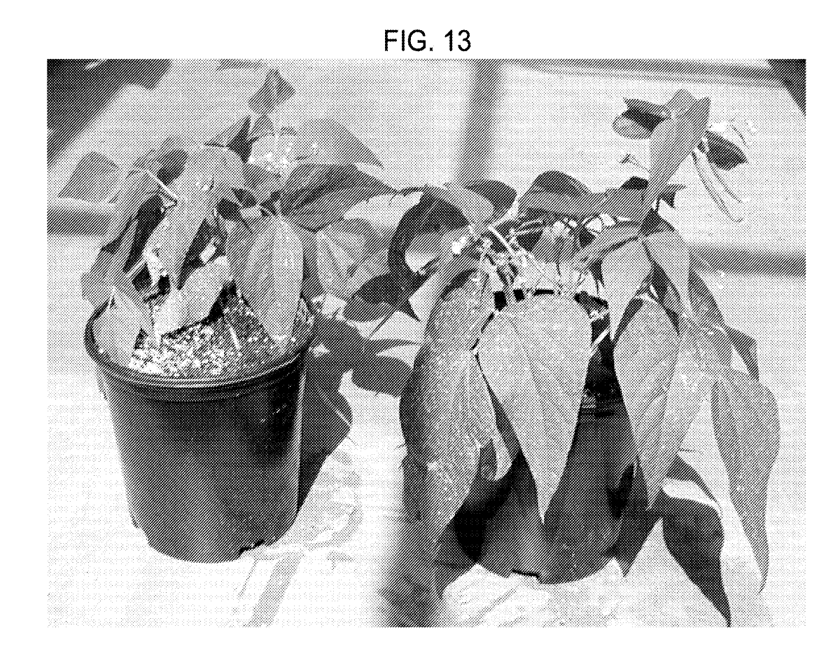

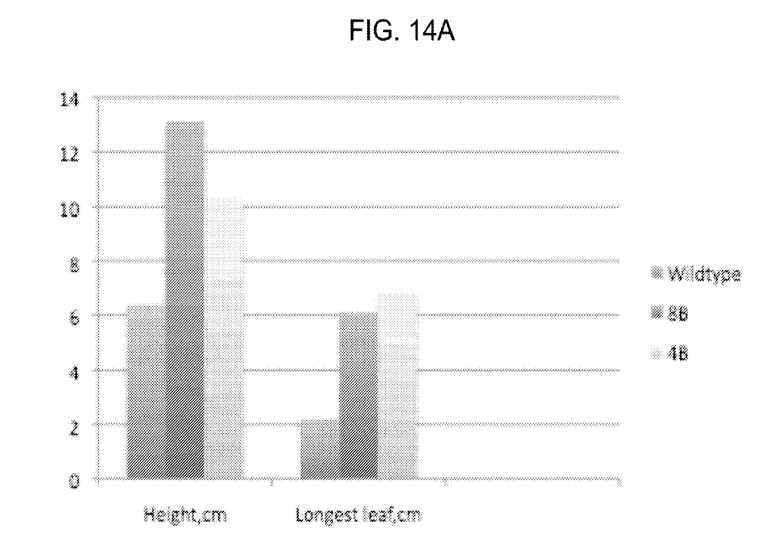

The results are presented in FIG. 12, FIG. 13 and Table VII.

FIG. 12 shows GPT+GS transgenic bean line G growth rate data relative to control plants, specifically including numbers of flower buds, flowers, and bean pods. These data show that the GPT+GS double transgenic bean plants outgrew their counterpart control plants. Notably, the transgenic plants produced substantially more bean pods that the wild type control plants.

TABLE VII
TRANSGENIC BEANS LINE G: POD YIELDS

| | Bean Pod | |
|---|---|---|
| | Yield | Antibiotic |
| Plant Type | FWt, g | Resistance |
| Wild type, avg | 157.9 | Negative |
| G1 | 200.5 | + |
| G2 | 178.3 | + |

WT Wildtype;
FWt Fresh Weight;
NM Not Measured

Table VII presents bean pod yield and antibiotic resistance status, in the transgenic lines compared to the wild type control (average of several robust control plants; control plants that did not grow well were excluded from the analyses). Referring to Table VII, double-transgene progeny plants showed substantial bean pod biomass increases (fresh pod weight) in comparison to the control plants, with bean pod biomass yields of 200.5 (line G1) and 178 grams (line G2) per individual transgenic plant, compared to an average of 158 grams per individual wild type plant, representing approximately a 27% increase in pod yield in the double transgene lines relative to control plants.

Lastly, FIG. 13 shows a photograph of a GPT+GS double transgenic bean plant compared to a control plant grown for the same time under identical conditions. The transgenic plant shows substantially increased size and biomass, larger leaves and a more mature flowering compared to the control plant.

### Example 11: Generation of Double Transgenic Cowpea Plants Carrying Arabidopsis GS1 and GPT Transgenes

In this example, common Cowpea plants were transformed with the Arabidopsis GPT full length coding sequence of SEQ ID NO: 1 under the control of the CMV 35S promoter within the expression vector pMON, and the Arabidopsis GS1 coding sequence included in SEQ ID NO: 6 under the control of the RuBisCo promoter within the expression vector pCambia 1201, using Agrobacterium-mediated transfer into flowers. Materials and methods were as in Example 9, supra.

Results:

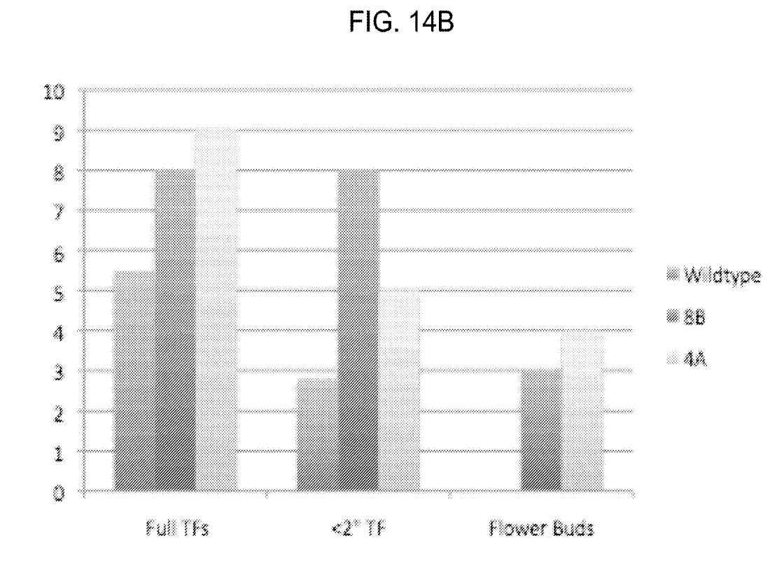

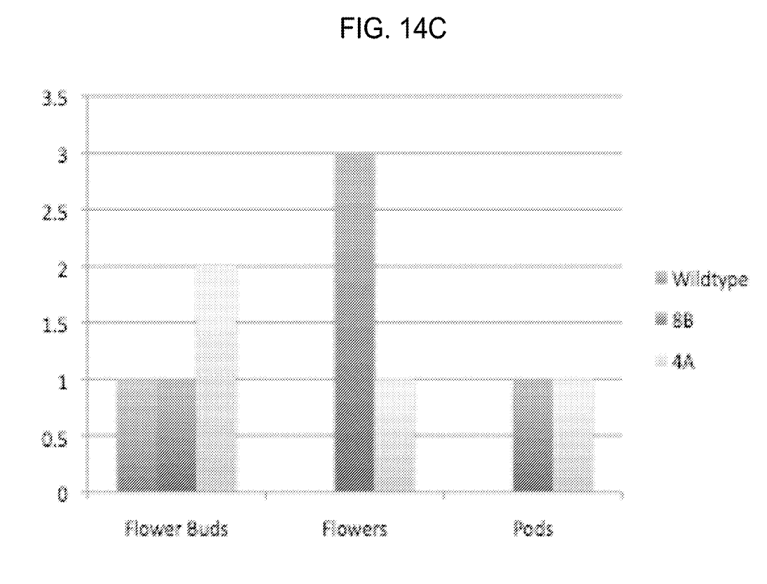

The results are presented in FIGS. 14A-C and 15, and Table VI. FIGS. 14A-C shows relative growth rates for the GPT+GS transgenic Cowpea line A and wild type control Cowpea at several intervals during cultivation, including (FIG. 14A) height and longest leaf measurements, (FIG. 14B) trifolate leafs and flower buds, and (FIG. 14C) flowers, flower buds and pea pods. These data show that the GPT+GS double transgenic Cowpea plants outgrew their counterpart control plants. The transgenic plants grew faster and taller, had longer leaves, and set flowers and pods sooner than wild type control plants.

TABLE VIII
TRANSGENIC COWPEA LINE A

| | | GPT | GS | |
|---|---|---|---|---|
| | Pea Pod | Activity | Activity | |
| | Yield, | nmoles/ | umol/ | Antibiotic |
| Plant Type | FWt, g | h/gFWt | min/gFWt | Resistance |
| Wildtype, avg | 74.7 | 44.4 | 28.3 | Negative |
| 4A | 112.8 | NM | 41.3 | + |
| 8B | 113.8 | 736.2 | 54.9 | + |

WT Wildtype;
FWt Fresh Weight;
NM Not Measured

Table VIII presents pea pod yield, GPT and GS activity, as well as antibiotic resistance status, in the transgenic lines compared to the wild type control (average of several robust control plants; control plants that did not grow well were excluded from the analyses).

Referring to Table VIII, double-transgene progeny plants showed substantial pea pod biomass increases (fresh pod weight) in comparison to the control plants, with average transgenic plant pea pod biomass yields nearly 52% greater than the yields measured in control plant(s).

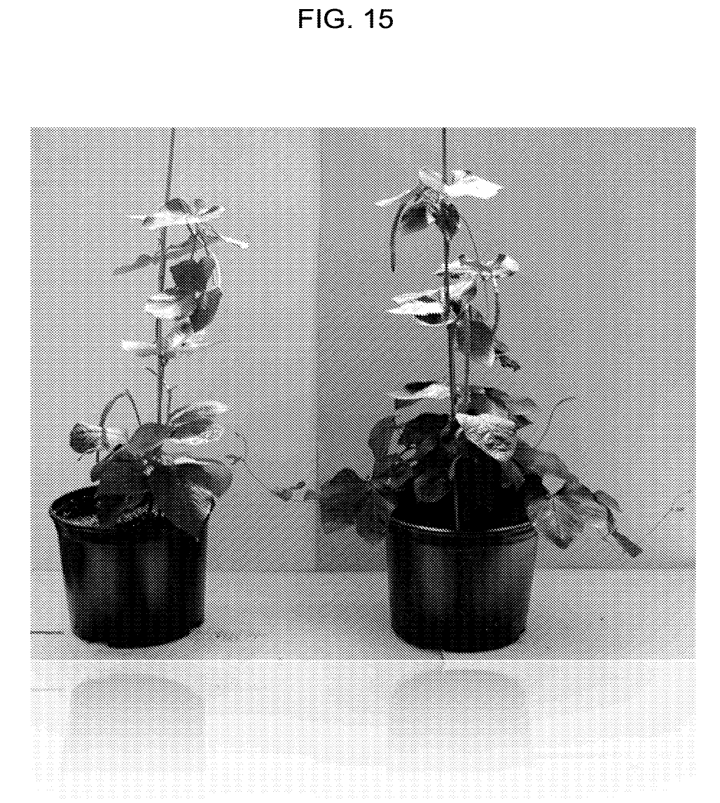

Lastly, FIG. 15 shows a photograph of a GPT+GS double transgenic bean plant compared to a control plant grown for the same time under identical conditions, showing increased biomass and pod yield in the transgenic plant relative to the wild type control plant.

### Example 12: Generation of Double Transgenic Cowpea Plants Carrying Arabidopsis GS1 and Grape GPT Transgenes

In this example, common Cowpea plants were transformed with the Grape GPT full length coding sequence included in SEQ ID NO: 8 under the control of the RuBisCo promoter within the expression vector pCambia 1305.1 (including construct of SEQ ID NO: 8), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer into flowers. Materials and methods were as in Example 11, supra.

Results:

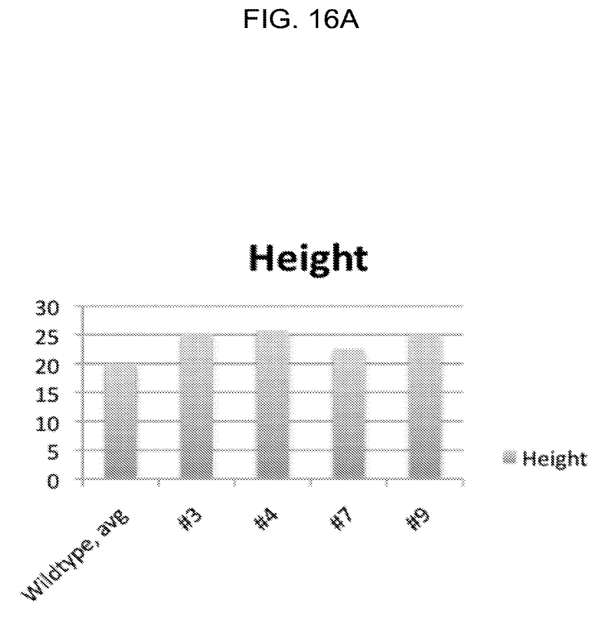

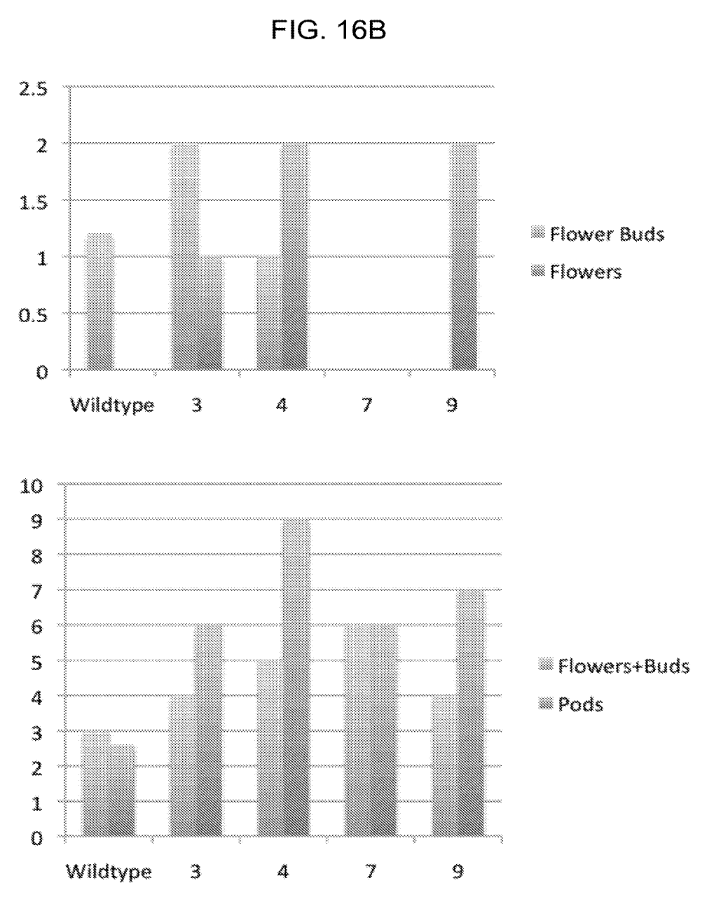

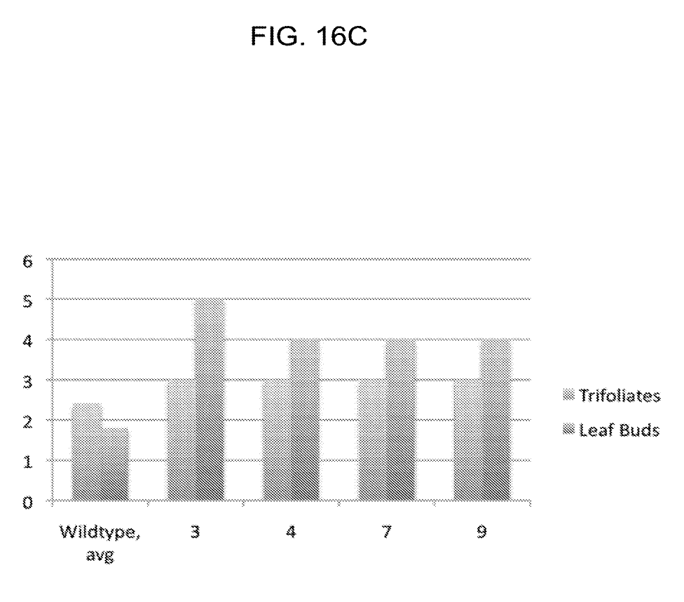

The results are presented in FIGS. 16A-C and 17, and Table IX.

FIGS. 16A-C shows relative growth rates for the GPT+GS transgenic Cowpea line G and wild type control Cowpea. These data show that the transgenic plants are consistently higher (FIG. 16A), produce substantially more flowers, flower buds and pea pods (FIG. 16B), and develop trifolates and leaf buds faster (FIG. 16C).

TABLE IX
TRANSGENIC COWPEA LINE G

| | | GPT | GS | |
|---|---|---|---|---|
| | Pod | Activity | Activity | |
| | Yield, | nmoles/ | umol/ | Antibiotic |
| Plant Type | FWt, g | h/gFWT | min/gFWt | Resistance |
| Wildtype, avg | 59.7 | 44.4 | 26.7 | Negative |
| G9 | 102.0 | 555.6 | 34.5 | + |

WT Wildtype;
FWt Fresh Weight;
NM Not Measured

Table IX presents pea pod yield, GPT and GS activity, as well as antibiotic resistance status, in the transgenic lines compared to the wild type control (average of several robust control plants; control plants that did not grow well were excluded from the analyses). Referring to Table IX, double-transgene progeny plants showed substantial pea pod biomass increases (fresh pod weight) in comparison to the control plants, with average pea pod biomass yields 70% greater in the transgenic plants compared to control plant(s).

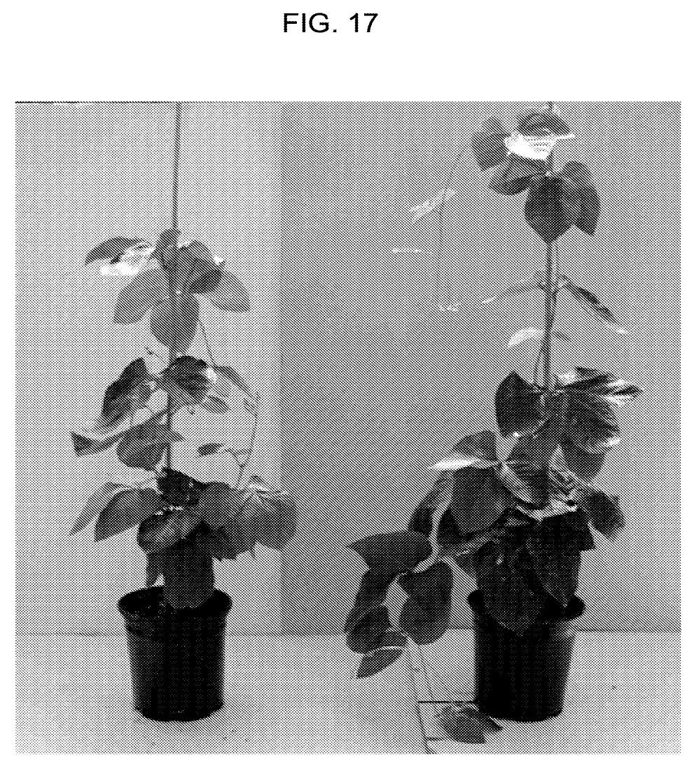

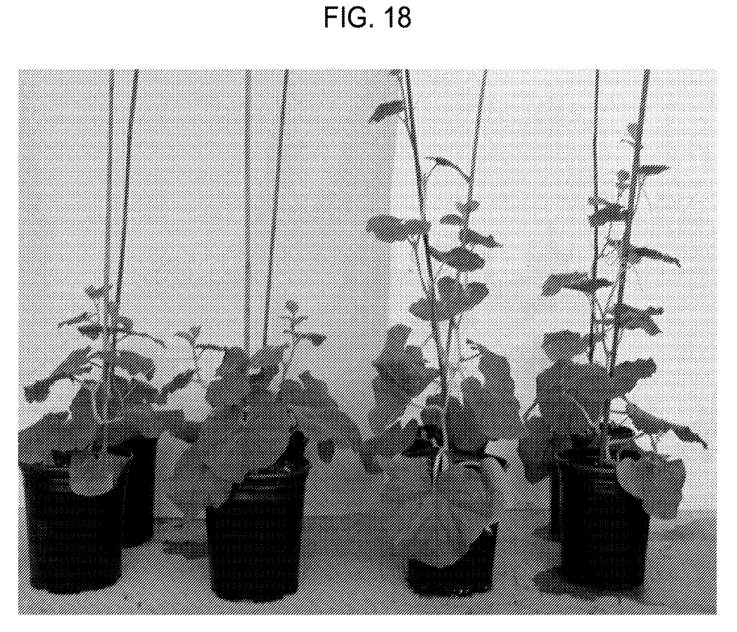

Lastly, FIG. 17 shows a photograph of a GPT+GS double transgenic pea plant compared to a control plant grown for the same time under identical conditions, showing increased height, biomass and leaf size in the transgenic plant relative to the wild type control plant.

### Example 13: Generation of Double Transgenic Alfalfa Plants Carrying Arabidopsis GS1 and GPT Transgenes

In this example, Alfalfa plants (Medicago sativa, var Ladak) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer into seedling plants. Agrobacterium vectors and mixtures were prepared for seedling inoculations as described in Example 11, supra.

Seedling Inoculations:

When Alfalfa seedlings were still less than about ½ inch tall, they were soaked in paper toweling that had been flooded with the Agrobacteria mixture containing both transgene constructs. The seedlings were left in the paper toweling for two to three days, removed and then planted in potting soil. Resulting T0 and control plants were then grown for the first 30 days in a growth chamber, thereafter cultivated in a greenhouse, and then harvested 42 days after sprouting. At this point, only the transgenic Alfalfa line displayed flowers, as the wild type plants only displayed immature flower buds. The plants were characterized as to flowering status and total biomass.

Results:

The results are presented in Table X. The data shows that the transgenic Alfalfa plants grew faster, flowered sooner, and yielded on average about a 62% biomass increase relative to the control plants.

TABLE X
TRANSGENIC ALFALFA VS. CONTROL

| | Biomass | |
|---|---|---|
| | at Sacrifice, | Flowering |
| Plant Type | g | Stage |
| Wildtype, avg | 6.03 | Small defined buds |
| | | No buds swelling. |
| | | No flowers |
| Transgene #5 | 10.38 | 4 Open flowers |
| Transgene #11 | 9.03 | Flower buds swelling |
| Transgene #13 | 9.95 | Flower buds swelling |

### Example 14: Generation of Double Transgenic Cantaloupe Plants Carrying Arabidopsis GS1 and GPT Transgenes

In this example, Cantaloupe plants (Cucumis melo var common) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer via injection into developing melons. Agrobacterium vectors and mixtures were prepared for intra-melon inoculations as described in Example 8, supra. Inoculations into developing melons were carried out essentially as described in Example 8. The plants were characterized as to flowering status and total biomass relative to control melon plants grown under identical conditions.

The results are presented in FIG. 18 and Table XI. Referring to Table XI, the transgenic plants showed substantial foliar plant biomass increases in comparison to the control plants, with an average increase in biomass of 63%. Moreover, a tremendous increase in flower and flower bud yields was observed in all five transgenic lines. Control plants displayed no flowers and only 5 buds at sacrifice, on average. In sharp contrast, the transgenic plants displayed between 2 and 5 flowers per plant, and between 21 and 30 flower buds, per plant, indicating a substantially higher growth rate and flower yield. Increased flower yield would be expected to translate into correspondingly higher melon yields in the transgenic plants. Referring to FIG. 18 (a photograph comparing transgenic Cantaloupe plants to control Cantaloupe plants), the transgenic Cantaloupe plants show dramatically increased height, overall biomass and flowering status relative to the control plants.

TABLE XI
TRANGENIC CANTALOUPE VERSUS CONTROL

| | Biomass | Flowers/ | |
|---|---|---|---|
| | Foliar | Flower Buds | Antibiotic |
| Plant Type | FWt, g | at Sacrifice | Resistance |
| Wildtype, avg | 22.8 | 0/5 | Negative |
| Line 1 | 37.0 | 3/21 | + |
| Line 2 | 35.0 | 2/30 | + |
| Line 3 | 37.1 | 3/27 | + |
| Line 4 | 40.6 | 5/26 | + |
| Line 5 | 35.7 | 4/30 | + |

FWt Fresh Weight

### Example 15: Generation of Double Transgenic Pumpkin Plants Carrying Arabidopsis GS1 and GPT Transgenes

In this example, common Pumpkin plants (Cucurbita maxima) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer via injection into developing pumpkins, essentially as described in Example 14, supra. The transgenic and control pumpkin plants were grown under identical conditions until the emergence of flower buds in the control plants, then all plants were characterized as to flowering status and total biomass.

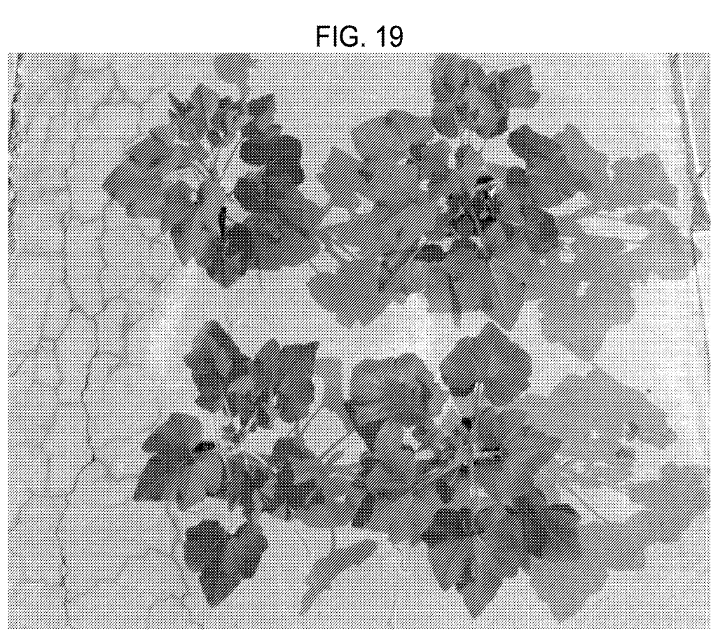

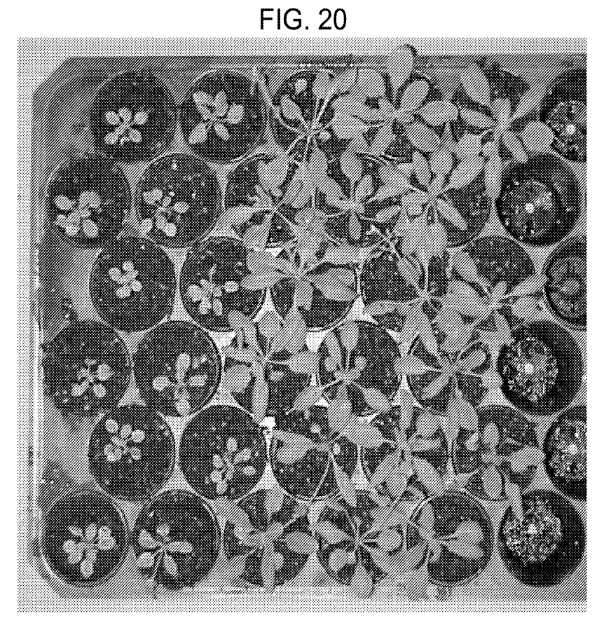

The results are presented in FIG. 19 and Table XII. Referring to Table XII, the transgenic plants showed substantial foliar plant biomass increases in comparison to the control plants, with an increase in average biomass yield of 67% over control plants. Moreover, an increase in flower bud yields was observed in four of the five transgenic lines in comparison to control. Control plants displayed only 4 buds at sacrifice (average). In contrast, four transgenic plant lines displayed between 8 and 15 flowers buds per plant, representing a two- to nearly four-fold yield increase.

TABLE XII
TRANGENIC PUMPKIN VERSUS CONTROL

| | Biomass | | |
|---|---|---|---|
| | Foliar | Flower Buds | Antibiotic |
| Plant Type | FWt, g | at Sacrifice | Resistance |
| Wildtype, avg | 47.7 | 4.2 | Negative |
| Line 1 (Photo) | 82.3 | 8 | |
| Line 2 | 74.3 | 8 | + |
| Line 3 | 80.3 | 9 | + |
| Line 4 (Photo) | 77.8 | 4 | + |
| Line 5 | 84.5 | 15 | + |

FWt Fresh Weight;

Referring to FIG. 19 (a photograph comparing transgenic pumpkin plants to control plants), the transgenic pumpkin plants show substantially increased plant size, overall biomass and leaf sizes and numbers relative to the control plants.

### Example 16: Generation of Double Transgenic Arabidopsis Plants Carrying Arabidopsis GS1 and GPT Transgenes

In this example, Arabidopsis thaliana plants were transformed with the truncated Arabidopsis GPT coding sequence of SEQ ID NO: 18 under the control of the CMV 35S promoter within the expression vector pMON316 (see Example 3, supra), and transgenic plants thereafter transformed with the Arabidopsis GS1 coding sequence included in SEQ ID NO: 6 under the control of the RuBisCo promoter within the expression vector pCambia 1201 (including construct of SEQ ID NO: 6), using Agrobacterium-mediated “floral dip” transfer as described (Harrison et al., 2006, Plant Methods 2:19-23; Clough and Bent, 1998, Plant J. 16:735-743). Agrobacterium vectors pMON316 carrying GPT and pCambia 1201 carrying GS1 were prepared as described in Examples 3 and 11, respectively.

Transformation of two different cultures of Agrobacterium with either a pMon 316+Arabidopsis GTP construct or with a Cambia 1201+Arabidopsis GS construct was done by electroporation using the method of Weigel and Glazebrook 2002. The transformed Agrobacterium were then grown under antibiotic selection, collected by centrifugation resuspended in LB broth with antibiotic and used in the floral dip of Arabidopsis inflorescence. Floral dipped Arabidopsis plants were taken to maturity and self-fertilized and seeds were collected. Seeds from twice dipped plants were first geminated on a media containing 20 ug/ml of kanamycin and by following regular selection procedures surviving seedlings were transferred to media containing 20 ug of hygromycin. Plants (3) surviving the selection process on both antibiotics were self-fertilized and seeds were collected. Seeds from the T₁ generation were germinated on MS media containing 20 ug/ml of hygromycin and surviving seedlings were taken to maturity, self-fertilized and seeds collected. This seed population the T₂ generation was then used for subsequent growth studies.

The results are presented in FIG. 20 and Table XIII. Referring to Table XIII, which shows data from 6 wild type and 6 transgenic Arabidopsis plants (averaged), the transgenic plants displayed increased levels of both GPT and GS activity. GPT activity was over twenty-fold higher than the control plants. Moreover, the transgenic plant fresh foliar weight average was well over four-fold that of the wild type control plant average. A photograph of young transgene Arabidopsis plants in comparison to wild type control Arabidopsis plants grown under identical conditions is shown in FIG. 20, and reveals a consistent and very significant growth/biomass increase in transgenic plants relative to the control plants.

TABLE XIII
TRANSGENIC ARABIDOPSIS VERSUS CONTROL

| | | GPT | GS | |
|---|---|---|---|---|
| | Biomass, | Activity | Activity | |
| | g Fresh | nmol/ | umol/ | Antibiotic |
| Plant type | foliar wt | h/gFWt | min/gFWt | Resistance |
| Wildtype, avg | 0.246 | 18.4 | 7.0 | Negative |
| Transgene | 1.106 | 395.6 | 18.2 | Positive |

### Example 17: Generation of Transgenic Tomato Plants Carrying Arabidopsis GPT and GS1 Transgenes

In this example, tomato plants (Solanum lycopersicon, “Money Maker” variety) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6). Single transgene (GPT) transgenic tomato plants were generated and grown to flowering essentially as described in Example 4. The Arabidopsis GS1 transgene was then introduced into the single-transgene T0 plants using Agrobacterium-mediated transfer via injection directly into flowers (as described in Example 8). The transgenic and control tomato plants were grown under identical conditions and characterized as to growth phenotype characteristics. Resulting To double-transgene plants were then grown to maturity, photographed along with control tomato plants, and phenotypically characterized.

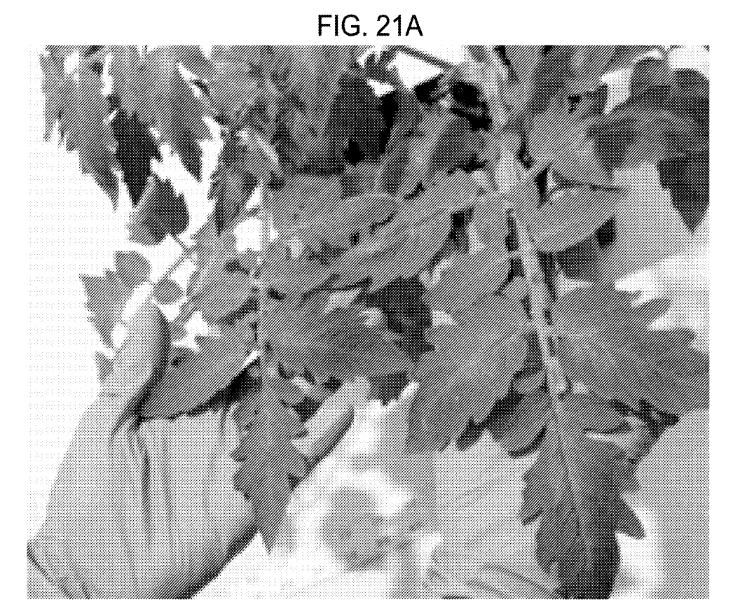

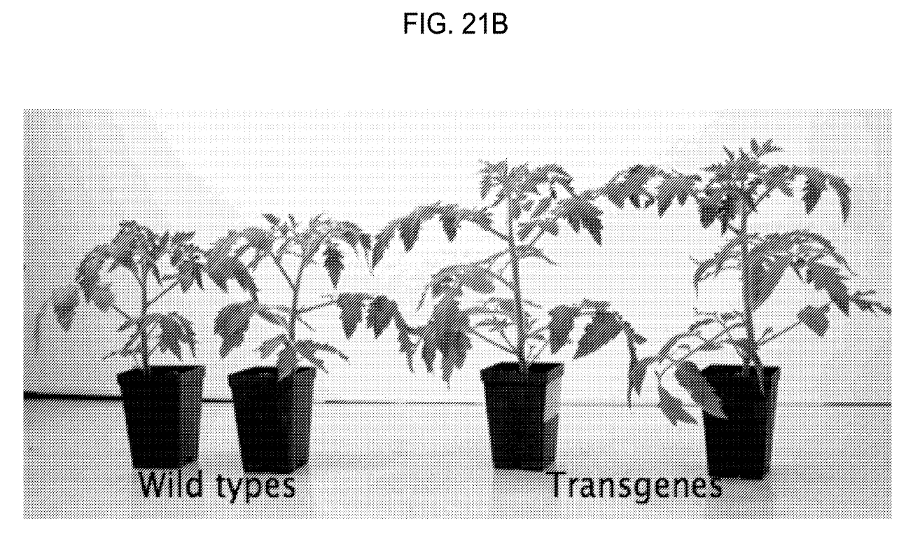

The results are presented in FIGS. 21A-B and in Table XIX. Referring to Table XIX, double-transgene tomato plants showed substantial foliar plant biomass increases in comparison to the control plants, with an increase in average biomass yield of 45% over control. Moreover, as much as a 70% increase in tomato fruit yield was observed in the transgenic lines compared to control plants (e.g., 51 tomatoes harvested from Line 4C, versus and average of approximately 30 tomatoes from control plants). A much higher level of GPT activity was observed in the transgenic plants (e.g., line 4C displaying an approximately 32-fold higher GPT activity in comparison to the average GPT activity measured in control plants). GS activity was also higher in the transgenic plants relative to control plants (almost double in Line 4C).

With respect to growth phenotype, and referring to FIGS. 21A-B, the transgenic tomato plants displayed substantially larger leaves compared to control plants (FIG. 21A). In addition, it can be seen that the transgenic tomato plants were substantially larger, taller and of a greater overall biomass (see FIG. 21B).

TABLE XIX
TRANSGENIC TOMATO GROWTH AND REPRODUCTION

| | | Total | | | |
|---|---|---|---|---|---|
| | | Tomatoes | GPT | GS | |
| | Biomass | Harvested | Activity | Activity | Transgene |
| | Foliar | until | nmoles/ | umoles/ | Presence |
| Plant Type | FWt, g | Sacrifice | h/gFWt | min/gFWt | Assay |
| Wildtype, avg | 891 | 30.2 | 287 | 14.27 | Negative |
| Line 6C | 1288 | 43 | 9181 | 18.3 | + |
| Line 4C | 1146 | 51 | 1718 | 26.4 | + |

### Example 18: Generation of Transgenic Camilena Plants Carrying Arabidopsis GPT and GS1 Transgenes

In this example, Camelina plants (Camelina sativa, Var MT 303) were transformed with the Arabidopsis GPT full length coding sequence of SEQ ID NO: 1 under the control of the RuBisCo promoter within the expression vector pCambia 1201, and the Arabidopsis GS1 coding sequence included in SEQ ID NO: 6 under the control of the RuBisCo promoter within the expression vector pCambia 1201, using Agrobacterium-mediated transfer into germinating seeds according to the method described in Chee et al., 1989, Plant Physiol. 91: 1212-1218. Agrobacterium vectors and mixtures were prepared for seed inoculations as described in Example 11, supra.

Transgenic and control Camelina plants were grown under identical conditions (30 days in a growth chamber and then moved to greenhouse cultivation) for 39 days, and characterized as to biomass, growth characteristics and flowering stage.

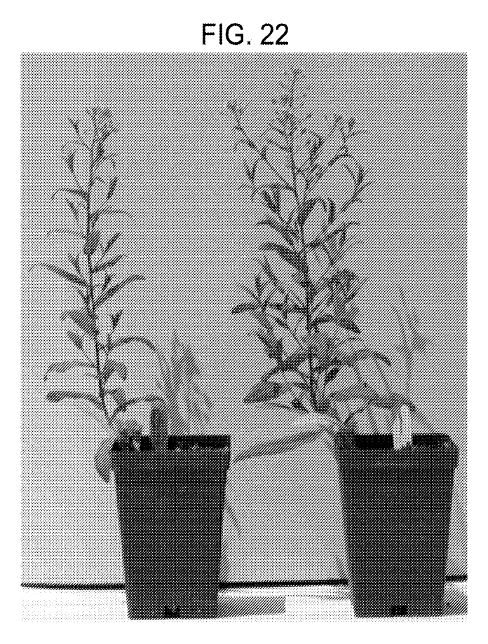

The results are presented in Table XX and FIG. 22. Referring to Table XX, it can be seen that total biomass in the transgenic plants was, on average, almost double control plant biomass. Canopy diameter was also significantly improved in the transgenic plants. FIG. 22 shows a photograph of transgenic Camelina compared to control. The transgenic plant is noticeably larger and displays more advanced flowering.

TABLE XX
TRANSGENIC CAMELINA VERSUS CONTROL

| | Height/Canopy | Biomass | Flowering |
|---|---|---|---|
| Plant Type | Diameter, inches | g | Stage |
| Wildtype, avg | 14/4 | 8.35 | Partial flowering |
| Transgene C-1 | 15.5/5 | 16.54 | Full flowering |
| Transgene C-3 | 14/7 | 14.80 | Initial flowering |

### Example 19: Activity of Barley GPT Transgene in Planta

In this example, the putative coding sequence for Barley GPT was isolated and expressed from a transgene construct using an in planta transient expression assay. Biologically active recombinant Barley GPT was produced, and catalyzed the increased synthesis of 2-oxoglutaramate, as confirmed by HPLC.

The Barley (Hordeum vulgare) GPT coding sequence was determined and synthesized. The DNA sequence of the Barley GPT coding sequence used in this example is provided in SEQ ID NO: 14, and the encoded GPT protein amino acid sequence is presented in SEQ ID NO: 15.

The coding sequence for Barley GPT was inserted into the 1305.1 cambia vector, and transferred to Agrobacterium tumefaciens strain LBA404 using a standard electroporation method (McCormac et al., 1998, Molecular Biotechnology 9:155-159), followed by plating on LB plates Agrobacterium containing hygromycin (50 micro gm/ml). Antibiotic resistant colonies of were selected for analysis.

The transient tobacco leaf expression assay consisted of injecting a suspension of transformed Agrobacterium (1.5-2.0 OD 650) into rapidly growing tobacco leaves. Intradermal injections were made in a grid across the leaf surface to assure that a significant amount of the leaf surface would be exposed to the Agrobacterium. The plant was then allowed to grow for 3-5 days when the tissue was extracted as described for all other tissue extractions and the GPT activity measured.

GPT activity in the inoculated leaf tissue (1217 nanomoles/gFWt/h) was three-fold the level measured in the control plant leaf tissue (407 nanomoles/gFWt/h), indicating that the Hordeum GPT construct directed the expression of biologically active GPT in a transgenic plant.

### Example 20: Isolation and Expression of Recombinant Rice GPT Gene Coding Sequence and Analysis of Biological Activity

In this example, the putative coding sequence for rice GPT was isolated and expressed in E. coli. Biologically active recombinant rice GPT was produced, and catalyzed the increased synthesis of 2-oxoglutaramate, as confirmed by HPLC.

Materials and Methods:

Rice GPT Coding Sequence and Expression in E. coli:

The rice (Oryza saliva) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×106 cells were then assayed for biological activity using the NMR assay, below. Untransformed, wild type E. coli cells were assayed as a control. An additional control used E. coli cells transformed with an empty vector.

The DNA sequence of the rice GPT coding sequence used in this example is provided in SEQ ID NO: 10, and the encoded GPT protein amino acid sequence is presented in SEQ ID NO: 11.

HPLC Assay for 2-Oxoglutaramate:

HPLC was used to determine 2-oxoglutaramate production in GPT-overexpressing E. coli cells, following a modification of Calderon et al., 1985, J Bacteriol 161(2): 807-809. Briefly, a modified extraction buffer consisting of 25 mM Tris-HCl pH 8.5, 1 mM EDTA, 20 μM Pyridoxal phosphate, 10 mM Cysteine, and -1.5% (v/v) Mercaptoethanol was used. Samples (lysate from E. coli cells, 25×106 cells) were added to the extraction buffer at approximately a 1/3 ratio (w/v), incubated for 30 minutes at 37° C., and stopped with 200 μl of 20% TCA. After about 5 minutes, the assay mixture is centrifuged and the supernatant used to quantify 2-oxoglutaramate by HPLC, using an ION-300 7.8 mm ID×30 cm L column, with a mobile phase in 0.01 N H₂SO₄, a flow rate of approximately 0.2 ml/min, at 40° C. Injection volume is approximately 20 μl, and retention time between about 38 and 39 minutes. Detection is achieved with 210 nm UV light.

NMR analysis comparison with authentic 2-oxoglutaramate was used to establish that the Arabidopisis full length sequence expresses a GPT with 2-oxoglutaramate synthesis activity. Briefly, authentic 2-oxoglutarmate (structure confirmed with NMR) made by chemical synthesis to validate the HPLC assay, above, by confirming that the product of the assay (molecule synthesized in response to the expressed GPT) and the authentic 2-oxoglutaramate elute at the same retention time. In addition, when mixed together the assay product and the authentic compound elute as a single peak. Furthermore, the validation of the HPLC assay also included monitoring the disappearance of the substrate glutamine and showing that there was a 1:1 molar stoichiometry between glutamine consumed to 2-oxoglutaramte produced. The assay procedure always included two controls, one without the enzyme added and one without the glutamine added. The first shows that the production of the 2-oxoglutaramate was dependent upon having the enzyme present, and the second shows that the production of the 2-oxoglutaramate was dependent upon the substrate glutamine.

Results:

Expression of the rice GPT coding sequence of SEQ ID NO: 10 resulted in the over-expression of recombinant GPT protein having 2-oxoglutaramate synthesis-catalyzing bioactivity. Specifically, 1.72 nanomoles of 2-oxoglutaramate activity was observed in the E. coli cells overexpressing the recombinant rice GPT, compared to only 0.02 nanomoles of 2-oxoglutaramate activity in control E. coli cells, an 86-fold activity level increase over control.

### Example 21: Isolation and Expression of Recombinant Soybean GPT Gene Coding Sequence and Analysis of Biological Activity

In this example, the putative coding sequence for soybean GPT was isolated and expressed in E. coli. Biologically active recombinant soybean GPT was produced, and catalyzed the increased synthesis of 2-oxoglutaramate, as confirmed by HPLC.

Materials and Methods:

Soybean GPT Coding Sequence and Expression in E. coli:

The soybean (Glycine max) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×10⁶ cells were then assayed for biological activity using the HPLC assay, below. Untransformed, wild type E. coli cells were assayed as a control. An additional control used E coli cells transformed with an empty vector.

The DNA sequence of the soybean GPT coding sequence used in this example is provided in SEQ ID NO: 12, and the encoded GPT protein amino acid sequence is presented in SEQ ID NO: 13.

HPLC Assay for 2-Oxoglutaramate:

HPLC was used to determine 2-oxoglutaramate production in GPT-overexpressing E. coli cells, as described in Example 6, supra.

Results:

Expression of the soybean GPT coding sequence of SEQ ID NO: 12 resulted in the over-expression of recombinant GPT protein having 2-oxoglutaramate synthesis-catalyzing bioactivity. Specifically, 31.9 nanomoles of 2-oxoglutaramate activity was observed in the E. coli cells overexpressing the recombinant soybean GPT, compared to only 0.02 nanomoles of 2-oxoglutaramate activity in control E. coli cells, a nearly 1,600-fold activity level increase over control.

### Example 22: Isolation and Expression of Recombinant Zebra Fish GPT Gene Coding Sequence and Analysis of Biological Activity

In this example, the putative coding sequence for Zebra fish GPT was isolated and expressed in E. coli. Biologically active recombinant Zebra fish GPT was produced, and catalyzed the increased synthesis of 2-oxoglutaramate, as confirmed by HPLC.

Materials and Methods:

Zebra Fish GPT Coding Sequence and Expression in E. coli:

The Zebra fish (Danio rerio) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×10⁶ cells were then assayed for biological activity using the HPLC assay, below. Untransformed, wild type E. coli cells were assayed as a control. An additional control used E coli cells transformed with an empty vector.

The DNA sequence of the Zebra fish GPT coding sequence used in this example is provided in SEQ ID NO: 16, and the encoded GPT protein amino acid sequence is presented in SEQ ID NO: 17.

HPLC Assay for 2-Oxoglutaramate:

HPLC was used to determine 2-oxoglutaramate production in GPT-overexpressing E. coli cells, as described in Example 6, supra.

Results:

Expression of the Zebra fish GPT coding sequence of SEQ ID NO: 16 resulted in the over-expression of recombinant GPT protein having 2-oxoglutaramate synthesis-catalyzing bioactivity. Specifically, 28.6 nanomoles of 2-oxoglutaramate activity was observed in the E. coli cells overexpressing the recombinant Zebra fish GPT, compared to only 0.02 nanomoles of 2-oxoglutaramate activity in control E. coli cells, a more than 1,400-fold activity level increase over control.

### Example 23: Generation and Expression of Recombinant Truncated Arabidopsis GPT Gene Coding Sequences and Analysis of Biological Activity

In this example, two different truncations of the Arabidopsis GPT coding sequence were designed and expressed in E. coli, in order to evaluate the activity of GPT proteins in which the putative chloroplast signal peptide is absent or truncated. Recombinant truncated GPT proteins corresponding to the full length Arabidopsis GPT amino acid sequence of SEQ ID NO: 2, truncated to delete either the first 30 amino-terminal amino acid residues, or the first 45 amino-terminal amino acid residues, were successfully expressed and showed biological activity in catalyzing the increased synthesis of 2-oxoglutaramate, as confirmed by HPLC.

Materials and Methods:

Truncated Arabidopsis GPT Coding Sequences and Expression in E. coli:

The DNA coding sequence of a truncation of the Arabidopsis thaliana GPT coding sequence of SEQ ID NO: 1 was designed, synthesized, inserted into a PET28 vector, and expressed in E. coli. The DNA sequence of the truncated Arabidopsis GPT coding sequence used in this example is provided in SEQ ID NO: 20 (˜45 AA construct), and the corresponding truncated GPT protein amino acid sequence is provided in SEQ ID NO: 21. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×10⁶ cells were then assayed for biological activity using HPLC as described in Example 20. Untransformed, wild type E. coli cells were assayed as a control. An additional control used E coli cells transformed with an empty vector.

Expression of the truncated −45 Arabidopsis GPT coding sequence of SEQ ID NO: 20 resulted in the over-expression of biologically active recombinant GPT protein (2-oxoglutaramate synthesis-catalyzing bioactivity). Specifically, 16.1 nanomoles of 2-oxoglutaramate activity was observed in the E. coli cells overexpressing the truncated −45 GPT, compared to only 0.02 nanomoles of 2-oxoglutaramate activity in control E. coli cells, a more than 800-fold activity level increase over control. For comparison, the full length Arabidopsis gene coding sequence expressed in the same E. coli assay generated 2.8 nanomoles of 2-oxoglutaramate activity, or roughly less than one-fifth the activity observed from the truncated recombinant GPT protein.

### Example 24: GPT+GS Transgenic Tobacco Seed Germination Tolerates High Salt Concentrations

In this example, seeds form the double transgene tobacco line XX-3 (Cross 3 in Table 4, see Example 7) were tested in a seed germination assay designed to evaluate tolerance to high salt concentrations.

Materials and Methods:

Tobacco seeds from the wild type and XX-3 populations were surfaced sterilized (5% bleach solution for 5 minutes followed by a 10% ethanol wash for 3 minutes) and rinsed with sterile distilled water. The surface sterilized seeds were then spread on Murashige and Skoog media (10% agarose) without sucrose and containing either 0 or 200 mM NaCl. The seeds were allowed to germinate in darkness for 2 days followed by 6 days under a 16:8 photoperiod at 24° C. On day eight the rate of germination was determined by measuring the percentage of seeds from the control or transgene plants that had germinated.

Results:

The results are tabulated in Table)(XI below. The rate of germination of the transgenic plant line seeds under zero salt conditions was the same as observed with wild type control plant seeds. In stark contrast, the germination rate of the transgenic plant line seeds under very high salt conditions far exceeded the rate seen in wild type control seeds. Whereas over 81% of the transgenic plant seeds had germinated under the high salt conditions, only about 9% of the wild type control plant seeds had germinated by the same time point. These data indicate that the transgenic seeds are capable of germinating very well under high salt concentrations, an important trait for plant growth in areas of increasingly high water and/or soil salinity.

TABLE XXI
TRANSGENIC TOBACCO PLANTS GERMINATE
AND TOLERATE HIGH SALT

| | Control (0 mM NaCl) | Test (200 mM NaCl) |
|---|---|---|
| Plant type | % Germination | % Germination |
| Wild type | 92, 87, 94 | 9, 11, 8 |
| Transgene line XX-3 | 92, 91, 94 | 84, 82, 78 |

### Example 25: Method for Generating Transgenic Maize Plants Carrying Hordeum GPT and GS1 Transgenes

This example provides a method for generating transgenic maize plants expressing GPT and GS1 transgenes. Maize (Zea mays, hybrid line Hi-II) type II callus is biolistically transformed with an expression cassette comprising the Hordeum glutamine synthetase (GS1) coding sequence of SEQ ID NO: 40 under the control of the rice RuBisCo small subunit promoter of SEQ ID NO: 39 (expression cassette of SEQ ID NO: 42), and the Hordeum GPT coding sequence of SEQ ID NO: 45 under the control of the corn ubiquitin (Ubil) promoter of SEQ ID NO: 44. Transformation of maize callus is achieved by particle bombardment.

Vector Constructs:

An expression cassette comprising the Hordeum GS1 and GPT genes, under the control of the rice RuBisCo small subunit and corn ubiquitin promoters, respectively, is cloned into the plasmid pAHC25 (Christensen and Quail, 1996, Transgenic Research 5:213-218) modified to include a bar gene conferring resistance to bialophos, or a similar vector, in order to generate the transgene expression vector.

Transformation and Regeneration:

The transgene expression vector is introduced into immature zygotic embryo source callus of parent maize hybrid line Hi-II (A1.88×B73 origin) (Armstrong et al., 1991, Maize Genetics Coop Newsletter 65:92-93) using particle bombardment, essentially as described (Frame et al., 2000, In Vitro Cell. Dev. Biol-Plant 36:21-29; this method was developed by and is routinely used at the Iowa State University Center for Plant Transformation).

More specifically, immature zygotic embryo source callus is prepared for transformation by serial culturing on a callus-initiating medium (N6E, Songstad et al., 1996, In vitro Cell Dev. Biol. —Plant 32:179-183). Washed gold particles are coated with the plasmid construct and used to bombard the callus with a PDS 1000/He biolistic gun as described (Sanford et al., 1993, Methods in Enzymology 217: 483-509). After 7-10 days on initiation medium, the callus is then transferred to selection medium containing bialophos (N6S, Songstad et al., 1996, supra) and allowed to grow. Following the development of bialophos resistant clones, callus pieces are transferred to a regeneration medium (Armstrong and Green, 1985, Planta 164:207-214) containing bialophos and allowed to grow for several weeks. Thereafter, the resulting plantlets are transferred to regeneration medium without the selection agent, and cultivated.

Transgenic corn plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 26: Method for Generating Transgenic Rice Plants Carrying Hordeum GPT and GS1 Transgenes

This example provides a method for generating transgenic rice plants expressing GPT and GS1 transgenes. Rice (Oryza sativa, Japonica cultivar Nipponbare) type II calus is transformed with the Hordeum glutamine synthetase (GS1) coding sequence of SEQ ID NO: 40 under the control of the rice RuBisCo small subunit promoter of SEQ ID NO: 39 (expression cassette of SEQ ID NO: 42), and the Hordeum GPT coding sequence of SEQ ID NO: 45 under the control of the corn ubiquitin (Ubil) promoter of SEQ ID NO: 44. Transformation is achieved by Agrobacterium-mediated transformation.

Vector Constructs:

An expression cassette comprising the Hordeum GS1 and GPT genes, under the control of the rice RuBisCo small subunit and corn ubiquitin promoters, respectively, is cloned into base vector pTF101.1, using standard molecular cloning methodologies, to generate the transgene expression vector. Base vector pTF101.1 is a derivative of the pPZP binary vector (Hajdukiewicz et al 1994, Plant Mol. Biol. 25:989-994), which includes the right and left T-DNA border fragments from a nopaline strain of A. tumefaciens, a broad host origin of replication (pVS1) and a spectinomycin-resistant marker gene (aadA) for bacterial selection. The plant selectable marker gene cassette includes the phosphinothricin acetyl transferase (bar) gene from Streptomyces hygroscopicus that confers resistance to the herbicides glufosinate and bialophos. The soybean vegetative storage protein terminator (Mason et al., 1993) follows the 3′ end of the bar gene.

Media:

YEP Medium: 5 g/L yeast extract, 10 g/L peptone, 5 g/L NaCl₂, 15 g/L Bacto-agar. pH to 6.8 with NaOH. After autoclaving, the appropriate antibiotics are added to the medium when it has cooled to 50° C.

Infection Medium: N6 salts and vitamins (Chu et al., 1975, Sci. Sinica 18: 659-668), 1.5 mg/L 2,4-dichlorophenoxyacetic acid (2,4-D), 0.7 g/L L-proline, 68.4 g/L sucrose, and 36 g/L glucose (pH 5.2). This medium is filter-sterilized and stored at 4° C. Acetosyringone (AS, 100 μM) is added just prior to use (prepared from 100 μM stocks of filter-sterilized AS, dissolved in DMSO to 200 mM then diluted 1:1 with water).

Callus Induction Medium: N6 salts and vitamins, 300 mg/L casamino acids, 2.8 g/L L-proline, 30 g/L sucrose, and 4 g/L gelrite (pH 5.8). Filter sterilized N6 Vitamins and 2 mg/L 2,4-D, are added to this medium after autoclaving.

Co-cultivation Medium (make fresh): N6 salts and vitamins, 300 mg/L casamino acids, 30 g/L sucrose, 10 g/L glucose, and 4 g/L gelrite (pH 5.8). Filter sterilized N6 vitamins, acetosyringone (AS) 100 μM and 2 mg/L 2,4-D are added to this medium after autoclaving.

Selection Medium: N6 salts and vitamins, 300 mg/L casamino acids, 2.8 g/L L-proline, 30 g/L sucrose, and 4 g/L gelrite (pH 5.8). Filter sterilized N6 vitamins, 2 mg/L 2,4-D, 2 mg/L Bialaphos (Shinyo Sangyo, Japan) and 500 mg/L carbenicillin are added to this medium after autoclaving.

Regeneration Medium I: MS salts and vitamins (Murashige and Skoog, 1962), 2 g/L casamino acids, 30 g/L sucrose, 30 g/L sorbitol, and 4 g/L gelrite (pH 5.8). Filter sterilized MS vitamins, 100 mg/L cefotaxime, 100 mg/L vancomycin, 0.02 mg/L NAA (naphthaleneacetic acid), 2 mg/L kinetin (Toki, 1997, supra) and 2 mg/Bialaphos are added to this medium after autoclaving.

Regeneration Medium II: MS Salts and vitamins, 100 mg/L myo-inositol, 30 g/L sucrose, 3 g/L gelrite, (pH 5.8).

Transformation and Regeneration:

Japonica rice cultivar Nipponbare is transformed with Agrobacterium tumefaciens strain EHA101 (Hood et al., 1986, J. Bacteriol. 168:1291-1301), transformed with the pTF101.1 transgene expression vector carrying the Hordeum GS1+GPT expression cassette. The vector system pTF101.1 in EHA101 is maintained on YEP medium (An et al., 1988) containing 100 mg/L spectinomycin (for pTF101.1) and 50 mg/L kanamycin (for EHA101).

Briefly, callus tissue derived from the mature rice embryo is used as the starting material for transformation. Callus induction, co-cultivation, selection and regeneration I media are based on those of Hiei et al., 1994, The Plant Journal 6 (2):271-282.

More specifically, calli are induced as follows. First, 15-20 rice seeds are dehusked and rinsed in 10 ml of 70% Ethanol (50 ml conical tube) by vigorously shaking the tube for one minute, followed by rinsing once with sterile water. Then, 10 ml of 50% commercial bleach (5.25% hypochlorite) is added and placed on a shaker for 30 minutes (low setting). The bleach solution is then poured-off and the seeds rinsed five times with ˜10 ml of sterilized water each time. With a small portion of the final rinse, the seeds are poured onto sterilized filter paper (in a sterile petri plate) and then allowed to dry. Using sterile forceps, several (i.e., 5) seeds are transferred to the surface of individual sterile petri plates containing callus induction medium. The plates are wrapped with vent tape and incubated in the light (16:8 photoperiod) at 29° C. Seeds are observed every few days and those showing signs of contamination are discarded.

After two to three weeks, developing callus is visible on the scutellum of the mature seed. Calli are then subcultured to fresh induction medium and allowed to proliferate. Four days prior to infection, the callus tissue is cut into 2-4 mm pieces and transferred to fresh induction medium.

The selection medium uses modifications from Toki (Toki, 1997, Plant Molecular Biology Reporter 15:16-21) whereby bialophos (2 mg/L) is employed for plant selection and carbenicillin (500 mg/L) for counter selection against Agrobacterium. Regeneration II medium is as described (Armstrong and Green, 1985, Planta 164:207-214).

Agrobacterium culture is grown (i.e., for 3 days at 19° C., or 2 days at 28° C.) on YEP medium amended with spectinomycin (100 mg/L) and kanamycin (50 mg/L). An aliquot of the culture is then suspended in ˜15 ml of liquid infection medium supplemented with 100 μM AS in a 50 ml conical tube (no pre-induction). The optical density is adjusted to <0.1 (OD₅₅₀=0.06-0.08) before use.

For infection, rice calli are first placed into bacteria-free infection medium+AS (50 ml conical). This pre-wash is removed and replaced with 10 ml of the prepared Agrobacterium suspension (OD₅₅₀<0.1). Then, the conical is fastened onto a vortex shaker (low setting) for two minutes. After infection, calli are poured out of the conical onto a stack of sterile filter paper in a 100×15 petri dish to blot dry. Then, they are transferred off the filter paper and onto the surface of co-cultivation medium with sterile forceps. Co-cultivation plates are wrapped with vent tape and incubated in the dark at 25° C. for three days. After three days of co-cultivation, the calli are washed five times with 5 ml of the liquid infection medium (no AS) supplemented with carbenicillin (500 mg/L) and vancomycin (100 mg/L). Calli are blotted dry on sterile filter paper as before. Individual callus pieces are transferred off the paper and onto selection medium containing 2 mg/L bialaphos. Selection plates are wrapped with parafilm and placed in the light at 29° C.

For selection of stable transformation events, plant tissue is cultured onto fresh selection medium every two weeks. This should be done with the aid of a microscope to look for any evidence of Agrobacterium overgrowth. If overgrowth is noted, the affected calli should be avoided (contaminated calli should not be transferred). The remaining tissue is then carefully transferred, preferably using newly sterilized forceps for each calli. Putative clones begin to appear after six to eight weeks on selection. A clone is recognized as white, actively growing callus and is distinguishable from the brown, unhealthy non-transformed tissue. Individual transgenic events are identified and the white, actively growing tissue is transferred to individual plates in order to produce enough tissue to take to regeneration. Regeneration of transgenic plants is accomplished by selecting new lobes of growth from the callus tissue and transferring them onto Regeneration Medium I (light, 25° C.). After two to three weeks, the maturing tissue is transferred to Regeneration Medium II for germination (light, 25° C.). When the leaves are approximately 4-6 cm long and have developed good-sized roots, the plantlets may be transferred (on an individual basis, typically 7-14 days after germination begins) to soilless mix using sterile conditions.

Transgenic rice plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 27: Method for Generating Transgenic Sugarcane Plants Carrying Hordeum GPT and GS1 Transgenes

This example provides a method for generating transgenic sugarcane plants expressing GPT and GS1 transgenes. Sugarcane (Saccharum spp L) is biolistically transformed with an expression cassette comprising the Hordeum glutamine synthetase (GS1) coding sequence of SEQ ID NO: 40 under the control of the rice RuBisCo small subunit promoter of SEQ ID NO: 39 (expression cassette of SEQ ID NO: 42), and the Hordeum GPT coding sequence of SEQ ID NO: 45 under the control of the corn ubiquitin (Ubil) promoter of SEQ ID NO: 44. Transformation of sugarcane callus is achieved by particle bombardment.

Vector Constructs:

An expression cassette comprising the Hordeum GS1 and GPT genes, under the control of the rice RuBisCo small subunit and corn ubiquitin promoters, respectively, are cloned into a small plasmid well established for sugarcane expression, such as pAHC20 (Thomson et al., 1987, EMBO J. 6:2519-2523), using standard molecular cloning methodologies, to generate the transgene expression vector. The plasmid used contains a selectable marker against either the phospinothricin family of herbicides or the antibiotics geneticin or kanamycin, each of which have been shown effective (Ingelbrecht et al., 1999, Plant Physiology 119:1187-1197; Gallo-Maegher & Irvine, 1996, Crop Science 36:1367-1374).

Transformation and Regeneration:

The plasmid containing the expression cassette encoding the Hordeum GS1 and GPT coding sequences is introduced into embryogenic callus prepared for transformation by the basic method of Gallo-Maegher and Irvine (Gallo-Maegher and Irvine, 1996, supra) and Ingelbrecht et al. (Ingelbrecht et al., 1999, supra) with the improved stimulation of shoot regeneration with thidiazuron (Gallo-Maegher et al., 2000, In vitro Cell Dev. Biol. —Plant 36:37-40). This particle bombardment method is effective in transforming sugarcane (see, for example, Gilbert et al., 2005, Crop Science 45:2060-2067; and see the foregoing references). Regenerable sugarcane varieties, such as the commercial varieties CP65-357 and CP72-1210, may be used to generate transgene events.

Briefly, 7- to 40-week old calli are bombarded with plasmid-coated tungsten or gold particles. Two days after bombardment the calli are transferred to selection medium. Four weeks later the resistant calli are transferred to shoot-induction medium containing the selection agent and sub-cultured every two weeks for approximately 12 weeks, at which time the shoots are transferred to Magenta boxes containing rooting medium with selection agent. The shoots are maintained on this medium for approximately 8 weeks, at which time those with good root development are transferred to potting mix and the adapted to atmospheric growth.

Transgenic sugarcane plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 28: Method for Generating Transgenic Wheat Plants Carrying Hordeum GPT and GS1 Transgenes

This example provides a method for generating transgenic wheat plants expressing GPT and GS1 transgenes. Wheat (Triticum spp.) is biolistically transformed with an expression cassette comprising the Hordeum glutamine synthetase (GS1) coding sequence of SEQ ID NO: 40 under the control of the rice RuBisCo small subunit promoter of SEQ ID NO: 39 (expression cassette of SEQ ID NO: 42), and the Hordeum GPT coding sequence of SEQ ID NO: 45 under the control of the corn ubiquitin (Ubil) promoter of SEQ ID NO: 44. Transformation of wheat callus is achieved by particle bombardment.

Vector Constructs:

An expression cassette comprising the Hordeum GS1 and GPT genes, under the control of the rice RuBisCo small subunit and corn (maize) ubiquitin promoters, respectively, are cloned into a plasmid such as pAHC17, which contains the bar gene to provide the desired resistance to the phosphinothricin-class of herbicides for selection of transformants, using standard molecular cloning methodologies, to generate the transgene expression vector.

Transformation and Regeneration:

Wheat is transformed biolistically, and transgenic events regenerated, essentially as described (Weeks et al., 1993, Plant Physiology. 102:1077-1084; Blechl and Anderson, 1996, Nat. Biotech. 14:875-879; Okubara et. al., 2002, Theoretical and Applied Genetics. 106:74-83). These methods were developed and are routinely practiced at the US Department of Agriculture, Agricultural Research Service, Western Regional Research Center (Albany Calif.). The highly regenerable hexaploid spring wheat cultivar ‘Bobwhite’ is used as the source of immature embryos for bombardment with plasmid-coated particles.

Bombarded embryos are cultured without selection for 1-3 weeks in the dark on MS media before transferring them to shoot induction medium (MS media plus hormones and selection agent bialophos (1, 1.5, 2, 3 mg/L)) for 2-8 weeks with subculturing weekly (Blechl et al., 2007, J Cereal Science 45:172-183). Shoots that formed are transferred to rooting medium also containing the selection agent (bialophos 3 mg/L) (Weeks et al., 1993, supra). Well-rooted plantlets are transferred to potting media and adapted to atmospheric growth conditions.

Transgenic wheat plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 29: Method for Generating Transgenic Sorghum Plants Carrying Hordeum GPT and GS1 Transgenes

This example provides a method for generating transgenic Sorghum plants expressing GPT and GS1 transgenes. Sorghum (Sorghum spp L) is transformed with Agrobacterium carrying an expression cassette encoding the Hordeum glutamine synthetase (GS1) coding sequence of SEQ ID NO: 40 under the control of the rice RuBisCo subunit promoter of SEQ ID NO: 39 (expression cassette of SEQ ID NO: 42), and the Hordeum GPT coding sequence of SEQ ID NO: 45 under the control of the corn ubiquitin (WI) promoter of SE ID NO: 44.

Vector Constructs:

An expression cassette comprising the Hordeum GS1 and GPT genes, under the control of the rice RuBisCo small subunit and corn ubiquitin promoters, respectively, is cloned into a stable binary vector such as pZY101 (Vega et al 2008, Plant Cell Rep. 27:297-305), using standard molecular cloning methodologies, to generate the transgene expression vector.

Transformation and Regeneration:

Agrobacterium-mediated transformation and recovery of transgenic Sorghum plants is as described (Lu et al., 2009, Plant Cell Tissue Organ Culture 99:97-108). These methods are routinely used by the University of Missouri Plant Transformation Core Facility. The public Sorghum line, P898012, is grown as described (Lu et al., 2009, supra) and transformed with Agrobacterium tumefaciens strain EHA101 (Hood et al., 1986, supra) transformed with the transgene expression vector.

More specifically, Agrobacterium (0.3-0.4 OD) harboring the transgene expression vector is used to inoculate immature Sorghum embryos for 5 minutes. The embryos are then transferred onto filter paper on top of their co-cultivation medium, containing acetosyringone to enhance the effectiveness of the infection. Embryos are incubated for 3-5 days and then transferred for another 4 days on resting medium (containing carbenicillin) and then transferred onto callus induction medium (with selection agent PPT) with weekly transfers. Once somatic embyrogenic cells develop they are transferred onto shooting medium (with carbenicillin and PPT) until shoots (2-5 cm long) develop. Shoots are transferred to Magenta boxes with rooting medium (with PPT) and maintained in 16 h light and 8 h darkness until 8-20 cm tall well-rooted plantlets are produced. They are then transferred to potting mix and adapted to atmospheric conditions.

Transgenic Sorghum plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 30: Method for Generating Transgenic Switchgrass Plants Carrying Hordeum GPT and GS1 Transgenes

This example provides a method for generating transgenic switchgrass plants expressing GPT and GS1 transgenes. Switchgrass (Panicum virgatum) is transformed with Agrobacterium carrying a transgene expression vector including an expression cassette encoding the Hordeum glutamine synthetase (GS1) coding sequence of SEQ ID NO: 40 under the control of the rice RuBisCo small subunit promoter of SEQ ID NO: 39 (expression cassette of SEQ ID NO: 42), and the Hordeum GPT coding sequence of SEQ ID NO: 45 under the control of the corn ubiquitin (Ubil) promoter of SE ID NO: 44.

Vector Constructs:

An expression cassette comprising the Hordeum GS1 and GPT genes, under the control of the rice RuBisCo small subunit and corn (maize) ubiquitin promoters, respectively, is cloned into a Cambia vector thirteen hundred series (i.e., 1305.1) containing the HPT gene which provides hygromycin resistance for selection of the Switchgrass events, using standard molecular cloning methodologies, to generate the transgene expression vector.

Transformation and Regeneration:

Agrobacterium-mediated transformation and recovery of transgenic switchgrass plants is essentially as described (Somleva et al., 2002, Crop Science 42:2080-2087; Somleva 2006, Switchgrass (Panicum virgatum L.) In Methods in Molecular Biology Vol 344. Agrobacterium Protocols 2/e, Volume 2. Ed K. Wang Humana Press Inc., Totowa, N.J.; Xi et al 2009, Bioengineering Research 2:275-283). These methods are routinely used by the Plant Biotechnology Resource and Outreach Center at Michigan State University.

Briefly, explants of embryonic callus from the mature caryopses of the public Switchgrass cv. Alamo are transformed with Agrobacterium tumefaciens strain EHA105 (Hood et al., 1986, supra) carrying the transgene expression vector. Agrobacterium (0.8-1.0 OD) harboring the transgene expression vector and pretreated with acetosynringone is used to inoculate the switchgrass callus for 10 minutes and then co-cultivated for 4-6 days in the dark. The explants are then washed free of the Agrobacterium and placed on selection medium containing the antibiotic timentin and hygromycin; selection requires 2-6 months. Subculturing is carried out at 4-week intervals. Regeneration is accomplished in 4-8 weeks on media containing GA3, timentin and hygromycin under a photoperiod of 16 h light and 8 dark. The plantlets are then transferred to Magenta boxes with regeneration medium containing GA3, timentin and hygromycin for another 4 weeks as before. The plants are then transferred to soil and adapted to atmospheric growth.

Transgenic switchgrass plants may be grown and evaluated through maturity, and seeds harvested for use in generating subsequent generations of an event. Various phenotypic characteristics may be observed in To events, as well as in Ti and subsequent generations, and used to select seed sources for the development of subsequent generations. High performing lines may be selfed to achieve trait homozygosity and/or crossed.

### Example 31: Method for Generating Transgenic Soybean Plants Carrying Arabidopsis GPT and GS1 Transgenes

This example provides a method for generating transgenic soybean plants expressing GPT and GS1 transgenes. Soybean (Glycine max) is transformed with Agrobacterium carrying a transgene expression vector including an expression cassette encoding the Arabidopsis glutamine synthetase (GS1) coding sequence of SEQ ID NO: 7 under the control of the tomato RuBisCo small subunit promoter of SEQ ID NO: 22 (expression cassette of SEQ ID NO: 47), and the Arabidopsis GPT coding sequence of SEQ ID NO: 1 under the control of the 35S cauliflower mosaic virus (CMV) promoter (expression cassette of SEQ ID NO: 27).

Vector Constructs:

An expression cassette comprising the Hordeum GS1 and GPT genes, under the control of the tomato RuBisCo small subunit and 35S CMV promoters, respectively, is cloned into pTF101.1, using standard molecular cloning methodologies, to generate the transgene expression vector. pTF101.1 is a derivative of the pPZP binary vector (Hajdukiewicz et al 1994, Plant Mol. Biol. 25:989-994), which includes the right and left T-DNA border fragments from a nopaline strain of A. tumefaciens, a broad host origin of replication (pVS1) and a spectinomycin-resistant marker gene (aadA) for bacterial selection. The plant selectable marker gene cassette includes the phosphinothricin acetyl transferase (bar) gene from Streptomyces hygroscopicus that confers resistance to the herbicides glufosinate and bialophos. The soybean vegetative storage protein terminator (Mason et al., 1993) follows the 3′ end of the bar gene.

Media:

YEP Solid Medium: 5 g/L Yeast extract, 10 g/L Peptone, 5 g/L NaCl₂, 12 g/L Bacto-agar. pH to 7.0 with NaOH. Appropriate antibiotics should be added to the medium after autoclaving. Pour into sterile 100×15 plates (˜25 ml per plate).

YEP Liquid Medium: 5 g/L Yeast extract, 10 g/L Peptone, 5 g/L NaCl₂, pH to 7.0 with NaOH. Appropriate antibiotics should be added to the medium prior to inoculation.

Co-cultivation Medium: 1/10×B5 major salts, 1/10×B5 minor salts, 2.8 mg/L Ferrous, 3.8 mg/L NaEDTA, 30 g/L Sucrose, 3.9 g/L MES, and 4.25 g/L Noble agar (pH 5.4). Filter sterilized 1×B5 vitamins, GA3 (0.25 mg/L), BAP (1.67 mg/L), Cysteine (400 mg/L), Dithiothrietol (154.2 mg/L), and 40 mg/L acetosyringone are added to this medium after autoclaving. Pour into sterile 100×15 mm plates (˜88 plates/L). When solidified, overlay the co-cultivation medium with sterile filter paper to reduce bacterial overgrowth during co-cultivation (Whatman #1, 70 mm).

Infection Medium: 1/10×B5 major salts, 1/10×B5 minor salts, 2.8 mg/L Ferrous, 3.8 mg/L NaEDTA, 30 g/L Sucrose, 3.9 g/L MES (pH 5.4). Filter sterilized 1×B5 vitamins, GA3 (0.25 mg/L), BAP (1.67 mg/L), and 40 mg/L acetosyringone are added to this medium after autoclaving.

Shoot Induction Washing Medium: 1×B5 major salts, 1×B5 minor salts, 28 mg/L Ferrous, 38 mg/L NaEDTA, 30 g/L Sucrose, and 0.59 g/L MES (pH 5.7). Filter sterilized 1×B5 vitamins, BAP (1.11 mg/L), Timentin (100 mg/L), Cefotaxime (200 mg/L), and Vancomycin (50 mg/L) are added to this medium after autoclaving.

Shoot Induction Medium I: 1×B5 major salts, 1×B5 minor salts, 28 mg/L Ferrous, 38 mg/L NaEDTA, 30 g/L Sucrose, 0.59 g/L MES, and 7 g/L Noble agar (pH 5.7). Filter sterilized 1×B5 vitamins, BAP (1.11 mg/L), Timentin (50 mg/L), Cefotaxime (200 mg/L), and Vancomycin (50 mg/L) are added to this medium after autoclaving. Pour into sterile 100×20 mm plates (26 plates/L).

Shoot Induction Medium II: 1×B5 major salts, 1×B5 minor salts, 28 mg/L Ferrous, 38 mg/L NaEDTA, 30 g/L Sucrose, 0.59 g/L MES, and 7 g/L Noble agar (pH 5.7). Filter sterilized 1×B5 vitamins, BAP (1.11 mg/L), Timentin (50 mg/L), Cefotaxime (200 mg/L), Vancomycin (50 mg/L) and Glufosinate (6 mg/L) are added to this medium after autoclaving. Pour into sterile 100×20 mm plates (26 plates/L).

Shoot Elongation Medium: 1×MS major salts, 1×MS minor salts, 28 mg/L Ferrous, 38 mg/L NaEDTA, 30 g/L Sucrose, 0.59 g/L MES, and 7 g/L Noble agar (pH 5.7). Filter sterilized 1×B5 vitamins, Asparagine (50 mg/L), L-Pyroglutamic Acid (100 mg/L), IAA (0.1 mg/L), GA3 (0.5 mg/L), Zeatin-R (1 mg/L), Timentin (50 mg/L), Cefotaxime (200 mg/L), Vancomycin (50 mg/L), and Glufosinate (6 mg/L) are added to this medium after autoclaving. Pour into sterile 100×25 mm plates (22 plates/L).

Rooting Medium: 1×MS major salts, 1×MS minor salts, 28 mg/L Ferrous, 38 mg/L NaEDTA, 20 g/L Sucrose, 0.59 g/L MES, and 7 g/L Noble agar (pH 5.6). Filter sterilized 1×B5 vitamins, Asparagine (50 mg/L), and L-Pyroglutamic Acid (100 mg/L) are added to this medium after autoclaving. Pour into sterile 150×25 mm vial (10 ml/vial).

Transformation and Regeneration:

Agrobacterium cultures are prepared for infecting seed explants as follows. The vector system, pTF102 in EHA101, is cultured on YEP medium (An et al., 1988) containing 100 mg/L spectinomycin (for pTF102), 50 mg/L kanamycin (for EHA101), and 25 mg/L chloramphenicol (for EHA101). 24 hours prior to infection a 2 ml culture of Agrobacterium is started by inoculating a loop of bacteria from the fresh YEP plate in YEP liquid medium amended with antibiotics. This culture is allowed to grow to saturation (8-10 hours) at 28° C. in a shaker incubator (˜250 rpm). Then 0.2 ml of starter culture is transferred to a 1 L flask containing 250 ml of YEP medium amended with antibiotics. The culture is allowed to grow overnight at 28° C., 250 rpm to log phase (OD₆₅₀=0.3-0.6 for EHA105) or late log phase (OD₆₅₀=1.0-1.2 for EHA101). The Agrobacterium culture is then pelleted at 3,500 rpm for 10 minutes at 20° C., and the pellet resuspended in infection medium by pipetting through the pellet. Bacterial cell densities are adjusted to a final OD₆₅₀=0.6 (for EHA105) or OD₆₅₀=0.6 to 1.0 (for EHA101). Agrobacteria-containing infection medium is shaken at 60 rpm for at least 30 minutes before use.

Explants are prepared for inoculation as follows. Seeds are sterilized, ideally with a combination of bleach solution and exposure to chlorine gas. Prior to infection, (˜20 hours), sees are imbibed with deionized sterile water in the dark. Imbibed soybean seeds are transferred to a sterile 100×15 petri plate for dissection. Using a scalpel (i.e., #15 blade), longitudinal cuts are made along the hilum to separate the cotyledons and remove the seed coat. The embryonic axis found at the nodal end of the cotyledons is excised, and any remaining axial shoots/buds attached to the cotyledonary node are also removed.

Agrobacterium-mediated transformation is conducted as follows. Half-seed explants are dissected into a 100×25 mm petri plate and 30 ml Agrobacterium-containing infection media added thereto, such that the explants are completely covered by the infection media. Explants are allowed to incubate at room temperature for a short period of time (i.e., 30 minutes), preferably with occasional gentle agitation.

After infection, the explants are transferred to co-cultivation medium, preferably so that the flat, axial side is touching the filter paper. These plates are typically wrapped in parafilm, and cultivated for 5 days at 24° C. under an 18:6 photoperiod. Following this co-cultivation, shoot growth is induced by first washing the explants in shoot induction washing medium at room temperature, followed by placing the explants in shoot induction medium I, such that the explants are oriented with the nodal end of the cotyledon imbedded in the medium and the regeneration region flush to the surface with flat side up (preferably at a 30-45° angle). Explants are incubated at 24° C., 18:6 photoperiod, for 14 days. Explants are thereafter transferred to shoot induction medium II and maintained under the same conditions for another 14 days.

Following shoot induction, explants are transferred to shoot elongation medium, as follows. First, cotyledons are removed from the explants. A fresh cut at the base of the shoot pad flush to the medium is made, and the explants transferred to shoot elongation medium (containing glufosinate) and incubated at 24° C., 18:6 photoperiod, for 2-8 weeks. Preferably, explant tissue is transferred to fresh shoot elongation medium every 2 weeks, and at transfer, a fresh horizontal slice at the base of the shoot pad is made.

When shoots surviving the glufosinate selection have reached ˜3 cm length, they are excised from the shoot pad, briefly dipped in indole-3-butyric acid (1 mg/ml, 1-2 minutes), then transferred to rooting medium for acclimatization (i.e., in 150×25 mm glass vials with the stems of the shoots embedded approximately ½ cm into the media). When well rooted, the shoots are transferred to soil and plantlets grown at 24° C., 18:6 photoperiod, for at least one week, watering as needed. When the plantlets have at least two healthy trifoliates, an herbicide paint assay may be applied to confirm resistance to glufosinate. Briefly, using a cotton swab, Liberty herbicide (150 mg I-1) is applied to the upper leaf surface along the midrib of two leaves on two different trifoliates. Painted plants are transferred to the greenhouse and covered with a humidome. Plantlets are scored 3-5 days after painting. Resistant plantlets may be transplanted immediately to larger pots (i.e., 2 gal).

### Example 32: Method for Generating Transgenic Potato Plants Carrying Arabidopsis GPT and GS1 Transgenes

This example provides a method for generating transgenic potato plants expressing GPT and GS1 transgenes. Potato (Solanum tuberosum, cultivar Desiree) is transformed with Agrobacterium carrying a transgene expression vector including an expression cassette encoding the Arabidopsis glutamine synthetase (GS1) coding sequence of SEQ ID NO: 7 under the control of the tomato RuBisCo small subunit promoter of SEQ ID NO: 22 (expression cassette of SEQ ID NO: 47), and the Arabidopsis GPT coding sequence of SEQ ID NO: 1 under the control of the 35S cauliflower mosaic virus (CMV) promoter (expression cassette of SEQ ID NO: 27).

Vector Constructs:

An expression cassette comprising the Hordeum GS1 and GPT genes, under the control of the tomato RuBisCo small subunit and 35S CMV promoters, respectively, is cloned into the Cambia 2201 vector which provides kanamycin resistance.

Transformation and Regeneration:

A suitable Agrobacterium tumefaciens strain such as UC-Riverside Agro-1 strain is employed and used for infecting potato explant tissue (see, Narvaez-Vasquez et al., 1992, Plant Mo. Biol. 20:1149-1157). Cultures are maintained at 28° C. in liquid medium containing 10 g/L Yeast extract, 10 g/L Peptone, 5 g/L NaCl₂, 10 mg/L kanamycin, 30 mg/L tetracycline, and 9.81 g/L Acetosyringone (50 mM). Overnight cultures are diluted with liquid MS medium (4.3 g/L MS salts, 20 g/L sucrose, 1 mg/L thiamine, 100 mg/L inositol and 7 g/L phytoagar, pH to 5.8) to 10⁸ Agrobacterium cells/ml for the infection of plant tissues (co-cultivation).

Potato leaf discs or tuber discs may be used as the explants to be inoculated. Discs are pre-conditioned by incubation on feeder plates for two to three days at 25° C. under dark conditions. Pre-conditioned explants are infected with Agrobacterium by soaking in 20 ml of sterile liquid MS medium (supra), containing 10⁸ Agrobacterium cells/ml for about 20 minutes. Before or during the co-cultivation, the explants are carefully punched with a syringe needle, or scalpel blade. Then, the explants are blotted dry with sterile filter paper, and incubated again in feeder plates for another two days. Explants are then transferred to liquid medium with transgene-transformed Agrobacterium, and incubated for three days at 28° C. under dark conditions for calli and shoot development (development (2-4 cm) in the presence of kanamycin (100 mg/L).

Following co-cultivation, supra, the explants are washed three times with sterile liquid medium and finally rinsed with the same medium containing 500 mg/I of cefotaxime. The explants are blotted dry with sterile filter paper and placed on shoot induction medium (4.3 g/L MS salts, 10 mg/L thiamine, 1 mg/L nicotinic acid, 1 mg/L pyridxine, 100 mg/L inositol, 30 g/L sucrose, 1 mg/L zeatin, 0.5 mg/L IAA, 7 g/L phytoagar, 250 mg/L Cefotaxime, 500 mg/L Carbenicillin, 100 mg/L Kanamycin) for 4-6 weeks. Thereafter, plantlets are transferred to rooting medium (4.3 g/L MS salts, 10 mg/L thiamine, 1 mg/L nicotinic acid, 1 mg/L pyridxine, 100 mg/L inositol, 20 g/L sucrose, 50 μg/L IAA, 7 g/L phytoagar, 50 mg/L Kanamycin and 500 mg/L Vancomycin) for 3-4 weeks.

All publications, patents, and patent applications cited in this specification are herein incorporated by reference as if each individual publication or patent application were specifically and individually indicated to be incorporated by reference.

The present invention is not to be limited in scope by the embodiments disclosed herein, which are intended as single illustrations of individual aspects of the invention, and any which are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE OF SEQUENCES
SEQ ID NO: 1 Arabidopsis glutamine phenylpyruvate transaminase DNA coding sequence:
ATGTACCTGGACATAAATGGTGTGATGATCAAACAGTTTAGCTTCAAAGCCTCTCTTCTCCC
ATTCTCTTCTAATTTCCGACAAAGCTCCGCCAAAATCCATCGTCCTATCGGAGCCACCATGA
CCACAGTTTCGACTCAGAACGAGTCTACTCAAAAACCCGTCCAGGTGGCGAAGAGATTAGA
GAAGTTCAAGACTACTATTTTCACTCAAATGAGCATATTGGCAGTTAAACATGGAGCGATCA
ATTTAGGCCAAGGCTTTCCCAATTTCGACGGTCCTGATTTTGTTAAAGAAGCTGCGATCCAA
GCTATTAAAGATGGTAAAAACCAGTATGCTCGTGGATACGGCATTCCTCAGCTCAACTCTG
CTATAGCTGCGCGGTTTCGTGAAGATACGGGTCTTGTTGTTGATCCTGAGAAAGAAGTTAC
TGTTACATCTGGTTGCACAGAAGCCATAGCTGCAGCTATGTTGGGTTTAATAAACCCTGGT
GATGAAGTCATTCTCTTTGCACCGTTTTATGATTCCTATGAAGCAACACTCTCTATGGCTGG
TGCTAAAGTAAAAGGAATCACTTTACGTCCACCGGACTTCTCCATCCCTTTGGAAGAGCTTA
AAGCTGCGGTAACTAACAAGACTCGAGCCATCCTTATGAACACTCCGCACAACCCGACCG
GGAAGATGTTCACTAGGGAGGAGCTTGAAACCATTGCATCTCTCTGCATTGAAAACGATGT
GCTTGTGTTCTCGGATGAAGTATACGATAAGCTTGCGTTTGAAATGGATCACATTTCTATAG
CTTCTCTTCCCGGTATGTATGAAAGAACTGTGACCATGAATTCCCTGGGAAAGACTTTCTCT
TTAACCGGATGGAAGATCGGCTGGGCGATTGCGCCGCCTCATCTGACTTGGGGAGTTCGA
CAAGCACACTCTTACCTCACATTCGCCACATCAACACCAGCACAATGGGCAGCCGTTGCAG
CTCTCAAGGCACCAGAGTCTTACTTCAAAGAGCTGAAAAGAGATTACAATGTGAAAAAGGA
GACTCTGGTTAAGGGTTTGAAGGAAGTCGGATTTACAGTGTTCCCATCGAGCGGGACTTAC
TTTGTGGTTGCTGATCACACTCCATTTGGAATGGAGAACGATGTTGCTTTCTGTGAGTATCT
TATTGAAGAAGTTGGGGTCGTTGCGATCCCAACGAGCGTCTTTTATCTGAATCCAGAAGAA
GGGAAGAATTTGGTTAGGTTTGCGTTCTGTAAAGACGAAGAGACGTTGCGTGGTGCAATTG
AGAGGATGAAGCAGAAGCTTAAGAGAAAAGTCTGA
SEQ ID NO: 2 Arabidopsis GPT amino acid sequence
MYLDINGVMIKQFSFKASLLPFSSNFRQSSAKIHRPIGATMTTVSTQNESTQKPVQVAKRLEKFK
TTIFTQMSILAVKHGAINLGQGFPNFDGPDFVKEAAIQAIKDGKNQYARGYGIPQLNSAIAARFR
EDTGLVVDPEKEVTVTSGCTEAIAAAMLGLINPGDEVILFAPFYDSYEATLSMAGAKVKGITLRP
PDFSIPLEELKAAVTNKTRAILMNTPHNPTGKMFTREELETIASLCIENDVLVFSDEVYDKLAFEM
DHISIASLPGMYERTVTMNSLGKTFSLTGWKIGWAIAPPHLTWGVRQAHSYLTFATSTPAQWA
AVAALKAPESYFKELKRDYNVKKETLVKGLKEVGFTVFPSSGTYFVVADHTPFGMENDVAFCE
YLIEEVGVVAIPTSVFYLNPEEGKNLVRFAFCKDEETLRGAIERMKQKLKRKV
SEQ ID NO: 3 Alfalfa GS1 DNAcoding sequence (upper case) with 5′ and 3′ untranslated
sequences (indicated in lower case).
atttccgttttcgttttcatttgattcattgaatcaaatcgaatcgaatctttaggattcaatacagattcctta
gattttactaagtttgaaaccaaaaccaaaacATGTCTCTCCTTTCAGATCTTATCAACC
TTGACCTCTCCGAAACCACCGAGAAAATCATCGCCGAATACATATGGATTG
GTGGATCTGGTTTGGACTTGAGGAGCAAAGCAAGGACTCTACCAGGACCA
GTTACTGACCCTTCACAGCTTCCCAAGTGGAACTATGATGGTTCCAGCACA
GGTCAAGCTCCTGGAGAAGATAGTGAAGTTATTATCTACCCACAAGCCATT
TTCAAGGACCCATTTAGAAGGGGTAACAATATCTTGGTTATGTGTGATGCA
TACACTCCAGCTGGAGAGCCCATTCCCACCAACAAGAGACATGCAGCTGC
CAAGATTTTCAGCCATCCTGATGTTGTTGCTGAAGTACCATGGTATGGTATT
GAGCAAGAATACACCTTGTTGCAGAAAGACATCAATTGGCCTCTTGGTTGG
CCAGTTGGTGGTTTTCCTGGACCTCAGGGACCATACTATTGTGGAGCTGGT
GCTGACAAGGCATTTGGCCGTGACATTGTTGACTCACATTACAAAGCCTGT
CTTTATGCCGGCATCAACATCAGTGGAATCAATGGTGAAGTGATGCCTGGT
CAATGGGAATTCCAAGTTGGTCCCTCAGTTGGTATCTCTGCTGGTGATGAG
ATATGGGTTGCTCGTTACATTTTGGAGAGGATCACTGAGGTTGCTGGTGTG
GTGCTTTCCTTTGACCCAAAACCAATTAAGGGTGATTGGAATGGTGCTGGT
GCTCACACAAATTACAGCACCAAGTCTATGAGAGAAGATGGTGGCTATGAA
GTCATCTTGAAAGCAATTGAGAAGCTTGGGAAGAAGCACAAGGAGCACATT
GCTGCTTATGGAGAAGGCAACGAGCGTAGATTGACAGGGCGACATGAGAC
AGCTGACATTAACACCTTCTTATGGGGTGTTGCAAACCGTGGTGCGTCGAT
TAGAGTTGGAAGGGACACAGAGAAAGCAGGGAAAGGTTATTTCGAGGATA
GGAGGCCATCATCTAACATGGATCCATATGTTGTTACTTCCATGATTGCAG
ACACCACCATTCTCTGGAAACCATAAgccaccacacacacatgcattgaagtatttgaaa
gtcattgttgattccgcattagaatttggtcattgttttttctaggatttggatttgtgttattgttatggttcaca
ctttgtttgtttgaatttgaggccttgttataggtttcatatttctttctcttgttctaagtaaatgtcagaataat
aatgtaat
SEQ ID NO: 4 Alfalfa GS1 amino acid sequence
MSLLSDLINLDLSETTEKIIAEYIWIGGSGLDLRSKARTLPGPVTDPSQLPKWNYDGSSTGQAPG
EDSEVIIYPQAIFKDPFRRGNNILVMCDAYTPAGEPIPTNKRHAAAKIFSHPDVVAEVPWYGIEQ
EYTLLQKDINWPLGWPVGGFPGPQGPYYCGAGADKAFGRDIVDSHYKACLYAGINISGINGEV
MPGQWEFQVGPSVGISAGDEIWVARYILERITEVAGVVLSFDPKPIKGDWNGAGAHTNYSTKS
MREDGGYEVILKAIEKLGKKHKEHIAAYGEGNERRLTGRHETADINTFLWGVANRGASIRVGRD
TEKAGKGYFEDRRPSSNMDPYVVTSMIADTTILWKP
SEQ ID NO: 5 Alfalfa GS1 DNAcoding sequence (upper case) with 5′ and 3′ untranslated
sequences (indicated in lower case) and vector sequences from ClaI to SmaI/SspI and
SspI/SmaI to SalI/XhoI (lower case, underlined).
atccgatgaattccgagctcggtacccatttccgttttcgttttcatttgattcattgaatcaaatcgaatcgaa
tctttaggattcaatacagattccttagattttactaagtttgaaaccaaaaccaaaacATGTCTCTC
CTTTCAGATCTTATCAACCTTGACCTCTCCGAAACCACCGAGAAAATCATC
GCCGAATACATATGGATTGGTGGATCTGGTTTGGACTTGAGGAGCAAAGCA
AGGACTCTACCAGGACCAGTTACTGACCCTTCACAGCTTCCCAAGTGGAAC
TATGATGGTTCCAGCACAGGTCAAGCTCCTGGAGAAGATAGTGAAGTTATT
ATCTACCCACAAGCCATTTTCAAGGACCCATTTAGAAGGGGTAACAATATC
TTGGTTATGTGTGATGCATACACTCCAGCTGGAGAGCCCATTCCCACCAAC
AAGAGACATGCAGCTGCCAAGATTTTCAGCCATCCTGATGTTGTTGCTGAA
GTACCATGGTATGGTATTGAGCAAGAATACACCTTGTTGCAGAAAGACATC
AATTGGCCTCTTGGTTGGCCAGTTGGTGGTTTTCCTGGACCTCAGGGACCA
TACTATTGTGGAGCTGGTGCTGACAAGGCATTTGGCCGTGACATTGTTGAC
TCACATTACAAAGCCTGTCTTTATGCCGGCATCAACATCAGTGGAATCAAT
GGTGAAGTGATGCCTGGTCAATGGGAATTCCAAGTTGGTCCCTCAGTTGGT
ATCTCTGCTGGTGATGAGATATGGGTTGCTCGTTACATTTTGGAGAGGATC
ACTGAGGTTGCTGGTGTGGTGCTTTCCTTTGACCCAAAACCAATTAAGGGT
GATTGGAATGGTGCTGGTGCTCACACAAATTACAGCACCAAGTCTATGAGA
GAAGATGGTGGCTATGAAGTCATCTTGAAAGCAATTGAGAAGCTTGGGAAG
AAGCACAAGGAGCACATTGCTGCTTATGGAGAAGGCAACGAGCGTAGATT
GACAGGGCGACATGAGACAGCTGACATTAACACCTTCTTATGGGGTGTTG
CAAACCGTGGTGCGTCGATTAGAGTTGGAAGGGACACAGAGAAAGCAGGG
AAAGGTTATTTCGAGGATAGGAGGCCATCATCTAACATGGATCCATATGTT
GTTACTTCCATGATTGCAGACACCACCATTCTCTGGAAACCATAAgccaccac
acacacatgcattgaagtatttgaaagtcattgttgattccgcattagaatttggtcattgttttttctaggat
ttggatttgtgttattgttatggttcacactttgtttgtttgaatttgaggccttgttataggtttcatatttctttct
cttgttctaagtaaatgtcagaataataatgtaatggggatcctctagagtcgag
SEQ ID NO: 6 Arabidopsis GS1 coding sequence
Cambia 1201 vector + rbcS3C + arabidopsis GS1 Bold ATG is the start site,
AAAAAAGAAAAAAAAAACATATCTTGTTTGTCAGTATGGGAAGTTTGAGATAAGGACGAGTG
AGGGGTTAAAATTCAGTGGCCATTGATTTTGTAATGCCAAGAACCACAAAATCCAATGGTTA
CCATTCCTGTAAGATGAGGTTTGCTAACTCTTTTTGTCCGTTAGATAGGAAGCCTTATCACT
ATATATACAAGGCGTCCTAATAACCTCTTAGTAACCAATTATTTCAGCACC [symbol] TCTCTGCT
CTCAGATCTCGTTAACCTCAACCTCACCGATGCCACCGGGAAAATCATCGCCGAATACATA
TGGATCGGTGGATCTGGAATGGATATCAGAAGCAAAGCCAGGACACTACCAGGACCAGTG
ACTGATCCATCAAAGCTTCCCAAGTGGAACTACGACGGATCCAGCACCGGTCAGGCTGCT
GGAGAAGACAGTGAAGTCATTCTATACCCTCAGGCAATATTCAAGGATCCCTTCAGGAAAG
GCAACAACATCCTGGTGATGTGTGATGCTTACACACCAGCTGGTGATCCTATTCCAACCAA
CAAGAGGCACAACGCTGCTAAGATCTTCAGCCACCCCGACGTTGCCAAGGAGGAGCCTTG
GTATGGGATTGAGCAAGAATACACTTTGATGCAAAAGGATGTGAACTGGCCAATTGGTTGG
CCTGTTGGTGGCTACCCTGGCCCTCAGGGACCTTACTACTGTGGTGTGGGAGCTGACAAA
GCCATTGGTCGTGACATTGTGGATGCTCACTACAAGGCCTGTCTTTACGCCGGTATTGGTA
TTTCTGGTATCAATGGAGAAGTCATGCCAGGCCAGTGGGAGTTCCAAGTCGGCCCTGTTG
AGGGTATTAGTTCTGGTGATCAAGTCTGGGTTGCTCGATACCTTCTCGAGAGGATCACTGA
GATCTCTGGTGTAATTGTCAGCTTCGACCCGAAACCAGTCCCGGGTGACTGGAATGGAGC
TGGAGCTCACTGCAACTACAGCACTAAGACAATGAGAAACGATGGAGGATTAGAAGTGATC
AAGAAAGCGATAGGGAAGCTTCAGCTGAAACACAAAGAACACATTGCTGCTTACGGTGAAG
GAAACGAGCGTCGTCTCACTGGAAAGCACGAAACCGCAGACATCAACACATTCTCTTGGG
GAGTCGCGAACCGTGGAGCGTCAGTGAGAGTGGGACGTGACACAGAGAAGGAAGGTAAA
GGGTACTTCGAAGACAGAAGGCCAGCTTCTAACATGGATCCTTACGTTGTCACCTCCATGA
TCGCTGAGACGACCATACTCGGTTGA
SEQ ID NO: 7 Arabidopsis GS1 amino acid sequence
Vector sequences at N-terminus in italics
MVDLRNRRTSMSLLSDLVNLNLTDATGKIIAEYIWIGGSGMDIRSKARTLPGPVTDPSKLPKWN
YDGSSTGQAAGEDSEVILYPQAIFKDPFRKGNNILVMCDAYTPAGDPIPTNKRHNAAKIFSHPD
VAKEEPWYGIEQEYTLMQKDVNWPIGWPVGGYPGPQGPYYCGVGADKAIGRDIVDAHYKACL
YAGIGISGINGEVMPGQWEFQVGPVEGISSGDQVWVARYLLERITEISGVIVSFDPKPVPGDWN
GAGAHCNYSTKTMRNDGGLEVIKKAIGKLQLKHKEHIAAYGEGNERRLTGKHETADINTFSWG
VANRGASVRVGRDTEKEGKGYFEDRRPASNMDPYVVTSMIAETTILG
SEQ ID NO: 8 Grape GPT coding DNA sequence
Showing Cambia 1305.1 with (3′ end of) rbcS3C + Vitis vinifera GPT (Grape). Bold ATG is the
start site, parentheses are the catI intron and the underlined actagt is the speI cloning site
used to splice in the GPT gene.
AAAAAAGAAAAAAAAAACATATCTTGTTTGTCAGTATGGGAAGTTTGAGATAAGGACGAGTG
AGGGGTTAAAATTCAGTGGCCATTGATTTTGTAATGCCAAGAACCACAAAATCCAATGGTTA
CCATTCCTGTAAGATGAGGTTTGCTAACTCTTTTTGTCCGTTAGATAGGAAGCCTTATCACT
ATATATACAAGGCGTCCTAATAACCTCTTAGTAACCAATTATTTCAGCA [symbol] TAGATCT
GAGG(GTAAATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGTGTAAATAT
TACATAGCTTTAACTGATAATCTGATTACTTTATTTCGTGTGTCTATGATGATGATGATAGTT
ACAG)AACCGACGA [symbol] ATGCAGCTCTCTCAATGTACCTGGACATTCCCAGAGTTGCT
TAAAAGACCAGCCTTTTTAAGGAGGAGTATTGATAGTATTTCGAGTAGAAGTAGGTCCAGC
TCCAAGTATCCATCTTTCATGGCGTCCGCATCAACGGTCTCCGCTCCAAATACGGAGGCTG
AGCAGACCCATAACCCCCCTCAACCTCTACAGGTTGCAAAGCGCTTGGAGAAATTCAAAAC
AACAATCTTTACTCAAATGAGCATGCTTGCCATCAAACATGGAGCAATAAACCTTGGCCAAG
GGTTTCCCAACTTTGATGGTCCTGAGTTTGTCAAAGAAGCAGCAATTCAAGCCATTAAGGA
TGGGAAAAACCAATATGCTCGTGGATATGGAGTTCCTGATCTCAACTCTGCTGTTGCTGAT
AGATTCAAGAAGGATACAGGACTCGTGGTGGACCCCGAGAAGGAAGTTACTGTTACTTCTG
GATGTACAGAAGCAATTGCTGCTACTATGCTAGGCTTGATAAATCCTGGTGATGAGGTGAT
CCTCTTTGCTCCATTTTATGATTCCTATGAAGCCACTCTATCCATGGCTGGTGCCCAAATAA
AATCCATCACTTTACGTCCTCCGGATTTTGCTGTGCCCATGGATGAGCTCAAGTCTGCAAT
CTCAAAGAATACCCGTGCAATCCTTATAAACACTCCCCATAACCCCACAGGAAAGATGTTC
ACAAGGGAGGAACTGAATGTGATTGCATCCCTCTGCATTGAGAATGATGTGTTGGTGTTTA
CTGATGAAGTTTACGACAAGTTGGCTTTCGAAATGGATCACATTTCCATGGCTTCTCTTCCT
GGGATGTACGAGAGGACCGTGACTATGAATTCCTTAGGGAAAACTTTCTCCCTGACTGGAT
GGAAGATTGGTTGGACAGTAGCTCCCCCACACCTGACATGGGGAGTGAGGCAAGCCCACT
CATTCCTCACGTTTGCTACCTGCACCCCAATGCAATGGGCAGCTGCAACAGCCCTCCGGG
CCCCAGACTCTTACTATGAAGAGCTAAAGAGAGATTACAGTGCAAAGAAGGCAATCCTGGT
GGAGGGATTGAAGGCTGTCGGTTTCAGGGTATACCCATCAAGTGGGACCTATTTTGTGGT
GGTGGATCACACCCCATTTGGGTTGAAAGACGATATTGCGTTTTGTGAGTATCTGATCAAG
GAAGTTGGGGTGGTAGCAATTCCGACAAGCGTTTTCTACTTACACCCAGAAGATGGAAAGA
ACCTTGTGAGGTTTACCTTCTGTAAAGACGAGGGAACTCTGAGAGCTGCAGTTGAAAGGAT
GAAGGAGAAACTGAAGCCTAAACAATAGGGGCACGTGA
SEQ ID NO: 9 Grape GPT amino acid sequence
MVDLRNRRTSMQLSQCTWTFPELLKRPAFLRRSIDSISSRSRSSSKYPSFMASASTVSAPNTE
AEQTHNPPQPLQVAKRLEKFKTTIFTQMSMLAIKHGAINLGQGFPNFDGPEFVKEAAIQAIKDGK
NQYARGYGVPDLNSAVADRFKKDTGLVVDPEKEVTVTSGCTEAIAATMLGLINPGDEVILFAPF
YDSYEATLSMAGAQIKSITLRPPDFAVPMDELKSAISKNTRAILINTPHNPTGKMFTREELNVIAS
LCIENDVLVFTDEVYDKLAFEMDHISMASLPGMYERTVTMNSLGKTFSLTGWKIGWTVAPPHLT
WGVRQAHSFLTFATCTPMQWAAATALRAPDSYYEELKRDYSAKKAILVEGLKAVGFRVYPSSG
TYFVVVDHTPFGLKDDIAFCEYLIKEVGVVAIPTSVFYLHPEDGKNLVRFTFCKDEGTLRAAVER
MKEKLKPKQ
SEQ ID NO: 10 Rice GPT DNA coding sequence
Rice GPTcodon optimized for E. coli expression; untranslated sequences shown in lower case
atgtggATGAACCTGGCAGGCTTTCTGGCAACCCCGGCAACCGCAACCGCAACCCGTCATGA
AATGCCGCTGAACCCGAGCAGCAGCGCGAGCTTTCTGCTGAGCAGCCTGCGTCGTAGCCT
GGTGGCGAGCCTGCGTAAAGCGAGCCCGGCAGCAGCAGCAGCACTGAGCCCGATGGCAA
GCGCAAGCACCGTGGCAGCAGAAAACGGTGCAGCAAAAGCAGCAGCAGAAAAACAGCAG
CAGCAGCCGGTGCAGGTGGCGAAACGTCTGGAAAAATTTAAAACCACCATTTTTACCCAGA
TGAGCATGCTGGCGATTAAACATGGCGCGATTAACCTGGGCCAGGGCTTTCC
GAACTTTGATGGCCCGGATTTTGTGAAAGAAGCGGCGATTCAGGCGATTAACGCGGGCAA
AAACCAGTATGCGCGTGGCTATGGCGTGCCGGAACTGAACAGCGCGATTGCGGAACGTTT
TCTGAAAGATAGCGGCCTGCAGGTGGATCCGGAAAAAGAAGTGACCGTGACCAGCGGCT
GCACCGAAGCGATTGCGGCGACCATTCTGGGCCTGATTAACCCGGGCGATGAAGTGATTC
TGTTTGCGCCGTTTTATGATAGCTATGAAGCGACCCTGAGCATGGCGGGCGCGAACGTGA
AAGCGATTACCCTGCGTCCGCCGGATTTTAGCGTGCCGCTGGAAGAACTGAAAGCGGCCG
TGAGCAAAAACACCCGTGCGATTATGATTAACACCCCGCATAACCCGACCGGCAAAATGTT
TACCCGTGAAGAACTGGAATTTATTGCGACCCTGTGCAAAGAAAACGATGTGCTGCTGTTT
GCGGATGAAGTGTATGATAAACTGGCGTTTGAAGCGGATCATATTAGCATGGCGAGCATTC
CGGGCATGTATGAACGTACCGTGACCATGAACAGCCTGGGCAAAACCTTTAGCCTGACCG
GCTGGAAAATTGGCTGGGCGATTGCGCCGCCGCATCTGACCTGGGGCGTGCGTCAGGCA
CATAGCTTTCTGACCTTTGCAACCTGCACCCCGATGCAGGCAGCCGCCGCAGCAGCACTG
CGTGCACCGGATAGCTATTATGAAGAACTGCGTCGTGATTATGGCGCGAAAAAAGCGCTG
CTGGTGAACGGCCTGAAAGATGCGGGCTTTATTGTGTATCCGAGCAGCGGCACCTATTTT
GTGATGGTGGATCATACCCCGTTTGGCTTTGATAACGATATTGAATTTTGCGAATATCTGAT
TCGTGAAGTGGGCGTGGTGGCGATTCCGCCGAGCGTGTTTTATCTGAACCCGGAAGATGG
CAAAAACCTGGTGCGTTTTACCTTTTGCAAAGATGATGAAACCCTGCGTGCGGCGGTGGAA
CGTATGAAAACCAAACTGCGTAAAAAAAAGCTTgcggccgcactcgagcaccaccaccaccaccactga
SEQ ID NO: 11 Rice GPT amino acid sequence
Includes amino terminal amino acids MW for cloning and His tag sequences from pet28 vector
in italics.
MWMNLAGFLATPATATATRHEMPLNPSSSASFLLSSLRRSLVASLRKASPAAAAALSPMASAS
TVAAENGAAKAAAEKQQQQPVQVAKRLEKFKTTIFTQMSMLAIKHGAINLGQGFPNFDGPDFV
KEAAIQAINAGKNQYARGYGVPELNSAIAERFLKDSGLQVDPEKEVTVTSGCTEAIAATILGLINP
GDEVILFAPFYDSYEATLSMAGANVKAITLRPPDFSVPLEELKAAVSKNTRAIMINTPHNPTGKM
FTREELEFIATLCKENDVLLFADEVYDKLAFEADHISMASIPGMYERTVTMNSLGKTFSLTGWKI
GWAIAPPHLTWGVRQAHSFLTFATCTPMQAAAAAALRAPDSYYEELRRDYGAKKALLVNGLKD
AGFIVYPSSGTYFVMVDHTPFGFDNDIEFCEYLIREVGVVAIPPSVFYLNPEDGKNLVRFTFCKD
DETLRAAVERMKTKLRKKKLAAALEHHHHHH
SEQ ID NO: 12 Soybean GP TDNA coding sequence
TOPO 151D WITH SOYBEAN for E. coli expression
From starting codon. Vector sequences are italicized
ATGCATCATCACCATCACCATGGTAAGCCTATCCCTAACCCTCTCCTCGGTCTCGATTCTA
CGGAAAACCTGTATTTTCAGGGAATTGATCCCTTCACCGCGAAACGTCTGGAAAAATTTCA
GACCACCATTTTTACCCAGATGAGCCTGCTGGCGATTAAACATGGCGCGATTAACCTGGGC
CAGGGCTTTCCGAACTTTGATGGCCCGGAATTTGTGAAAGAAGCGGCGATTCAGGCGATT
CGTGATGGCAAAAACCAGTATGCGCGTGGCTATGGCGTGCCGGATCTGAACATTGCGATT
GCGGAACGTTTTAAAAAAGATACCGGCCTGGTGGTGGATCCGGAAAAAGAAATTACCGTG
ACCAGCGGCTGCACCGAAGCGATTGCGGCGACCATGATTGGCCTGATTAACCCGGGCGA
TGAAGTGATTATGTTTGCGCCGTTTTATGATAGCTATGAAGCGACCCTGAGCATGGCGGGC
GCGAAAGTGAAAGGCATTACCCTGCGTCCGCCGGATTTTGCGGTGCCGCTGGAAGAACTG
AAAAGCACCATTAGCAAAAACACCCGTGCGATTCTGATTAACACCCCGCATAACCCGACCG
GCAAAATGTTTACCCGTGAAGAACTGAACTGCATTGCGAGCCTGTGCATTGAAAACGATGT
GCTGGTGTTTACCGATGAAGTGTATGATAAACTGGCGTTTGATATGGAACATATTAGCATG
GCGAGCCTGCCGGGCATGTTTGAACGTACCGTGACCCTGAACAGCCTGGGCAAAACCTTT
AGCCTGACCGGCTGGAAAATTGGCTGGGCGATTGCGCCGCCGCATCTGAGCTGGGGCGT
GCGTCAGGCGCATGCGTTTCTGACCTTTGCAACCGCACATCCGTTTCAGTGCGCAGCAGC
AGCAGCACTGCGTGCACCGGATAGCTATTATGTGGAACTGAAACGTGATTATATGGCGAAA
CGTGCGATTCTGATTGAAGGCCTGAAAGCGGTGGGCTTTAAAGTGTTTCCGAGCAGCGGC
ACCTATTTTGTGGTGGTGGATCATACCCCGTTTGGCCTGGAAAACGATGTGGCGTTTTGCG
AATATCTGGTGAAAGAAGTGGGCGTGGTGGCGATTCCGACCAGCGTGTTTTATCTGAACC
CGGAAGAAGGCAAAAACCTGGTGCGTTTTACCTTTTGCAAAGATGAAGAAACCATTCGTAG
CGCGGTGGAACGTATGAAAGCGAAACTGCGTAAAGTCGACTAA
SEQ ID NO: 13 Soybean GPT amino acid sequence
Translated protein product, vector sequences italicized
MHHHHHHGKPIPNPLLGLDSTENLYFQGIDPFTAKRLEKFQTTIFTQMSLLAIKHGAINLGQGFP
NFDGPEFVKEAAIQAIRDGKNQYARGYGVPDLNIAIAERFKKDTGLVVDPEKEITVTSGCTEAIA
ATMIGLINPGDEVIMFAPFYDSYEATLSMAGAKVKGITLRPPDFAVPLEELKSTISKNTRAILINTP
HNPTGKMFTREELNCIASLCIENDVLVFTDEVYDKLAFDMEHISMASLPGMFERTVTLNSLGKT
FSLTGWKIGWAIAPPHLSWGVRQAHAFLTFATAHPFQCAAAAALRAPDSYYVELKRDYMAKRA
ILIEGLKAVGFKVFPSSGTYFVVVDHTPFGLENDVAFCEYLVKEVGVVAIPTSVFYLNPEEGKNL
VRFTFCKDEETIRSAVERMKAKLRKVD
SEQ ID NO: 14 Barley GPT DNA coding sequence
Coding sequence from start with intron removed [symbol]
TAGATCTGAGGAACCGACGA [symbol] ATGGCATCCGCCCCCGCCTCCGCCTCCGC
GGCCCTCTCCACCGCCGCCCCCGCCGACAACGGGGCCGCCAAGCCCACGGAGCAGCGG
CCGGTACAGGTGGCTAAGCGATTGGAGAAGTTCAAAACAACAATTTTCACACAGATGAGCA
TGCTCGCAGTGAAGCATGGAGCAATAAACCTTGGACAGGGGTTTCCCAATTTTGATGGCCC
TGACTTTGTCAAAGATGCTGCTATTGAGGCTATCAAAGCTGGAAAGAATCAGTATGCAAGA
GGATATGGTGTGCCTGAATTGAACTCAGCTGTTGCTGAGAGATTTCTCAAGGACAGTGGAT
TGCACATCGATCCTGATAAGGAAGTTACTGTTACATCTGGGTGCACAGAAGCAATAGCTGC
AACGATATTGGGTCTGATCAACCCTGGGGATGAAGTCATACTGTTTGCTCCATTCTATGATT
CTTATGAGGCTACACTGTCCATGGCTGGTGCGAATGTCAAAGCCATTACACTCCGCCCTCC
GGACTTTGCAGTCCCTCTTGAAGAGCTAAAGGCTGCAGTCTCGAAGAATACCAGAGCAATA
ATGATTAATACACCTCACAACCCTACCGGGAAAATGTTCACAAGGGAGGAACTTGAGTTCA
TTGCTGATCTCTGCAAGGAAAATGACGTGTTGCTCTTTGCCGATGAGGTCTACGACAAGCT
GGCGTTTGAGGCGGATCACATATCAATGGCTTCTATTCCTGGCATGTATGAGAGGACCGTC
ACTATGAACTCCCTGGGGAAGACGTTCTCCTTGACCGGATGGAAGATCGGCTGGGCGATA
GCACCACCGCACCTGACATGGGGCGTAAGGCAGGCACACTCCTTCCTCACATTCGCCACC
TCCACGCCGATGCAATCAGCAGCGGCGGCGGCCCTGAGAGCACCGGACAGCTACTTTGA
GGAGCTGAAGAGGGACTACGGCGCAAAGAAAGCGCTGCTGGTGGACGGGCTCAAGGCG
GCGGGCTTCATCGTCTACCCTTCGAGCGGAACCTACTTCATCATGGTCGACCACACCCCG
TTCGGGTTCGACAACGACGTCGAGTTCTGCGAGTACTTGATCCGCGAGGTCGGCGTCGTG
GCCATCCCGCCAAGCGTGTTCTACCTGAACCCGGAGGACGGGAAGAACCTGGTGAGGTT
CACCTTCTGCAAGGACGACGACACGCTAAGGGCGGCGGTGGACAGGATGAAGGCCAAGC
TCAGGAAGAAATGA
SEQ ID NO: 15 Barley GPT amino acid sequence
Translated sequence from start site (intron removed)
MVDLRNRRTSMASAPASASAALSTAAPADNGAAKPTEQRPVQVAKRLEKFKTTIFTQMSMLAV
KHGAINLGQGFPNFDGPDFVKDAAIEAIKAGKNQYARGYGVPELNSAVAERFLKDSGLHIDPDK
EVTVTSGCTEAIAATILGLINPGDEVILFAPFYDSYEATLSMAGANVKAITLRPPDFAVPLEELKAA
VSKNTRAIMINTPHNPTGKMFTREELEFIADLCKENDVLLFADEVYDKLAFEADHISMASIPGMY
ERTVTMNSLGKTFSLTGWKIGWAIAPPHLTWGVRQAHSFLTFATSTPMQSAAAAALRAPDSYF
EELKRDYGAKKALLVDGLKAAGFIVYPSSGTYFIMVDHTPFGFDNDVEFCEYLIREVGVVAIPPS
VFYLNPEDGKNLVRFTFCKDDDTLRAAVDRMKAKLRKK
SEQ ID NO: 16 Zebra fish GPT DNA coding sequence
Danio rerio sequence designed for expression in E coli. Bold, italicized nucleotides added for
cloning or from pET28b vector. [symbol]
GTGGCGAAACGTCTGGAAAAATTTAAAACCACCATTTTTACCCAGATGAGCATGC
TGGCGATTAAACATGGCGCGATTAACCTGGGCCAGGGCTTTCCGAACTTTGATGGCCCGG
ATTTTGTGAAAGAAGCGGCGATTCAGGCGATTCGTGATGGCAACAACCAGTATGCGCGTG
GCTATGGCGTGCCGGATCTGAACATTGCGATTAGCGAACGTTATAAAAAAGATACCGGCCT
GGCGGTGGATCCGGAAAAAGAAATTACCGTGACCAGCGGCTGCACCGAAGCGATTGCGG
CGACCGTGCTGGGCCTGATTAACCCGGGCGATGAAGTGATTGTGTTTGCGCCGTTTTATG
ATAGCTATGAAGCGACCCTGAGCATGGCGGGCGCGAAAGTGAAAGGCATTACCCTGCGTC
CGCCGGATTTTGCGCTGCCGATTGAAGAACTGAAAAGCACCATTAGCAAAAACACCCGTG
CGATTCTGCTGAACACCCCGCATAACCCGACCGGCAAAATGTTTACCCCGGAAGAACTGA
ACACCATTGCGAGCCTGTGCATTGAAAACGATGTGCTGGTGTTTAGCGATGAAGTGTATGA
TAAACTGGCGTTTGATATGGAACATATTAGCATTGCGAGCCTGCCGGGCATGTTTGAACGT
ACCGTGACCATGAACAGCCTGGGCAAAACCTTTAGCCTGACCGGCTGGAAAATTGGCTGG
GCGATTGCGCCGCCGCATCTGACCTGGGGCGTGCGTCAGGCGCATGCGTTTCTGACCTTT
GCAACCAGCAACCCGATGCAGTGGGCAGCAGCAGTGGCACTGCGTGCACCGGATAGCTA
TTATACCGAACTGAAACGTGATTATATGGCGAAACGTAGCATTCTGGTGGAAGGCCTGAAA
GCGGTGGGCTTTAAAGTGTTTCCGAGCAGCGGCACCTATTTTGTGGTGGTGGATCATACC
CCGTTTGGCCATGAAAACGATATTGCGTTTTGCGAATATCTGGTGAAAGAAGTGGGCGTGG
TGGCGATTCCGACCAGCGTGTTTTATCTGAACCCGGAAGAAGGCAAAAACCTGGTGCGTTT
TACCTTTTGCAAAGATGAAGGCACCCTGCGTGCGGCGGTGGATCGTATGAAAGAAAAACT
GCGTAAA [symbol] [symbol] [symbol] [symbol] [symbol]
SEQ ID NO: 17 Zebra fish GPR amino acid sequence
Amino acid sequence of Danio rerio cloned and expressed in E. coli (bold, italicized amino
acids are added from vector/ cloning and His tag on C-terminus) [symbol]
VAKRLEKFKTTIFTQMSMLAIKHGAINLGQGFPNFDGPDFVKEAAIQAIRDGNNQYARGYGV
PDLNIAISERYKKDTGLAVDPEKEITVTSGCTEAIAATVLGLINPGDEVIVFAPFYDSYEATLSMA
GAKVKGITLRPPDFALPIEELKSTISKNTRAILLNTPHNPTGKMFTPEELNTIASLCIENDVLVFSD
EVYDKLAFDMEHISIASLPGMFERTVTMNSLGKTFSLTGWKIGWAIAPPHLTWGVRQAHAFLTF
ATSNPMQWAAAVALRAPDSYYTELKRDYMAKRSILVEGLKAVGFKVFPSSGTYFVVVDHTPFG
HENDIAFCEYLVKEVGVVAIPTSVFYLNPEEGKNLVRFTFCKDEGTLRAAVDRMKEKLRK [symbol] [symbol]
-
SEQ ID NO: 18 Arabidopsis truncated GPT-30 construct DNA sequence
Arabidopsis GPT coding sequence with 30 amino acids removed from the targeting sequence.
ATGGCCAAAATCCATCGTCCTATCGGAGCCACCATGACCACAGTTTCGACTCAGAACGAGT
CTACTCAAAAACCCGTCCAGGTGGCGAAGAGATTAGAGAAGTTCAAGACTACTATTTTCAC
TCAAATGAGCATATTGGCAGTTAAACATGGAGCGATCAATTTAGGCCAAGGCTTTCCCAATT
TCGACGGTCCTGATTTTGTTAAAGAAGCTGCGATCCAAGCTATTAAAGATGGTAAAAACCA
GTATGCTCGTGGATACGGCATTCCTCAGCTCAACTCTGCTATAGCTGCGCGGTTTCGTGAA
GATACGGGTCTTGTTGTTGATCCTGAGAAAGAAGTTACTGTTACATCTGGTTGCACAGAAG
CCATAGCTGCAGCTATGTTGGGTTTAATAAACCCTGGTGATGAAGTCATTCTCTTTGCACC
GTTTTATGATTCCTATGAAGCAACACTCTCTATGGCTGGTGCTAAAGTAAAAGGAATCACTT
TACGTCCACCGGACTTCTCCATCCCTTTGGAAGAGCTTAAAGCTGCGGTAACTAACAAGAC
TCGAGCCATCCTTATGAACACTCCGCACAACCCGACCGGGAAGATGTTCACTAGGGAGGA
GCTTGAAACCATTGCATCTCTCTGCATTGAAAACGATGTGCTTGTGTTCTCGGATGAAGTAT
ACGATAAGCTTGCGTTTGAAATGGATCACATTTCTATAGCTTCTCTTCCCGGTATGTATGAA
AGAACTGTGACCATGAATTCCCTGGGAAAGACTTTCTCTTTAACCGGATGGAAGATCGGCT
GGGCGATTGCGCCGCCTCATCTGACTTGGGGAGTTCGACAAGCACACTCTTACCTCACAT
TCGCCACATCAACACCAGCACAATGGGCAGCCGTTGCAGCTCTCAAGGCACCAGAGTCTT
ACTTCAAAGAGCTGAAAAGAGATTACAATGTGAAAAAGGAGACTCTGGTTAAGGGTTTGAA
GGAAGTCGGATTTACAGTGTTCCCATCGAGCGGGACTTACTTTGTGGTTGCTGATCACACT
CCATTTGGAATGGAGAACGATGTTGCTTTCTGTGAGTATCTTATTGAAGAAGTTGGGGTCG
TTGCGATCCCAACGAGCGTCTTTTATCTGAATCCAGAAGAAGGGAAGAATTTGGTTAGGTT
TGCGTTCTGTAAAGACGAAGAGACGTTGC
GTGGTGCAATTGAGAGGATGAAGCAGAAGCTTAAGAGAAAAGTCTGA
SEQ ID NO: 19 Arabidopsis truncated GPT-30 construct amino acid sequence
MAKIHRPIGATMTTVSTQNESTQKPVQVAKRLEKFKTTIFTQMSILAVKHGAINLGQGFPNFDGP
DFVKEAAIQAIKDGKNQYARGYGIPQLNSAIAARFREDTGLVVDPEKEVTVTSGCTEAIAAAMLG
LINPGDEVILFAPFYDSYEATLSMAGAKVKGITLRPPDFSIPLEELKAAVTNKTRAILMNTPHNPT
GKMFTREELETIASLCIENDVLVFSDEVYDKLAFEMDHISIASLPGMYERTVTMNSLGKTFSLTG
WKIGWAIAPPHLTWGVRQAHSYLTFATSTPAQWAAVAALKAPESYFKELKRDYNVKKETLVKG
LKEVGFTVFPSSGTYFVVADHTPFGMENDVAFCEYLIEEVGVVAIPTSVFYLNPEEGKNLVRFA
FCKDEETLRGAIERMKQKLKRKV
SEQ ID NO: 20: Arabidopsis truncated GPT-45 construct DNA sequence
Arabidopsis GPT coding sequence with 45 residues in the targeting sequence removed
ATGGCGACTCAGAACGAGTCTACTCAAAAACCCGTCCAGGTGGCGAAGAGATTAGAGAAG
TTCAAGACTACTATTTTCACTCAAATGAGCATATTGGCAGTTAAACATGGAGCGATCAATTT
AGGCCAAGGCTTTCCCAATTTCGACGGTCCTGATTTTGTTAAAGAAGCTGCGATCCAAGCT
ATTAAAGATGGTAAAAACCAGTATGCTCGTGGATACGGCATTCCTCAGCTCAACTCTGCTAT
AGCTGCGCGGTTTCGTGAAGATACGGGTCTTGTTGTTGATCCTGAGAAAGAAGTTACTGTT
ACATCTGGTTGCACAGAAGCCATAGCTGCAGCTATGTTGGGTTTAATAAACCCTGGTGATG
AAGTCATTCTCTTTGCACCGTTTTATGATTCCTATGAAGCAACACTCTCTATGGCTGGTGCT
AAAGTAAAAGGAATCACTTTACGTCCACCGGACTTCTCCATCCCTTTGGAAGAGCTTAAAG
CTGCGGTAACTAACAAGACTCGAGCCATCCTTATGAACACTCCGCACAACCCGACCGGGA
AGATGTTCACTAGGGAGGAGCTTGAAACCATTGCATCTCTCTGCATTGAAAACGATGTGCT
TGTGTTCTCGGATGAAGTATACGATAAGCTTGCGTTTGAAATGGATCACATTTCTATAGCTT
CTCTTCCCGGTATGTATGAAAGAACTGTGACCATGAATTCCCTGGGAAAGACTTTCTCTTTA
ACCGGATGGAAGATCGGCTGGGCGATTGCGCCGCCTCATCTGACTTGGGGAGTTCGACAA
GCACACTCTTACCTCACATTCGCCACATCAACACCAGCACAATGGGCAGCCGTTGCAGCTC
TCAAGGCACCAGAGTCTTACTTCAAAGAGCTGAAAAGAGATTACAATGTGAAAAAGGAGAC
TCTGGTTAAGGGTTTGAAGGAAGTCGGATTTACAGTGTTCCCATCGAGCGGGACTTACTTT
GTGGTTGCTGATCACACTCCATTTGGAATGGAGAACGATGTTGCTTTCTGTGAGTATCTTAT
TGAAGAAGTTGGGGTCGTTGCGATCCCAACGAGCGTCTTTTATCTGAATCCAGAAGAAGG
GAAGAATTTGGTTAGGTTTGCGTTCTGTAAAGACGAAGAGACGTTGCGTGGTGCAATTGAG
AGGATGAAGCAGAAGCTTAAGAGAAAAGTCTGA
SEQ ID NO: 21: Arabidopsis truncated GPT-45 construct amino acid sequence
MATQNESTQKPVQVAKRLEKFKTTIFTQMSILAVKHGAINLGQGFPNFDGPDFVKEAAIQAIKDG
KNQYARGYGIPQLNSAIAARFREDTGLVVDPEKEVTVTSGCTEAIAAAMLGLINPGDEVILFAPF
YDSYEATLSMAGAKVKGITLRPPDFSIPLEELKAAVTNKTRAILMNTPHNPTGKMFTREELETIA
SLCIENDVLVFSDEVYDKLAFEMDHISIASLPGMYERTVTMNSLGKTFSLTGWKIGWAIAPPHLT
WGVRQAHSYLTFATSTPAQWAAVAALKAPESYFKELKRDYNVKKETLVKGLKEVGFTVFPSSG
TYFVVADHTPFGMENDVAFCEYLIEEVGVVAIPTSVFYLNPEEGKNLVRFAFCKDEETLRGAIER
MKQKLKRKV
SEQ ID NO: 22: Tomato Rubisco promoter
TOMATO RuBisCo rbcS3C promoter sequence from KpnI to NcoI
GGTACCGTTTGAATCCTCCTTAAAGTTTTTCTCTGGAGAAACTGTAGTAATTTTACTTTGTTG
TGTTCCCTTCATCTTTTGAATTAATGGCATTTGTTTTAATACTAATCTGCTTCTGAAACTTGTA
ATGTATGTATATCAGTTTCTTATAATTTATCCAAGTAATATCTTCCATTCTCTATGCAATTGCC
TGCATAAGCTCGACAAAAGAGTACATCAACCCCTCCTCCTCTGGACTACTCTAGCTAAACTT
GAATTTCCCCTTAAGATTATGAAATTGATATATCCTTAACAAACGACTCCTTCTGTTGGAAAA
TGTAGTACTTGTCTTTCTTCTTTTGGGTATATATAGTTTATATACACCATACTATGTACAACAT
CCAAGTAGAGTGAAATGGATACATGTACAAGACTTATTTGATTGATTGATGACTTGAGTTGC
CTTAGGAGTAACAAATTCTTAGGTCAATAAATCGTTGATTTGAAATTAATCTCTCTGTCTTAG
ACAGATAGGAATTATGACTTCCAATGGTCCAGAAAGCAAAGTTCGCACTGAGGGTATACTT
GGAATTGAGACTTGCACAGGTCCAGAAACCAAAGTTCCCATCGAGCTCTAAAATCACATCT
TTGGAATGAAATTCAATTAGAGATAAGTTGCTTCATAGCATAGGTAAAATGGAAGATGTGAA
GTAACCTGCAATAATCAGTGAAATGACATTAATACACTAAATACTTCATATGTAATTATCCTT
TCCAGGTTAACAATACTCTATAAAGTAAGAATTATCAGAAATGGGCTCATCAAACTTTTGTA
CTATGTATTTCATATAAGGAAGTATAACTATACATAAGTGTATACACAACTTTATTCCTATTTT
GTAAAGGTGGAGAGACTGTTTTCGATGGATCTAAAGCAATATGTCTATAAAATGCATTGATA
TAATAATTATCTGAGAAAATCCAGAATTGGCGTTGGATTATTTCAGCCAAATAGAAGTTTGT
ACCATACTTGTTGATTCCTTCTAAGTTAAGGTGAAGTATCATTCATAAACAGTTTTCCCCAAA
GTACTACTCACCAAGTTTCCCTTTGTAGAATTAACAGTTCAAATATATGGCGCAGAAATTAC
TCTATGCCCAAAACCAAACGAGAAAGAAACAAAATACAGGGGTTGCAGACTTTATTTTCGT
GTTAGGGTGTGTTTTTTCATGTAATTAATCAAAAAATATTATGACAAAAACATTTATACATATT
TTTACTCAACACTCTGGGTATCAGGGTGGGTTGTGTTCGACAATCAATATGGAAAGGAAGT
ATTTTCCTTATTTTTTTAGTTAATATTTTCAGTTATACCAAACATACCTTGTGATATTATTTTTA
AAAATGAAAAACTCGTCAGAAAGAAAAAGCAAAAGCAACAAAAAAATTGCAAGTATTTTTTA
AAAAAGAAAAAAAAAACATATCTTGTTTGTCAGTATGGGAAGTTTGAGATAAGGACGAGTGA
GGGGTTAAAATTCAGTGGCCATTGATTTTGTAATGCCAAGAACCACAAAATCCAATGGTTAC
CATTCCTGTAAGATGAGGTTTGCTAACTCTTTTTGTCCGTTAGATAGGAAGCCTTATCACTA
TATATACAAGGCGTCCTAATAACCTCTTAGTAACCAATTATTTCAGCACCATGG
SEQ ID NO: 23: Bamboo GPT DNA coding sequence
ATGGCCTCCGCGGCCGTCTCCACCGTCGCCACCGCCGCCGACGGCGTCGCGAAGCCGA
CGGAGAAGCAGCCGGTACAGGTCGCAAAGCGTTTGGAAAAGTTTAAGACAACAATTTTCAC
ACAGATGAGCATGCTTGCCATCAAGCATGGAGCAATAAACCTCGGCCAGGGCTTTCCGAA
TTTTGATGGCCCTGACTTTGTGAAAGAAGCTGCTATTCAAGCTATCAATGCTGGGAAGAAT
CAGTATGCAAGAGGATATGGTGTGCCTGAACTGAACTCGGCTGTTGCTGAAAGGTTCCTGA
AGGACAGTGGCTTGCAAGTCGATCCCGAGAAGGAAGTTACTGTCACATCTGGGTGCACGG
AAGCGATAGCTGCAACGATATTGGGTCTTATCAACCCTGGCGATGAAGTGATCTTGTTTGC
TCCATTCTATGATTCATACGAGGCTACGCTGTCGATGGCTGGTGCCAATGTAAAAGCCATT
ACTCTCCGTCCTCCAGATTTTGCAGTCCCTCTTGAGGAGCTAAAGGCCACAGTCTCTAAGA
ACACCAGAGCGATAATGATAAACACACCACACAATCCTACTGGGAAAATGTTTTCTAGGGA
AGAACTTGAATTCATTGCTACTCTCTGCAAGAAAAATGATGTGTTGCTTTTTGCTGATGAGG
TCTATGACAAGTTGGCATTTGAGGCAGATCATATATCAATGGCTTCTATTCCTGGCATGTAT
GAGAGGACTGTGACTATGAACTCTCTGGGGAAGACATTCTCTCTAACAGGATGGAAGATCG
GTTGGGCAATAGCACCACCACACCTGACATGGGGTGTAAGGCAGGCACACTCATTCCTCA
CATTTGCCACCTGCACACCAATGCAATCGGCGGCGGCGGCGGCTCTTAGAGCACCAGATA
GCTACTATGGGGAGCTGAAGAGGGATTACGGTGCAAAGAAAGCGATACTAGTCGACGGAC
TCAAGGCTGCAGGTTTTATTGTTTACCCTTCAAGTGGAACATACTTTGTCATGGTCGATCAC
ACCCCGTTTGGTTTCGACAATGATATTGAGTTCTGCGAGTATTTGATCCGCGAAGTCGGTG
TTGTCGCCATACCACCAAGCGTATTTTATCTCAACCCTGAGGATGGGAAGAACTTGGTGAG
GTTCACCTTCTGCAAGGATGATGATACGCTGAGAGCCGCAGTTGAGAGGATGAAGACAAA
GCTCAGGAAAAAATGA
SEQ ID NO: 24: Bamboo GPT amino acid sequence
MASAAVSTVATAADGVAKPTEKQPVQVAKRLEKFKTTIFTQMSMLAIKHGAINLGQGFPNFDGP
DFVKEAAIQAINAGKNQYARGYGVPELNSAVAERFLKDSGLQVDPEKEVTVTSGCTEAIAATILG
LINPGDEVILFAPFYDSYEATLSMAGANVKAITLRPPDFAVPLEELKATVSKNTRAIMINTPHNPT
GKMFSREELEFIATLCKKNDVLLFADEVYDKLAFEADHISMASIPGMYERTVTMNSLGKTFSLTG
WKIGWAIAPPHLTWGVRQAHSFLTFATCTPMQSAAAAALRAPDSYYGELKRDYGAKKAILVDG
LKAAGFIVYPSSGTYFVMVDHTPFGFDNDIEFCEYLIREVGVVAIPPSVFYLNPEDGKNLVRFTF
CKDDDTLRAAVERMKTKLRKK
SEQ ID NO: 25: 1305.1 + rbcS3C promoter + catI intron with rice GPT gene.
Cambia1305.1 with (3′ end of) rbcS3C + rice GPT coding sequence. Underlined ATG is start
site, parentheses are the catI intron and the underlined actagt is the speI cloning site used to
splice in the rice gene.
AAAAAAGAAAAAAAAAACATATCTTGTTTGTCAGTATGGGAAGTTTGAGATAAGGACGAGTG
AGGGGTTAAAATTCAGTGGCCATTGATTTTGTAATGCCAAGAACCACAAAATCCAATGGTTA
CCATTCCTGTAAGATGAGGTTTGCTAACTCTTTTTGTCCGTTAGATAGGAAGCCTTATCACT
ATATATACAAGGCGTCCTAATAACCTCTTAGTAACCAATTATTTCAGCA [symbol] TAGATCT
GAGG(GTAAATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGTGTAAATAT
TACATAGCTTTAACTGATAATCTGATTACTTTATTTCGTGTGTCTATGATGATGATGATAGTT
ACAG)AACCGACGA [symbol] ATGAATCTGGCCGGCTTTCTCGCCACGCCCGCGACCGCGA
CCGCGACGCGGCATGAGATGCCGTTAAATCCCTCCTCCTCCGCCTCCTTCCTCCTCTCCT
CGCTCCGCCGCTCGCTCGTCGCGTCGCTCCGGAAGGCCTCGCCGGCGGCGGCCGCGGC
GCTCTCCCCCATGGCCTCCGCGTCCACCGTCGCCGCCGAGAACGGCGCCGCCAAGGCG
GCGGCGGAGAAGCAGCAGCAGCAGCCTGTGCAGGTTGCAAAGCGGTTGGAAAAGTTTAA
GACGACCATTTTCACACAGATGAGTATGCTTGCCATCAAGCATGGAGCAATAAACCTTGGC
CAGGGTTTTCCGAATTTCGATGGCCCTGACTTTGTAAAAGAGGCTGCTATTCAAGCTATCA
ATGCTGGGAAGAATCAGTACGCAAGAGGATATGGTGTGCCTGAACTGAACTCAGCTATTGC
TGAAAGATTCCTGAAGGACAGCGGACTGCAAGTCGATCCGGAGAAGGAAGTTACTGTCAC
ATCTGGATGCACAGAAGCTATAGCTGCAACAATTTTAGGTCTAATTAATCCAGGCGATGAA
GTGATATTGTTTGCTCCATTCTATGATTCATATGAGGCTACCCTGTCAATGGCTGGTGCCAA
CGTAAAAGCCATTACTCTCCGTCCTCCAGATTTTTCAGTCCCTCTTGAAGAGCTAAAGGCT
GCAGTCTCGAAGAACACCAGAGCTATTATGATAAACACCCCGCACAATCCTACTGGGAAAA
TGTTTACAAGGGAAGAACTTGAGTTTATTGCCACTCTCTGCAAGGAAAATGATGTGCTGCTT
TTTGCTGATGAGGTCTACGACAAGTTAGCTTTTGAGGCAGATCATATATCAATGGCTTCTAT
TCCTGGCATGTATGAGAGGACCGTGACCATGAACTCTCTTGGGAAGACATTCTCTCTTACA
GGATGGAAGATCGGTTGGGCAATCGCACCGCCACACCTGACATGGGGTGTAAGGCAGGC
ACACTCATTCCTCACGTTTGCGACCTGCACACCAATGCAAGCAGCTGCAGCTGCAGCTCTG
AGAGCACCAGATAGCTACTATGAGGAACTGAGGAGGGATTATGGAGCTAAGAAGGCATTG
CTAGTCAACGGACTCAAGGATGCAGGTTTCATTGTCTATCCTTCAAGTGGAACATACTTCGT
CATGGTCGACCACACCCCATTTGGTTTCGACAATGATATTGAGTTCTGCGAGTATTTGATTC
GCGAAGTCGGTGTTGTCGCCATACCACCTAGTGTATTTTATCTCAACCCTGAGGATGGGAA
GAACTTGGTGAGGTTCACCTTTTGCAAGGATGATGAGACGCTGAGAGCCGCGGTTGAGAG
GATGAAGACAAAGCTCAGGAAAAAATGA
SEQ ID NO: 26: HORDEUM GPT SEQUENCE INVECTOR
Cambia1305.1 with (3′ end of) rbcS3C + hordeum (IDI4) coding sequence. Underlined ATG is
start site, parentheses are the catI intron and the underlined actagt is the speI cloning site
used to splice in the hordeum gene.
AAAAAAGAAAAAAAAAACATATCTTGTTTGTCAGTATGGGAAGTTTGAGATAAGGACGAGTG
AGGGGTTAAAATTCAGTGGCCATTGATTTTGTAATGCCAAGAACCACAAAATCCAATGGTTA
CCATTCCTGTAAGATGAGGTTTGCTAACTCTTTTTGTCCGTTAGATAGGAAGCCTTATCACT
ATATATACAAGGCGTCCTAATAACCTCTTAGTAACCAATTATTTCAGCA [symbol] TAGATCT
GAGG(GTAAATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGTGTAAATAT
TACATAGCTTTAACTGATAATCTGATTACTTTATTTCGTGTGTCTATGATGATGATGATAGTT
ACAG)AACCGACGA [symbol] ATGGCATCCGCCCCCGCCTCCGCCTCCGCGGCCCTCTCCA
CCGCCGCCCCCGCCGACAACGGGGCCGCCAAGCCCACGGAGCAGCGGCCGGTACAGGT
GGCTAAGCGATTGGAGAAGTTCAAAACAACAATTTTCACACAGATGAGCATGCTCGCAGTG
AAGCATGGAGCAATAAACCTTGGACAGGGGTTTCCCAATTTTGATGGCCCTGACTTTGTCA
AAGATGCTGCTATTGAGGCTATCAAAGCTGGAAAGAATCAGTATGCAAGAGGATATGGTGT
GCCTGAATTGAACTCAGCTGTTGCTGAGAGATTTCTCAAGGACAGTGGATTGCACATCGAT
CCTGATAAGGAAGTTACTGTTACATCTGGGTGCACAGAAGCAATAGCTGCAACGATATTGG
GTCTGATCAACCCTGGGGATGAAGTCATACTGTTTGCTCCATTCTATGATTCTTATGAGGCT
ACACTGTCCATGGCTGGTGCGAATGTCAAAGCCATTACACTCCGCCCTCCGGACTTTGCA
GTCCCTCTTGAAGAGCTAAAGGCTGCAGTCTCGAAGAATACCAGAGCAATAATGATTAATA
CACCTCACAACCCTACCGGGAAAATGTTCACAAGGGAGGAACTTGAGTTCATTGCTGATCT
CTGCAAGGAAAATGACGTGTTGCTCTTTGCCGATGAGGTCTACGACAAGCTGGCGTTTGA
GGCGGATCACATATCAATGGCTTCTATTCCTGGCATGTATGAGAGGACCGTCACTATGAAC
TCCCTGGGGAAGACGTTCTCCTTGACCGGATGGAAGATCGGCTGGGCGATAGCACCACC
GCACCTGACATGGGGCGTAAGGCAGGCACACTCCTTCCTCACATTCGCCACCTCCACGCC
GATGCAATCAGCAGCGGCGGCGGCCCTGAGAGCACCGGACAGCTACTTTGAGGAGCTGA
AGAGGGACTACGGCGCAAAGAAAGCGCTGCTGGTGGACGGGCTCAAGGCGGCGGGCTTC
ATCGTCTACCCTTCGAGCGGAACCTACTTCATCATGGTCGACCACACCCCGTTCGGGTTCG
ACAACGACGTCGAGTTCTGCGAGTACTTGATCCGCGAGGTCGGCGTCGTGGCCATCCCGC
CAAGCGTGTTCTACCTGAACCCGGAGGACGGGAAGAACCTGGTGAGGTTCACCTTCTGCA
AGGACGACGACACGCTAAGGGCGGCGGTGGACAGGATGAAGGCCAAGCTCAGGAAGAAA
TGATTGAGGGGCG [symbol]
SEQ ID NO: 27 Expression cassette, Arabidopsis GPT coding sequence (ATG underlined)
under control of CMV 35S promoter (italics; promoter from Cambia 1201)
CATGGAGTCAAAGATTCAAATAGAGGACCTAACAGAACTCGCCGTAAAGACTGGCGAACA
GTTCATACAGAGTCTCTTACGACTCAATGACAAGAAGAAAATCTTCGTCAACATGGTGGAG
CACGACACACTTGTCTACTCCAAAAATATCAAAGATACAGTCTCAGAAGACCAAAGGGCAA
TTGAGACTTTTCAACAAAGGGTAATATCCGGAAACCTCCTCGGATTCCATTGCCCAGCTAT
CTGTCACTTTATTGTGAAGATAGTGGAAAAGGAAGGTGGCTCCTACAAATGCCATCATTGC
GATAAAGGAAAGGCCATCGTTGAAGATGCCTCTGCCGACAGTGGTCCCAAAGATGGACCC
CCACCCACGAGGAGCATCGTGGAAAAAGAAGACGTTCCAACCACGTCTTCAAAGCAAGTG
GATTGATGTGATATCTCCACTGACGTAAGGGATGACGCACAATCCCACTATCCTTCGCAAG
ACCCTTCCTCTATATAAGGAAGTTCATTTCATTTGGAGAGAACACGGGGGACTCTTGACCA
TGTACCTGGACATAAATGGTGTGATGATCAAACAGTTTAGCTTCAAAGCCTCTCTTCTCCCA
TTCTCTTCTAATTTCCGACAAAGCTCCGCCAAAATCCATCGTCCTATCGGAGCCACCATGA
CCACAGTTTCGACTCAGAACGAGTCTACTCAAAAACCCGTCCAGGTGGCGAAGAGATTAGA
GAAGTTCAAGACTACTATTTTCACTCAAATGAGCATATTGGCAGTTAAACATGGAGCGATCA
ATTTAGGCCAAGGCTTTCCCAATTTCGACGGTCCTGATTTTGTTAAAGAAGCTGCGATCCAA
GCTATTAAAGATGGTAAAAACCAGTATGCTCGTGGATACGGCATTCCTCAGCTCAACTCTG
CTATAGCTGCGCGGTTTCGTGAAGATACGGGTCTTGTTGTTGATCCTGAGAAAGAAGTTAC
TGTTACATCTGGTTGCACAGAAGCCATAGCTGCAGCTATGTTGGGTTTAATAAACCCTGGT
GATGAAGTCATTCTCTTTGCACCGTTTTATGATTCCTATGAAGCAACACTCTCTATGGCTGG
TGCTAAAGTAAAAGGAATCACTTTACGTCCACCGGACTTCTCCATCCCTTTGGAAGAGCTTA
AAGCTGCGGTAACTAACAAGACTCGAGCCATCCTTATGAACACTCCGCACAACCCGACCG
GGAAGATGTTCACTAGGGAGGAGCTTGAAACCATTGCATCTCTCTGCATTGAAAACGATGT
GCTTGTGTTCTCGGATGAAGTATACGATAAGCTTGCGTTTGAAATGGATCACATTTCTATAG
CTTCTCTTCCCGGTATGTATGAAAGAACTGTGACCATGAATTCCCTGGGAAAGACTTTCTCT
TTAACCGGATGGAAGATCGGCTGGGCGATTGCGCCGCCTCATCTGACTTGGGGAGTTCGA
CAAGCACACTCTTACCTCACATTCGCCACATCAACACCAGCACAATGGGCAGCCGTTGCAG
CTCTCAAGGCACCAGAGTCTTACTTCAAAGAGCTGAAAAGAGATTACAATGTGAAAAAGGA
GACTCTGGTTAAGGGTTTGAAGGAAGTCGGATTTACAGTGTTCCCATCGAGCGGGACTTAC
TTTGTGGTTGCTGATCACACTCCATTTGGAATGGAGAACGATGTTGCTTTCTGTGAGTATCT
TATTGAAGAAGTTGGGGTCGTTGCGATCCCAACGAGCGTCTTTTATCTGAATCCAGAAGAA
GGGAAGAATTTGGTTAGGTTTGCGTTCTGTAAAGACGAAGAGACGTTGCGTGGTGCAATTG
AGAGGATGAAGCAGAAGCTTAAGAGAAAAGTCTGA
SEQ ID NO: 28 Cambia p1305.1 with (3′ end of) rbcS3C + Arabidopsis GPT coding sequence.
Underlined ATG is start site, parentheses are the catI intron and the underlined actagt is the
speI cloning site used to splice in the Arabidopsis gene.
AAAAAAGAAAAAAAAAACATATCTTGTTTGTCAGTATGGGAAGTTTGAGATAAGGACGAGTG
AGGGGTTAAAATTCAGTGGCCATTGATTTTGTAATGCCAAGAACCACAAAATCCAATGGTTA
CCATTCCTGTAAGATGAGGTTTGCTAACTCTTTTTGTCCGTTAGATAGGAAGCCTTATCACT
ATATATACAAGGCGTCCTAATAACCTCTTAGTAACCAATTATTTCAGCA [symbol] TAGATCT
GAGG(GTAAATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGTGTAAATAT
TACATAGCTTTAACTGATAATCTGATTACTTTATTTCGTGTGTCTATGATGATGATGATAGTT
ACAG)AACCGACGA [symbol] ATGTACCTGGACATAAATGGTGTGATGATCAAACAGTTTAGC
TTCAAAGCCTCTCTTCTCCCATTCTCTTCTAATTTCCGACAAAGCTCCGCCAAAATCCATCG
TCCTATCGGAGCCACCATGACCACAGTTTCGACTCAGAACGAGTCTACTCAAAAACCCGTC
CAGGTGGCGAAGAGATTAGAGAAGTTCAAGACTACTATTTTCACTCAAATGAGCATATTGG
CAGTTAAACATGGAGCGATCAATTTAGGCCAAGGCTTTCCCAATTTCGACGGTCCTGATTTT
GTTAAAGAAGCTGCGATCCAAGCTATTAAAGATGGTAAAAACCAGTATGCTCGTGGATACG
GCATTCCTCAGCTCAACTCTGCTATAGCTGCGCGGTTTCGTGAAGATACGGGTCTTGTTGT
TGATCCTGAGAAAGAAGTTACTGTTACATCTGGTTGCACAGAAGCCATAGCTGCAGCTATG
TTGGGTTTAATAAACCCTGGTGATGAAGTCATTCTCTTTGCACCGTTTTATGATTCCTATGA
AGCAACACTCTCTATGGCTGGTGCTAAAGTAAAAGGAATCACTTTACGTCCACCGGACTTC
TCCATCCCTTTGGAAGAGCTTAAAGCTGCGGTAACTAACAAGACTCGAGCCATCCTTATGA
ACACTCCGCACAACCCGACCGGGAAGATGTTCACTAGGGAGGAGCTTGAAACCATTGCAT
CTCTCTGCATTGAAAACGATGTGCTTGTGTTCTCGGATGAAGTATACGATAAGCTTGCGTTT
GAAATGGATCACATTTCTATAGCTTCTCTTCCCGGTATGTATGAAAGAACTGTGACCATGAA
TTCCCTGGGAAAGACTTTCTCTTTAACCGGATGGAAGATCGGCTGGGCGATTGCGCCGCC
TCATCTGACTTGGGGAGTTCGACAAGCACACTCTTACCTCACATTCGCCACATCAACACCA
GCACAATGGGCAGCCGTTGCAGCTCTCAAGGCACCAGAGTCTTACTTCAAAGAGCTGAAA
AGAGATTACAATGTGAAAAAGGAGACTCTGGTTAAGGGTTTGAAGGAAGTCGGATTTACAG
TGTTCCCATCGAGCGGGACTTACTTTGTGGTTGCTGATCACACTCCATTTGGAATGGAGAA
CGATGTTGCTTTCTGTGAGTATCTTATTGAAGAAGTTGGGGTCGTTGCGATCCCAACGAGC
GTCTTTTATCTGAATCCAGAAGAAGGGAAGAATTTGGTTAGGTTTGCGTTCTGTAAAGACGA
AGAGACGTTGCGTGGTGCAATTGAGAGGATGAAGCAGAAGCTTAAGAGAAAAGTCTGA
SEQ ID NO: 29 Arabidpsis GPT coding sequence (mature protein, no targeting sequence)
GTGGCGAAGAGATTAGAGAAGTTCAAGACTACTATTTTCACTCAAATGAGCATATTGGCAG
TTAAACATGGAGCGATCAATTTAGGCCAAGGCTTTCCCAATTTCGACGGTCCTGATTTTGTT
AAAGAAGCTGCGATCCAAGCTATTAAAGATGGTAAAAACCAGTATGCTCGTGGATACGGCA
TTCCTCAGCTCAACTCTGCTATAGCTGCGCGGTTTCGTGAAGATACGGGTCTTGTTGTTGA
TCCTGAGAAAGAAGTTACTGTTACATCTGGTTGCACAGAAGCCATAGCTGCAGCTATGTTG
GGTTTAATAAACCCTGGTGATGAAGTCATTCTCTTTGCACCGTTTTATGATTCCTATGAAGC
AACACTCTCTATGGCTGGTGCTAAAGTAAAAGGAATCACTTTACGTCCACCGGACTTCTCC
ATCCCTTTGGAAGAGCTTAAAGCTGCGGTAACTAACAAGACTCGAGCCATCCTTATGAACA
CTCCGCACAACCCGACCGGGAAGATGTTCACTAGGGAGGAGCTTGAAACCATTGCATCTC
TCTGCATTGAAAACGATGTGCTTGTGTTCTCGGATGAAGTATACGATAAGCTTGCGTTTGAA
ATGGATCACATTTCTATAGCTTCTCTTCCCGGTATGTATGAAAGAACTGTGACCATGAATTC
CCTGGGAAAGACTTTCTCTTTAACCGGATGGAAGATCGGCTGGGCGATTGCGCCGCCTCA
TCTGACTTGGGGAGTTCGACAAGCACACTCTTACCTCACATTCGCCACATCAACACCAGCA
CAATGGGCAGCCGTTGCAGCTCTCAAGGCACCAGAGTCTTACTTCAAAGAGCTGAAAAGA
GATTACAATGTGAAAAAGGAGACTCTGGTTAAGGGTTTGAAGGAAGTCGGATTTACAGTGT
TCCCATCGAGCGGGACTTACTTTGTGGTTGCTGATCACACTCCATTTGGAATGGAGAACGA
TGTTGCTTTCTGTGAGTATCTTATTGAAGAAGTTGGGGTCGTTGCGATCCCAACGAGCGTC
TTTTATCTGAATCCAGAAGAAGGGAAGAATTTGGTTAGGTTTGCGTTCTGTAAAGACGAAGA
GACGTTGCGTGGTGCAATTGAGAGGATGAAGCAGAAGCTTAAGAGAAAAGTCTGA
SEQ ID NO: 30 Arabidpsis GPT amino acid sequence (mature protein, no targeting sequence)
VAKRLEKFKTTIFTQMSILAVKHGAINLGQGFPNFDGPDFVKEAAIQAIKDGKNQYARGYGIPQL
NSAIAARFREDTGLVVDPEKEVTVTSGCTEAIAAAMLGLINPGDEVILFAPFYDSYEATLSMAGA
KVKGITLRPPDFSIPLEELKAAVTNKTRAILMNTPHNPTGKMFTREELETIASLCIENDVLVFSDE
VYDKLAFEMDHISIASLPGMYERTVTMNSLGKTFSLTGWKIGWAIAPPHLTWGVRQAHSYLTFA
TSTPAQWAAVAALKAPESYFKELKRDYNVKKETLVKGLKEVGFTVFPSSGTYFVVADHTPFGM
ENDVAFCEYLIEEVGVVAIPTSVFYLNPEEGKNLVRFAFCKDEETLRGAIERMKQKLKRKV
SEQ ID NO: 31 Grape GPT amino acid sequence (mature protein, no targeting sequence)
VAKRLEKFKTTIFTQMSMLAIKHGAINLGQGFPNFDGPEFVKEAAIQAIKDGKNQYARGYGVPD
LNSAVADRFKKDTGLVVDPEKEVTVTSGCTEAIAATMLGLINPGDEVILFAPFYDSYEATLSMAG
AQIKSITLRPPDFAVPMDELKSAISKNTRAILINTPHNPTGKMFTREELNVIASLCIENDVLVFTDE
VYDKLAFEMDHISMASLPGMYERTVTMNSLGKTFSLTGWKIGWTVAPPHLTWGVRQAHSFLT
FATCTPMQWAAATALRAPDSYYEELKRDYSAKKAILVEGLKAVGFRVYPSSGTYFVVVDHTPF
GLKDDIAFCEYLIKEVGVVAIPTSVFYLHPEDGKNLVRFTFCKDEGTLRAAVERMKEKLKPKQ
SEQ ID NO: 32 Rice GPT amino acid sequence (mature protein, no targeting sequence)
VAKRLEKFKTTIFTQMSMLAIKHGAINLGQGFPNFDGPDFVKEAAIQAINAGKNQYARGYGVPE
LNSAIAERFLKDSGLQVDPEKEVTVTSGCTEAIAATILGLINPGDEVILFAPFYDSYEATLSMAGA
NVKAITLRPPDFSVPLEELKAAVSKNTRAIMINTPHNPTGKMFTREELEFIATLCKENDVLLFADE
VYDKLAFEADHISMASIPGMYERTVTMNSLGKTFSLTGWKIGWAIAPPHLTWGVRQAHSFLTFA
TCTPMQAAAAAALRAPDSYYEELRRDYGAKKALLVNGLKDAGFIVYPSSGTYFVMVDHTPFGF
DNDIEFCEYLIREVGVVAIPPSVFYLNPEDGKNLVRFTFCKDDETLRAAVERMKTKLRKK
SEQ ID NO: 33 Soybean GPT amino acid sequence (−1 mature protein, no targeting sequence)
AKRLEKFQTTIFTQMSLLAIKHGAINLGQGFPNFDGPEFVKEAAIQAIRDGKNQYARGYGVPDLN
IAIAERFKKDTGLVVDPEKEITVTSGCTEAIAATMIGLINPGDEVIMFAPFYDSYEATLSMAGAKV
KGITLRPPDFAVPLEELKSTISKNTRAILINTPHNPTGKMFTREELNCIASLCIENDVLVFTDEVYD
KLAFDMEHISMASLPGMFERTVTLNSLGKTFSLTGWKIGWAIAPPHLSWGVRQAHAFLTFATA
HPFQCAAAAALRAPDSYYVELKRDYMAKRAILIEGLKAVGFKVFPSSGTYFVVVDHTPFGLEND
VAFCEYLVKEVGVVAIPTSVFYLNPEEGKNLVRFTFCKDEETIRSAVERMKAKLRKVD
SEQ ID NO: 34 Barley GPT amino acid sequence (mature protein, no targeting sequence)
VAKRLEKFKTTIFTQMSMLAVKHGAINLGQGFPNFDGPDFVKDAAIEAIKAGKNQYARGYGVPE
LNSAVAERFLKDSGLHIDPDKEVTVTSGCTEAIAATILGLINPGDEVILFAPFYDSYEATLSMAGA
NVKAITLRPPDFAVPLEELKAAVSKNTRAIMINTPHNPTGKMFTREELEFIADLCKENDVLLFADE
VYDKLAFEADHISMASIPGMYERTVTMNSLGKTFSLTGWKIGWAIAPPHLTWGVRQAHSFLTFA
TSTPMQSAAAAALRAPDSYFEELKRDYGAKKALLVDGLKAAGFIVYPSSGTYFIMVDHTPFGFD
NDVEFCEYLIREVGVVAIPPSVFYLNPEDGKNLVRFTFCKDDDTLRAAVDRMKAKLRKK
SEQ ID NO: 35 Zebra fish GPT amino acid sequence (mature protein, no targeting sequence)
VAKRLEKFKTTIFTQMSMLAIKHGAINLGQGFPNFDGPDFVKEAAIQAIRDGNNQYARGYGVPD
LNIAISERYKKDTGLAVDPEKEITVTSGCTEAIAATVLGLINPGDEVIVFAPFYDSYEATLSMAGAK
VKGITLRPPDFALPIEELKSTISKNTRAILLNTPHNPTGKMFTPEELNTIASLCIENDVLVFSDEVY
DKLAFDMEHISIASLPGMFERTVTMNSLGKTFSLTGWKIGWAIAPPHLTWGVRQAHAFLTFATS
NPMQWAAAVALRAPDSYYTELKRDYMAKRSILVEGLKAVGFKVFPSSGTYFVVVDHTPFGHEN
DIAFCEYLVKEVGVVAIPTSVFYLNPEEGKNLVRFTFCKDEGTLRAAVDRMKEKLRK
SEQ ID NO: 36 Bamboo GPT amino acid sequence (mature protein, no targeting sequence)
VAKRLEKFKTTIFTQMSMLAIKHGAINLGQGFPNFDGPDFVKEAAIQAINAGKNQYARGYGVPE
LNSAVAERFLKDSGLQVDPEKEVTVTSGCTEAIAATILGLINPGDEVILFAPFYDSYEATLSMAGA
NVKAITLRPPDFAVPLEELKATVSKNTRAIMINTPHNPTGKMFSREELEFIATLCKKNDVLLFADE
VYDKLAFEADHISMASIPGMYERTVTMNSLGKTFSLTGWKIGWAIAPPHLTWGVRQAHSFLTFA
TCTPMQSAAAAALRAPDSYYGELKRDYGAKKAILVDGLKAAGFIVYPSSGTYFVMVDHTPFGF
DNDIEFCEYLIREVGVVAIPPSVFYLNPEDGKNLVRFTFCKDDDTLRAAVERMKTKLRKK
SEQ ID NO: 39 Rice rubisco promoter deposited in NCBI GenBank: AF143510.1
PstI cloning sites in bold; NcoI cloning site in italics, catl intron and part of Gus plus
protein from Cambia 1305.1 vector in bold underline (sequence removed and not translated),
3′ terminal SpeI cloning site in double underline. The construct also includes a PmlI 1305.1
cloning site CACGTG (also cuts in rice rbsc promoter), and a ZraI cloning site GACGTC, which
can be added by PCR to clone into PmlI site of vector).
CTGCAGCAAAGAAACGTTATTAGTTGGTGCTTTTGGTGGTAGGAATGTAGTTTTCTGACAAA
GTCAATTACTGAATATAAAAAAAATCTGCACAGCTCTGCGTCAACAGTTGTCCAAGGGATG
CCTCAAAAATCTGTGCAGATTATCAGTCGTCACGCAGAAGCAGAACATCATGGTGTGCTAG
GTCAGCTTCTTGCATTGGGCCATGAATCCGGTTGGTTGTTAATCTCTCCTCTCTTATTCTCT
TATATTAAGATGCATAACTCTTTTATGTAGTCTAAAAAAAAATCCAGTGGATCGGATAGTAGT
ACGTCATGGTGCCATTAGGTACCGTTGAACCTAACAGATATTTATGCATGTGTATATATATA
GCTATATAGACAAAATTGATGCCGATTATAGACCCAAAAGCAATAGGTATATATAATATAATA
CAGACCACACCACCAAACTAAGAATCGATCAAATAGACAAGGCATGTCTCCAAATTGTCTTA
AACTATTTCCGTAGGTTCAGCCGTTCAGGAGTCGAATCAGCCTCTGCCGGCGTTTTCTTTG
CACGTACGACGGACACACATGGGCATACCATATAGCTGGTCCATGACATTAGGAGAGAGA
ACGTACGTGTTGACCTGTAGCTGAGATATAACAAGGTTGATTATAATATCACCAAACATGAA
ATCATCCAAGGATGACCCATAACTATCACTACTATAGTACTGCATCTGGTAAAAGAAATTGT
ATAGACTCTATTTCGAGCACTACCACATAACGCCTGCAATGTGACACCCTACCTATTCACTA
ATGTGCCTCTTCCCACACGCTTTCCACCCGTACTGCTCACAGCTTTAAGAACCAGAACAAA
TGAGTAATATTAGTGTCGGTTCATGGCTAAAACCAGCACTGATGTACATGACCACATATGTC
AAATGCTGCTTCTAGGCATGACCCGCTCTTACTAATACCTACTCATCGCTAGAAGAATTTTC
GGCTGATAAATTTTCAATTTAAGCAAGAGTTATCTGCGTTGGTTCATAACTCAAACTGATGG
CCCCAACCATATTAGTGCAAATTTCACATATGATCATAACCTTTTCATATGAAATCGGATCG
AGATGAACTTTATATAAACATTGTAGCTGTCGATGATACCTACAATTTTATAGTTCACAACCT
TTTTATTTCAAGTCATTTAAATGCCCAAATAGGTGTTTCAAATCTCAGATAGAAATGTTCAAA
AGTAAAAAAGGTCCCTATCATAACATAATTGATATGTAAGTGAGTTGGAAAAAGATAAGTAC
GTGTGAGAGAGATCGGGGATCAAATTCTGGTGTAATAATGTATGTATTTCAGTCATAAAAAT
TGGTAGCAGTAGTTGGGGCTCTGTATATATACCGGTAAGGATGGGATGGTAGTAGAATAAT
TCTTTTTTTGTTTTTAGTTTTTTCTGGTCCAAAATTTCAAATTTGGATCCCTTACTTGTACCAA
CTAATATTAATGAGTGTTGAGGGTAGTAGAGGTGCAACTTTACCATAATCCCTCTGTTTCAG
GTTATAAGACGTTTTGACTTTAAATTTGACCAAGTTTATGCGCAAATATAGTAATATTTATAA
TACTATATTAGTTTCATTAAATAAATAATTGAATATATTTTCATAATAAATTTGTGTTGAGTTC
AAAATATTATTAATTTTTTCTACAAACTTGGTCAAACTTGAAGCAGTTTGACTTTGACCAAAG
TCAAAACGTCTTATAACTTGAAACGGATGGATTACTTTTTTTGTGGGGACAAGTTTACAATG
TTTAATAAAGCACAATCCATCTTAATGTTTTCAAGCTGAATATTGTAAAATTCATGGATAAAC
CAGCTTCTAAATGTTTAACCGGGAAAATGTCGAACGACAAATTAATATTTTTAAGTGATGGG
GAGTATTAATTAAGGAGTGACAACTCAACTTTCAATATCGTACTAAACTGTGGGATTTATTTT
CTAAAATTTTATACCCTGCCAATTCACGTGTTGTAGATCTTTTTTTTTCACTAACCGACACCA
GGTATATCAATTTTATTGAATATAGCAGCAAAAAGAATGTGTTGTACTTGTAAACAAAAAGCA
AACTGTACATAAAAAAAAATGCACTCCTATATAATTAAGCTCATAAAGATGCTTTGCTTCGTG
AGGGCCCAAGTTTTGATGACCTTTTGCTTGATCTCGAAATTAAAATTTAAGTACTGTTAAGG
GAGTTCACACCACCATCAATTTTCAGCCTGAAGAAACAGTTAAACAACGACCCCGATGACC
AGTCTACTGCTCTCCACATACTAGCTGCATTATTGATCACAAAACAAAACAAAACGAAATAA
AAATCAGCAGCGAGAGTGTGCAGAGAGAGACAAAGGTGATCTGGCGTGGATATCTCCCCA
TCCATCCTCACCCGCGCTGCCCATCACTCGCCGCCGCATACTCCATCATGTGGAGAGAGG
AAGACGAGGACCACAGCCAGAGCCCGGGTCGAGATGCCACCACGGCCACAACCCACGAG
CCCGGCGCGACACCACCGCGCGCGCGTGAGCCAGCCACAAACGCCCGCGGATAGGCGC
GCGCACGCCGGCCAATCCTACCACATCCCCGGCCTCCGCGGCTCGCGAGCGCCGCTGCC
ATCCGATCCGCTGAGTTTTGGCTATTTATACGTACCGCGGGAGCCTGTGTGCAGAGCAGT
GCATCTCAAGAAGTACTCGAGCAAAGAAGGAGAGAGCTTGGTGAGCTGCAGCCATGGTAG
ATCTGAGGGTAAATTTCTAGTTTTTCTCCTTCATTTTCTTGGTTAGGACCCTTTTCTCTTTTT
ATTTTTTTGAGCTTTGATCTTTCTTTAAACTGATCTATTTTTTAATTGATTGGTTATGGTGTA
AATATTACATAGCTTTAACTGATAATCTGATTACTTTATTTCGTGTGTCTATGATGATGATG
ATAGTTACAGAACCGACGAACTAGT
SEQ ID NO: 40 Horeum GS1 coding sequence
GCGCAGGCGGTTGTGCAGGCGATGCAGTGCCAGGTGGGGGTGAGGGGCAGGACGGCCG
TCCCGGCGAGGCAGCCCGCGGGCAGGGTGTGGGGCGTCAGGAGGGCCGCCCGCGCCA
CCTCCGGGTTCAAGGTGCTGGCGCTCGGCCCGGAGACCACCGGGGTCATCCAGAGGATG
CAGCAGCTGCTCGACATGGACACCACGCCCTTCACCGACAAGATCATCGCCGAGTACATC
TGGGTTGGAGGATCTGGAATTGACCTCAGAAGCAAATCAAGGACGATTTCGAAGCCAGTG
GAGGACCCGTCAGAGCTGCCGAAATGGAACTACGACGGATCGAGCACGGGGCAGGCTCC
TGGGGAAGACAGTGAAGTCATCCTATACCCACAGGCCATATTCAAGGACCCATTCCGAGG
AGGCAACAACATACTGGTTATCTGTGACACCTACACACCACAGGGGGAACCCATCCCTACT
AACAAACGCCACATGGCTGCACAAATCTTCAGTGACCCCAAGGTCACTTCACAAGTGCCAT
GGTTCGGAATCGAACAGGAGTACACTCTGATGCAGAGGGATGTGAACTGGCCTCTTGGCT
GGCCTGTTGGAGGGTACCCTGGCCCCCAGGGTCCATACTACTGCGCCGTAGGATCAGAC
AAGTCATTTGGCCGTGACATATCAGATGCTCACTACAAGGCGTGCCTTTACGCTGGAATTG
AAATCAGTGGAACAAACGGGGAGGTCATGCCTGGTCAGTGGGAGTACCAGGTTGGACCCA
GCGTTGGTATTGATGCAGGAGACCACATATGGGCTTCCAGATACATTCTCGAGAGAATCAC
GGAGCAAGCTGGTGTGGTGCTCACCCTTGACCCAAAACCAATCCAGGGTGACTGGAACGG
AGCTGGCTGCCACACAAACTACAGCACATTGAGCATGCGCGAGGATGGAGGTTTCGACGT
GATCAAGAAGGCAATCCTGAACCTTTCACTTCGCCATGACTTGCACATAGCCGCATATGGT
GAAGGAAACGAGCGGAGGTTGACAGGGCTACACGAGACAGCTAGCATATCAGACTTCTCA
TGGGGTGTGGCGAACCGTGGCTGCTCTATTCGTGTGGGGCGAGACACCGAGGCGAAGGG
CAAAGGATACCTGGAGGACCGTCGCCCGGCCTCCAACATGGACCCGTACACCGTGACGG
CGCTGCTGGCCGAGACCACGATCCTGTGGGAGCCGACCCTCGAGGCGGAGGCCCTCGCT
GCCAAGAAGCTGGCGCTGAAGGTATGA
SEQ ID NO: 41 Horeum GS1 amino acid sequence
AQAVVQAMQCQVGVRGRTAVPARQPAGRVWGVRRAARATSGFKVLALGPETTGVIQRMQQL
LDMDTTPFTDKIIAEYIWVGGSGIDLRSKSRTISKPVEDPSELPKWNYDGSSTGQAPGEDSEVIL
YPQAIFKDPFRGGNNILV6ICDTYTPQGEPIPTNKRHMAAQIFSDPKVTSQVPWFGIEQEYTLMQ
RDVNWPLGWPVGGYPGPQGPYYCAVGSDKSFGRDISDAHYKACLYAGIEISGTNGEVMPGQ
WEYQVGPSVGIDAGDHIWASRYILERITEQAGVVLTLDPKPIQGDWNGAGCHTNYSTLSMRED
GGFDVIKKAILNLSLRHDLHIAAYGEGNERRLTGLHETASISDFSWGVANRGCSIRVGRDTEAK
GKGYLEDRRPASNMDPYTVTALLAETTILWEPTLEAEALAAKKLALKV
SEQ ID NO: 42: Expression cassette combining SEQ ID NO: 39 (5′) and SEQ ID NO: 40 (3′),
encoding the Rice rubisco promoter, catI intron and part of Gus plus protein, and hordeum GS1.
Features shown as in SEQ ID NO: 39. Hordeum GS1 coding sequence begins after SpeI
cloning site (double underline).
CTGCAGCAAAGAAACGTTATTAGTTGGTGCTTTTGGTGGTAGGAATGTAGTTTTCTGACAAA
GTCAATTACTGAATATAAAAAAAATCTGCACAGCTCTGCGTCAACAGTTGTCCAAGGGATG
CCTCAAAAATCTGTGCAGATTATCAGTCGTCACGCAGAAGCAGAACATCATGGTGTGCTAG
GTCAGCTTCTTGCATTGGGCCATGAATCCGGTTGGTTGTTAATCTCTCCTCTCTTATTCTCT
TATATTAAGATGCATAACTCTTTTATGTAGTCTAAAAAAAAATCCAGTGGATCGGATAGTAGT
ACGTCATGGTGCCATTAGGTACCGTTGAACCTAACAGATATTTATGCATGTGTATATATATA
GCTATATAGACAAAATTGATGCCGATTATAGACCCAAAAGCAATAGGTATATATAATATAATA
CAGACCACACCACCAAACTAAGAATCGATCAAATAGACAAGGCATGTCTCCAAATTGTCTTA
AACTATTTCCGTAGGTTCAGCCGTTCAGGAGTCGAATCAGCCTCTGCCGGCGTTTTCTTTG
CACGTACGACGGACACACATGGGCATACCATATAGCTGGTCCATGACATTAGGAGAGAGA
ACGTACGTGTTGACCTGTAGCTGAGATATAACAAGGTTGATTATAATATCACCAAACATGAA
ATCATCCAAGGATGACCCATAACTATCACTACTATAGTACTGCATCTGGTAAAAGAAATTGT
ATAGACTCTATTTCGAGCACTACCACATAACGCCTGCAATGTGACACCCTACCTATTCACTA
ATGTGCCTCTTCCCACACGCTTTCCACCCGTACTGCTCACAGCTTTAAGAACCAGAACAAA
TGAGTAATATTAGTGTCGGTTCATGGCTAAAACCAGCACTGATGTACATGACCACATATGTC
AAATGCTGCTTCTAGGCATGACCCGCTCTTACTAATACCTACTCATCGCTAGAAGAATTTTC
GGCTGATAAATTTTCAATTTAAGCAAGAGTTATCTGCGTTGGTTCATAACTCAAACTGATGG
CCCCAACCATATTAGTGCAAATTTCACATATGATCATAACCTTTTCATATGAAATCGGATCG
AGATGAACTTTATATAAACATTGTAGCTGTCGATGATACCTACAATTTTATAGTTCACAACCT
TTTTATTTCAAGTCATTTAAATGCCCAAATAGGTGTTTCAAATCTCAGATAGAAATGTTCAAA
AGTAAAAAAGGTCCCTATCATAACATAATTGATATGTAAGTGAGTTGGAAAAAGATAAGTAC
GTGTGAGAGAGATCGGGGATCAAATTCTGGTGTAATAATGTATGTATTTCAGTCATAAAAAT
TGGTAGCAGTAGTTGGGGCTCTGTATATATACCGGTAAGGATGGGATGGTAGTAGAATAAT
TCTTTTTTTGTTTTTAGTTTTTTCTGGTCCAAAATTTCAAATTTGGATCCCTTACTTGTACCAA
CTAATATTAATGAGTGTTGAGGGTAGTAGAGGTGCAACTTTACCATAATCCCTCTGTTTCAG
GTTATAAGACGTTTTGACTTTAAATTTGACCAAGTTTATGCGCAAATATAGTAATATTTATAA
TACTATATTAGTTTCATTAAATAAATAATTGAATATATTTTCATAATAAATTTGTGTTGAGTTC
AAAATATTATTAATTTTTTCTACAAACTTGGTCAAACTTGAAGCAGTTTGACTTTGACCAAAG
TCAAAACGTCTTATAACTTGAAACGGATGGATTACTTTTTTTGTGGGGACAAGTTTACAATG
TTTAATAAAGCACAATCCATCTTAATGTTTTCAAGCTGAATATTGTAAAATTCATGGATAAAC
CAGCTTCTAAATGTTTAACCGGGAAAATGTCGAACGACAAATTAATATTTTTAAGTGATGGG
GAGTATTAATTAAGGAGTGACAACTCAACTTTCAATATCGTACTAAACTGTGGGATTTATTTT
CTAAAATTTTATACCCTGCCAATTCACGTGTTGTAGATCTTTTTTTTTCACTAACCGACACCA
GGTATATCAATTTTATTGAATATAGCAGCAAAAAGAATGTGTTGTACTTGTAAACAAAAAGCA
AACTGTACATAAAAAAAAATGCACTCCTATATAATTAAGCTCATAAAGATGCTTTGCTTCGTG
AGGGCCCAAGTTTTGATGACCTTTTGCTTGATCTCGAAATTAAAATTTAAGTACTGTTAAGG
GAGTTCACACCACCATCAATTTTCAGCCTGAAGAAACAGTTAAACAACGACCCCGATGACC
AGTCTACTGCTCTCCACATACTAGCTGCATTATTGATCACAAAACAAAACAAAACGAAATAA
AAATCAGCAGCGAGAGTGTGCAGAGAGAGACAAAGGTGATCTGGCGTGGATATCTCCCCA
TCCATCCTCACCCGCGCTGCCCATCACTCGCCGCCGCATACTCCATCATGTGGAGAGAGG
AAGACGAGGACCACAGCCAGAGCCCGGGTCGAGATGCCACCACGGCCACAACCCACGAG
CCCGGCGCGACACCACCGCGCGCGCGTGAGCCAGCCACAAACGCCCGCGGATAGGCGC
GCGCACGCCGGCCAATCCTACCACATCCCCGGCCTCCGCGGCTCGCGAGCGCCGCTGCC
ATCCGATCCGCTGAGTTTTGGCTATTTATACGTACCGCGGGAGCCTGTGTGCAGAGCAGT
GCATCTCAAGAAGTACTCGAGCAAAGAAGGAGAGAGCTTGGTGAGCTGCAGCCATGGTAG
ATCTGAGGGTAAATTTCTAGTTTTTCTCCTTCATTTTCTTGGTTAGGACCCTTTTCTCTTTTTA
TTTTTTTGAGCTTTGATCTTTCTTTAAACTGATCTATTTTTTAATTGATTGGTTATGGTGTAAA
TATTACATAGCTTTAACTGATAATCTGATTACTTTATTTCGTGTGTCTATGATGATGATGATA
GTTACAGAACCGACGAACTAGTGCGCAGGCGGTTGTGCAGGCGATGCAGTGCCAGGTGG
GGGTGAGGGGCAGGACGGCCGTCCCGGCGAGGCAGCCCGCGGGCAGGGTGTGGGGCG
TCAGGAGGGCCGCCCGCGCCACCTCCGGGTTCAAGGTGCTGGCGCTCGGCCCGGAGAC
CACCGGGGTCATCCAGAGGATGCAGCAGCTGCTCGACATGGACACCACGCCCTTCACCG
ACAAGATCATCGCCGAGTACATCTGGGTTGGAGGATCTGGAATTGACCTCAGAAGCAAATC
AAGGACGATTTCGAAGCCAGTGGAGGACCCGTCAGAGCTGCCGAAATGGAACTACGACG
GATCGAGCACGGGGCAGGCTCCTGGGGAAGACAGTGAAGTCATCCTATACCCACAGGCC
ATATTCAAGGACCCATTCCGAGGAGGCAACAACATACTGGTTATCTGTGACACCTACACAC
CACAGGGGGAACCCATCCCTACTAACAAACGCCACATGGCTGCACAAATCTTCAGTGACC
CCAAGGTCACTTCACAAGTGCCATGGTTCGGAATCGAACAGGAGTACACTCTGATGCAGA
GGGATGTGAACTGGCCTCTTGGCTGGCCTGTTGGAGGGTACCCTGGCCCCCAGGGTCCA
TACTACTGCGCCGTAGGATCAGACAAGTCATTTGGCCGTGACATATCAGATGCTCACTACA
AGGCGTGCCTTTACGCTGGAATTGAAATCAGTGGAACAAACGGGGAGGTCATGCCTGGTC
AGTGGGAGTACCAGGTTGGACCCAGCGTTGGTATTGATGCAGGAGACCACATATGGGCTT
CCAGATACATTCTCGAGAGAATCACGGAGCAAGCTGGTGTGGTGCTCACCCTTGACCCAA
AACCAATCCAGGGTGACTGGAACGGAGCTGGCTGCCACACAAACTACAGCACATTGAGCA
TGCGCGAGGATGGAGGTTTCGACGTGATCAAGAAGGCAATCCTGAACCTTTCACTTCGCC
ATGACTTGCACATAGCCGCATATGGTGAAGGAAACGAGCGGAGGTTGACAGGGCTACACG
AGACAGCTAGCATATCAGACTTCTCATGGGGTGTGGCGAACCGTGGCTGCTCTATTCGTGT
GGGGCGAGACACCGAGGCGAAGGGCAAAGGATACCTGGAGGACCGTCGCCCGGCCTCC
AACATGGACCCGTACACCGTGACGGCGCTGCTGGCCGAGACCACGATCCTGTGGGAGCC
GACCCTCGAGGCGGAGGCCCTCGCTGCCAAGAAGCTGGCGCTGAAGGTATGA
SEQ ID NO: 43 Amino acid sequence of translation product of SEQ ID NO: 42.
Amino-terminal bold residues from Gusplus and SpeI cloning site (intron removed)
MVDLRNRRTSAQAVVQAMQCQVGVRGRTAVPARQPAGRVWGVRRAARATSGFKVLALGPE
TTGVIQRMQQLLDMDTTPFTDKIIAEYIWVGGSGIDLRSKSRTISKPVEDPSELPKVVNYDGSST
GQAPGEDSEVILYPQAIFKDPFRGGNNILVICDTYTPQGEPIPTNKRHMAAQIFSDPKVTSQVP
WFGIEQEYTLMQRDVNWPLGWPVGGYPGPQGPYYCAVGSDKSFGRDISDAHYKACLYAGIEI
SGTNGEVMPGQWEYQVGPSVGIDAGDHIWASRYILERITEQAGVVLTLDPKPIQGDWNGAGC
HTNYSTLSMREDGGFDVIKKAILNLSLRHDLHIAAYGEGNERRLTGLHETASISDFSWGVANRG
CSIRVGRDTEAKGKGYLEDRRPASNMDPYTVTALLAETTILWEPTLEAEALAAKKLALKV
SEQ ID NO: 44 Maize ubiI promoter: 5′UTR intron shown in italics, TATA box at −30 is
underlined, 5′ and 3′ PstI cloning sites in bold
CTGCAGTGCAGCGTGACCCGGTCGTGCCCCTCTCTAGAGATAATGAGCATTGCATGTCTA
AGTTATAAAAAATTACCACATATTTTTTTTGTCACACTTGTTTGAAGTGCAGTTTATCTATCTT
TATACATATATTTAAACTTTACTCTACGAATAATATAATCTATAGTACTACAATAATATCAGTG
TTTTAGAGAATCATATAAATGAACAGTTAGACATGGTCTAAAGGACAATTGAGTATTTTGAC
AACAGGACTCTACAGTTTTATCTTTTTAGTGTGCATGTGTTCTCCTTTTTTTTTGCAAATAGC
TTCACCTATATAATACTTCATCCATTTTATTAGTACATCCATTTAGGGTTTAGGGTTAATGGT
TTTTATAGACTAATTTTTTTAGTACATCTATTTTATTCTATTTTAGCCTCTAAATTAAGAAAACT
AAAACTCTATTTTAGTTTTTTTATTTAATAATTTAGATATAAAATAGAATAAAATAAAGTGACT
AAAAATTAAACAAATACCCTTTAAGAAATTAAAAAAACTAAGGAAACATTTTTCTTGTTTCGA
GTAGATAATGCCAGCCTGTTAAACGCCGTCGACGAGTCTAACGGACACCAACCAGCGAAC
CAGCAGCGTCGCGTCGGGCCAAGCGAAGCAGACGGCACGGCATCTCTGTCGCTGCCTCT
GGACCCCTCTCGAGAGTTCCGCTCCACCGTTGGACTTGCTCCGCTGTCGGCATCCAGAAA
TTGCGTGGCGGAGCGGCAGACGTGAGCCGGCACGGCAGGCGGCCTCCTCCTCCTCTCAC
GGCACGGCAGCTACGGGGGATTCCTTTCCCACCGCTCCTTCGCTTTCCCTTCCTCGCCCG
CCGTAATAAATAGACACCCCCTCCACACCCTCTTTCCCCAACCTCGTGTTGTTCGGAGCGC
ACACACACACAACCAGATCTCCCCCAAATCCACCCGTCGGCACCTCCGCTTCAAGGTACG
CCGCTCGTCCTCCCCCCCCCCCCCTCTCTACCTTCTCTAGATCGGCGTTCCGGTCCATGG
TTAGGGCCCGGTAGTTCTACTTCTGTTCATGTTTGTGTTAGATCCGTGTTTGTGTTAGATCC
GTGCTGCTAGCGTTCGTACACGGATGCGACCTGTACGTCAGACACGTTCTGATTGCTAACT
TGCCAGTGTTTCTCTTTGGGGAATCCTGGGATGGCTCTAGCCGTTCCGCAGACGGGATCG
ATTTCATGATTTTTTTTGTTTCGTTGCATAGGGTTTGGTTTGCCCTTTTCCTTTATTTCAATAT
ATGCCGTGCACTTGTTTGTCGGGTCATCTTTTCATGCTTTTTTTTGTCTTGGTTGTGATGAT
GTGGTCTGGTTGGGCGGTCGTTCTAGATCGGAGTAGAATTCTGTTTCAAACTACCTGGTGG
ATTTATTAATTTTGGATCTGTATGTGTGTGCCATACATATTCATAGTTACGAATTGAAGATGA
TGGATGGAAATATCGATCTAGGATAGGTATACATGTTGATGCGGGTTTTACTGATGCATATA
CAGAGATGCTTTTTGTTCGCTTGGTTGTGATGATGTGGTGTGGTTGGGCGGTCGTTCATTC
GTTCTAGATCGGAGTAGAATACTGTTTCAAACTACCTGGTGTATTTATTAATTTTGGAACTGT
ATGTGTGTGTCATACATCTTCATAGTTACGAGTTTAAGATGGATGGAAATATCGATCTAGGA
TAGGTATACATGTTGATGTGGGTTTTACTGATGCATATACATGATGGCATATGCAGCATCTA
TTCATATGCTCTAACCTTGAGTACCTATCTATTATAATAAACAAGTATGTTTTATAATTATTTT
GATCTTGATATACTTGGATGATGGCATATGCAGCAGCTATATGTGGATTTTTTTAGCCCTGC
CTTCATACGCTATTTATTTGCTTGGTACTGTTTCTTTTGTCGATGCTCACCCTGTTGTTTGGT
GTTACTTCTGCAG
SEQ ID NO: 45 Hordeum GPT DNA coding sequence, including targeting sequence coding
domain
ATGGCATCCGCCCCCGCCTCCGCCTCCGCGGCCCTCTCCACCGCCGCCCCCGCCGACAA
CGGGGCCGCCAAGCCCACGGAGCAGCGGCCGGTACAGGTGGCTAAGCGATTGGAGAAG
TTCAAAACAACAATTTTCACACAGATGAGCATGCTCGCAGTGAAGCATGGAGCAATAAACC
TTGGACAGGGGTTTCCCAATTTTGATGGCCCTGACTTTGTCAAAGATGCTGCTATTGAGGC
TATCAAAGCTGGAAAGAATCAGTATGCAAGAGGATATGGTGTGCCTGAATTGAACTCAGCT
GTTGCTGAGAGATTTCTCAAGGACAGTGGATTGCACATCGATCCTGATAAGGAAGTTACTG
TTACATCTGGGTGCACAGAAGCAATAGCTGCAACGATATTGGGTCTGATCAACCCTGGGGA
TGAAGTCATACTGTTTGCTCCATTCTATGATTCTTATGAGGCTACACTGTCCATGGCTGGTG
CGAATGTCAAAGCCATTACACTCCGCCCTCCGGACTTTGCAGTCCCTCTTGAAGAGCTAAA
GGCTGCAGTCTCGAAGAATACCAGAGCAATAATGATTAATACACCTCACAACCCTACCGGG
AAAATGTTCACAAGGGAGGAACTTGAGTTCATTGCTGATCTCTGCAAGGAAAATGACGTGT
TGCTCTTTGCCGATGAGGTCTACGACAAGCTGGCGTTTGAGGCGGATCACATATCAATGG
CTTCTATTCCTGGCATGTATGAGAGGACCGTCACTATGAACTCCCTGGGGAAGACGTTCTC
CTTGACCGGATGGAAGATCGGCTGGGCGATAGCACCACCGCACCTGACATGGGGCGTAA
GGCAGGCACACTCCTTCCTCACATTCGCCACCTCCACGCCGATGCAATCAGCAGCGGCGG
CGGCCCTGAGAGCACCGGACAGCTACTTTGAGGAGCTGAAGAGGGACTACGGCGCAAAG
AAAGCGCTGCTGGTGGACGGGCTCAAGGCGGCGGGCTTCATCGTCTACCCTTCGAGCGG
AACCTACTTCATCATGGTCGACCACACCCCGTTCGGGTTCGACAACGACGTCGAGTTCTGC
GAGTACTTGATCCGCGAGGTCGGCGTCGTGGCCATCCCGCCAAGCGTGTTCTACCTGAAC
CCGGAGGACGGGAAGAACCTGGTGAGGTTCACCTTCTGCAAGGACGACGACACGCTAAG
GGCGGCGGTGGACAGGATGAAGGCCAAGCTCAGGAAGAAATGA
SEQ ID NO: 46: Hordeum GPT amino acid sequence, including putative targeting sequence (in
italics).
MASAPASASAALSTAAPADNGAAKPTEQRPVQVAKRLEKFKTTIFTQMSMLAVKHGAINLGQG
FPNFDGPDFVKDAAIEAIKAGKNQYARGYGVPELNSAVAERFLKDSGLHIDPDKEVTVTSGCTE
AIAATILGLINPGDEVILFAPFYDSYEATLSMAGANVKAITLRPPDFAVPLEELKAAVSKNTRAIMI
NTPHNPTGKMFTREELEFIADLCKENDVLLFADEVYDKLAFEADHISMASIPGMYERTVTMNSL
GKTFSLTGWKIGWAIAPPHLTWGVRQAHSFLTFATSTPMQSAAAAALRAPDSYFEELKRDYGA
KKALLVDGLKAAGFIVYPSSGTYFIMVDHTPFGFDNDVEFCEYLIREVGVVAIPPSVFYLNPEDG
KNLVRFTFC KD D DTLRAAVD RMKAKLRKK
SEQ ID NO: 47 Tomato rubisco small subunit (rbcS3C) promoter + Arabidopsis GS1 DNA
coding sequence; NcoI/AflIII splice site shown in bold, ATG start of GS1 underlined.
GTTTGAATCCTCCTTAAAGTTTTTCTCTGGAGAAACTGTAGTAATTTTACTTTGTTGTGTTCC
CTTCATCTTTTGAATTAATGGCATTTGTTTTAATACTAATCTGCTTCTGAAACTTGTAATGTAT
GTATATCAGTTTCTTATAATTTATCCAAGTAATATCTTCCATTCTCTATGCAATTGCCTGCAT
AAGCTCGACAAAAGAGTACATCAACCCCTCCTCCTCTGGACTACTCTAGCTAAACTTGAATT
TCCCCTTAAGATTATGAAATTGATATATCCTTAACAAACGACTCCTTCTGTTGGAAAATGTAG
TACTTGTCTTTCTTCTTTTGGGTATATATAGTTTATATACACCATACTATGTACAACATCCAA
GTAGAGTGAAATGGATACATGTACAAGACTTATTTGATTGATTGATGACTTGAGTTGCCTTA
GGAGTAACAAATTCTTAGGTCAATAAATCGTTGATTTGAAATTAATCTCTCTGTCTTAGACAG
ATAGGAATTATGACTTCCAATGGTCCAGAAAGCAAAGTTCGCACTGAGGGTATACTTGGAA
TTGAGACTTGCACAGGTCCAGAAACCAAAGTTCCCATCGAGCTCTAAAATCACATCTTTGG
AATGAAATTCAATTAGAGATAAGTTGCTTCATAGCATAGGTAAAATGGAAGATGTGAAGTAA
CCTGCAATAATCAGTGAAATGACATTAATACACTAAATACTTCATATGTAATTATCCTTTCCA
GGTTAACAATACTCTATAAAGTAAGAATTATCAGAAATGGGCTCATCAAACTTTTGTACTATG
TATTTCATATAAGGAAGTATAACTATACATAAGTGTATACACAACTTTATTCCTATTTTGTAAA
GGTGGAGAGACTGTTTTCGATGGATCTAAAGCAATATGTCTATAAAATGCATTGATATAATA
ATTATCTGAGAAAATCCAGAATTGGCGTTGGATTATTTCAGCCAAATAGAAGTTTGTACCAT
ACTTGTTGATTCCTTCTAAGTTAAGGTGAAGTATCATTCATAAACAGTTTTCCCCAAAGTACT
ACTCACCAAGTTTCCCTTTGTAGAATTAACAGTTCAAATATATGGCGCAGAAATTACTCTAT
GCCCAAAACCAAACGAGAAAGAAACAAAATACAGGGGTTGCAGACTTTATTTTCGTGTTAG
GGTGTGTTTTTTCATGTAATTAATCAAAAAATATTATGACAAAAACATTTATACATATTTTTAC
TCAACACTCTGGGTATCAGGGTGGGTTGTGTTCGACAATCAATATGGAAAGGAAGTATTTT
CCTTATTTTTTTAGTTAATATTTTCAGTTATACCAAACATACCTTGTGATATTATTTTTAAAAAT
GAAAAACTCGTCAGAAAGAAAAAGCAAAAGCAACAAAAAAATTGCAAGTATTTTTTAAAAAA
GAAAAAAAAAACATATCTTGTTTGTCAGTATGGGAAGTTTGAGATAAGGACGAGTGAGGGG
TTAAAATTCAGTGGCCATTGATTTTGTAATGCCAAGAACCACAAAATCCAATGGTTACCATT
CCTGTAAGATGAGGTTTGCTAACTCTTTTTGTCCGTTAGATAGGAAGCCTTATCACTATATA
TACAAGGCGTCCTAATAACCTCTTAGTAACCAATTATTTCAGCACCATGTCTCTGCTCTCAG
ATCTCGTTAACCTCAACCTCACCGATGCCACCGGGAAAATCATCGCCGAATACATATGGAT
CGGTGGATCTGGAATGGATATCAGAAGCAAAGCCAGGACACTACCAGGACCAGTGACTGA
TCCATCAAAGCTTCCCAAGTGGAACTACGACGGATCCAGCACCGGTCAGGCTGCTGGAGA
AGACAGTGAAGTCATTCTATACCCTCAGGCAATATTCAAGGATCCCTTCAGGAAAGGCAAC
AACATCCTGGTGATGTGTGATGCTTACACACCAGCTGGTGATCCTATTCCAACCAACAAGA
GGCACAACGCTGCTAAGATCTTCAGCCACCCCGACGTTGCCAAGGAGGAGCCTTGGTATG
GGATTGAGCAAGAATACACTTTGATGCAAAAGGATGTGAACTGGCCAATTGGTTGGCCTGT
TGGTGGCTACCCTGGCCCTCAGGGACCTTACTACTGTGGTGTGGGAGCTGACAAAGCCAT
TGGTCGTGACATTGTGGATGCTCACTACAAGGCCTGTCTTTACGCCGGTATTGGTATTTCT
GGTATCAATGGAGAAGTCATGCCAGGCCAGTGGGAGTTCCAAGTCGGCCCTGTTGAGGGT
ATTAGTTCTGGTGATCAAGTCTGGGTTGCTCGATACCTTCTCGAGAGGATCACTGAGATCT
CTGGTGTAATTGTCAGCTTCGACCCGAAACCAGTCCCGGGTGACTGGAATGGAGCTGGAG
CTCACTGCAACTACAGCACTAAGACAATGAGAAACGATGGAGGATTAGAAGTGATCAAGAA
AGCGATAGGGAAGCTTCAGCTGAAACACAAAGAACACATTGCTGCTTACGGTGAAGGAAA
CGAGCGTCGTCTCACTGGAAAGCACGAAACCGCAGACATCAACACATTCTCTTGGGGAGT
CGCGAACCGTGGAGCGTCAGTGAGAGTGGGACGTGACACAGAGAAGGAAGGTAAAGGGT
ACTTCGAAGACAGAAGGCCAGCTTCTAACATGGATCCTTACGTTGTCACCTCCATGATCGC
TGAGACGACCATACTCGGTTGA
SEQ ID NO: 48: Putative Clementine orange GPT coding sequence
Derived from BioChain (Hayward, CAorange cDNAlibrary, cat# C1634340;
Derived from clementine PCR primers:
5′-ggccacatgtccgttgctaagtgcttggagaagttta-3′ (AflIII oligo) [SEQ ID NO: __]
5′-cgggcacgtgtcattttctcctcagcttctccttcatcct-3′ (PmlI oligo) [SEQ ID NO: __]
ATG start site in bold, AflIII oligo binding site (start of putative mature coding sequence) is
underlined; terminator sequence italicized
ATGCTTAAGCCGTCCGCCTTCGGGTCTTCTTTTTCTTCCTCAGCTCTGCTTTCGTTTTCGAA
GCATTTGCATACAATAAGCATTACTGATTCTGTCAACACCAGAAGAAGAGGAATCAGTACC
GCTTGCCCTAGGTACCCTTCTCTCATGGCGAGCTTGTCCACCGTTTCCACCAATCAAAGCG
ACACCATCCAGAAGACCAATCTTCAGCCTCAACAGGTTGCTAAGTGCTTGGAGAAGTTTAA
AACTACAATCTTTACACAAATGAGTATGCTTGCCATCAAACATGGAGCTATAAATCTTGGTC
AAGGCTTTCCCAACTTTGATGGCCCAGATTTTGTTAAAGATGCAGCGATTCAAGCCATAAG
GGATGGGAAGAATCAATATGCTCGTGGACATGGGGTTCCAGAGTTCAACTCTGCCATTGCT
TCCCGGTTTAAGAAAGATTCTGGGCTCGAGGTTGACCCTGAAAAGGAAGTTACTGTTACCT
CTGGGTGCACCGAAGCCATTGCTGCAACCATCTTAGGTTTGATTAATCCTGGAGATGAGGT
GATCCTTTTTGCACCTTTCTATGATTCCTATGAAGCTACTCTCTCCATGGCTGGTGCTAAAA
TTAAATGCATCACATTGCGCCCTCCAGAATTTGCCATCCCCATTGAAGAGCTCAAGTCTACA
ATCTCAAAAAATACTCGTGCAATTCTTATGAACACTCCACATAACCCCACTGGAAAGATGTT
CACTAGGGAGGAACTTAATGTTATTGCATCTCTTTGCATTGAGAATGATGTGTTGGTTTTTA
GTGATGAGGTCTATGATAAGTTGGCTTTTGAAATGGATCACATTTCCATAGCCTCTCTTCCT
GGAATGTATGAGCGTACTGTAACCATGAATTCCTTAGGGAAGACATTCTCTTTAACAGGGT
GGAAGATCGGGTGGGCAATAGCTCCACCGCACCTTACATGGGGGGTGCGGCAGGCACAC
TCTTTTCTCACGTTTGCCACATCCACTCCAATGCAGTGGGCAGCTACAGCAGCCCTTAGAG
CTCCGGAGACGTACTATGAGGAGCTAAAGAGAGATTACTCGGCAAAGAAGGCAATTTTGGT
GGAGGGATTGAATGCTGTTGGTTTCAAGGTATTCCCATCTAGTGGGACATACTTTGTGGTT
GTAGATCACACCCCATTTGGGCACGAAACTGATATTGCATTTTGTGAATATCTGATCAAGGA
AGTTGGGGTTGTGGCAATTCCGACCAGCGTATTTTACTTGAATCCAGAGGATGGAAAGAAT
TTGGTGAGATTTACCTTCTGCAAAGATGAAGGAACTTTGAGGTCTGCAGTTGACAGGATGA
AGGAGAAGCTGAGGAGAAAATGA
SEQ ID NO: 49: Putative Clementine orange GPT amino acid sequence; putative mature
protein sequence begins at VAK shown in bold underline.
MLKPSAFGSSFSSSALLSFSKHLHTISITDSVNTRRRGISTACPRYPSLMASLSTVSTNQSDTIQ
KTNLQPQQVAKCLEKFKTTIFTQMSMLAIKHGAINLGQGFPNFDGPDFVKDAAIQAIRDGKNQY
ARGHGVPEFNSAIASRFKKDSGLEVDPEKEVTVTSGCTEAIAATILGLINPGDEVILFAPFYDSYE
ATLSMAGAKIKCITLRPPEFAIPIEELKSTISKNTRAILMNTPHNPTGKMFTREELNVIASLCIENDV
LVFSDEVYDKLAFEMDHISIASLPGMYERTVTMNSLGKTFSLTGWKIGWAIAPPHLTWGVRQAH
SFLTFATSTPMQWAATAALRAPETYYEELKRDYSAKKAILVEGLNAVGFKVFPSSGTYFVVVDH
TPFGHETDIAFCEYLIKEVGVVAIPTSVFYLNPEDGKNLVRFTFCKDEGTLRSAVDRMKEKLRRK

What is claimed:

1. A method for producing a plant having enhanced growth properties relative to an analogous wild type or untransformed plant, comprising:
(a) introducing into the plant a glutamine phenylpyruvate transaminase (GPT) transgene that encodes a GPT polypeptide having an amino acid sequence that has at least 80% sequence identity to SEQ ID NO: 2, SEQ ID NO: 11, SEQ ID NO: 13, or SEQ ID NO: 15 and GPT catalytic activity; and,
(b) expressing said GPT transgene in said transgenic plant or progeny thereof, wherein GPT polypeptides are overexpressed in said transgenic plant or said progeny thereof relative to an analogous wild type or untransformed plant of the same species,
wherein said transgenic plant has an increased biomass yield relative to an analogous wild type or untransformed plant of the same species.

2. The method according to claim 1, wherein the plant has at least one additional enhanced growth property selected from the group consisting of increased nitrogen use efficiency, earlier flowering, earlier budding, increased plant height, increased flowering, increased budding, larger leaves, increased fruit or pod yield and increased seed yield.

3. The method according to claim 1, further comprising propagating the plant and harvesting a seed therefrom.

4. The method according to claim 1, wherein the glutamine phenylpyruvate transaminase (GPT) transgene encodes a GPT polypeptide having an amino acid sequence that has at least 85% sequence identity to SEQ ID NO: 2, SEQ ID NO: 11, SEQ ID NO: 13, or SEQ ID NO: 15 and GPT catalytic activity.

5. The method according to claim 1, wherein the glutamine phenylpyruvate transaminase (GPT) transgene encodes a GPT polypeptide having an amino acid sequence that has at least 90% sequence identity to SEQ ID NO: 2, SEQ ID NO: 11, SEQ ID NO: 13, or SEQ ID NO: 15 and GPT catalytic activity.

6. The method according to claim 1, wherein the glutamine phenylpyruvate transaminase (GPT) transgene encodes a GPT polypeptide having an amino acid sequence that has at least 95% sequence identity to SEQ ID NO: 2, SEQ ID NO: 11, SEQ ID NO: 13, or SEQ ID NO: 15 and GPT catalytic activity.

7. The method according to claim 1, wherein the GPT transgene encodes a polypeptide having an amino acid sequence selected from the group consisting of (a) SEQ ID NO: 2, SEQ ID NO: 11, SEQ ID NO: 13, and SEQ ID NO: 15.

8. The method according to claim 1, wherein the GPT transgene is incorporated into the genome of the plant.

9. The method according to claim 1, wherein the plant is a monocotyledonous plant.

10. The method according to claim 1, wherein the plant is a dicotyledonous plant

11. The method according to claim 1, wherein the transgenic plant is selected from the group consisting of plants of the families Poaceae, Gossypium, Fabaceae, Rutaceae, Rubiaceae, Cucurbitaceae, Rosaceae, Asteraceae, Amaranthaceae or Brassicaceae.

12. The method according to claim 1, wherein the transgenic plant is selected from the group consisting of plants of the genera Avena, Hordeum, Oryza, Panicum, Phleum, Saccharum, Secale, Sorghum, Triticum, Zea, Pennisetum, Lycopersicon, Capiscum, Fagopyrum, Triticosecale, Chenopodium, Digitaria, Manihot, Ipomoea, Olea, Daucus, Pastinaca, Raphanus, Dioscorea, Armoracia, Elaeis, Linum, Carthamus, Sesamum, Vitis, or Solanum.

13. The method according to claim 1, wherein said transgenic plant produces more 2-oxo-glutaramate relative to an analogous wild type or untransformed plant of the same species.

14. The method according to claim 1, wherein the transgenic plant has an increased leaf-to-root ratio of GS activity in comparison to an analogous wild type or untransformed plant of the same species.

15. The method according to claim 1, wherein the transgenic plant has an increased leaf-to-root ratio of 2-oxo-glutaramate in comparison to an analogous wild type or untransformed plant of the same species.

16. The method according to claim 1, wherein the GPT transgene encodes a polypeptide having the amino acid sequence according to SEQ ID NO: 2.

17. A method for generating and selecting transgenic plants having increased production of 2-oxo-glutaramate, comprising:
(a) introducing into a plurality of plant cells a glutamine phenylpyruvate transaminase (GPT) transgene that encodes a GPT polypeptide having an amino acid sequence that has at least 80% sequence identity to SEQ ID NO: 2, SEQ ID NO: 11, SEQ ID NO: 13, or SEQ ID NO: 15 and has GPT catalytic activity,
(b) generating a plurality of transgenic plants from the plurality of plant cells; and,
(c) selecting a transgenic plant from said plurality of transgenic plants that produces more 2-oxo-glutaramate relative to an analogous wild type or untransformed plant.

18. The method according to claim 17, wherein the step of introducing said GPT transgene into said plurality of plant cells comprises inoculation of said plurality of plants cells with Agrobacterium transformed with said GPT transgene.

19. The method according to claim 18, further comprising the step of using a selectable marker to identify Agrobacterium transformed with said GPT transgene prior to inoculation of said plurality of plant cells.

20. The method according to claim 17, wherein the plant has at least one enhanced growth characteristic selected from the group consisting of increased biomass yield, increased nitrogen use efficiency, earlier flowering, earlier budding, increased plant height, increased flowering, increased budding, larger leaves, increased fruit or pod yield and increased seed yield.

21. The method according to claim 17, wherein each transgene is linked to a plant promoter that is heterologous to the plurality of plant cells.

22. The method according to claim 17, wherein the glutamine phenylpyruvate transaminase (GPT) transgene encodes a GPT polypeptide having an amino acid sequence that has at least 85% sequence identity to SEQ ID NO: 2, SEQ ID NO: 11, SEQ ID NO: 13, or SEQ ID NO: 15 and GPT catalytic activity.

23. The method according to claim 17, wherein the glutamine phenylpyruvate transaminase (GPT) transgene encodes a GPT polypeptide having an amino acid sequence that has at least 90% sequence identity to SEQ ID NO: 2, SEQ ID NO: 11, SEQ ID NO: 13, or SEQ ID NO: 15 and GPT catalytic activity.

24. The method according to claim 17, wherein the glutamine phenylpyruvate transaminase (GPT) transgene encodes a GPT polypeptide having an amino acid sequence that has at least 95% sequence identity to SEQ ID NO: 2, SEQ ID NO: 11, SEQ ID NO: 13, or SEQ ID NO: 15 and GPT catalytic activity.

25. The method according to claim 17, wherein the GPT transgene encodes a polypeptide having an amino acid sequence selected from the group consisting of (a) SEQ ID NO: 2, SEQ ID NO: 11, SEQ ID NO: 13, and SEQ ID NO: 15.

26. The method according to claim 17, wherein the GPT transgene is incorporated into the genome of the plant.

27. The method according to claim 17, wherein the plant is a monocotyledonous plant.

28. The method according to claim 17, wherein the plant is a dicotyledonous plant.

29. The method according to claim 17, wherein the transgenic plant is selected from the group consisting of plants of the families Poaceae, Gossypium, Fabaceae, Rutaceae, Rubiaceae, Cucurbitaceae, Rosaceae, Asteraceae, Amaranthaceae or Brassicaceae.

30. The method according to claim 17, wherein the transgenic plant is selected from the group consisting of plants of the genera Avena, Hordeum, Oryza, Panicum, Phleum, Saccharum, Secale, Sorghum, Triticum, Zea, Pennisetum, Lycopersicon, Capiscum, Fagopyrum, Triticosecale, Chenopodium, Digitaria, Manihot, Ipomoea, Olea, Daucus, Pastinaca, Raphanus, Dioscorea, Armoracia, Elaeis, Linum, Carthamus, Sesamum, Vitis, or Solanum.

31. The method according to claim 17, wherein said transgenic plant has an increased leaf-to-root ratio of GS activity in comparison to an analogous wild type or untransformed plant of the same species.

32. The method according to claim 17, wherein the transgenic plant has an increased leaf-to-root ratio of 2-oxo-glutaramate in comparison to an analogous wild type or untransformed plant of the same species.

33. The method according to claim 17, wherein the GPT transgene encodes a polypeptide having the amino acid sequence according to SEQ ID NO: 2.

34. The method according to claim 17, further comprising propagating the plant so selected and harvesting a seed therefrom.


SEQUENCE LISTING (60 sequences)

SEQ ID NO: 1 - DNA, 1323 bp, Aradopsis thaliana
atgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 2 - protein, 440 aa, Aradopsis thaliana
MYLDINGVMIKQFSFKASLLPFSSNFRQSSAKIHRPIGATMTTVSTQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 3 - DNA, 1374 bp, Medicago sativa
atttccgttttcgttttcatttgattcattgaatcaaatcgaatcgaatctttaggattcaatacagattccttagattttactaagtttgaaaccaaaaccaaaacatgtctctcctttcagatcttatcaaccttgacctctccgaaaccaccgagaaaatcatcgccgaatacatatggattggtggatctggtttggacttgaggagcaaagcaaggactctaccaggaccagttactgacccttcacagcttcccaagtggaactatgatggttccagcacaggtcaagctcctggagaagatagtgaagttattatctacccacaagccattttcaaggacccatttagaaggggtaacaatatcttggttatgtgtgatgcatacactccagctggagagcccattcccaccaacaagagacatgcagctgccaagattttcagccatcctgatgttgttgctgaagtaccatggtatggtattgagcaagaatacaccttgttgcagaaagacatcaattggcctcttggttggccagttggtggttttcctggacctcagggaccatactattgtggagctggtgctgacaaggcatttggccgtgacattgttgactcacattacaaagcctgtctttatgccggcatcaacatcagtggaatcaatggtgaagtgatgcctggtcaatgggaattccaagttggtccctcagttggtatctctgctggtgatgagatatgggttgctcgttacattttggagaggatcactgaggttgctggtgtggtgctttcctttgacccaaaaccaattaagggtgattggaatggtgctggtgctcacacaaattacagcaccaagtctatgagagaagatggtggctatgaagtcatcttgaaagcaattgagaagcttgggaagaagcacaaggagcacattgctgcttatggagaaggcaacgagcgtagattgacagggcgacatgagacagctgacattaacaccttcttatggggtgttgcaaaccgtggtgcgtcgattagagttggaagggacacagagaaagcagggaaaggttatttcgaggataggaggccatcatctaacatggatccatatgttgttacttccatgattgcagacaccaccattctctggaaaccataagccaccacacacacatgcattgaagtatttgaaagtcattgttgattccgcattagaatttggtcattgttttttctaggatttggatttgtgttattgttatggttcacactttgtttgtttgaatttgaggccttgttataggtttcatatttctttctcttgttctaagtaaatgtcagaataataatgtaat

SEQ ID NO: 4 - protein, 356 aa, Medicago sativa
MSLLSDLINLDLSETTEKIIAEYIWIGGSGLDLRSKARTLPGPVTDPSQLPKWNYDGSSTGQAPGEDSEVIIYPQAIFKDPFRRGNNILVMCDAYTPAGEPIPTNKRHAAAKIFSHPDVVAEVPWYGIEQEYTLLQKDINWPLGWPVGGFPGPQGPYYCGAGADKAFGRDIVDSHYKACLYAGINISGINGEVMPGQWEFQVGPSVGISAGDEIWVARYILERITEVAGVVLSFDPKPIKGDWNGAGAHTNYSTKSMREDGGYEVILKAIEKLGKKHKEHIAAYGEGNERRLTGRHETADINTFLWGVANRGASIRVGRDTEKAGKGYFEDRRPSSNMDPYVVTSMIADTTILWKP

SEQ ID NO: 5 - DNA, 1419 bp, Medicago sativa
atcgatgaattcgagctcggtacccatttccgttttcgttttcatttgattcattgaatcaaatcgaatcgaatctttaggattcaatacagattccttagattttactaagtttgaaaccaaaaccaaaacatgtctctcctttcagatcttatcaaccttgacctctccgaaaccaccgagaaaatcatcgccgaatacatatggattggtggatctggtttggacttgaggagcaaagcaaggactctaccaggaccagttactgacccttcacagcttcccaagtggaactatgatggttccagcacaggtcaagctcctggagaagatagtgaagttattatctacccacaagccattttcaaggacccatttagaaggggtaacaatatcttggttatgtgtgatgcatacactccagctggagagcccattcccaccaacaagagacatgcagctgccaagattttcagccatcctgatgttgttgctgaagtaccatggtatggtattgagcaagaatacaccttgttgcagaaagacatcaattggcctcttggttggccagttggtggttttcctggacctcagggaccatactattgtggagctggtgctgacaaggcatttggccgtgacattgttgactcacattacaaagcctgtctttatgccggcatcaacatcagtggaatcaatggtgaagtgatgcctggtcaatgggaattccaagttggtccctcagttggtatctctgctggtgatgagatatgggttgctcgttacattttggagaggatcactgaggttgctggtgtggtgctttcctttgacccaaaaccaattaagggtgattggaatggtgctggtgctcacacaaattacagcaccaagtctatgagagaagatggtggctatgaagtcatcttgaaagcaattgagaagcttgggaagaagcacaaggagcacattgctgcttatggagaaggcaacgagcgtagattgacagggcgacatgagacagctgacattaacaccttcttatggggtgttgcaaaccgtggtgcgtcgattagagttggaagggacacagagaaagcagggaaaggttatttcgaggataggaggccatcatctaacatggatccatatgttgttacttccatgattgcagacaccaccattctctggaaaccataagccaccacacacacatgcattgaagtatttgaaagtcattgttgattccgcattagaatttggtcattgttttttctaggatttggatttgtgttattgttatggttcacactttgtttgtttgaatttgaggccttgttataggtttcatatttctttctcttgttctaagtaaatgtcagaataataatgtaatggggatcctctagagtcgag

SEQ ID NO: 6 - DNA, 1302 bp, Artificial Sequence
  Synthetic plasmid vector sequence
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatgtctctgctctcagatctcgttaacctcaacctcaccgatgccaccgggaaaatcatcgccgaatacatatggatcggtggatctggaatggatatcagaagcaaagccaggacactaccaggaccagtgactgatccatcaaagcttcccaagtggaactacgacggatccagcaccggtcaggctgctggagaagacagtgaagtcattctataccctcaggcaatattcaaggatcccttcaggaaaggcaacaacatcctggtgatgtgtgatgcttacacaccagctggtgatcctattccaaccaacaagaggcacaacgctgctaagatcttcagccaccccgacgttgccaaggaggagccttggtatgggattgagcaagaatacactttgatgcaaaaggatgtgaactggccaattggttggcctgttggtggctaccctggccctcagggaccttactactgtggtgtgggagctgacaaagccattggtcgtgacattgtggatgctcactacaaggcctgtctttacgccggtattggtatttctggtatcaatggagaagtcatgccaggccagtgggagttccaagtcggccctgttgagggtattagttctggtgatcaagtctgggttgctcgataccttctcgagaggatcactgagatctctggtgtaattgtcagcttcgacccgaaaccagtcccgggtgactggaatggagctggagctcactgcaactacagcactaagacaatgagaaacgatggaggattagaagtgatcaagaaagcgatagggaagcttcagctgaaacacaaagaacacattgctgcttacggtgaaggaaacgagcgtcgtctcactggaaagcacgaaaccgcagacatcaacacattctcttggggagtcgcgaaccgtggagcgtcagtgagagtgggacgtgacacagagaaggaaggtaaagggtacttcgaagacagaaggccagcttctaacatggatccttacgttgtcacctccatgatcgctgagacgaccatactcggttga

SEQ ID NO: 7 - protein, 364 aa, Arabidopsis thaliana
  SITE (1)..(9): amino terminal 9 amino acids vector encoded sequence
MVDLRNRRTSMSLLSDLVNLNLTDATGKIIAEYIWIGGSGMDIRSKARTLPGPVTDPSKLPKWNYDGSSTGQAAGEDSEVILYPQAIFKDPFRKGNNILVMCDAYTPAGDPIPTNKRHNAAKIFSHPDVAKEEPWYGIEQEYTLMQKDVNWPIGWPVGGYPGPQGPYYCGVGADKAIGRDIVDAHYKACLYAGIGISGINGEVMPGQWEFQVGPVEGISSGDQVWVARYLLERITEISGVIVSFDPKPVPGDWNGAGAHCNYSTKTMRNDGGLEVIKKAIGKLQLKHKEHIAAYGEGNERRLTGKHETADINTFSWGVANRGASVRVGRDTEKEGKGYFEDRRPASNMDPYVVTSMIAETTILG

SEQ ID NO: 8 - DNA, 1817 bp, Artificial Sequence
  Synthetic plasmid vector sequence including vitis vinifera GPT coding sequence
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgcagctctctcaatgtacctggacattcccagagttgcttaaaagaccagcctttttaaggaggagtattgatagtatttcgagtagaagtaggtccagctccaagtatccatctttcatggcgtccgcatcaacggtctccgctccaaatacggaggctgagcagacccataacccccctcaacctctacaggttgcaaagcgcttggagaaattcaaaacaacaatctttactcaaatgagcatgcttgccatcaaacatggagcaataaaccttggccaagggtttcccaactttgatggtcctgagtttgtcaaagaagcagcaattcaagccattaaggatgggaaaaaccaatatgctcgtggatatggagttcctgatctcaactctgctgttgctgatagattcaagaaggatacaggactcgtggtggaccccgagaaggaagttactgttacttctggatgtacagaagcaattgctgctactatgctaggcttgataaatcctggtgatgaggtgatcctctttgctccattttatgattcctatgaagccactctatccatggctggtgcccaaataaaatccatcactttacgtcctccggattttgctgtgcccatggatgagctcaagtctgcaatctcaaagaatacccgtgcaatccttataaacactccccataaccccacaggaaagatgttcacaagggaggaactgaatgtgattgcatccctctgcattgagaatgatgtgttggtgtttactgatgaagtttacgacaagttggctttcgaaatggatcacatttccatggcttctcttcctgggatgtacgagaggaccgtgactatgaattccttagggaaaactttctccctgactggatggaagattggttggacagtagctcccccacacctgacatggggagtgaggcaagcccactcattcctcacgtttgctacctgcaccccaatgcaatgggcagctgcaacagccctccgggccccagactcttactatgaagagctaaagagagattacagtgcaaagaaggcaatcctggtggagggattgaaggctgtcggtttcagggtatacccatcaagtgggacctattttgtggtggtggatcacaccccatttgggttgaaagacgatattgcgttttgtgagtatctgatcaaggaagttggggtggtagcaattccgacaagcgttttctacttacacccagaagatggaaagaaccttgtgaggtttaccttctgtaaagacgagggaactctgagagctgcagttgaaaggatgaaggagaaactgaagcctaaacaataggggcacgtga

SEQ ID NO: 9 - protein, 459 aa, Vitis vinifera
MVDLRNRRTSMQLSQCTWTFPELLKRPAFLRRSIDSISSRSRSSSKYPSFMASASTVSAPNTEAEQTHNPPQPLQVAKRLEKFKTTIFTQMSMLAIKHGAINLGQGFPNFDGPEFVKEAAIQAIKDGKNQYARGYGVPDLNSAVADRFKKDTGLVVDPEKEVTVTSGCTEAIAATMLGLINPGDEVILFAPFYDSYEATLSMAGAQIKSITLRPPDFAVPMDELKSAISKNTRAILINTPHNPTGKMFTREELNVIASLCIENDVLVFTDEVYDKLAFEMDHISMASLPGMYERTVTMNSLGKTFSLTGWKIGWTVAPPHLTWGVRQAHSFLTFATCTPMQWAAATALRAPDSYYEELKRDYSAKKAILVEGLKAVGFRVYPSSGTYFVVVDHTPFGLKDDIAFCEYLIKEVGVVAIPTSVFYLHPEDGKNLVRFTFCKDEGTLRAAVERMKEKLKPKQ

SEQ ID NO: 10 - DNA, 1446 bp, Artificial Sequence
  Synthetic DNA encoding Oryza sativa GPT protein, codons optimized for expression in E. coli
atgtggatgaacctggcaggctttctggcaaccccggcaaccgcaaccgcaacccgtcatgaaatgccgctgaacccgagcagcagcgcgagctttctgctgagcagcctgcgtcgtagcctggtggcgagcctgcgtaaagcgagcccggcagcagcagcagcactgagcccgatggcaagcgcaagcaccgtggcagcagaaaacggtgcagcaaaagcagcagcagaaaaacagcagcagcagccggtgcaggtggcgaaacgtctggaaaaatttaaaaccaccatttttacccagatgagcatgctggcgattaaacatggcgcgattaacctgggccagggctttccgaactttgatggcccggattttgtgaaagaagcggcgattcaggcgattaacgcgggcaaaaaccagtatgcgcgtggctatggcgtgccggaactgaacagcgcgattgcggaacgttttctgaaagatagcggcctgcaggtggatccggaaaaagaagtgaccgtgaccagcggctgcaccgaagcgattgcggcgaccattctgggcctgattaacccgggcgatgaagtgattctgtttgcgccgttttatgatagctatgaagcgaccctgagcatggcgggcgcgaacgtgaaagcgattaccctgcgtccgccggattttagcgtgccgctggaagaactgaaagcggccgtgagcaaaaacacccgtgcgattatgattaacaccccgcataacccgaccggcaaaatgtttacccgtgaagaactggaatttattgcgaccctgtgcaaagaaaacgatgtgctgctgtttgcggatgaagtgtatgataaactggcgtttgaagcggatcatattagcatggcgagcattccgggcatgtatgaacgtaccgtgaccatgaacagcctgggcaaaacctttagcctgaccggctggaaaattggctgggcgattgcgccgccgcatctgacctggggcgtgcgtcaggcacatagctttctgacctttgcaacctgcaccccgatgcaggcagccgccgcagcagcactgcgtgcaccggatagctattatgaagaactgcgtcgtgattatggcgcgaaaaaagcgctgctggtgaacggcctgaaagatgcgggctttattgtgtatccgagcagcggcacctattttgtgatggtggatcataccccgtttggctttgataacgatattgaattttgcgaatatctgattcgtgaagtgggcgtggtggcgattccgccgagcgtgttttatctgaacccggaagatggcaaaaacctggtgcgttttaccttttgcaaagatgatgaaaccctgcgtgcggcggtggaacgtatgaaaaccaaactgcgtaaaaaaaagcttgcggccgcactcgagcaccaccaccaccaccactga

SEQ ID NO: 11 - protein, 481 aa, Artificial Sequence
  Oryza sativa GPT protein sequence with amino- and carboxyl-terminal vector sequences
MWMNLAGFLATPATATATRHEMPLNPSSSASFLLSSLRRSLVASLRKASPAAAAALSPMASASTVAAENGAAKAAAEKQQQQPVQVAKRLEKFKTTIFTQMSMLAIKHGAINLGQGFPNFDGPDFVKEAAIQAINAGKNQYARGYGVPELNSAIAERFLKDSGLQVDPEKEVTVTSGCTEAIAATILGLINPGDEVILFAPFYDSYEATLSMAGANVKAITLRPPDFSVPLEELKAAVSKNTRAIMINTPHNPTGKMFTREELEFIATLCKENDVLLFADEVYDKLAFEADHISMASIPGMYERTVTMNSLGKTFSLTGWKIGWAIAPPHLTWGVRQAHSFLTFATCTPMQAAAAAALRAPDSYYEELRRDYGAKKALLVNGLKDAGFIVYPSSGTYFVMVDHTPFGFDNDIEFCEYLIREVGVVAIPPSVFYLNPEDGKNLVRFTFCKDDETLRAAVERMKTKLRKKKLAAALEHHHHHH

SEQ ID NO: 12 - DNA, 1251 bp, Artificial Sequence
  Synthetic DNA encoding Glycine max GPT protein, codons optimized for expression in E. coli
atgcatcatcaccatcaccatggtaagcctatccctaaccctctcctcggtctcgattctacggaaaacctgtattttcagggaattgatcccttcaccgcgaaacgtctggaaaaatttcagaccaccatttttacccagatgagcctgctggcgattaaacatggcgcgattaacctgggccagggctttccgaactttgatggcccggaatttgtgaaagaagcggcgattcaggcgattcgtgatggcaaaaaccagtatgcgcgtggctatggcgtgccggatctgaacattgcgattgcggaacgttttaaaaaagataccggcctggtggtggatccggaaaaagaaattaccgtgaccagcggctgcaccgaagcgattgcggcgaccatgattggcctgattaacccgggcgatgaagtgattatgtttgcgccgttttatgatagctatgaagcgaccctgagcatggcgggcgcgaaagtgaaaggcattaccctgcgtccgccggattttgcggtgccgctggaagaactgaaaagcaccattagcaaaaacacccgtgcgattctgattaacaccccgcataacccgaccggcaaaatgtttacccgtgaagaactgaactgcattgcgagcctgtgcattgaaaacgatgtgctggtgtttaccgatgaagtgtatgataaactggcgtttgatatggaacatattagcatggcgagcctgccgggcatgtttgaacgtaccgtgaccctgaacagcctgggcaaaacctttagcctgaccggctggaaaattggctgggcgattgcgccgccgcatctgagctggggcgtgcgtcaggcgcatgcgtttctgacctttgcaaccgcacatccgtttcagtgcgcagcagcagcagcactgcgtgcaccggatagctattatgtggaactgaaacgtgattatatggcgaaacgtgcgattctgattgaaggcctgaaagcggtgggctttaaagtgtttccgagcagcggcacctattttgtggtggtggatcataccccgtttggcctggaaaacgatgtggcgttttgcgaatatctggtgaaagaagtgggcgtggtggcgattccgaccagcgtgttttatctgaacccggaagaaggcaaaaacctggtgcgttttaccttttgcaaagatgaagaaaccattcgtagcgcggtggaacgtatgaaagcgaaactgcgtaaagtcgactaa

SEQ ID NO: 13 - protein, 416 aa, Artificial Sequence
  Glycine max GPT amino acid sequence and amino-terminal vector sequence
MHHHHHHGKPIPNPLLGLDSTENLYFQGIDPFTAKRLEKFQTTIFTQMSLLAIKHGAINLGQGFPNFDGPEFVKEAAIQAIRDGKNQYARGYGVPDLNIAIAERFKKDTGLVVDPEKEITVTSGCTEAIAATMIGLINPGDEVIMFAPFYDSYEATLSMAGAKVKGITLRPPDFAVPLEELKSTISKNTRAILINTPHNPTGKMFTREELNCIASLCIENDVLVFTDEVYDKLAFDMEHISMASLPGMFERTVTLNSLGKTFSLTGWKIGWAIAPPHLSWGVRQAHAFLTFATAHPFQCAAAAALRAPDSYYVELKRDYMAKRAILIEGLKAVGFKVFPSSGTYFVVVDHTPFGLENDVAFCEYLVKEVGVVAIPTSVFYLNPEEGKNLVRFTFCKDEETIRSAVERMKAKLRKVD

SEQ ID NO: 14 - DNA, 1278 bp, Hordeum vulgare
atggtagatctgaggaaccgacgaactagtatggcatccgcccccgcctccgcctccgcggccctctccaccgccgcccccgccgacaacggggccgccaagcccacggagcagcggccggtacaggtggctaagcgattggagaagttcaaaacaacaattttcacacagatgagcatgctcgcagtgaagcatggagcaataaaccttggacaggggtttcccaattttgatggccctgactttgtcaaagatgctgctattgaggctatcaaagctggaaagaatcagtatgcaagaggatatggtgtgcctgaattgaactcagctgttgctgagagatttctcaaggacagtggattgcacatcgatcctgataaggaagttactgttacatctgggtgcacagaagcaatagctgcaacgatattgggtctgatcaaccctggggatgaagtcatactgtttgctccattctatgattcttatgaggctacactgtccatggctggtgcgaatgtcaaagccattacactccgccctccggactttgcagtccctcttgaagagctaaaggctgcagtctcgaagaataccagagcaataatgattaatacacctcacaaccctaccgggaaaatgttcacaagggaggaacttgagttcattgctgatctctgcaaggaaaatgacgtgttgctctttgccgatgaggtctacgacaagctggcgtttgaggcggatcacatatcaatggcttctattcctggcatgtatgagaggaccgtcactatgaactccctggggaagacgttctccttgaccggatggaagatcggctgggcgatagcaccaccgcacctgacatggggcgtaaggcaggcacactccttcctcacattcgccacctccacgccgatgcaatcagcagcggcggcggccctgagagcaccggacagctactttgaggagctgaagagggactacggcgcaaagaaagcgctgctggtggacgggctcaaggcggcgggcttcatcgtctacccttcgagcggaacctacttcatcatggtcgaccacaccccgttcgggttcgacaacgacgtcgagttctgcgagtacttgatccgcgaggtcggcgtcgtggccatcccgccaagcgtgttctacctgaacccggaggacgggaagaacctggtgaggttcaccttctgcaaggacgacgacacgctaagggcggcggtggacaggatgaaggccaagctcaggaagaaatga

SEQ ID NO: 15 - protein, 425 aa, Hordeum vulgare
MVDLRNRRTSMASAPASASAALSTAAPADNGAAKPTEQRPVQVAKRLEKFKTTIFTQMSMLAVKHGAINLGQGFPNFDGPDFVKDAAIEAIKAGKNQYARGYGVPELNSAVAERFLKDSGLHIDPDKEVTVTSGCTEAIAATILGLINPGDEVILFAPFYDSYEATLSMAGANVKAITLRPPDFAVPLEELKAAVSKNTRAIMINTPHNPTGKMFTREELEFIADLCKENDVLLFADEVYDKLAFEADHISMASIPGMYERTVTMNSLGKTFSLTGWKIGWAIAPPHLTWGVRQAHSFLTFATSTPMQSAAAAALRAPDSYFEELKRDYGAKKALLVDGLKAAGFIVYPSSGTYFIMVDHTPFGFDNDVEFCEYLIREVGVVAIPPSVFYLNPEDGKNLVRFTFCKDDDTLRAAVDRMKAKLRKK

SEQ ID NO: 16 - DNA, 1200 bp, Artificial Sequence
  Synthetic DNA encoding Danio rerio GPT protein, codons optimized for expression in E. coli, including 5′ and 3′ vector sequences
atgtccgtggcgaaacgtctggaaaaatttaaaaccaccatttttacccagatgagcatgctggcgattaaacatggcgcgattaacctgggccagggctttccgaactttgatggcccggattttgtgaaagaagcggcgattcaggcgattcgtgatggcaacaaccagtatgcgcgtggctatggcgtgccggatctgaacattgcgattagcgaacgttataaaaaagataccggcctggcggtggatccggaaaaagaaattaccgtgaccagcggctgcaccgaagcgattgcggcgaccgtgctgggcctgattaacccgggcgatgaagtgattgtgtttgcgccgttttatgatagctatgaagcgaccctgagcatggcgggcgcgaaagtgaaaggcattaccctgcgtccgccggattttgcgctgccgattgaagaactgaaaagcaccattagcaaaaacacccgtgcgattctgctgaacaccccgcataacccgaccggcaaaatgtttaccccggaagaactgaacaccattgcgagcctgtgcattgaaaacgatgtgctggtgtttagcgatgaagtgtatgataaactggcgtttgatatggaacatattagcattgcgagcctgccgggcatgtttgaacgtaccgtgaccatgaacagcctgggcaaaacctttagcctgaccggctggaaaattggctgggcgattgcgccgccgcatctgacctggggcgtgcgtcaggcgcatgcgtttctgacctttgcaaccagcaacccgatgcagtgggcagcagcagtggcactgcgtgcaccggatagctattataccgaactgaaacgtgattatatggcgaaacgtagcattctggtggaaggcctgaaagcggtgggctttaaagtgtttccgagcagcggcacctattttgtggtggtggatcataccccgtttggccatgaaaacgatattgcgttttgcgaatatctggtgaaagaagtgggcgtggtggcgattccgaccagcgtgttttatctgaacccggaagaaggcaaaaacctggtgcgttttaccttttgcaaagatgaaggcaccctgcgtgcggcggtggatcgtatgaaagaaaaactgcgtaaagtcgacaagcttgcggccgcactcgagcaccaccaccaccaccactga

SEQ ID NO: 17 - protein, 399 aa, Danio rerio
  MISC_FEATURE (1)..(399): Amino- and carboxy-terminal amino acids shown
MSVAKRLEKFKTTIFTQMSMLAIKHGAINLGQGFPNFDGPDFVKEAAIQAIRDGNNQYARGYGVPDLNIAISERYKKDTGLAVDPEKEITVTSGCTEAIAATVLGLINPGDEVIVFAPFYDSYEATLSMAGAKVKGITLRPPDFALPIEELKSTISKNTRAILLNTPHNPTGKMFTPEELNTIASLCIENDVLVFSDEVYDKLAFDMEHISIASLPGMFERTVTMNSLGKTFSLTGWKIGWAIAPPHLTWGVRQAHAFLTFATSNPMQWAAAVALRAPDSYYTELKRDYMAKRSILVEGLKAVGFKVFPSSGTYFVVVDHTPFGHENDIAFCEYLVKEVGVVAIPTSVFYLNPEEGKNLVRFTFCKDEGTLRAAVDRMKEKLRKVDKLAAALEHHHHHH

SEQ ID NO: 18 - DNA, 1236 bp, Arabidopsis thaliana
atggccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 19 - protein, 411 aa, Arabidopsis thaliana
MAKIHRPIGATMTTVSTQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 20 - DNA, 1194 bp, Arabidopsis thaliana
atggcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 21 - protein, 397 aa, Arabidopsis thaliana
MATQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 22 - DNA, 1680 bp, Lycopersicon esculentum
ggtaccgtttgaatcctccttaaagtttttctctggagaaactgtagtaattttactttgttgtgttcccttcatcttttgaattaatggcatttgttttaatactaatctgcttctgaaacttgtaatgtatgtatatcagtttcttataatttatccaagtaatatcttccattctctatgcaattgcctgcataagctcgacaaaagagtacatcaacccctcctcctctggactactctagctaaacttgaatttccccttaagattatgaaattgatatatccttaacaaacgactccttctgttggaaaatgtagtacttgtctttcttcttttgggtatatatagtttatatacaccatactatgtacaacatccaagtagagtgaaatggatacatgtacaagacttatttgattgattgatgacttgagttgccttaggagtaacaaattcttaggtcaataaatcgttgatttgaaattaatctctctgtcttagacagataggaattatgacttccaatggtccagaaagcaaagttcgcactgagggtatacttggaattgagacttgcacaggtccagaaaccaaagttcccatcgagctctaaaatcacatctttggaatgaaattcaattagagataagttgcttcatagcataggtaaaatggaagatgtgaagtaacctgcaataatcagtgaaatgacattaatacactaaatacttcatatgtaattatcctttccaggttaacaatactctataaagtaagaattatcagaaatgggctcatcaaacttttgtactatgtatttcatataaggaagtataactatacataagtgtatacacaactttattcctattttgtaaaggtggagagactgttttcgatggatctaaagcaatatgtctataaaatgcattgatataataattatctgagaaaatccagaattggcgttggattatttcagccaaatagaagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatgg

SEQ ID NO: 23 - DNA, 1230 bp, Phyllostachys bambusoides
atggcctccgcggccgtctccaccgtcgccaccgccgccgacggcgtcgcgaagccgacggagaagcagccggtacaggtcgcaaagcgtttggaaaagtttaagacaacaattttcacacagatgagcatgcttgccatcaagcatggagcaataaacctcggccagggctttccgaattttgatggccctgactttgtgaaagaagctgctattcaagctatcaatgctgggaagaatcagtatgcaagaggatatggtgtgcctgaactgaactcggctgttgctgaaaggttcctgaaggacagtggcttgcaagtcgatcccgagaaggaagttactgtcacatctgggtgcacggaagcgatagctgcaacgatattgggtcttatcaaccctggcgatgaagtgatcttgtttgctccattctatgattcatacgaggctacgctgtcgatggctggtgccaatgtaaaagccattactctccgtcctccagattttgcagtccctcttgaggagctaaaggccacagtctctaagaacaccagagcgataatgataaacacaccacacaatcctactgggaaaatgttttctagggaagaacttgaattcattgctactctctgcaagaaaaatgatgtgttgctttttgctgatgaggtctatgacaagttggcatttgaggcagatcatatatcaatggcttctattcctggcatgtatgagaggactgtgactatgaactctctggggaagacattctctctaacaggatggaagatcggttgggcaatagcaccaccacacctgacatggggtgtaaggcaggcacactcattcctcacatttgccacctgcacaccaatgcaatcggcggcggcggcggctcttagagcaccagatagctactatggggagctgaagagggattacggtgcaaagaaagcgatactagtcgacggactcaaggctgcaggttttattgtttacccttcaagtggaacatactttgtcatggtcgatcacaccccgtttggtttcgacaatgatattgagttctgcgagtatttgatccgcgaagtcggtgttgtcgccataccaccaagcgtattttatctcaaccctgaggatgggaagaacttggtgaggttcaccttctgcaaggatgatgatacgctgagagccgcagttgagaggatgaagacaaagctcaggaaaaaatga

SEQ ID NO: 24 - protein, 409 aa, Phyllostachys bambusoides
MASAAVSTVATAADGVAKPTEKQPVQVAKRLEKFKTTIFTQMSMLAIKHGAINLGQGFPNFDGPDFVKEAAIQAINAGKNQYARGYGVPELNSAVAERFLKDSGLQVDPEKEVTVTSGCTEAIAATILGLINPGDEVILFAPFYDSYEATLSMAGANVKAITLRPPDFAVPLEELKATVSKNTRAIMINTPHNPTGKMFSREELEFIATLCKKNDVLLFADEVYDKLAFEADHISMASIPGMYERTVTMNSLGKTFSLTGWKIGWAIAPPHLTWGVRQAHSFLTFATCTPMQSAAAAALRAPDSYYGELKRDYGAKKAILVDGLKAAGFIVYPSSGTYFVMVDHTPFGFDNDIEFCEYLIREVGVVAIPPSVFYLNPEDGKNLVRFTFCKDDDTLRAAVERMKTKLRKK

SEQ ID NO: 25 - DNA, 1858 bp, Oryza sativa
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgaatctggccggctttctcgccacgcccgcgaccgcgaccgcgacgcggcatgagatgccgttaaatccctcctcctccgcctccttcctcctctcctcgctccgccgctcgctcgtcgcgtcgctccggaaggcctcgccggcggcggccgcggcgctctcccccatggcctccgcgtccaccgtcgccgccgagaacggcgccgccaaggcggcggcggagaagcagcagcagcagcctgtgcaggttgcaaagcggttggaaaagtttaagacgaccattttcacacagatgagtatgcttgccatcaagcatggagcaataaaccttggccagggttttccgaatttcgatggccctgactttgtaaaagaggctgctattcaagctatcaatgctgggaagaatcagtacgcaagaggatatggtgtgcctgaactgaactcagctattgctgaaagattcctgaaggacagcggactgcaagtcgatccggagaaggaagttactgtcacatctggatgcacagaagctatagctgcaacaattttaggtctaattaatccaggcgatgaagtgatattgtttgctccattctatgattcatatgaggctaccctgtcaatggctggtgccaacgtaaaagccattactctccgtcctccagatttttcagtccctcttgaagagctaaaggctgcagtctcgaagaacaccagagctattatgataaacaccccgcacaatcctactgggaaaatgtttacaagggaagaacttgagtttattgccactctctgcaaggaaaatgatgtgctgctttttgctgatgaggtctacgacaagttagcttttgaggcagatcatatatcaatggcttctattcctggcatgtatgagaggaccgtgaccatgaactctcttgggaagacattctctcttacaggatggaagatcggttgggcaatcgcaccgccacacctgacatggggtgtaaggcaggcacactcattcctcacgtttgcgacctgcacaccaatgcaagcagctgcagctgcagctctgagagcaccagatagctactatgaggaactgaggagggattatggagctaagaaggcattgctagtcaacggactcaaggatgcaggtttcattgtctatccttcaagtggaacatacttcgtcatggtcgaccacaccccatttggtttcgacaatgatattgagttctgcgagtatttgattcgcgaagtcggtgttgtcgccataccacctagtgtattttatctcaaccctgaggatgggaagaacttggtgaggttcaccttttgcaaggatgatgagacgctgagagccgcggttgagaggatgaagacaaagctcaggaaaaaatga

SEQ ID NO: 26 - DNA, 1724 bp, Artificial Sequence
  Synthetic DNA encoding Hordeum vulgare GPT protein
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatggcatccgcccccgcctccgcctccgcggccctctccaccgccgcccccgccgacaacggggccgccaagcccacggagcagcggccggtacaggtggctaagcgattggagaagttcaaaacaacaattttcacacagatgagcatgctcgcagtgaagcatggagcaataaaccttggacaggggtttcccaattttgatggccctgactttgtcaaagatgctgctattgaggctatcaaagctggaaagaatcagtatgcaagaggatatggtgtgcctgaattgaactcagctgttgctgagagatttctcaaggacagtggattgcacatcgatcctgataaggaagttactgttacatctgggtgcacagaagcaatagctgcaacgatattgggtctgatcaaccctggggatgaagtcatactgtttgctccattctatgattcttatgaggctacactgtccatggctggtgcgaatgtcaaagccattacactccgccctccggactttgcagtccctcttgaagagctaaaggctgcagtctcgaagaataccagagcaataatgattaatacacctcacaaccctaccgggaaaatgttcacaagggaggaacttgagttcattgctgatctctgcaaggaaaatgacgtgttgctctttgccgatgaggtctacgacaagctggcgtttgaggcggatcacatatcaatggcttctattcctggcatgtatgagaggaccgtcactatgaactccctggggaagacgttctccttgaccggatggaagatcggctgggcgatagcaccaccgcacctgacatggggcgtaaggcaggcacactccttcctcacattcgccacctccacgccgatgcaatcagcagcggcggcggccctgagagcaccggacagctactttgaggagctgaagagggactacggcgcaaagaaagcgctgctggtggacgggctcaaggcggcgggcttcatcgtctacccttcgagcggaacctacttcatcatggtcgaccacaccccgttcgggttcgacaacgacgtcgagttctgcgagtacttgatccgcgaggtcggcgtcgtggccatcccgccaagcgtgttctacctgaacccggaggacgggaagaacctggtgaggttcaccttctgcaaggacgacgacacgctaagggcggcggtggacaggatgaaggccaagctcaggaagaaatgattgaggggcgcacgtgtga

SEQ ID NO: 27 - DNA, 1868 bp, Artificial Sequence
  Synthetic DNA encoding Arabidopsis thaliana GPT protein
catggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 28 - DNA, 1780 bp, Artificial Sequence
  Synthetic DNA encoding Arabidopsis thaliana GPT protein
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 29 - DNA, 1155 bp, Arabidopsis thaliana
gtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 30 - protein, 384 aa, Arabidopsis thaliana
VAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 31 - protein, 384 aa, Vitis vinifera
VAKRLEKFKTTIFTQMSMLAIKHGAINLGQGFPNFDGPEFVKEAAIQAIKDGKNQYARGYGVPDLNSAVADRFKKDTGLVVDPEKEVTVTSGCTEAIAATMLGLINPGDEVILFAPFYDSYEATLSMAGAQIKSITLRPPDFAVPMDELKSAISKNTRAILINTPHNPTGKMFTREELNVIASLCIENDVLVFTDEVYDKLAFEMDHISMASLPGMYERTVTMNSLGKTFSLTGWKIGWTVAPPHLTWGVRQAHSFLTFATCTPMQWAAATALRAPDSYYEELKRDYSAKKAILVEGLKAVGFRVYPSSGTYFVVVDHTPFGLKDDIAFCEYLIKEVGVVAIPTSVFYLHPEDGKNLVRFTFCKDEGTLRAAVERMKEKLKPKQ

SEQ ID NO: 32 - protein, 383 aa, Oryza sativa
VAKRLEKFKTTIFTQMSMLAIKHGAINLGQGFPNFDGPDFVKEAAIQAINAGKNQYARGYGVPELNSAIAERFLKDSGLQVDPEKEVTVTSGCTEAIAATILGLINPGDEVILFAPFYDSYEATLSMAGANVKAITLRPPDFSVPLEELKAAVSKNTRAIMINTPHNPTGKMFTREELEFIATLCKENDVLLFADEVYDKLAFEADHISMASIPGMYERTVTMNSLGKTFSLTGWKIGWAIAPPHLTWGVRQAHSFLTFATCTPMQAAAAAALRAPDSYYEELRRDYGAKKALLVNGLKDAGFIVYPSSGTYFVMVDHTPFGFDNDIEFCEYLIREVGVVAIPPSVFYLNPEDGKNLVRFTFCKDDETLRAAVERMKTKLRKK

SEQ ID NO: 33 - protein, 383 aa, Glycine max
AKRLEKFQTTIFTQMSLLAIKHGAINLGQGFPNFDGPEFVKEAAIQAIRDGKNQYARGYGVPDLNIAIAERFKKDTGLVVDPEKEITVTSGCTEAIAATMIGLINPGDEVIMFAPFYDSYEATLSMAGAKVKGITLRPPDFAVPLEELKSTISKNTRAILINTPHNPTGKMFTREELNCIASLCIENDVLVFTDEVYDKLAFDMEHISMASLPGMFERTVTLNSLGKTFSLTGWKIGWAIAPPHLSWGVRQAHAFLTFATAHPFQCAAAAALRAPDSYYVELKRDYMAKRAILIEGLKAVGFKVFPSSGTYFVVVDHTPFGLENDVAFCEYLVKEVGVVAIPTSVFYLNPEEGKNLVRFTFCKDEETIRSAVERMKAKLRKVD

SEQ ID NO: 34 - protein, 383 aa, Hordeum vulgare
VAKRLEKFKTTIFTQMSMLAVKHGAINLGQGFPNFDGPDFVKDAAIEAIKAGKNQYARGYGVPELNSAVAERFLKDSGLHIDPDKEVTVTSGCTEAIAATILGLINPGDEVILFAPFYDSYEATLSMAGANVKAITLRPPDFAVPLEELKAAVSKNTRAIMINTPHNPTGKMFTREELEFIADLCKENDVLLFADEVYDKLAFEADHISMASIPGMYERTVTMNSLGKTFSLTGWKIGWAIAPPHLTWGVRQAHSFLTFATSTPMQSAAAAALRAPDSYFEELKRDYGAKKALLVDGLKAAGFIVYPSSGTYFIMVDHTPFGFDNDVEFCEYLIREVGVVAIPPSVFYLNPEDGKNLVRFTFCKDDDTLRAAVDRMKAKLRKK

SEQ ID NO: 35 - protein, 382 aa, Danio rerio
VAKRLEKFKTTIFTQMSMLAIKHGAINLGQGFPNFDGPDFVKEAAIQAIRDGNNQYARGYGVPDLNIAISERYKKDTGLAVDPEKEITVTSGCTEAIAATVLGLINPGDEVIVFAPFYDSYEATLSMAGAKVKGITLRPPDFALPIEELKSTISKNTRAILLNTPHNPTGKMFTPEELNTIASLCIENDVLVFSDEVYDKLAFDMEHISIASLPGMFERTVTMNSLGKTFSLTGWKIGWAIAPPHLTWGVRQAHAFLTFATSNPMQWAAAVALRAPDSYYTELKRDYMAKRSILVEGLKAVGFKVFPSSGTYFVVVDHTPFGHENDIAFCEYLVKEVGVVAIPTSVFYLNPEEGKNLVRFTFCKDEGTLRAAVDRMKEKLRK

SEQ ID NO: 36 - protein, 383 aa, Phyllostachys bambusoides
VAKRLEKFKTTIFTQMSMLAIKHGAINLGQGFPNFDGPDFVKEAAIQAINAGKNQYARGYGVPELNSAVAERFLKDSGLQVDPEKEVTVTSGCTEAIAATILGLINPGDEVILFAPFYDSYEATLSMAGANVKAITLRPPDFAVPLEELKATVSKNTRAIMINTPHNPTGKMFSREELEFIATLCKKNDVLLFADEVYDKLAFEADHISMASIPGMYERTVTMNSLGKTFSLTGWKIGWAIAPPHLTWGVRQAHSFLTFATCTPMQSAAAAALRAPDSYYGELKRDYGAKKAILVDGLKAAGFIVYPSSGTYFVMVDHTPFGFDNDIEFCEYLIREVGVVAIPPSVFYLNPEDGKNLVRFTFCKDDDTLRAAVERMKTKLRKK

SEQ ID NO: 37 - DNA, 34 bp, Artificial Sequence
  Synthetic primer sequence
cccatcgatgtacctggacataaatggtgtgatg

SEQ ID NO: 38 - DNA, 37 bp, Artificial Sequence
  Synthetic primer sequence
gatggtacctcagacttttctcttaagcttctgcttc

SEQ ID NO: 39 - DNA, 2992 bp, Artificial Sequence
  Synthetic expression cassette
ctgcagcaaagaaacgttattagttggtgcttttggtggtaggaatgtagttttctgacaaagtcaattactgaatataaaaaaaatctgcacagctctgcgtcaacagttgtccaagggatgcctcaaaaatctgtgcagattatcagtcgtcacgcagaagcagaacatcatggtgtgctaggtcagcttcttgcattgggccatgaatccggttggttgttaatctctcctctcttattctcttatattaagatgcataactcttttatgtagtctaaaaaaaaatccagtggatcggatagtagtacgtcatggtgccattaggtaccgttgaacctaacagatatttatgcatgtgtatatatatagctatatagacaaaattgatgccgattatagacccaaaagcaataggtatatataatataatacagaccacaccaccaaactaagaatcgatcaaatagacaaggcatgtctccaaattgtcttaaactatttccgtaggttcagccgttcaggagtcgaatcagcctctgccggcgttttctttgcacgtacgacggacacacatgggcataccatatagctggtccatgacattaggagagagaacgtacgtgttgacctgtagctgagatataacaaggttgattataatatcaccaaacatgaaatcatccaaggatgacccataactatcactactatagtactgcatctggtaaaagaaattgtatagactctatttcgagcactaccacataacgcctgcaatgtgacaccctacctattcactaatgtgcctcttcccacacgctttccacccgtactgctcacagctttaagaaccagaacaaatgagtaatattagtgtcggttcatggctaaaaccagcactgatgtacatgaccacatatgtcaaatgctgcttctaggcatgacccgctcttactaatacctactcatcgctagaagaattttcggctgataaattttcaatttaagcaagagttatctgcgttggttcataactcaaactgatggccccaaccatattagtgcaaatttcacatatgatcataaccttttcatatgaaatcggatcgagatgaactttatataaacattgtagctgtcgatgatacctacaattttatagttcacaacctttttatttcaagtcatttaaatgcccaaataggtgtttcaaatctcagatagaaatgttcaaaagtaaaaaaggtccctatcataacataattgatatgtaagtgagttggaaaaagataagtacgtgtgagagagatcggggatcaaattctggtgtaataatgtatgtatttcagtcataaaaattggtagcagtagttggggctctgtatatataccggtaaggatgggatggtagtagaataattctttttttgtttttagttttttctggtccaaaatttcaaatttggatcccttacttgtaccaactaatattaatgagtgttgagggtagtagaggtgcaactttaccataatccctctgtttcaggttataagacgttttgactttaaatttgaccaagtttatgcgcaaatatagtaatatttataatactatattagtttcattaaataaataattgaatatattttcataataaatttgtgttgagttcaaaatattattaattttttctacaaacttggtcaaacttgaagcagtttgactttgaccaaagtcaaaacgtcttataacttgaaacggatggattactttttttgtggggacaagtttacaatgtttaataaagcacaatccatcttaatgttttcaagctgaatattgtaaaattcatggataaaccagcttctaaatgtttaaccgggaaaatgtcgaacgacaaattaatatttttaagtgatggggagtattaattaaggagtgacaactcaactttcaatatcgtactaaactgtgggatttattttctaaaattttataccctgccaattcacgtgttgtagatctttttttttcactaaccgacaccaggtatatcaattttattgaatatagcagcaaaaagaatgtgttgtacttgtaaacaaaaagcaaactgtacataaaaaaaaatgcactcctatataattaagctcataaagatgctttgcttcgtgagggcccaagttttgatgaccttttgcttgatctcgaaattaaaatttaagtactgttaagggagttcacaccaccatcaattttcagcctgaagaaacagttaaacaacgaccccgatgaccagtctactgctctccacatactagctgcattattgatcacaaaacaaaacaaaacgaaataaaaatcagcagcgagagtgtgcagagagagacaaaggtgatctggcgtggatatctccccatccatcctcacccgcgctgcccatcactcgccgccgcatactccatcatgtggagagaggaagacgaggaccacagccagagcccgggtcgagatgccaccacggccacaacccacgagcccggcgcgacaccaccgcgcgcgcgtgagccagccacaaacgcccgcggataggcgcgcgcacgccggccaatcctaccacatccccggcctccgcggctcgcgagcgccgctgccatccgatccgctgagttttggctatttatacgtaccgcgggagcctgtgtgcagagcagtgcatctcaagaagtactcgagcaaagaaggagagagcttggtgagctgcagccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagt

SEQ ID NO: 40 - DNA, 1281 bp, Hordeum vulgare
gcgcaggcggttgtgcaggcgatgcagtgccaggtgggggtgaggggcaggacggccgtcccggcgaggcagcccgcgggcagggtgtggggcgtcaggagggccgcccgcgccacctccgggttcaaggtgctggcgctcggcccggagaccaccggggtcatccagaggatgcagcagctgctcgacatggacaccacgcccttcaccgacaagatcatcgccgagtacatctgggttggaggatctggaattgacctcagaagcaaatcaaggacgatttcgaagccagtggaggacccgtcagagctgccgaaatggaactacgacggatcgagcacggggcaggctcctggggaagacagtgaagtcatcctatacccacaggccatattcaaggacccattccgaggaggcaacaacatactggttatctgtgacacctacacaccacagggggaacccatccctactaacaaacgccacatggctgcacaaatcttcagtgaccccaaggtcacttcacaagtgccatggttcggaatcgaacaggagtacactctgatgcagagggatgtgaactggcctcttggctggcctgttggagggtaccctggcccccagggtccatactactgcgccgtaggatcagacaagtcatttggccgtgacatatcagatgctcactacaaggcgtgcctttacgctggaattgaaatcagtggaacaaacggggaggtcatgcctggtcagtgggagtaccaggttggacccagcgttggtattgatgcaggagaccacatatgggcttccagatacattctcgagagaatcacggagcaagctggtgtggtgctcacccttgacccaaaaccaatccagggtgactggaacggagctggctgccacacaaactacagcacattgagcatgcgcgaggatggaggtttcgacgtgatcaagaaggcaatcctgaacctttcacttcgccatgacttgcacatagccgcatatggtgaaggaaacgagcggaggttgacagggctacacgagacagctagcatatcagacttctcatggggtgtggcgaaccgtggctgctctattcgtgtggggcgagacaccgaggcgaagggcaaaggatacctggaggaccgtcgcccggcctccaacatggacccgtacaccgtgacggcgctgctggccgagaccacgatcctgtgggagccgaccctcgaggcggaggccctcgctgccaagaagctggcgctgaaggtatga

SEQ ID NO: 41 - protein, 426 aa, Hordeum vulgare
AQAVVQAMQCQVGVRGRTAVPARQPAGRVWGVRRAARATSGFKVLALGPETTGVIQRMQQLLDMDTTPFTDKIIAEYIWVGGSGIDLRSKSRTISKPVEDPSELPKWNYDGSSTGQAPGEDSEVILYPQAIFKDPFRGGNNILVICDTYTPQGEPIPTNKRHMAAQIFSDPKVTSQVPWFGIEQEYTLMQRDVNWPLGWPVGGYPGPQGPYYCAVGSDKSFGRDISDAHYKACLYAGIEISGTNGEVMPGQWEYQVGPSVGIDAGDHIWASRYILERITEQAGVVLTLDPKPIQGDWNGAGCHTNYSTLSMREDGGFDVIKKAILNLSLRHDLHIAAYGEGNERRLTGLHETASISDFSWGVANRGCSIRVGRDTEAKGKGYLEDRRPASNMDPYTVTALLAETTILWEPTLEAEALAAKKLALKV

SEQ ID NO: 42 - DNA, 4273 bp, Artificial Sequence
  Synthetic expression cassette
ctgcagcaaagaaacgttattagttggtgcttttggtggtaggaatgtagttttctgacaaagtcaattactgaatataaaaaaaatctgcacagctctgcgtcaacagttgtccaagggatgcctcaaaaatctgtgcagattatcagtcgtcacgcagaagcagaacatcatggtgtgctaggtcagcttcttgcattgggccatgaatccggttggttgttaatctctcctctcttattctcttatattaagatgcataactcttttatgtagtctaaaaaaaaatccagtggatcggatagtagtacgtcatggtgccattaggtaccgttgaacctaacagatatttatgcatgtgtatatatatagctatatagacaaaattgatgccgattatagacccaaaagcaataggtatatataatataatacagaccacaccaccaaactaagaatcgatcaaatagacaaggcatgtctccaaattgtcttaaactatttccgtaggttcagccgttcaggagtcgaatcagcctctgccggcgttttctttgcacgtacgacggacacacatgggcataccatatagctggtccatgacattaggagagagaacgtacgtgttgacctgtagctgagatataacaaggttgattataatatcaccaaacatgaaatcatccaaggatgacccataactatcactactatagtactgcatctggtaaaagaaattgtatagactctatttcgagcactaccacataacgcctgcaatgtgacaccctacctattcactaatgtgcctcttcccacacgctttccacccgtactgctcacagctttaagaaccagaacaaatgagtaatattagtgtcggttcatggctaaaaccagcactgatgtacatgaccacatatgtcaaatgctgcttctaggcatgacccgctcttactaatacctactcatcgctagaagaattttcggctgataaattttcaatttaagcaagagttatctgcgttggttcataactcaaactgatggccccaaccatattagtgcaaatttcacatatgatcataaccttttcatatgaaatcggatcgagatgaactttatataaacattgtagctgtcgatgatacctacaattttatagttcacaacctttttatttcaagtcatttaaatgcccaaataggtgtttcaaatctcagatagaaatgttcaaaagtaaaaaaggtccctatcataacataattgatatgtaagtgagttggaaaaagataagtacgtgtgagagagatcggggatcaaattctggtgtaataatgtatgtatttcagtcataaaaattggtagcagtagttggggctctgtatatataccggtaaggatgggatggtagtagaataattctttttttgtttttagttttttctggtccaaaatttcaaatttggatcccttacttgtaccaactaatattaatgagtgttgagggtagtagaggtgcaactttaccataatccctctgtttcaggttataagacgttttgactttaaatttgaccaagtttatgcgcaaatatagtaatatttataatactatattagtttcattaaataaataattgaatatattttcataataaatttgtgttgagttcaaaatattattaattttttctacaaacttggtcaaacttgaagcagtttgactttgaccaaagtcaaaacgtcttataacttgaaacggatggattactttttttgtggggacaagtttacaatgtttaataaagcacaatccatcttaatgttttcaagctgaatattgtaaaattcatggataaaccagcttctaaatgtttaaccgggaaaatgtcgaacgacaaattaatatttttaagtgatggggagtattaattaaggagtgacaactcaactttcaatatcgtactaaactgtgggatttattttctaaaattttataccctgccaattcacgtgttgtagatctttttttttcactaaccgacaccaggtatatcaattttattgaatatagcagcaaaaagaatgtgttgtacttgtaaacaaaaagcaaactgtacataaaaaaaaatgcactcctatataattaagctcataaagatgctttgcttcgtgagggcccaagttttgatgaccttttgcttgatctcgaaattaaaatttaagtactgttaagggagttcacaccaccatcaattttcagcctgaagaaacagttaaacaacgaccccgatgaccagtctactgctctccacatactagctgcattattgatcacaaaacaaaacaaaacgaaataaaaatcagcagcgagagtgtgcagagagagacaaaggtgatctggcgtggatatctccccatccatcctcacccgcgctgcccatcactcgccgccgcatactccatcatgtggagagaggaagacgaggaccacagccagagcccgggtcgagatgccaccacggccacaacccacgagcccggcgcgacaccaccgcgcgcgcgtgagccagccacaaacgcccgcggataggcgcgcgcacgccggccaatcctaccacatccccggcctccgcggctcgcgagcgccgctgccatccgatccgctgagttttggctatttatacgtaccgcgggagcctgtgtgcagagcagtgcatctcaagaagtactcgagcaaagaaggagagagcttggtgagctgcagccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtgcgcaggcggttgtgcaggcgatgcagtgccaggtgggggtgaggggcaggacggccgtcccggcgaggcagcccgcgggcagggtgtggggcgtcaggagggccgcccgcgccacctccgggttcaaggtgctggcgctcggcccggagaccaccggggtcatccagaggatgcagcagctgctcgacatggacaccacgcccttcaccgacaagatcatcgccgagtacatctgggttggaggatctggaattgacctcagaagcaaatcaaggacgatttcgaagccagtggaggacccgtcagagctgccgaaatggaactacgacggatcgagcacggggcaggctcctggggaagacagtgaagtcatcctatacccacaggccatattcaaggacccattccgaggaggcaacaacatactggttatctgtgacacctacacaccacagggggaacccatccctactaacaaacgccacatggctgcacaaatcttcagtgaccccaaggtcacttcacaagtgccatggttcggaatcgaacaggagtacactctgatgcagagggatgtgaactggcctcttggctggcctgttggagggtaccctggcccccagggtccatactactgcgccgtaggatcagacaagtcatttggccgtgacatatcagatgctcactacaaggcgtgcctttacgctggaattgaaatcagtggaacaaacggggaggtcatgcctggtcagtgggagtaccaggttggacccagcgttggtattgatgcaggagaccacatatgggcttccagatacattctcgagagaatcacggagcaagctggtgtggtgctcacccttgacccaaaaccaatccagggtgactggaacggagctggctgccacacaaactacagcacattgagcatgcgcgaggatggaggtttcgacgtgatcaagaaggcaatcctgaacctttcacttcgccatgacttgcacatagccgcatatggtgaaggaaacgagcggaggttgacagggctacacgagacagctagcatatcagacttctcatggggtgtggcgaaccgtggctgctctattcgtgtggggcgagacaccgaggcgaagggcaaaggatacctggaggaccgtcgcccggcctccaacatggacccgtacaccgtgacggcgctgctggccgagaccacgatcctgtgggagccgaccctcgaggcggaggccctcgctgccaagaagctggcgctgaaggtatga

SEQ ID NO: 43 - protein, 436 aa, Artificial Sequence
  Translation product of SEQ ID NO 42 DNA
MVDLRNRRTSAQAVVQAMQCQVGVRGRTAVPARQPAGRVWGVRRAARATSGFKVLALGPETTGVIQRMQQLLDMDTTPFTDKIIAEYIWVGGSGIDLRSKSRTISKPVEDPSELPKWNYDGSSTGQAPGEDSEVILYPQAIFKDPFRGGNNILVICDTYTPQGEPIPTNKRHMAAQIFSDPKVTSQVPWFGIEQEYTLMQRDVNWPLGWPVGGYPGPQGPYYCAVGSDKSFGRDISDAHYKACLYAGIEISGTNGEVMPGQWEYQVGPSVGIDAGDHIWASRYILERITEQAGVVLTLDPKPIQGDWNGAGCHTNYSTLSMREDGGFDVIKKAILNLSLRHDLHIAAYGEGNERRLTGLHETASISDFSWGVANRGCSIRVGRDTEAKGKGYLEDRRPASNMDPYTVTALLAETTILWEPTLEAEALAAKKLALKV

SEQ ID NO: 44 - DNA, 1992 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 45 - DNA, 1248 bp, Hordeum vulgare
atggcatccgcccccgcctccgcctccgcggccctctccaccgccgcccccgccgacaacggggccgccaagcccacggagcagcggccggtacaggtggctaagcgattggagaagttcaaaacaacaattttcacacagatgagcatgctcgcagtgaagcatggagcaataaaccttggacaggggtttcccaattttgatggccctgactttgtcaaagatgctgctattgaggctatcaaagctggaaagaatcagtatgcaagaggatatggtgtgcctgaattgaactcagctgttgctgagagatttctcaaggacagtggattgcacatcgatcctgataaggaagttactgttacatctgggtgcacagaagcaatagctgcaacgatattgggtctgatcaaccctggggatgaagtcatactgtttgctccattctatgattcttatgaggctacactgtccatggctggtgcgaatgtcaaagccattacactccgccctccggactttgcagtccctcttgaagagctaaaggctgcagtctcgaagaataccagagcaataatgattaatacacctcacaaccctaccgggaaaatgttcacaagggaggaacttgagttcattgctgatctctgcaaggaaaatgacgtgttgctctttgccgatgaggtctacgacaagctggcgtttgaggcggatcacatatcaatggcttctattcctggcatgtatgagaggaccgtcactatgaactccctggggaagacgttctccttgaccggatggaagatcggctgggcgatagcaccaccgcacctgacatggggcgtaaggcaggcacactccttcctcacattcgccacctccacgccgatgcaatcagcagcggcggcggccctgagagcaccggacagctactttgaggagctgaagagggactacggcgcaaagaaagcgctgctggtggacgggctcaaggcggcgggcttcatcgtctacccttcgagcggaacctacttcatcatggtcgaccacaccccgttcgggttcgacaacgacgtcgagttctgcgagtacttgatccgcgaggtcggcgtcgtggccatcccgccaagcgtgttctacctgaacccggaggacgggaagaacctggtgaggttcaccttctgcaaggacgacgacacgctaagggcggcggtggacaggatgaaggccaagctcaggaagaaatga

SEQ ID NO: 46 - protein, 415 aa, Hordeum vulgare
MASAPASASAALSTAAPADNGAAKPTEQRPVQVAKRLEKFKTTIFTQMSMLAVKHGAINLGQGFPNFDGPDFVKDAAIEAIKAGKNQYARGYGVPELNSAVAERFLKDSGLHIDPDKEVTVTSGCTEAIAATILGLINPGDEVILFAPFYDSYEATLSMAGANVKAITLRPPDFAVPLEELKAAVSKNTRAIMINTPHNPTGKMFTREELEFIADLCKENDVLLFADEVYDKLAFEADHISMASIPGMYERTVTMNSLGKTFSLTGWKIGWAIAPPHLTWGVRQAHSFLTFATSTPMQSAAAAALRAPDSYFEELKRDYGAKKALLVDGLKAAGFIVYPSSGTYFIMVDHTPFGFDNDVEFCEYLIREVGVVAIPPSVFYLNPEDGKNLVRFTFCKDDDTLRAAVDRMKAKLRKK

SEQ ID NO: 47 - DNA, 2735 bp, Artificial Sequence
  Synthetic expression cassette
gtttgaatcctccttaaagtttttctctggagaaactgtagtaattttactttgttgtgttcccttcatcttttgaattaatggcatttgttttaatactaatctgcttctgaaacttgtaatgtatgtatatcagtttcttataatttatccaagtaatatcttccattctctatgcaattgcctgcataagctcgacaaaagagtacatcaacccctcctcctctggactactctagctaaacttgaatttccccttaagattatgaaattgatatatccttaacaaacgactccttctgttggaaaatgtagtacttgtctttcttcttttgggtatatatagtttatatacaccatactatgtacaacatccaagtagagtgaaatggatacatgtacaagacttatttgattgattgatgacttgagttgccttaggagtaacaaattcttaggtcaataaatcgttgatttgaaattaatctctctgtcttagacagataggaattatgacttccaatggtccagaaagcaaagttcgcactgagggtatacttggaattgagacttgcacaggtccagaaaccaaagttcccatcgagctctaaaatcacatctttggaatgaaattcaattagagataagttgcttcatagcataggtaaaatggaagatgtgaagtaacctgcaataatcagtgaaatgacattaatacactaaatacttcatatgtaattatcctttccaggttaacaatactctataaagtaagaattatcagaaatgggctcatcaaacttttgtactatgtatttcatataaggaagtataactatacataagtgtatacacaactttattcctattttgtaaaggtggagagactgttttcgatggatctaaagcaatatgtctataaaatgcattgatataataattatctgagaaaatccagaattggcgttggattatttcagccaaatagaagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatgtctctgctctcagatctcgttaacctcaacctcaccgatgccaccgggaaaatcatcgccgaatacatatggatcggtggatctggaatggatatcagaagcaaagccaggacactaccaggaccagtgactgatccatcaaagcttcccaagtggaactacgacggatccagcaccggtcaggctgctggagaagacagtgaagtcattctataccctcaggcaatattcaaggatcccttcaggaaaggcaacaacatcctggtgatgtgtgatgcttacacaccagctggtgatcctattccaaccaacaagaggcacaacgctgctaagatcttcagccaccccgacgttgccaaggaggagccttggtatgggattgagcaagaatacactttgatgcaaaaggatgtgaactggccaattggttggcctgttggtggctaccctggccctcagggaccttactactgtggtgtgggagctgacaaagccattggtcgtgacattgtggatgctcactacaaggcctgtctttacgccggtattggtatttctggtatcaatggagaagtcatgccaggccagtgggagttccaagtcggccctgttgagggtattagttctggtgatcaagtctgggttgctcgataccttctcgagaggatcactgagatctctggtgtaattgtcagcttcgacccgaaaccagtcccgggtgactggaatggagctggagctcactgcaactacagcactaagacaatgagaaacgatggaggattagaagtgatcaagaaagcgatagggaagcttcagctgaaacacaaagaacacattgctgcttacggtgaaggaaacgagcgtcgtctcactggaaagcacgaaaccgcagacatcaacacattctcttggggagtcgcgaaccgtggagcgtcagtgagagtgggacgtgacacagagaaggaaggtaaagggtacttcgaagacagaaggccagcttctaacatggatccttacgttgtcacctccatgatcgctgagacgaccatactcggttga

SEQ ID NO: 48 - DNA, 1371 bp, Citrus reticulata
atgcttaagccgtccgccttcgggtcttctttttcttcctcagctctgctttcgttttcgaagcatttgcatacaataagcattactgattctgtcaacaccagaagaagaggaatcagtaccgcttgccctaggtacccttctctcatggcgagcttgtccaccgtttccaccaatcaaagcgacaccatccagaagaccaatcttcagcctcaacaggttgctaagtgcttggagaagtttaaaactacaatctttacacaaatgagtatgcttgccatcaaacatggagctataaatcttggtcaaggctttcccaactttgatggcccagattttgttaaagatgcagcgattcaagccataagggatgggaagaatcaatatgctcgtggacatggggttccagagttcaactctgccattgcttcccggtttaagaaagattctgggctcgaggttgaccctgaaaaggaagttactgttacctctgggtgcaccgaagccattgctgcaaccatcttaggtttgattaatcctggagatgaggtgatcctttttgcacctttctatgattcctatgaagctactctctccatggctggtgctaaaattaaatgcatcacattgcgccctccagaatttgccatccccattgaagagctcaagtctacaatctcaaaaaatactcgtgcaattcttatgaacactccacataaccccactggaaagatgttcactagggaggaacttaatgttattgcatctctttgcattgagaatgatgtgttggtttttagtgatgaggtctatgataagttggcttttgaaatggatcacatttccatagcctctcttcctggaatgtatgagcgtactgtaaccatgaattccttagggaagacattctctttaacagggtggaagatcgggtgggcaatagctccaccgcaccttacatggggggtgcggcaggcacactcttttctcacgtttgccacatccactccaatgcagtgggcagctacagcagcccttagagctccggagacgtactatgaggagctaaagagagattactcggcaaagaaggcaattttggtggagggattgaatgctgttggtttcaaggtattcccatctagtgggacatactttgtggttgtagatcacaccccatttgggcacgaaactgatattgcattttgtgaatatctgatcaaggaagttggggttgtggcaattccgaccagcgtattttacttgaatccagaggatggaaagaatttggtgagatttaccttctgcaaagatgaaggaactttgaggtctgcagttgacaggatgaaggagaagctgaggagaaaatga

SEQ ID NO: 49 - protein, 456 aa, Citrus reticulata
MLKPSAFGSSFSSSALLSFSKHLHTISITDSVNTRRRGISTACPRYPSLMASLSTVSTNQSDTIQKTNLQPQQVAKCLEKFKTTIFTQMSMLAIKHGAINLGQGFPNFDGPDFVKDAAIQAIRDGKNQYARGHGVPEFNSAIASRFKKDSGLEVDPEKEVTVTSGCTEAIAATILGLINPGDEVILFAPFYDSYEATLSMAGAKIKCITLRPPEFAIPIEELKSTISKNTRAILMNTPHNPTGKMFTREELNVIASLCIENDVLVFSDEVYDKLAFEMDHISIASLPGMYERTVTMNSLGKTFSLTGWKIGWAIAPPHLTWGVRQAHSFLTFATSTPMQWAATAALRAPETYYEELKRDYSAKKAILVEGLNAVGFKVFPSSGTYFVVVDHTPFGHETDIAFCEYLIKEVGVVAIPTSVFYLNPEDGKNLVRFTFCKDEGTLRSAVDRMKEKLRRK

SEQ ID NO: 50 - DNA, 37 bp, Artificial Sequence
  Synthetic primer sequence
ggccacatgtccgttgctaagtgcttggagaagttta

SEQ ID NO: 51 - DNA, 40 bp, Artificial Sequence
  Synthetic primer sequence
cgggcacgtgtcattttctcctcagcttctccttcatcct

SEQ ID NO: 52 - protein, 457 aa, Zea mays
MNLAAFSSTLATLPWYEMPSINSSATFSSSLLRRSLCASLRTISHMASAAAPTSAPVATTENGAAKAIEQRPVQVAERLEKFKTTIFTQMSMLAIKHGAINLGQGFPNFDGPDFVKEAAIQAINAGKNQYARGFGVPELNSAIAERFLKDSGLQVDPDKEVTVTSGCTEAIAATILGLINPGDEVILFAPFYDSYEATLSMAGANVKAITLRAPDFAVPLEELEAAVSKDTKAIMINTPHNPTGKMFTREELESIAALCKENDVLLFSDEVYDKLVFEADHISMASIPGMYERTVTMNSLGKTFSLTGWKIGWAIAPPHLTWGLRQAHSFLTFATCTPMQAAAAAALRAPDSYYDELKRDYSAKKAILLEGLEAAGFIVYPSSGTYYIMVDHTPFGFDSDVEFCEYLIREVGVCAIPPSVFYLDPEEGKKLVRFTFSKDEGTLRAAVERLKAKLRRK

SEQ ID NO: 53 - protein, 424 aa, Gossypium hirsutum
MQAAECTWTHFEMLRPLCFKSPSTTPLFFNFSKHFQKGFSDSSFFRSNRRISNYPSFMATISSLSTHKDPVSTHDATPNITHQPVQVAKRLEKFKTTIFTQMSMLAIKHGAINLGQGFPNFDGPDFVKGAAIQAIKDGKNQYARGYGVPDFNNAIAARFKKDTGLVIDPEKEVTVTSGCTEAIAATMLGLINPGDEVILFAPFYDSYEATLSMAGAKVKCITLCPPDFAVPIDELKSTISKNTRAILINTPHNPTGKMFTREELNTIASLCIENDVLVFTDEVYDKLAFEMDHISMASLPGMYERTVTMNSLGKTFSLTGWKIGWAIAPPHLTWGVRQAHSFLTFATSTPMQYAATVALQAPDSYFAELKRDYMAKKAILVQGLKDVGFKVFPSSGTYFVVVDHTPFGLENDIAFCEYLIKEVG

SEQ ID NO: 54 - protein, 462 aa, Ricinus communis
MQSQCTWTGTRMPLPIILKPSTFSILKHLPTKRTNLFSTRSPISNYPSLMATFSTASTTEKDAPSGQNDSTQKSQQPLQVAKRLEKFKTTIFTQMSSLAIKHGAINLGQGFPNFDGPEFVKEAAIQAIRDGKNQYARGYGVPDFNSAIVDRFKKDTGLVVDPEKEVTVTSGCTEAIAATILGLIDPGDEVILFAPFYDSYEATLSMAGAKIKCVTLQPPDFAVPIDELKSIISKNTRAILINTPHNPTGKMFTREELTTIASCCIENDVLVFTDEVYDKLAFEMDHISMASLPGMYERTVTLNSLGKTFSLTGWKIGWAIAPPHLTWGVRQAHAFLTFATSTPMQWAASVALRAPDSYFEELKRDYMAKKAILVEGLKAVGFKVFPSSGTYFVVVDHTPFGLENDIAFCEHLIKEVGVVAIPTSVFYLNPEEGKNLVRFTFCKDEGTLRTAVERMKEKLKRK

SEQ ID NO: 55 - protein, 409 aa, Populus trichocarpa
MASSPSLKDAVSTQNESTQKTQQPLQVAKRLEKFKTTIFTQMSSLAIKHGAINLGQGFPNFDGPEFVKEAAIQAIKDGKNQYARGYGVPDFSSAIAERFKKDTGLVVDPEKEITVTSGCTEAIAATMLGLINPGDEVILFAPFYDSYEATLSMAGAKIKCITLHPPDFAVPIDELKSAITQDTRAVLINTPHNPTGKMFSREELSTIASLCIENDVLVFTDEVYDKLAFELDHISMASLPGMYERTVTLNSLGKTFSLTGWKIGWAIAPPHLTWGVRQAHSFLTFATSTPMQWAAAVALRAPESYFVELKRDYMAKKEILVEGLKAVGFKVFPSSGTYFVVVDHTPFGLENDIAFCEYLIKEVGVVAIPTSVFYLNPEDGKNLVRFTFCKDEGTLRAAVDRMKEKLKRK

SEQ ID NO: 56 - protein, 439 aa, Glycine max
MKFTPSSKFLGFSNHFHSLLAPSFSPTPKFSSSFSATMSTLSTQNDTVTHKTQQPLQIAKRLEKFQTTIFTQMSLLAIKHGAINLGQGFPNFDGPEFVKEAAIQAIRDGKNQYARGYGVPDLNIAIAERFKKDTGLVVDPEKEITVTSGCTEAIAATMIGLINPGDEVIMFAPFYDSYEATLSMAGAKVKGITLRPPDFAVPLEELKSTISKNTRAILINTPHNPTGKMFTREELNCIASLCIENDVLVFTDEVYDKLAFDMEHISMASLPGMFERTVTLNSLGKTFSLTGWKIGWAIAPPHLSWGVRQAHAFLTFATAHPFQCAAAAALRAPDSYYVELKRDYMAKRAILIEGLKAVGFKVFPSSGTYFVVVDHTPFGLENDVAFCEYLVKEVGVVAIPTSVFYLNPEEGKNLVRFTFCKDEETIRSAVERMKAKLRK

SEQ ID NO: 57 - protein, 418 aa, Physcomitrella patens
MASLSLSINGVAQESAMPASQNSDPPRVQVAKRLEQFKTTIFTEISILASKHNAINLGQGFPNFDGPEFVKNAAIEAIRDGGKNQYARGFGVPQLNAAIAESFNKESGIVVDPETHVTVTSGCTEAIAATVLGLVNPGDEIIVFEPFYDSYQATVSMSGAILKTVTMRAPEFAVPEEELRAAFSSKTRAILVNTPHNPTGKVFPRHELELIASLCKEHNTLAFCDEVYNKLVFKGEHVSLASLDGMYERTVTMNSLGKTFSLTGWKIGWAVAPPHLTRGIRLAHSYLTFATATPLQWASVEALRAPDSFYAELIKSYSAKKDILVEGLNSVGFEVYEPEGTYFVMVDHTPFGFENDVAFCKYLIEEVGIAAIPPSVFYTNPEDGKNLVRFAFCKDEETLKTAVERLRTKLKKAVSLSS

SEQ ID NO: 58 - protein, 407 aa, Chlamydomonas sp.
MAPPEAGATAAAEPSKPLNELFSSLPTTIFEVMSKLAMEHASVNLGQGFPDAEGPEAMKQIASASMYDFHNQYPSLEGVPELRQAVAAHSEREQGILVDWATETLITVGATEGLASAFLGLINPGDEVIMFDPMYDSYTSMAKRSGAVIVPVRLRLPDFSVPLEELAAAVTPRTKMIMINTPHNPSGKVFTRPELEAIAELCVRHDLIALSDEVYEHLVFGGAAHVSLRSLPGMKERCVRLGSAGKTFSFTAWKVGWMTGPARLLNPIVKAHQFLVFTVPSSLQRAVAHGLDKEADFYHSLGPSLEAKRRYLEAELTALGFDCLPAHGAYFLVADFQRPGEDDADFAKRLTAEGGVTTIPISGFYVGPRPPTHLVRFCYCKEDIKLQAAVERLKAYVGPGGKGAPQV

SEQ ID NO: 59 - protein, 279 aa, Artificial Sequence
  Consensus Sequence
VAKRLEKFKTTIFTQMSLAKHGAINLGQGFPNFDGPFVKAAIAIGNQYARGGVPNAARFKDGLVDPKETVTSGCTEAIAATLGLINPGDEVIFAPFYDSYEATISMAGAKTLPDFVPELTRAINTPHNPTGKMFTREELIALCENDVLFDEVYDKLFHISASPGMERTVTNSLGKTFSLTGWKIGWAAPPHLTWGRQAHFLTFATPQAAALAPSYELRDYAKLGLGFVPSSGTYFVDHTPFGDFCEYLEVGVAIPSVFYPEGKLVRFFCKDTLAVRKKL

SEQ ID NO: 60 - protein, 319 aa, Artificial Sequence
  Consensus Sequence
QVAKRLEKFKTTIFTQMSLAIKHGAINLGQGFPNFDGPFVKEAAIQAIGKNQYARGYGVPNAIARFKDGLVDPEKETVTSGCTEAIAATLGLINPGDEVILFAPFYDSYEATLSMAGAKITLPPDFAVPELKSKTRAIINTPHNPTGKMFTREELIALCENDVLFDEVYDKLAFEDHISMASPGMYERTVTNSLGKTFSLTGWKIGWAIAPPHLTWGVRQAHFLTFATTPMQAAALRAPDSYELKRDYAKKLVGLKGFVPSSGTYFVVDHTPFGNDFCEYLIEVGVVAIPSVFYLPEGKNLVRFTFCKDTLRAVRMKKL